SEQUENCE LISTING (10 sequences)

SEQ ID NO: 1 - protein, 30 aa, synthetic construct
  REGION 1..30: note = Synthetic
GTTTGGAGAGGGGTTTTGAAGAATGTGTAG

SEQ ID NO: 2 - protein, 28 aa, synthetic construct
  REGION 1..28: note = Synthetic
ATCCCACCCACTAACCTTAACCTCTAAC

SEQ ID NO: 3 - protein, 25 aa, synthetic construct
  REGION 1..25: note = Synthetic
TGTTTGGAGAATTTTATGGATAGAG

SEQ ID NO: 4 - protein, 25 aa, synthetic construct
  REGION 1..25: note = Synthetic
CCAATTAAATCATCAAACCTAACTC

SEQ ID NO: 5 - protein, 30 aa, synthetic construct
  REGION 1..30: note = Synthetic
TAGGTGGTTATAGGAGATGTATTTTTGATT

SEQ ID NO: 6 - protein, 30 aa, synthetic construct
  REGION 1..30: note = Synthetic
TTATAAATAAAACTCAACCATACTTAACCC

SEQ ID NO: 7 - protein, 20 aa, synthetic construct
  REGION 1..20: note = Synthetic
TGTGGGCAAAGAGGCTTCAT

SEQ ID NO: 8 - protein, 20 aa, synthetic construct
  REGION 1..20: note = Synthetic
CAAAGCTGACTTAGCCTCAG

SEQ ID NO: 9 - protein, 26 aa, synthetic construct
  REGION 1..26: note = Synthetic
GATTGTTGATCCCACAGAAGGCAATC

SEQ ID NO: 10 - protein, 26 aa, synthetic construct
  REGION 1..26: note = Synthetic
GAACTTTCAAGCAGCTGAGGCATTTA

(19) United States
(12) Patent Application Publication
(10) Pub. No.: US 2026/0337553 A1
(43) Pub. Date: Oct. 29, 2026

(54) COMPOSITIONS AND METHODS FOR GENERATION OF BOVINE PLURIPOTENT EMBRYONIC STEM CELLS

(71) Applicant: University of Connecticut, Farmington, CT (US)

(72) Inventors: Young TANG, Willington, CT (US); Yue Su, Willington, CT (US); Xiuchun Tian, Storrs, CT (US); Ruifeng Zhao, Willington, CT (US)

(21) Appl. No.: 19/486,421

(22) PCT Filed: May 24, 2024
(86) PCT No.: PCT/US2024/030927

§ 371 Date: Nov. 21, 2025

Related U.S. Application Data
(60) Provisional application No. 63/468,697, filed on May 24, 2023.

(51) Int. Cl. C12N 5/0735

(52) U.S. Cl. CPC C12N 5/0606; C12N 2501/01; C12N 2501/115; C12N 2501/235; C12N 2501/415; C12N 2501/727

(57) ABSTRACT

The present disclosure relates to compositions and methods for growing a cell line which can be used to generate bovine pluripotent embryonic stem cells. The composition comprises a cell culture medium and at least one supplement, wherein the composition maintains pluripotency and self-renewal capacity of the cell/s. The methods employ the compositions to produce bovine embryonic stem cells which maintain their pluripotency and self-renewing capacity

### RELATED APPLICATION INFORMATION

This application claims priority to U.S. Patent Application No. 63/468,697, filed on May 24, 2023, the contents of which are herein incorporated by reference.

### GOVERNMENT SUPPORT

This invention was made with government support under 58-8042-0-028 awarded by the Agricultural Research Service, and 2019-67015-29413 awarded by the National Institute of Food and Agriculture. The government has certain rights in the invention.

### SEQUENCE LISTING STATEMENT

The contents of the electronic sequence listing titled UCONN-43084-601.xml (Size: 13,431 bytes; and Date of Creation: May 20, 2024) is herein incorporated by reference in its entirety.

### FIELD OF THE DISCLOSURE

Described herein are bovine pluripotent embryonic stem cells, the composition (also called as TiF2 and TiF3 medium), and methods for the generation of the bovine pluripotent stem cells, the unique medium allows bovine pluripotent stem cells to maintain their pluripotent and self-renewal properties, and uses thereof. More particularly, the invention relates to the generation of bovine pluripotent stem cells from bovine pre-implantation embryos produced in vitro or in vivo.

### BACKGROUND

The studies and applications of embryonic stem cells (ESCs), derived from the inner cell mass (ICM) of the blastocysts, have advanced dramatically since their first establishment in mice and humans. Due to their origins, unlimited self-renewing capacity and pluripotent differentiation potentials, ESCs can be used to determine the mechanisms of pluripotency maintenance, create disease models for causation investigation and drug screening, understand early embryogenesis, differentiation and development, and create interspecies chimeras for research and potential xenotransplantation applications. As the economically most important domesticated ungulates, bovine provides up to 45% of the global animal protein supply. Generating ESCs from the bovine would not only contribute to the development of new agricultural biotechnology such as cultivated meat, improved production efficiency, quality and safety of animal products, disease-resistance through molecular breeding and genome editing, but also permit the development of stem cell- and animal-based models to study the pathogenesis and treatment to important zoonotic diseases such as bovine spongiform encephalopathy, bovine tuberculosis, and Toxoplasma gondii infection. The bovine ESCs are also good biomedical models for human regenerative medicine and tissue/organ replacement therapy.

While much effort has been devoted to deriving bovine ESCs (bESCs), most of the cells showed irregular colony morphology, limited pluripotent genes expression, failure in long-term culture, and lack of in vivo differentiation and chimeric animal testing. The primed-state bESCs also exhibited undefined colony morphology and lacked chimeric capacity determination, while bovine expanded potential stem cells (bEPSCs) only showed low efficiency and minor contribution in interspecies chimera with mice. Furthermore, no direct germline contribution and in vitro PGC induction has been reported for any reported bovine stem cells. Therefore, reliable methods to generate bESCs and the bonafide bESCs are highly desired.

### SUMMARY

In one embodiment, the present disclosure relates to a composition to grow a cell line. In some embodiments, the composition comprises:
i. a cell culture medium; and
ii. a supplement comprising:
a) a Wingless/Integrated (WNT) inhibitor;
b) a histone methyltransferase DOT1L inhibitor (iDOT1L);
c) an adenyl cyclase activator;
d) a Mitogen-activated protein kinase kinases 1 and 2 (MEK1/2) inhibitor;
e) a fibroblast growth factor receptor (FGFR) inhibitor;
f) a glycogen synthase kinase (GSK-3) inhibitor; and
g) a leukemia inhibitory factor (LIF),
wherein the composition maintains pluripotency and self-renew capacity of the cell line.

In some embodiments, the supplement further comprises: bovine serum albumin (BSA); a basic fibroblast growth factor (bFGF); and a transforming growth factor β1 (TGF β1).

In some embodiments, the culture medium used in the composition is a serum free culture medium for culturing stem cell lines. In other embodiments, the culture medium used in the composition is mTeSR-plus or N2B27.

In some embodiments, when the culture medium is N2B27, the supplement further comprises:
bovine serum albumin (BSA); a basic fibroblast growth factor (bFGF); and a transforming growth factor β1 (TGF β1).

In still other embodiments, the composition comprises at least 99% of the cell culture medium and about 1% of the supplement.

In yet still other embodiments, the supplement in the composition comprises:
a) from about 0.1 μM to about 25 μM of the WNT inhibitor;
b) from about 0.1 μM to about 25 μM of the histone methyltransferase DOT1L inhibitor;
c) from about 0.1 μM to about 10 μM of the MEK1/2 inhibitor;
d) about 0.1 μM to about 10 μM of the FGFR inhibitor;
e) from about 0.5 μM to about 10 μM of the GSK-3 inhibitor;
f) from about 1 μM to about 50 μM of the adenyl cyclase activator; and
g) about 100 U/ml to about 5000 U/ml of LIF.

In some embodiments, when the supplement also comprises BSA, bFGF, and TGF β1, the supplement in the composition comprises: from about 0.01 to about 5% (v/v) of BSA; from about 10 to about 500 ng/ml of bFGF; and from about 0.1 to about 5 ng/ml of TGF β1.

In still further aspects, in the supplement:
a) the WNT inhibitor is IWR1;
b) the histone methyltransferase DOT1L inhibitor is EPZ004777;
c) the adenyl cyclase activator is forskolin;
d) the MEK1/2 inhibitor is PD184352;
e) the FGFR inhibitor is SU5402;
f) the GSK-3 inhibitor is CHIR99021; and
g) the LIF is a human LIF or a bovine LIF.

In some embodiments, when the supplement also comprises BSA, bFGF, and TGF β1: the bFGF is a human bFGF or a bovine bFGF; and the TGF β1 is a human TGF β1 or a bovine TGF β1.

In yet further embodiments, the IWR1 is present in an amount of about 2.5 μM; the EPZ004777 is present in an amount of about 3.3 μM; the forskolin is present in an amount of about 10 μM; the PD184352 is present in an amount of about 0.8 μM; the SU5402 is present in an amount of about 2 μM; the CHIR99021 is present in an amount of about 3 μM; and the human LIF or bovine LIF is present in an amount of 1000 U/mL. In still yet further embodiments, when BSA, bFGF and TGF β1 are present in the supplement, the BSA is present in an amount of about 1% (v/v); the human bFGF or bovine bFGF is present in an amount of about 100 ng/mL; and the human TGF β1 or bovine TGF β1 is present in an amount of about 1.74 ng/mL.

In yet another embodiment, the present disclosure relates to a method of producing stem cells. The method comprises the steps of:
a) providing bovine pre-implantation embryos;
b) treating the bovine pre-implantation embryos with an enzyme to remove the zone pellucida;
c) culturing treated bovine pre-implantation embryos with a composition to form a culture, the composition comprising:
i) a cell culture medium; and
ii) a supplement comprising:
a) a Wingless/Integrated (WNT) inhibitor;
b) a histone methyltransferase DOT1L inhibitor (iDOT1L);
c) an adenyl cyclase activator;
d) a Mitogen-activated protein kinase kinases 1 and 2 (MEK1/2) inhibitor;
e) a fibroblast growth factor receptor (FGFR) inhibitor;
f) a glycogen synthase kinase (GSK-3) inhibitor; and
g) a leukemia inhibitory factor (LIF); and
d) harvesting, collecting, or isolating stem cells resulting from the culture.

In some embodiments of the above method, the enzyme is a pronase, a hyaluronidase, an acrosin or any combinations thereof.

In some embodiments of the above method, the supplement further comprises: bovine serum albumin (BSA); a basic fibroblast growth factor (bFGF); and a transforming growth factor β1 (TGF β1).

In some embodiments of the above method, the culture medium used in the composition is a serum free culture medium for culturing stem cell lines. In other embodiments, the culture medium used in the composition is mTeSR-plus or N2B27.

In some embodiments of the above method, when the culture medium is N2B27, the supplement further comprises: bovine serum albumin (BSA); a basic fibroblast growth factor (bFGF); and a transforming growth factor β1 (TGF β1).

In still other embodiments of the above method, the composition comprises at least 99% of the cell culture medium and about 1% of the supplement.

In yet still other embodiments of the above method, the supplement in the composition comprises:
a) from about 0.1 μM to about 25 μM of the WNT inhibitor;
b) from about 0.1 μM to about 25 μM of the histone methyltransferase DOT1L inhibitor;
c) from about 0.1 μM to about 10 μM of the MEK1/2 inhibitor;
d) about 0.1 μM to about 10 μM of the FGFR inhibitor;
e) from about 0.5 μM to about 10 μM of the GSK-3 inhibitor;
f) from about 1 μM to about 50 μM of the adenyl cyclase activator; and
g) about 100 U/ml to about 5000 U/ml of LIF.

In some embodiments of the above method, when the supplement also comprises BSA, bFGF, and TGF β1, the supplement in the composition comprises: from about 0.01 to about 5% (v/v) of BSA; from about 10 to about 500 ng/ml of bFGF; and from about 0.1 to about 5 ng/ml of TGF β1.

In still further aspects of the above method, in the supplement:
a) the WNT inhibitor is IWR1;
b) the histone methyltransferase DOT1L inhibitor is EPZ004777;
c) the adenyl cyclase activator is forskolin;
d) the MEK1/2 inhibitor is PD184352;
e) the FGFR inhibitor is SU5402;
f) the GSK-3 inhibitor is CHIR99021; and
g) the LIF is a human LIF or a bovine LIF.

In some embodiments of the above method, when the supplement also comprises BSA, bFGF, and TGF β1: the bFGF is a human bFGF or a bovine bFGF; and the TGF β1 is a human TGF β1 or a bovine TGF β1.

In yet further embodiments of the above method, the IWR1 is present in an amount of about 2.5 μM; the EPZ004777 is present in an amount of about 3.3 μM; the forskolin is present in an amount of about 10 μM; the PD184352 is present in an amount of about 0.8 μM; the SU5402 is present in an amount of about 2 μM; the CHIR99021 is present in an amount of about 3 μM; and the human LIF or bovine LIF is present in an amount of 1000 U/mL. In still yet further embodiments of the above method, when BSA, bFGF and TGF β1 are present in the supplement, the BSA is present in an amount of about 1% (v/v); the human bFGF or bovine bFGF is present in an amount of about 100 ng/mL; and the human TGF β1 or bovine TGF β1 is present in an amount of about 1.74 ng/mL.

In still other embodiments, also disclosed is a stem cell line comprising a bovine embryonic stem cell (bESC), wherein the bESC maintains its pluripotency and self-renewal capacity.

These and other aspects of the present invention are described in more detail below.

### BRIEF DESCRIPTION OF THE DRAWINGS

The accompanying drawings are included to provide a further understanding of the methods, compositions of the disclosure, and are incorporated in and constitute a part of this specification. The drawings illustrate one or more embodiment(s) of the disclosure, and together with the description serve to explain the principles and operation of the disclosure.

FIG. 1: Derivation and characterization of bESCs in TiF2 medium. (A) Derivation of JY-bESCs cultured on feeder layers in TiF2 medium. (B) Colony morphology of JY-bESCs in TiF2 medium. (C) SSEA4 live staining of emerged colony from passaged bovine blastocyst (P2) in TiF2 medium. (D) Proliferation rate of JY-bESCs. Mean±sd, n=3. (E) Karyotypes of JY-bESC-1 and JY-bESC-4 at passages 63 and 64, respectively. (F) Relative pluripotent genes expression of bMSC and JY-bESCs at early (P8 to P12) and late (P42 to P50) passages. Mean±sd, n=3. (G) Representative immunostaining images of JY-bESCs in TiF2 medium for OCT4, NANOG, SOX2, SSEA3, SSEA4, TRA-1-60. (H) AP staining on JY-bESC-1 at passage 12. (I) Bisulfite sequencing analysis of the bovine OCT4 distal enhancer (left 2 panels), proximal promoter (middle 2 panels) and NANOG promoter (right 2 panels) regions. Open and closed circles represent unmethylated and methylated CpGs, respectively. The percentage of methylated CpG is shown above each sample. (J) Immunostaining of passaged EBs derived from JY-bESCs for markers of the three germ layers (GATA4 for endoderm, SMA for mesoderm, and TUJ1 for ectoderm). (K) H&E staining of teratoma derived from JY-bESCs revealed representative tissues from the ectoderm (glial cells), mesoderm (cartilage), and endoderm (epithelial tissue with acinar complex).

FIG. 2: Comparative Transcriptomic and X Chromosome Inactivation (Xi) Analyses of PSCs. (A) PCA comparison of JY-bESCs to previously published bovine embryos (ICM, APE, ERSE [GSE53387] and blastocyst [BL; GSE52415 and GSE110036]) and pluripotent stem cells (bpESC [GSE110036], bEPSC [GSE129760] and biPSC [GSE169624]). (B) Heatmap comparison of JY-bESC, biPSC_Pillai, and bpESC_Bogliotti. Key genes related to pluripotency are listed on the left. Significantly enriched Gene Ontology Biological Process (GO:BP) terms are displayed on the right. (C) Dot plots of marker genes representing different pluripotency states in four bovine PSCs: JY-bESC, bEPSC_Zhao, biPSC_Pillai, and bpESC_Bogliotti. (D) PCA plot of RNA-seq data from bovine, human, and mouse embryos and PSCs. Dot colors represent different cell types of various pluripotency states, while dot shapes indicate species. The gray arrows suggest changes in pluripotency within species. (E) PCA projection of JY-bESCs' pluripotency onto that of human PSCs. (F) 3D PCA plot of bovine, human, and mouse embryos and PSCs. Species differences are revealed in PC3. (G) XIST and HUWE1 RNA FISH signals and (H) representative H3K27me3 immunostaining images in female JY-bESCs before (top) and after (bottom) 6 additional passages. (I) Positive ratios of H3K27me3 and XIST in colonies established from single cells before and after an additional 6 passages. No significant difference (p>0.05) was observed before and after the additional passages. The data were analyzed by Student's t-test, n=5. (J) X:A median expression ratios and 95% confidence intervals of four JY-bESC lines. The lower line indicates failure of dosage compensation. (K) X:A allelic ratios of female JY-bESC-8 line.

FIG. 3: Direct in vitro Differentiation of bESCs to Neural Progenitor Cells (NPCs) and Primordial Germ Cell-Like Cells (PGCLCs). (A) The morphology of NPC 19 days after induction. (B) Representative immunostaining images of JY-bESCs derived NPC (upper row) and JY-bESCs (lower row) for SOX2, NESTIN, and PAX6. (C) Relative expression of NPC-related genes by JY-bESCs and the induced NPCs at D19. Mean±sd, n=2. (D) The morphology of PGCLCs four days after induction. (E) Relative expression of PGC-related genes by JY-bESCs and D4 PGCLC induced with BMP4, and BMP4 and BMP8a. Mean±sd, n=3. (F) Representative immunostaining images of PGCLCs and JY-bESCs for TFAP2C, PRDM1 and SOX17 four days after induction (first row: one layer of PGCLCs; second row: 3D projection of Z-stack scanning of PGCLCs; third row: JY-bESCs). (G) Representative immunostaining images of PGCLCs for H3K27me3, H3K9me2 and PRDM1 four days after induction.

FIG. 4: Chimeric Embryos Generated from bESCs carrying the GFP marker gene. (A) A representative mouse-bovine chimeric blastocyst 24 h after aggregation. (B) Whole-mount immunofluorescence of mouse-bovine chimeric blastocysts. (C) IHC staining of mouse-bovine E7.5 embryo using the GFP antibody. The arrows point to the positive regions. (D) Summary of PCR results on JY-bESCs' contribution in mouse chimeric embryo proper and decidua. (E) Bovine-bovine chimeric blastocysts developed from aggregated 8-cell (left) and morula stage (right) embryos. (F) Whole-mount immunofluorescence of bovine chimeric. (G) Day 25 bovine chimeric embryonic disc (the first and second rows) and extraembryonic tissue (the third row) with visible GFP signals. (H) UMAP clustering of all GFP+ cells from two D25 bovine fetuses and spiked-in cells from one extraembryonic sample. (I) Projection of GFP+ (GFP read count>0) and GFP-(GFP read count=0) cells in UMAP.

FIG. 5: Components screening and characterization of bESCs in TiF3 medium. (A) Morphological changes of bESCs in TiF2 medium with individual component elimination over three-days of culture. (B) The morphology of bESCs in TiF2 and TiF3 medium after five passages. (C) Relative expression of pluripotency genes by bESCs in TiF2 and TiF3 medium. Mean±sd, n=3. (D) Immunostaining of passaged EBs derived from JY-bESCs in TiF3 medium for markers of the three germ layers (GATA4 for endoderm, SMA for mesoderm, and TUJ1 for ectoderm).

FIG. 6: Derivation and Characterization of bESCs. (A) Relative expression of pluripotency genes by biPSCs in TiF and TiF2 medium. Mean±sd, n=3. (B) The morphology of biPSCs in TiF2 medium. (C) Flow cytometry analysis of CRIPSR-Cas9 edited JY-bESCs with GFP and frame-corrected Cas9 reporter (top right). (D) JY-bESC GFP+ line with Cas9 out-of-frame reporter after CRISPR-Cas9 editing. (E) EBs formed by JY-bESCs. (F) Negative control immunostaining of JY-bESCs with only secondary antibodies. (G) Negative control immunostaining of EBs with only secondary antibodies. (H) A representative teratoma formed by JY-bESCs. (I) One layer of the Z-stack scanning for immunostaining images of PGCLCs for TFAP2C, PRDM1 and SOX17 four days after induction (Z=18).

FIG. 7: Comparative Transcriptomic Analysis of PSCs. (A) PCA comparison of JY-bESCs to previously published bovine embryos (ICM, APE, ERSE [GSE53387] and blastocyst [BL; GSE52415 and GSE110036]). (B) Heatmap of pluripotency markers across four mouse cell lines: mESC_Yang (mouse naive ESC [GSE89303]), mpESC_Liu (mouse primed ESC [GSE92635]), mFormative_Kinoshita (mouse formative ESC [GSE131556]), and mEPSC_Yang (mouse extended ESC [GSE89303]). (C) Heatmap of pluripotency markers across four human cell lines: hnESC_Bi (human naive ESC [GSE174771]), hpESC_Bi (human primed ESC [GSE174771]), hFormative_Yu (human formative [GSE135989]), and hEPSC_Yang (human extended ESC [GSE89303]). (D) Heatmap comparison of DEGs between JY-bESCs and bMSCs (GSE180931).

FIG. 8: Comparative Transcriptomic Analysis of PSCs. (A) PCA projection of eY-bESCs' pluripotency onto that of mouse PSCs. (B) Heatmap comparison of JY-bESCs, hFormatives_Kioshita (GSE131551), and mFormatives_Kioshita (GSE131556). Key genes related to pluripotency are listed on the left. Significantly enriched GO:BP terms are displayed on the right. (C) Heatmap comparison of JY-bESCs, hpESC_Bi (GSE174771), and mpESC_Liu (GSE92635). Key genes related to pluripotency are listed on the left. Significantly enriched GO:BP terms are displayed on the right. (D) Venn diagram showing the overlap of unique genes in JY-bESCs and bovine embryos. JY-bESCs unique genes were identified through a comparative analysis of JY-bESCs, hFormatives_Kinoshita, and mFormatives_Kinoshita. Bovine embryo unique genes were identified by comparing previously published RNA-seq data of bovine, human, and mouse embryos. Differential gene expression analysis was performed between each pair within their respective three groups, identifying genes that were highly expressed exclusively in JY-bESCs or bovine embryos as unique genes.

FIG. 9: Species-specific Metabolic Pathway Analysis. (A) Bar plot of KEGG pathways enriched by the top 2,000 genes primarily contributing to the variance of PC3 in the 3D PCA. (B-F) Boxplot of Log 2(TPM+1) expression levels for genes involved in various KEGG pathways across different species (cattle, human, and mouse), identified via KEGG analysis of the top 500 genes primarily contributing to PC3 in PCA. (B) Carbon Metabolism; (C) N-glycan Biosynthesis, (D) Reactive Oxygen Species Processes, (E) Biosynthesis of Cofactors, and (F) Purine Synthesis. Cattle (n=31), human (n=19), and mouse (n=25). (G) Boxplot of Log 2(TPM+1) expression levels for genes involved in glycerolipids across different species (cattle, human, and mouse). (H) Barplot of Log 2(TPM+1) expression levels of TPI1 and GPD1 in different bovine embryos and PSCs.

FIG. 10: X Chromosome Inactivation (XCI) Analysis of PSCs. (A) Karyotypes of JY-bESC-8 displaying two X chromosomes. (B) Comparison of the sum of five Y-linked genes (OFD1Y, USP9Y, ZRSR2Y, DDX3Y, and EIF2S3Y) between a female line (JY-bESC-8) and three male lines (JY-bESC-1, JY-bESC-3, JY-bESC-4). (C) Expression of the XIST gene in four JY-bESC lines.

FIG. 11: Inter- and Intra-species Chimeric Embryos Generated from JY-bESCs. (A) E7.5 and E8.5 mouse-bovine chimeric embryos generated with GFP positive bESCs. (B) Serial IHC staining images of E7.5 mouse-bovine chimeric embryo using the GFP antibody. The arrows point to the positive regions. (C) PCR analysis of JY-bESCs' contribution in E7.5 and 8.5 mouse decidual tissues and embryo proper using bovine- or mouse-specific primers. (D) Additional images of whole-mount immunofluorescence of bovine chimeric blastocysts. (E) A flushed Day 25 bovine chimeric embryo showing the fetus and extraembryonic tissue. Each square=16×16 mm. (F) Flow cytometry analysis of GFP positive (P4) and negative (P5) cells in Day 25 in vivo developed bovine chimeric fetuses.

FIG. 12: 10× scRNA-seq Analysis. (A) Dot plot of expression levels of representative markers in each cluster. (B) Pie chart of the proportions of different cell types in GFP+ cells of two Day 25 bovine chimeric embryos. (C) Heatmap of the DEGs from different cell types s in GFP+ cells of two Day 25 bovine chimeric embryos.

### DETAILED DESCRIPTION

Provided herein are compositions and methods for producing bovine pluripotent stem cells (bPSCs), including bovine embryonic stem cells (bESCs), bovine induced pluripotent stem cells (biPSCs), and bovine expanded potential stem cells (bEPSCs).

In one embodiment, provided herein are compositions. In some embodiments, the compositions comprise a cell culture media. In some embodiments, the cell culture medium can be used in methods for generating bPSCs (such as bESCs, biPSCs, and bEPSCs). In some embodiments, the cell culture medium further comprises a suitable serum-free, commercially available stem cell culture medium, such as mTeSR™, mTeSR™-plus (available from StemCell Technologies), or N2B27. The mTeSR™, mTeSR™-plus media are based on the mTeSR™ 1 formulation (described in Ludwig T E et al. (2006). Any suitable serum-free stem cell culture medium can be used in the composition. Derivation of human embryonic stem cells in defined conditions. Nat Biotechnol 24(2): 185-7 and Ludwig T E et al. (2006) Feeder-independent culture of human embryonic stem cells. Nat Methods 3(8): 637-46)). The N2B27 medium comprises Dulbecco's Modified Eagle Medium/Nutrient Mixture F-12 (DMEM/F12) and Neurobasal medium combined with a N2 supplement (such as that commercially available from Thermo Scientific) and a B27 supplement (available from Thermo Scientific).

In some embodiments, in addition to the cell culture medium, the compositions also comprise one or more supplements. In an embodiment, the composition includes at least 90% to 99.9% (v/v) of the cell culture medium. In some embodiments, the composition includes at least 99% (v/v) of the cell culture medium and about 1% (v/v) of a supplement. In an embodiment, the composition includes at least 99% to 99.9% (v/v) of the cell culture medium. For example, the composition includes at least 99%, 99.1%, 99.2%, 99.3%, 99.4%, 99.5%, 99.6%, 99.7%, 99.8%, or at least 99.9% (v/v) of the cell culture medium. In an embodiment, the composition includes about 0.1% to about 1% (v/v) of the supplement. For example, the composition includes about 0.1%, 0.2%, 0.3%, 0.4%, 0.5%, 0.6%, 0.7%, 0.8%, or about 0.9% (v/v) of the supplement.

In some embodiments, the supplement is a fibroblast growth factor receptor (FGFR) inhibitor. The phrase “fibroblast growth factor receptor (FGFR) inhibitor” refers to an agent (e.g., such as a small molecule, peptide, protein, biologic or any combination thereof) that inhibits the FGF/fibroblast growth factor receptor (FG FR) pathway. Fibroblast growth factor receptors are the receptors that bind members of the fibroblast growth factor family of proteins and some of these receptors are involved in pathological conditions, such as cancer. An example of a FGFR inhibitor that can be used includes SU5402 having the chemical name 2-[(1,2-dihydro-2-oxo-3H-indol-3-ylidene)methyl]-4-methyl-1H-pyrrole-3-propanoic acid and the below structure:

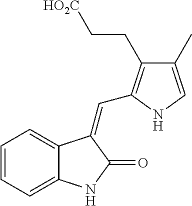 [SMILES: Cc1c[nH]c(/C=C2\C(=O)Nc3ccccc32)c1CCC(=O)O]

In some embodiments, the composition comprises the FGFR inhibitor at a concentration of about 0.1 μM to about 10 μM. In some embodiments, the composition comprises the FGFR inhibitor at a concentration of about 1 μM to about 5 μM. For example, in some embodiments, the composition comprises the FGFR inhibitor at a concentration of about 0.1 μM, about 0.5 μM, about 0.75 μM, about 1 μM, about 2 μM, about 3 μM, about 4 μM, about 5 μM, about 6 μM, about 7 μM, about 8 μM, about 9 μM, about 10 μM. In some embodiments, the composition comprises the FGFR inhibitor at a concentration of about 2 μM.

In addition to the FGFR inhibitor, the composition can further comprise the supplements bovine serum albumin (BSA), a basic fibroblast growth factor (bFGF), and transforming growth factor β1 (TGF β1). In some embodiments, the bFGF is human bFGF or bovine bFGF. In some embodiments, the TGF β1 is human TGF β1 or bovine TGF β1.

In some embodiments, the BSA, bFGF and TGF (31 can be supplied by the cell culture medium. For example, certain serum-free, commercially available stem cell culture medium, such as mTeSR™, mTeSR™-plus, may contain BSA, bFGF and TGF β1 in suitable amounts for use in the compositions described herein. Alternatively, in some embodiments, the BSA, bFGF and TGF β1 can be added as a supplement to the composition. In some embodiments, the amount of BSA used is from about 0.01 to about 5% (v/v). In other embodiments, the amount of BSA used is from about 0.1 to about 2% (v/v). In still other embodiments, the amount of BSA used is about 1% (v/v). In some embodiments, the amount of bFGF used is from about 10 to about 500 ng/mL. In still other embodiments, the amount of bFGF used is from about 20 to about 200 ng/mL. In still other embodiments, the amount of bFGF used is about 100 ng/mL. In still other embodiments, the amount of TGF β1 used is from about 0.1 to about 5 ng/mL. In still other embodiments, the amount of TGF β1 used is from about 0.5 to about 3 ng/mL. In yet still other embodiments, the amount of TGF 11 used is about 1.74 ng/mL.

At a minimum, the compositions described herein comprise a cell culture medium and a FGFR inhibitor and, if not supplied by the cell culture medium, BSA, bFGF, and TGF β1. In some embodiments, the composition can also contain one or more of the following additional supplements: Wingless/Integrated (WNT) inhibitor, a histone methyltransferase DOT1L inhibitor, an adenyl cyclase activator, a mitogen-activated protein-kinase kinases 1 and 2 (MEK1/2) inhibitor, a glycogen synthase kinase (GSK-3) inhibitor, a leukemia inhibitor factor (LIF), or any combination thereof. In some embodiments, the composition contains two or more of a WNT inhibitor, a histone methyltransferase DOT1L inhibitor, an adenyl cyclase activator, a mitogen-activated protein-kinase kinases 1 and 2 (MEK1/2) inhibitor, a glycogen synthase kinase (GSK-3) inhibitor, a leukemia inhibitor factor (LIF), or any combination thereof. In some embodiments, the composition contains three or more of a WNT inhibitor, a histone methyltransferase DOT1L inhibitor, an adenyl cyclase activator, a mitogen-activated protein-kinase kinases 1 and 2 (MEK1/2) inhibitor, a glycogen synthase kinase (GSK-3) inhibitor, a leukemia inhibitor factor (LIF), or any combination thereof. In some embodiments, the composition contains four or more of a WNT inhibitor, a histone methyltransferase DOT1L inhibitor, an adenyl cyclase activator, a mitogen-activated protein-kinase kinases 1 and 2 (MEK1/2) inhibitor, a glycogen synthase kinase (GSK-3) inhibitor, a leukemia inhibitor factor (LIF), or any combination thereof. In some embodiments, the composition contains five or more of a WNT inhibitor, a histone methyltransferase DOT1L inhibitor, an adenyl cyclase activator, a mitogen-activated protein-kinase kinases 1 and 2 (MEK1/2) inhibitor, a glycogen synthase kinase (GSK-3) inhibitor, a leukemia inhibitor factor (LIF), or any combination thereof. In yet other embodiments, the composition contains a WNT inhibitor, a histone methyltransferase DOT1L inhibitor, an adenyl cyclase activator, a mitogen-activated protein-kinase kinases 1 and 2 (MEK1/2) inhibitor, a glycogen synthase kinase (GSK-3) inhibitor, and a leukemia inhibitor factor (LIF).

Any suitable WNT inhibitor may be used in the composition. The terms “WNT inhibitor” WNT pathway inhibitor”, and “WNT signaling inhibitor” are used interchangeably herein to refer to an agent that inhibits one or more aspects of the WNT signaling pathway. In some embodiments, the WNT inhibitor impacts one or more components of the canonical Wnt/β-catenin signaling pathway. In some embodiments, the WNT inhibitor is a small molecule inhibitor. In some embodiments, the WNT inhibitor is selected from XAV-939, Wnt-C59, IWR1, ICG-001, IWP-2, LGK-974, CCT251545, FHF 535, IPW-4, JW 67, JW 74, KYA 1797K, NLS-StAx-H, TAK 715, WIC1, and derivatives thereof. In some embodiments, the WNT inhibitor is IWR1 or a derivative thereof. IWR1 is also referred to as IWR-1-endo, endo-IWR 1, or IRW-1 and has the structure:

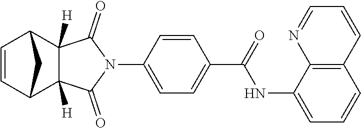 [SMILES: O=C(Nc1cccc2cccnc12)c1ccc(N2C(=O)[C@@H]3[C@H](C2=O)[C@@H]2C=C[C@H]3C2)cc1]

In some embodiments, the composition comprises more than one WNT inhibitor. For example, in some embodiments the composition comprises two, three, four, or more than four WNT inhibitors. In some embodiments, the composition comprises a combination of two or more WNT inhibitors selected from XAV-939, Wnt-C59, IWR1, ICG-001, IWP-2, LGK-974, CCT251545, FHF 535, IPW-4, JW 67, JW 74, KYA 1797K, NLS-StAx-H, TAK 715, WIC1, and derivatives thereof.

In some embodiments, the composition comprises the WNT inhibitor at a concentration of about 0.1 μM to about 25 μM. In some embodiments, the composition comprises the WNT inhibitor at a concentration of about 1 μM to about 20 μM. For example, in some embodiments, the composition comprises the WNT inhibitor at a concentration of about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, or about 20 μM. In some embodiments, the composition comprises the WNT inhibitor at a concentration of about 0.1 μM to about 10 μM. In some embodiments, the composition comprises the WNT inhibitor at a concentration of about 0.5 to about 5 μM. In some embodiments, the composition comprises the WNT inhibitor at a concentration of about 2.5 μM. In some embodiments, the composition comprises the WNT inhibitor at a concentration of about 5 μM to about 15 μM. In some embodiments, the composition comprises the WNT inhibitor at a concentration of about 8 μM to about 12 μM. In some embodiments, the composition comprises the WNT inhibitor at a concentration of about 10 μM.

In other embodiments, the composition comprises a DOT1L inhibitor. The term “DOT1L inhibitor” refers to an inhibitor of the histone methyltransferase DOT1L, which methylates H3K79 using S-adenosyl-L-methionine (SAM) as a co-factor. Any suitable DOT1L inhibitor may be used. In some embodiments, the DOT1L inhibitor is a SAM competitive inhibitor of DOT1L. In some embodiments, the DOT1L inhibitor is a small molecule inhibitor. In some embodiments, the DOT1L inhibitor is EPZ004777 (also referred to herein as “iDOT1L”) or a derivative thereof. EPZ004777 has the chemical name 7-[5-Deoxy-5-[[3-[[[[4-(1,1-dimethylethyl)phenyl]amino]carbonyl]amino]propyl](1-methylethyl)amino]-3-D-ribofuranosyl]-7H-pyrrolo[2,3-d]pyrimidin-4-amine Formic Acid Salt, and the structure:

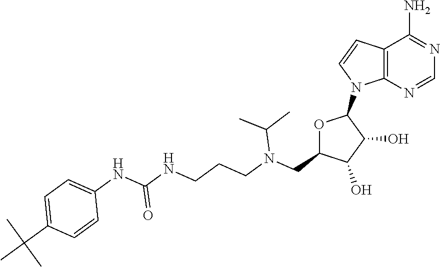 [SMILES: CC(C)N(CCCNC(=O)Nc1ccc(C(C)(C)C)cc1)C[C@H]1O[C@@H](n2ccc3c(N)ncnc32)[C@H](O)[C@@H]1O]

In some embodiments, the DOT1L inhibitor is EPZ-5676 or a derivative thereof. EPZ-5675 is also referred to as pinometostat, and has the chemical name (2R,3R,4S,5R)-2-(6-amino-9H-purin-9-yl)-5-((((1R,3S)-3-(2-(5-(tert-butyl)-1H-benzo[d]imidazol-2-yl)ethyl)cyclobutyl)(isopropyl)amino)methyl)tetrahydrofuran-3,4-diol and the structure:

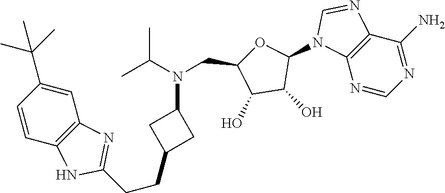

In some embodiments, the composition comprises more than one DOT1L inhibitor. For example, in some embodiments the composition comprises two DOT1L inhibitors.

In some embodiments, the composition comprises the DOT1L inhibitor (e.g. EPZ004777, EPZ-5676) at a concentration of about 0.5 μM to about 30 μM. In some embodiments, the composition comprises the DOT1L inhibitor at a concentration of about 1 μM to about 25 μM. In some embodiments, the composition comprises the DOT1L inhibitor at a concentration of about 1 μM to about 10 μM. In some embodiments, the composition comprises the DOT1L inhibitor at a concentration of about 1 μM to about 5 μM. For example, in some embodiments the composition comprises the DOT1L inhibitor at a concentration of about 1 μM, about 2 μM, about 3 μM, about 4 μM, or about 5 μM. In some embodiments, the composition comprises the DOT1L inhibitor at a concentration of about 1 μM, about 1.5 μM, about 2 μM, about 2.5 μM, about 2.6 μM, about 2.7 μM, about 2.8 μM, about 2.9 μM, about 3 μM, about 3.1 μM, about 3.2 μM, about 3.3 μM, about 3.4 μM, about 3.5 μM, about 3.6 μM, about 3.7 μM, about 3.8 μM, about 3.9 μM, about 4 μM, about 5.5 μM, or about 5 μM. In some embodiments, the DOT1L inhibitor is EPZ004777.

In yet other embodiments, the composition comprises a leukemia inhibitory factor (LIF). The LIF used in the composition can be human LIF or bovine LIF or a combination thereof. In some embodiments, the LIF is human LIF. In other embodiments, the LIF is bovine LIF. In some embodiments, the cell culture medium comprises about 100 U/mL to about 5000 U/mL human LIF or bovine LIF. In some embodiments, the cell culture medium comprises about 100 U/mL to about 2000 U/mL human LIF. In some embodiments, the cell culture medium comprises about 100 U/ml, about 200 U/mL, about 300 U/ml, about 400 U/mL, about 500 U/ml, about 600 U/mL, about 700 U/ml, about 800 U/mL, about 900 U/ml, about 1000 U/mL, about 1100 U/ml, about 1200 U/mL, about 1300 U/ml, about 1400 U/mL, about 1500 U/ml, about 1600 U/mL, about 1700 U/ml, about 1800 U/mL, about 1900 U/ml, or about 2000 U/mL human LIF or bovine LIF. In some embodiments, the cell culture medium comprises 1000 U/mL human LIF or bovine LIF.

In still further embodiments, the composition comprises at least one glycogen synthase kinase 3 (GSK-3) inhibitor. In some embodiments, the GSK-3 inhibitor is a small molecule inhibitor. In some embodiments, the GSK-3 inhibitor is selected from CHIR-99021, CHIR-98014, LY2090314, BIO, TWS119, Tideglusib, SB216763, and derivatives thereof. In some embodiments, the GSK-3 inhibitor is CHIR-99021 or a derivative thereof. CHIR-99021 is also referred to as Laduviglusib, and has the structure:

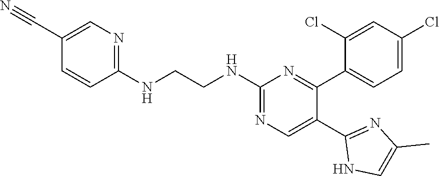 [SMILES: Cc1c[nH]c(-c2cnc(NCCNc3ccc(C#N)cn3)nc2-c2ccc(Cl)cc2Cl)n1]

In some embodiments, the composition comprises the GSK-3 inhibitor at a concentration of about 0.5 μM to about 10 μM. In some embodiments, the composition comprises the GSK-3 inhibitor at a concentration of about 1 μM to about 5 μM. For example, in some embodiments the composition comprises the GSK-3 inhibitor at a concentration of about 1 μM, about 2 μM, about 3 μM, about 4 μM, or about 5 μM. In some embodiments, the composition comprises the GSK-3 inhibitor at a concentration of about 1 μM, about 1.5 μM, about 2 μM, about 2.5 μM, about 2.6 μM, about 2.7 μM, about 2.8 μM, about 2.9 μM, about 3 μM, about 3.1 μM, about 3.2 μM, about 3.3 μM, about 3.4 μM, about 3.5 μM, about 3.6 μM, about 3.7 μM, about 3.8 μM, about 3.9 μM, about 4 μM, about 5.5 μM, or about 5 μM. In some embodiments, the GSK-3 inhibitor is CHIR-99021.

In yet still other embodiments, the composition comprises at least one mitogen-activated protein kinase inhibitor. In some embodiments, the at least one mitogen-activated protein kinase inhibitor inhibits MEK1/2 (i.e. inhibits MEK1 and MEK2). In some embodiments, the MEK1/2 inhibitor is selected from Trametinib, PD0325901, Selumetinib, UO126-EtOH, PD989059, and derivatives thereof. In some embodiments, the MEK1/2 inhibitor is PD0325901 or a derivative thereof. PD0325901 is also referred to as Mirdametinib, and has the structure:

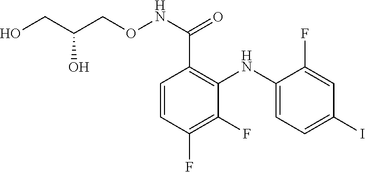 [SMILES: O=C(NOC[C@H](O)CO)c1ccc(F)c(F)c1Nc1ccc(I)cc1F]

In some embodiments, the composition comprises the MEK1/2 inhibitor at a concentration of about 0.1 μM to about 10 μM. In some embodiments, the composition comprises the MEK1/2 inhibitor at a concentration of about 0.25 μM to about 7.5 μM. In some embodiments, the composition comprises the MEK1/2 inhibitor at a concentration of about 0.5 μM to about 5 μM. In some embodiments, the composition comprises the MEK1/2 inhibitor at a concentration of about 0.75 μM to about 2.5 μM. In some embodiments, the composition comprises the MEK1/2 inhibitor at a concentration of about 1 μM. In some embodiments, the composition comprises the MEK1/2 inhibitor at a concentration of about 0.1 μM, about 0.2 μM, about 0.3 μM, about 0.4 μM, about 0.5 μM, about 0.6 μM, about 0.7 μM, about 0.8 μM, about 0.9 μM, about 1.0 μM, about 1.1 μM, about 1.2 μM, about 1.3 μM, about 1.4 μM, about 1.5 μM, about 1.6 μM, about 1.7 μM, about 1.8 μM, about 1.9 μM, about 2.0 μM, about 2.1 μM, about 2.2 μM, about 2.3 μM, about 2.4 μM, or about 2.5 μM. In some embodiments, the MEK1/2 inhibitor is PD0325901.

In still further embodiments, the composition comprises an adenyl cyclase activator. In some embodiments, the adenyl cyclase activator is Forskolin. In some embodiments, the composition comprises the adenyl cyclase activator (e.g. Forskolin) at a concentration of about 0.1 μM to about 20 μM. In some embodiments, the composition comprises the adenyl cyclase activator (e.g. Forskolin) at a concentration of about 1 μM to about 15 μM. In some embodiments, the composition comprises the adenyl cyclase activator (e.g. Forskolin) at a concentration of about 1 μM, about 2 μM, about 3 μM, about 4 μM, about 5 μM, about 6 μM, about 7 μM, about 8 μM, about 9 μM, about 10 μM, about 11 μM, about 12 μM, about 13 μM, about 14 μM, or about 15 μM. in some embodiments, the composition comprises about 10 μM Forskolin.

In some embodiments, disclosed herein is a composition (also called as TiF2 medium) which can be used to derive, grow and maintain the pluripotency and self-renewal capacity of a cell line, such as bovine embryonic stem cells (bESCs), bovine induced pluripotent stem cells (biPSCs), and bovine expanded potential stem cells (bEPSCs).

In an embodiment, the composition (e.g., TiF2) includes a cell culture medium such as mTeSR-plus media which supplies BSA, bFGF, and TGF β1, and the following supplements: a WNT inhibitor (such as IWR1); a histone methyltransferase DOT1L inhibitor (such as EPZ004777); an adenyl cyclase activator (such as forskolin); a MEK1/2 inhibitor (such as PD184352); a FGFR inhibitor (such as SU5402); a GSK-3 inhibitor (such as CHIR99021); and a LIF (such as a human or bovine LIF).

In some embodiments, the TiF2 medium comprises a mTesR-plus media (which contains BSA, bFGF and TGF β1) and the supplements:
a) IWR1;
b) EPZ004777;
c) forskolin;
d) PD184352;
e) SU5402;
f) CHIR99021; and
g) human LIF or a bovine LIF.

In yet other embodiments, disclosed is a composition (also called as TiF3 medium) to derive, grow and maintain the pluripotency and self-renew capacity of a cell line, such as bovine embryonic stem cells (bESCs), bovine induced pluripotent stem cells (biPSCs), and bovine expanded potential stem cells (bEPSCs). This composition (e.g., TiF3) includes: a cell culture medium (such as N2B27 media); a WNT inhibitor (such as IWR1); a histone methyltransferase DOT1L inhibitor (such as EPZ004777); an adenyl cyclase activator (such as forskolin); a MEK1/2 inhibitor (such as PD184352); a FGFR inhibitor (such as SU5402); a GSK-3 inhibitor (such as CHIR99021); a LIF (such as a human or bovine LIF); BSA, a bFGF (such as human or bovine bFGF); and a TGF β1 (such as human or bovine TGF β1).

In some embodiments, the TiF3 medium comprises a N2B27 media and the supplements:
a) IWR1;
b) EPZ004777;
c) forskolin;
d) PD184352;
e) SU5402;
f) CHIR99021;
g) human LIF or a bovine LIF;
h) BSA;
i) human bFGF or a bovine bFGF; and
j) human TGF β1 or a bovine TGF β1.

In some embodiments, the TiF2 and TiF3 media contain from about 0.1 μM to about 25 μM of a WNT inhibitor; from about 0.1 μM to about 25 μM of a histone methyltransferase DOT1L inhibitor; from about 0.1 μM to about 10 μM of a MEK1/2 inhibitor; about 0.1 μM to about 10 μM of a FGFR inhibitor; from about 0.5 μM to about 10 μM of a GSK-3 inhibitor; from about 1 μM to about 50 μM of an adenyl cyclase activator; about 100 U/ml to about 5000 U/ml of LIF, from about 0.01 to about 5% of BSA (v/v); from about 10 to about 500 ng/ml of bFGF; and from about 0.1 to about 5 ng/ml of TGF β1.

In still other embodiments, the TiF2 and TiF3 media contain from about 0.1 to about M or from about 0.1 to about 5 μM of a WNT inhibitor; from about 0.1 to about 10 μM or from about 0.1 to about 5 μM of a histone methyltransferase DOT1L inhibitor; from about 0.1 to about 2 μM of a MEK1/2 inhibitor; from about 0.1 to about 5 μM of a FGFR inhibitor; from about 0.5 to about 5 μM of a GSK-3 inhibitor; from about 5 to about 20 μM, or from about 5 to about 15 μM of an adenyl cyclase activator; from about 100 to about 2000 U/ml of LIF, from about 0.1 to about 2% of BSA (v/v); from about 20 to about 200 ng/ml of bFGF; and from about 0.5 to about 3 ng/ml of TGF β1.

In still other embodiments, the TiF2 and TiF3 media contain about 2.5 μM of a WNT inhibitor; about 0.8 μM of a MEK1/2 inhibitor; about 2 μM of a FGFR inhibitor; and about 3 μM of a GSK-3 inhibitor; about 3.3 μM of a DOT1L inhibitor; about 10 μM of an adenyl cyclase activator; about 1000 U/ml of a LIF, about 1% of BSA (v/v), about 100 ng/ml of bFGF and about 1.74 ng/ml of TGF β1.

Both the TiF2 and TiF3 media can be used to maintain the pluripotency and self-renew capacity of a cell line. In some embodiments, the cell line includes pluripotent stem cells. In other embodiments, the pluripotent stem cells are bovine embryonic stem cells (bESCs), bovine induced pluripotent stem cells (biPSCs), and bovine expanded potential stem cells (bEPSCs). In still other embodiments, the bovine embryonic stem cells have specific application in bovine and biomedical research. Moreover, the TiF2 and TiF3 media described herein provide improved pluripotency and colony morphology compared to the TiF media known in the art and described in WO 2023/122571. Specifically, the TiF2 and TiF3 media are capable of sustaining a normal bovine embryonic stem cell phenotype, such as remaining in an un-natural state of un-differentiation following single-cell passaging for more than eighty passages (at least nine months of in vitro culturing) made using the methods described herein.

In another embodiment, provided herein are methods for producing stem cells, such as bovine PSCs, such as bovine embryonic stem cells (bESCs), bovine induced pluripotent stem cells (biPSCs), and bovine expanded potential stem cells (bEPSCs). In some embodiments, provided herein are bESCs. In some embodiments, provided herein are biPSCs. In yet other embodiments, provided herein are bEPSCs. In some embodiments, the, the bPSCs, such as the bESCs, biPSCs and/or bEPSCs, are produced by a method described herein.

The methods for producing bovine PSCs involve obtaining or providing bovine pre-implantation embryos. The bovine pre-implantation embryos can be obtained or provided using techniques known in the art. Once bovine pre-implantation embryos are obtained or provided, the pre-implantation embryos are treated with at least one enzyme to remove the zone pellucida. Any enzyme that is capable of removing the zone pellucida can be used. In some embodiments, the enzyme is pronase, a hyaluronidase, an acrosin or any combinations thereof. Once the zone pellucida is removed, the treated (namely, enzyme treated) embryos are cultured on composition as described herein, such as, for example, a TiF2 or TiF3 composition. The bovine PSCs are then harvested, collected and/or isolated from the culture using techniques known in the art.

In an aspect, disclosed is a stem cell line including a bESC (such as a bESC, biPSC, and/or bEPSC), wherein the bESC maintains pluripotency. In an aspect, disclosed is a bovine embryonic stem cell line being capable of sustaining a normal bovine PSC phenotype such as the un-natural morphology of dome-shaped colonies in vitro cultured for at least eight months in vitro. In an aspect, disclosed is a bovine embryonic stem cell line being capable of sustaining the un-natural property of single-colony expansion in vitro following at least nine months of in vitro culturing. For example, a bovine embryonic stem cell line being capable of sustaining the un-natural property of single-colony expansion in vitro following about 10 months, about 11 months, about 12 months, about 13 months, about 14 months, or about 14 months of in vitro culturing.

In an aspect, disclosed is a bovine embryonic stem cell line being capable of sustaining a normal bovine embryonic stem cell phenotype such as remaining in an un-natural state of un-differentiation following single-cell passaging for more than eighty passages (at least nine months of in vitro culturing) made by the method disclosed herein. In an aspect, disclosed is a bovine embryonic stem cell line capable of sustaining a normal bovine embryonic stem cell phenotype following single-cell passaging for more than eighty passages (at least nine months of in vitro culturing) made by using the composition disclosed herein.

In an embodiment, the JY-bESCs (which are derived from bovine pre-implantation embryos, see Example 4) disclosed herein can be passaged from single cells. In an embodiment, the JY-bESCs disclosed herein show a doubling time of about 15 hours to about 25 hours. For example, the JY-bESCs disclosed herein show a doubling time of about 15 hours, about 16 hours, about 17 hours, about 18 hours, about 19 hours, about 20 hours, about 21 hours, about 22 hours, about 23 hours, about 24 hours, or about 25 hours. In an embodiment, the JY-bESCs disclosed herein show a doubling time of about 19.12±0.84 hours. In an embodiment, the JY-bESCs disclosed herein can spontaneously differentiate into three germ layers in vitro and in vivo. In an embodiment, the JY-bESCs disclosed herein can be directly induced to neural progenitor cells (NPCs) and primordial germ cell-like cells (PGCLCs). In an embodiment, the JY-bESCs show high incorporation rate to mouse-bovine chimeric embryos. In an embodiment, the JY-bESCs show high incorporation rate to bovine chimeric embryos and have great potential to generate germline chimeric embryos. In certain embodiments, the JY-bESCs can be used to generate interspecies and intraspecies chimeric embryos by aggregating with ZP-free embryos.

In some embodiments, the method involves obtaining or providing bovine pre-implantation embryos. Such bovine pre-implantation embryos can be generated and obtained using techniques known in the art. Once such bovine pre-implantation embryos are obtained or provided, the zone pellucida is removed from the embryos by treating the embryos with an enzyme. An example of enzymes that can be used to remove the zone pellucida is a pronase, hyaluronidase, acrosin or any combinations thereof. Once the zone pellucida is removed, the zone pellucida-free embryos can be cultured on compositions described herein. For example, in some embodiments, the zone pellucida-free embryos can be cultured with a TiF2 medium. In other embodiments, the zone pellucida-free embryos can be cultured with a TiF3 medium. In still other embodiments, the medium can be replaced daily, every other day, every third day, every fourth day, or every fifth day.

The zone pellucida-free embryos are cultured on the compositions described herein, such as, for example, a TiF2 or TiF3 media, for as long as necessary to obtain bESCs, biPSCs and/or bEPSCs, (e.g. harvest). The cells may then be cultured in the compositions described herein (e.g., the TiF2 or TiF3 media) for a suitable duration (e.g. number of days or number of passages) until bovine induced pluripotent stem cells are generated. Successful generation of bESCs, biPSCs and/or bEPSCs, can be verified by measuring expression of genes/markers in the cells, including pluripotent markers such as OCT4, NANOG, LIN28A/B, SALL4, DNMT3A/3B.

Unlike other methods known in the art, such as, for example, those described in WO 2023/122571, the methods described herein do not require any reprogramming the stem cell, such as a bovine mesenchymal stem cell. In some methods, reprogramming of the stem cell is required by overexpressing a plurality of reprogramming factors using techniques known in the art. Not having to reprogram the stem cells saves time and cost thus making the methods described herein cost effective and efficient compared to other methods known in the art. The bovine embryonic stem cells produced as described herein can be used for a number of applications, such as, for example, in cultured meat production and clinical trials.

The present disclosure is illustrated and further described in more detail with reference to the following non-limiting examples. Section headings as used in this section and the entire disclosure herein are merely for organizational purposes and are not intended to be limiting.

### EXAMPLES

### Materials and Methods

### Example 1: Bovine Embryo Production and Culture

Generating bESC from blastocysts by in vitro fertilization (IVF). Bovine cumulus oocyte complexes (COCs) were purchased from commercial vendors. The maturation process was conducted in the HEPES-buffered IVM medium at 38.5 °C during shipping. Twenty-one to 24 h after maturation, COCs were transferred to BO-IVF medium in 4-well dishes covered with mineral oil and incubated in a 38.5 °C incubator under 5.5% CO₂. Frozen bovine semen was used for in vitro fertilization. The straws were thawed in a 37 °C-water bath for 1-2 min and washed twice by centrifugation at 328 g for 5 min. The supernatant was discarded, and a final concentration of 1×10⁶ sperm was added (Day 0) to each IVF well at 38.5 °C and 5.5% CO₂ for 16-20 h. The remaining cumulus cells of the presumptive zygotes were removed by incubating 1 ml 0.76 mg/ml hyaluronidase for 2 min at 38.5 °C. After digestion, 800 μl supernatant was discarded, and the precipitated embryos were vortexed for 5 min to denude embryos. Denuded embryos were rinsed three times in BO-wash and collected in BO-IVC culture droplets. Embryos were incubated at 38.5 °C, 5.5% CO₂, and 6% 02 for further development.

### Example 2: Derivation and Culture of bESCs

Bovine blastocysts produced by IVF from Day 7 to 8 were used for bESC derivation. Unhatched blastocysts were treated with freshly made 2 mg/mL Pronase (Merck Millipore, 10165921001) at 38.5 °C for 2 min to remove the zona pellucida (ZP) and then thoroughly washed in in vitro culture (IVC) medium three times to remove traces of the enzyme. ZP-free blastocysts were transferred to a 24-well plate with mitomycin C treated MEF feeders in TiF2 medium that contains mTeSR-plus media (STEMCELL Technologies, 100-0276) supplemented with 2.5 μM IWR1 (AOBIOUS, AOB33702), 3.3 μM iDOT1L (AOBIOUS, AOB1922), 0.8 μM PD184352 (ApexBio, A1792), 2 μM SU5402 (ApexBio, A3843), 3 μM CHIR99021 (AOBIOUS, AOB3866), 10 μM Forskolin (AOBIOUS, AOB6380), and 1,000 U/ml human LIF (R&D Systems, 7734LF025), with the presence of 10 μM ROCKi (AOBIOUS, AOB3877) and incubated at 37 °C and 5% CO₂ for 48 h without disruption. After 7-8 days, the outgrowths of the embryos were passaged using TrypLE Express (Gibco, 12604013) to a 12-well plate with feeder layer in the presence of 10 μM ROCKi. The distinguishable dome-shaped colonies started to appear from P1 to P2 and were evaluated by SSEA4 live staining. The SSEA4-positive cells were sorted using a BD FACSAria II Cell Sorter (BD Bioscience) and replated to fresh feeder layer with 10 μM ROCKi. Then the bESCs were passaged every three days at a ratio of 1:8 to 1:6 by TrypLE Express in TiF2 medium with 10 μM ROCKi. TiF3 medium contains N2B27 medium supplemented with 2.5 μM IWR1, 3.3 μM iDOT1L, 0.8 μM PD184352, 2 μM SU5402, 3 μM CHIR99021, 10 μM Forskolin, 1,000 U/ml human LIF, 100 ng/ml FGF2, 1.74 ng/ml TGFβ1 and 1% BSA. N2B27 medium consisted of a mixture of 1:1 DMEM/F12 medium and Neurobasal medium, 0.5% v/v N-2 Supplement, 0.5% v/v B-27 Supplement, 2 mM MEM Non-Essential Amino Acid Solution, 1% v/v GlutaMAX Supplement, 0.1 mM 2-mercaptoethanol, 100 U/mL Penicillin, and 100 μg/mL Streptomycin, as previous reported.

Quantitative Reverse Transcription-PCR (qRT-PCR) Analysis

Total RNA was isolated with RNeasy mini kits (Qiagen, 74106). Genomic DNAs were removed by DNase I (Qiagen, 79254) incubation. A total of 0.5 g RNAs were then reverse transcribed into cDNA using iScript reverse transcription supermix (Bio-Rad Laboratories, 170-8891). qRT-PCR reactions were performed with SYBR Green supermix (Bimake, B21203) using the ABI 7500 Fast platform (Thermo Fisher Scientific). GAPDH was used as the housekeeping gene for quantification.

### Embryoid Body Differentiation

To induce EB formation for in vitro differentiation demonstration, bESC lines were grown to 70-80% confluency and mainly as middle-size colonies, the cells were treated with freshly prepared 1 mg/mL collagenase for 30 min and removed from the plate by pipetting. After three washes with DMEM/F12, the cells were plated onto low-adhesive petri dishes in EB formation medium (DMEM/F12 with 20% KSR and DMEM with 10% FBS 1:1 medium supplemented with 3 μM CHIR99021 and 20 ng/ml Activin (R&D Systems, 338AC010)). Half of the medium was changed to DMEM with 10% FBS every other day. EBs were treated by 0.05% Trypsin (Gibco, 25200056) 5 days later and plated onto gelatin-coated plates. The cells were subjected to immunofluorescence staining after another 7 days of culture.

### Neural Progenitor Cell (NPC) Differentiation

Bovine NPC direct differentiation protocol was modified based on the instruction of STEMdiff™ SMADi Neural Induction Kit (Stem Cell Technologies, 08581). Briefly, EBs were generated as mentioned above while the cells were cultured in STEMdiff™ Neural Induction Medium with SMAD inhibitors and 10 μM ROCKi for 4 days. The EBs were transferred to Matrigel coated wells and cultured in STEMdiff™ Neural Progenitor Medium for another 15 days when the cells were subjected to immunostaining.

### Primordial Germ Cell-Like Cell (PGCLC) Induction

Bovine PGCLCs were induced directly from JY-bESCs following a published protocol (9). Briefly, 3.5×10³ JY-bESCs were seeded into each well of ultra-low attachment round-bottom 96-well plates (Corning, 7007) in GK15 medium containing GMEM, 15% (v/v) KSR, 1× sodium pyruvate, 1×NEAA, 1× β-mercaptoethanol, 1× Glutamax and 0.5× penicillin and streptomycin, supplemented with 500 ng/mL BMP4 (GIBCO, PHC9534), 500 ng/mL BMP8a (R&D Systems, 1073BP010), 1000 U/mL LIF, 100 ng/mL SCF (R&D, 255SC010), 50 ng/mL EGF (Sigma, E9644) and 10 μM ROCKi. The cells were subjected to RNA extraction and immunostaining four days later.

### Immunostaining

The cells were first fixed in 4% PFA for 15 min at room temperature and treated with 0.5% Triton X-100 in PBS for 15 min membrane permeabilization. After blocking with 5% goat serum (Cell Signaling Technology, 5425S) in PBS, the cells were incubated in goat serum containing primary antibodies for 2 h at 37 °C, followed by secondary antibodies at room temperature for 1 h. Similarly, the aggregated embryos at blastocyst stage were fixed in 4% PFA for 20 min and permeabilized in 0.5% Triton X-100 for 30 min at room temperature. The embryos were blocked with goat serum and incubated with primary antibodies at 4 °C overnight and secondary antibody at room temperature for 1 h. Cells and embryos were counter-stained with DAPI and mounted on slides in Slow Fade® Gold antifade reagent (Invitrogen, S36938). The slides were imaged under a Nikon AIR confocal microscope.

Primary antibodies including OCT4 (Santa Crutz, sc-9081), SOX2 (Merck Millipore, AB5603), NANOG (Invitrogen, 14-5768-80), GATA4 (Cell Signaling Technology, 36966), TUJ1 (Invitrogen, A25532), SMA (Invitrogen, A25531), SOX2 (Invitrogen, 14-9811-80), NESTIN (Abcam, ab5968-100), PAX6 (Santa Crutz, sc-81649), PRDM1 (Invitrogen, 14-5963-82), TFAP2C (Sigma, HPA055179) and SOX17 (Origene, TA500044S), H3K27me3 (Merck Millipore, 07-449) were diluted according to the manufacturer's instructions. Alexa Fluor 488, 594 or 647 conjugated goat anti-rabbit or goat anti-mouse secondary antibody (Cell Signaling Technology) were used in 1:1000 dilution. For cell surface marker staining, the cells were stained with NL557-conjugated TRA-1-60 (R&D Systems, NLLC4770R), SSEA-3 (R&D Systems, MAB1434) and Alexa Fluor 594 conjugated SSEA-4 (BioLegend, 330414) according to the manufacturer's protocol.

### RNA Fluorescence In Situ Hybridization (FISH)

Bovine XIST probes with Quasar 570 Dye and HUWE1 probes with Quasar 670 Dye were purchased from Biosearch Technologies (SMF-1063-5 and SMF-1065-5). Cell fixation and hybridization were performed by following the manufacture's recommended protocol.

### Teratoma Assay

The JY-bESCs were treated with 10 μM ROCKi for 2 h before TrypLE digestion. After digestion and centrifugation, the cells were resuspended with 30% chilled Matrigel (Corning, 354230) in DMEM/F12 and 10 μM ROCKi. 5×10⁶ of JY-bESCs were injected into the hint leg of 10-week-old male NOD-SCID mice (Charles River, 394NODSCID) intramuscularly (100 μl per injection). Six to eight weeks after the injection, the teratomas were dissected and fixed in 10% formalin. Paraffin-embedded teratomas were sectioned and stained with hematoxylin and eosin.

### RNA-Seq and Data Analysis

Total RNA was isolated from JY-bESCs using miRNeasy Mini kit (Qiagen, 217004). The quality of total RNA was examined by Nanodrop, Agarose Gel Electrophoresis and the Aglient 2100 bioanalyzer. Ribosomal RNA was then removed by using Ribo-Zero-rRNA Removal kit (Epicentre). To prepared sequencing library, first, the mRNA was fragmented randomly by adding fragmentation buffer, then the cDNA was synthesized by using mRNA template and random hexamers primer, after which a custom second-strand synthesis buffer (Illumina), dNTPs, RNase H and DNA polymerase I were added to initiate the second-strand synthesis. Second, after a series of terminal repairs, ligation and sequencing adaptor ligation, the double-stranded cDNA library was completed through size selection and PCR enrichment. Finally, sequencing libraries were quantified by using Agilent 2100 bioanalyzer and then fed into Illumina sequencers.

The RNA-seq data analysis was conducted at usegalaxy.org. The published datasets including bMSC (1), bEPSC (2), primed bESC (3), and bovine embryos at 16-cell (10) and blastocyst (3) stages were used for comparison. Sequencing adapters and reads with low quality were trimmed using Cutadapt before mapping. The quality of reads after filtering was examined using fastQC. For mapping, bovine genomic sequence and RefSeq gene coordinate (ARS-UCD1.2/bosTau9) were downloaded from the UCSC genome browser. All filtered reads were aligned to bovine reference genome by RNA STAR (Galaxy Version 2.7.8a) with default parameters. The number of reads per gene was counted by featureCounts (Galaxy Version 2.0.1). Differentially expressed genes between different samples were identified using default parameters in DESeq2 (Galaxy Version 2.11.40.6+galaxy1), which generated a principal component analysis plot and the heatmap of the sample-to-sample distance matrix. The normalized counts for each sample were also used for GSEA analysis (gsea-msigdb.org), with the absolute log 2(FC)>1 values used for IPA analysis (Qiagen).

### Karyotyping

Karyotyping was carried out on different bESC lines. bESCs were first incubated with 20 μg/ml Demecolcine (Sigma-Aldrich, D7385) at 37 °C for 2 h, following which the cells were harvested by trypsinization. The cells were then incubated in hypotonic solution (0.56% KCl solution) for 15 min at 37 °C. After washing in the fixative solution three times (methanol/glacial acetic acid 3:1), the cells were dropped onto wet and ice-cold glass slides. Giemsa (Sigma-Aldrich, G3032) at 1:20 dilution was applied onto the dried slides for staining. The nuclei were visualized with an Olympus microscope under a 100× oil objective lens.

### Bisulfite Sequencing

For bisulfite sequencing, genomic DNAs were extracted and bisulfite converted using the EpiTeck Bisulfite Kit (Qiagen, 59104). Bovine OCT4 and NANOG proximal promoter regions were amplified using PCR primers previously reported (11), and OCT4 distal primers were designed on MethPrimer. The sequences of the primers are as follows. For OCT4 proximal promoter region primers, forward primer: 5′-GTTTGGAGAGGGGTTTTGAAGAATGTGTAG-3′ (SEQ ID NO:1), and reverse primer: 5′-ATCCCACCCACTAACCTTAACCTCTAAC-3′ (SEQ ID NO:2). For OCT4 distal enhancer region primers, forward primer: 5′-TGTTTGGAGAATTTTATGGATAGAG-3′ (SEQ ID NO:3), and reverse primer: 5′-CCAATTAAATCATCAAACCTAACTC-3′ (SEQ ID NO:4). For NANOG proximal promoter region primers, forward primer: 5′-TAGGTGGTTATAGGAGATGTATTTTTGATT-3′ (SEQ ID NO:5), and reverse primer: 5′-TTATAAATAAAACTCAACCATACTTAACCC-3′ (SEQ ID NO:6). PCR were performed with CloneAmp HiFi PCR Premix (Takara Bio USA, 639298) and cloned using the In-Fusion HD Cloning System (Clontech, 638910) into pIRES2-DsRed vector digested by BglII and EcoRI (New England Biolabs). Clones were picked, cultured in 6 mL LB medium with antibiotics overnight, and plasmid DNAs were extracted using a Qiaprep Miniprep Kit (Qiagen, 27106) and subjected to Sanger DNA sequencing. The results were then analyzed using the BiQ Analyzer.

### In Vivo Inter- and Intra-Species Chimera Assays

We first generated a bESC fluorescent line using piggyBac based PB-CAG-eGFP and pCAG-PBase plasmids. Chimeric embryos were then generated by aggregating mouse embryos with the GFP-positive bESC line as described previously (6). E2.5 mouse morulae were isolated from CD-1 females (Charles River). Zona pellucidae were removed by brief exposure to acidic Tyrode's solution (Sigma-Aldrich, T1788) followed by three washes in KSOM embryo medium (Millipore, MR-101-D). Zona-free embryos were placed individually in micro-wells with KSOM embryo medium in an aggregation plate covered with light mineral oil (Fisher Scientific, 01211). Bovine ESCs were fed 2 h before aggregation by replacing media. Cells were washed 2× with PBS, dislodged from the plates by brief exposure to 0.05% Trypsin (Sigma, SM-2003-C) to obtain clumps of 8 to 12 cells. Two clumps of cells were then placed with a zona-free embryo in the micro-well and co-cultivated in a 37 °C incubator with 6% CO₂, 5% O₂ and 89% N₂. After an overnight incubation, bESC-mouse embryo aggregates that did not develop into blastocyst were discarded and 90 to 92 blastocysts from each line were then transferred into pseudopregnant females for subsequent development. Embryos were harvested on E7.5 and E8.5 and fixed in 4% PFA for further analysis.

For the generation of bovine-bovine chimeric embryos, the embryos were produced by IVF as described before. Zona pellucidae were removed from Day 3 (Day 0=fertilization) 8-cell stage embryos and Day 5 morulae by the treatment of 2 mg/mL Pronase for 2 min at 38.5 °C followed by three washes in IVC medium. Similarly, the ZP-free embryos were individually cultured with three clumps of eight to twelve bESCs in the micro-wells at 38.5 °C, 5% 02 and 5% CO₂ until Day 7 in IVC medium supplemented with 20 ng/mL LIF, 40 ng/mL bFGF (Future Fields) and 20 ng/mL IGF-1. The aggregated bovine embryos were collected on Day 7 and 20 positive ones were shipped to TransOva Genetics. The embryos were transferred to five cows on Day 8 (Day 0=estrus) for further development. Embryos were flushed non-surgically on Day 25 for downstream analysis.

### Genotyping of the Chimeric Embryos

Genomic DNA was isolated from mouse embryos or extraembryonic tissue using QIAamp DNA FFPE Tissue Kit (Qiagen, 56404). A total of 100 ng of genomic DNA was used for each PCR reaction with primers specific for mouse (forward primer: 5′-TGTGGGCAAAGAGGCTTCAT-3′ (SEQ ID NO:7), reverse primer: 5′-CAAAGCTGACTTAGCCTCAG-3′ (SEQ ID NO:8)), and bovine (forward primer: 5′-GATTGTTGATCCCACAGAAGGCAATC-3′ (SEQ ID NO:9), reverse primer: 5′-GAACTTTCAAGCAGCTGAGGCATTTA-3′ (SEQ ID NO:10)) sequences using TikTaq Hot Start PCR Master Mix (Monte Biotech, 560001).

### Immunohistochemistry (IHC)

For IHC analysis, E7.5 and E8.5 mouse chimeric embryos were isolated with their decidua from the uterus of pregnant mice. The embryos observed with obvious GFP signals were fixed in 4% paraformaldehyde and sent to Yale Pathology Tissue Services in 70% ETOH for further embedding, sectioning and IHC staining. In general, the embryos were embedded in paraffin and sectioned to 6 μm sections. Three sections per sample were made with 50 μm between each section. The sections were then treated with GFP antibody (Abcam, ab290). Chromogenic DAB was used as a substrate and the slides were counterstained with hematoxylin.

CRISPR/Cas9-Mediated Gene Editing in bESCs

A CRISPR-Cas9 reporter vector containing the target sequence followed by an out-of-frame sequence of dTomato gene (12) was used to test CRISPR/Cas9-mediated gene editing in bESCs. Briefly, this reporter vector was packaged into lentiviruses and introduced into the GFP-bESC line by spin infection as previously (1). The Sp-Cas9 protein was expressed and purified following the published method (13). To prepare CAS9, the Sp-Cas9 construct was transformed into Escherichia coli strain BL21 (DE3) (New England Biolabs, MA, USA) and induced by 1 mM isopropylb-D-1-thiogalactopyranoside at optical density of 0.6-0.8. After overnight incubation at 23 °C, cells were centrifuged and resuspended in xTractor Buffer containing DNase I, lysozyme solution, and Protease Inhibitor Cocktail (Takara Bio USA, Inc., CA). The supernatant of the sonicated cells was incubated with equilibrated TALON Metal Affinity Resin (Takara Bio USA, Inc., CA, USA) for 20 min on ice. The proteins were eluted from resin with the elution buffer (pH 7.0, 150 mM imidazole, 50 mM NaH2PO4, and 300 mM NaCl). The eluted proteins were concentrated and with buffer exchanged to phosphate-buffered saline (pH 7.4) using Protein Concentrator PES, 3K MWCO (Pierce Biotechnology, PA, USA). The purified Cas9 protein (50 μmol) was used to form the ribonucleoproteins (RNP) complex with sgRNA (250 μmol) in 37 °C for 10 min. Then the RNP was transfected into 5×10⁵ bESCs with CRISPR-Cas9 reporter by electroporation. The fluorescence was observed 48 h after the transduction.

### Electroporation

Bovine ESCs (5×10⁵) in 180 μL TiF2 medium were mixed with 20 μL RNP (50 pmol Cas9 and 250 pmol sgRNA) or 10 μg PB-CAG-eGFP and pCAG-PBase plasmids (Addgene) in 20 μL Opti-MEM and added into 4 mm gene pulser cuvettes (total volume 200 μL). The voltage and capacitance used for electroporation were 200V and 500 μF. After electroporation, the cells were left in cuvette for 30 s and transferred into culture medium.

### Statistics Analysis

One way-ANOVA with Tukey's multiple comparison test or Student's t-test was used for data analysis. The figures were presented as mean±standard deviation (sd). A p-value<0.05(*) or 0.01(**) was considered statistically significant. All data were analyzed with SPSS.

The JY-bESCs are pluripotent stem cells, which are derived from bovine pre-implantation embryos. They exhibit long-term propagation and in vivo and in vitro differentiation abilities. Due to their origins and properties, the cells can be used to study gene regulation and development/differentiation in early bovine embryos. With the single-cell colonization ability and long-term propagation ability, the JY-bESCs are ideal for gene editing especially multi-genes editing to study gene functions and to produce genetically engineered animals by nuclear transfer, germ-line chimera production, tetraploid complementation or artificial gametes induction to improve production efficiency, disease-resistance, and quality/safety of animal products. The JY-bESCs are also good candidates for pharmaceutical protein and cultivated meat production due to their large-scale expansion and differentiation ability without transgenes. The direct differentiation ability, such as the NPC conversion, also allows the establishment of disease models for mechanism study and drug screening. The PGC direct induction from JY-bESCs makes the in vitro produced gametes possible for infertility treatment and faster molecular breeding. The high interspecies chimeric contribution of JY-bESCs would foster to generate biomedical models for human regenerative medicine and zoonotic disease prevention.

### Example 3: Preparation of TiF2 and TiF3 Medium

TiF2 medium is prepared by combining mTeSR-plus media (STEMCELL Technologies, 100-0276) supplemented with 2.5 μM IWR1 (AOBIOUS, A0B33702), 3.3 μM iDOT1L (AOBIOUS, AOB1922), 0.8 μM PD184352 (ApexBio, A1792), 2 μM SU5402 (ApexBio, A3843), 3 μM CHIR99021 (AOBIOUS, AOB3866), 10 μM Forskolin (AOBIOUS, AOB6380), and 1,000 U/ml human LIF (R&D Systems, 7734LF025), with the presence of 10 μM ROCKi (AOBIOUS, AOB3877) when passaging the cells.

TiF3 medium is prepared by combining N2B27 media supplemented with 2.5 μM IWR1 (AOBIOUS, AOB33702), 3.3 μM iDOT1L (AOBIOUS, AOB1922), 0.8 μM PD184352 (ApexBio, A1792), 2 μM SU5402 (ApexBio, A3843), 3 μM CHIR99021 (AOBIOUS, A0B3866), 10 μM Forskolin (AOBIOUS, AOB6380), 1,000 U/ml human LIF (R&D Systems, 7734LF025), 1% BSA, 100 ng/ml bFGF and 1.74 ng/ml TGF (1, with the presence of 10 M ROCKi (AOBIOUS, A0B3877) when passaging the cells.

### Example 4: Derivation and Characterization of bESCs from Bovine In Vitro Blastocysts

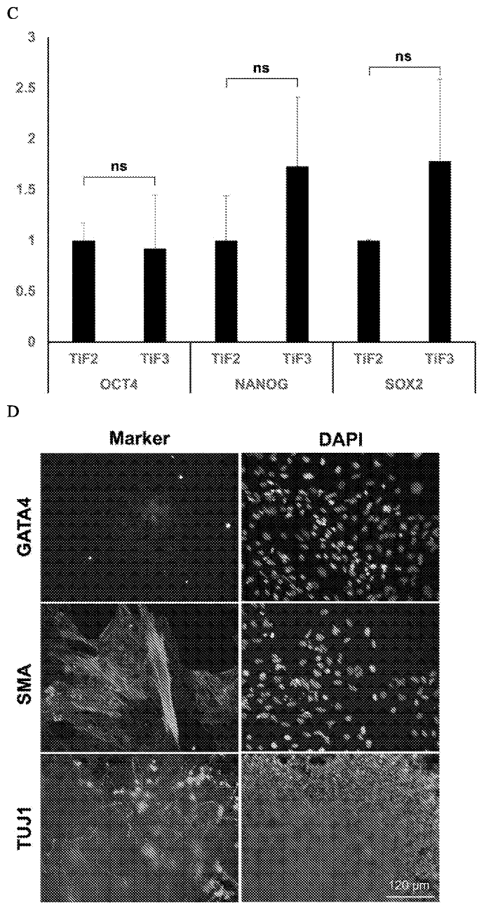

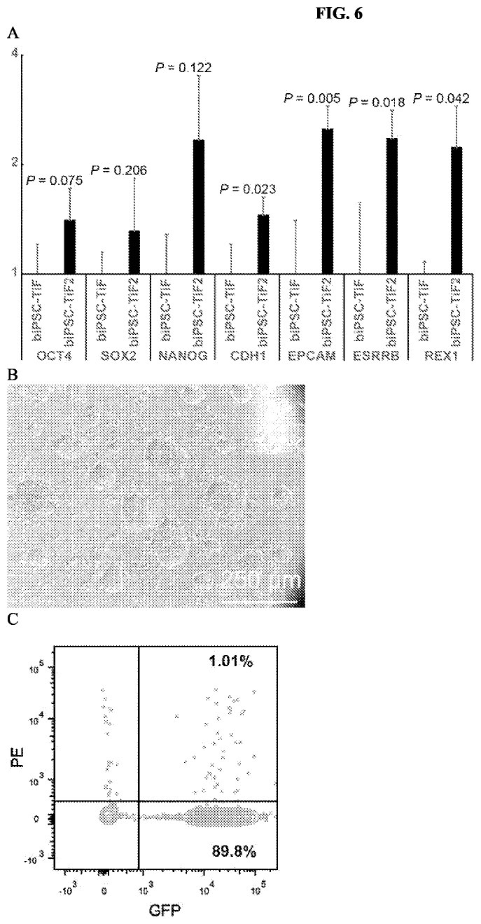

Previously, established bovine induced pluripotent stem cells (biPSCs) (described in WO 2023/122571) in mTeSR plus medium supplemented with H3K79 methyltransferase inhibitor iDOT1L were established tankyrase inhibitor IWR1, MEK inhibitor PD0325901, glycogen synthase inhibitor CHIR99021 (2i)/LIF, and PKA agonist forskolin (TiF medium). Replacing PD0325901 with mild MEK/ERK inhibitors (PD184352 and SU5402), which supported the ground state mouse ESC capable of germline transmission (referred to as the TiF2 medium herein) resulted in thicker biPSC colonies and improved pluripotent gene expression (FIGS. 6A and 6B).

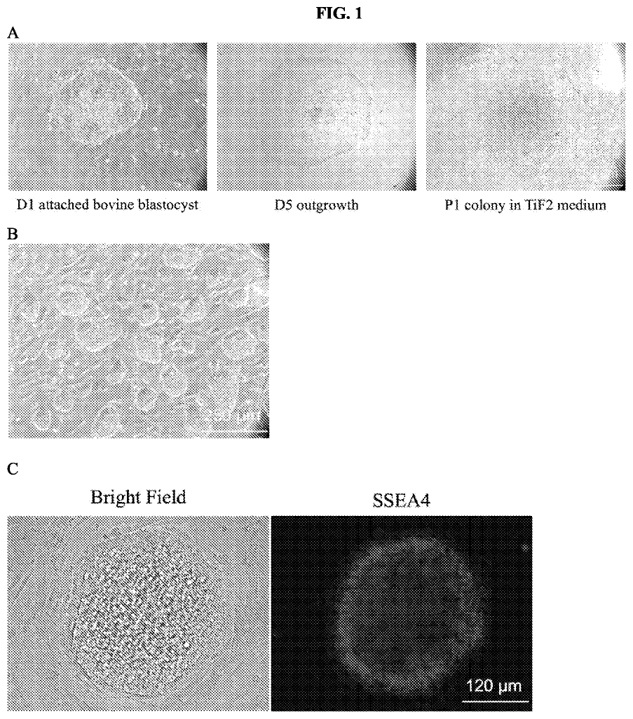

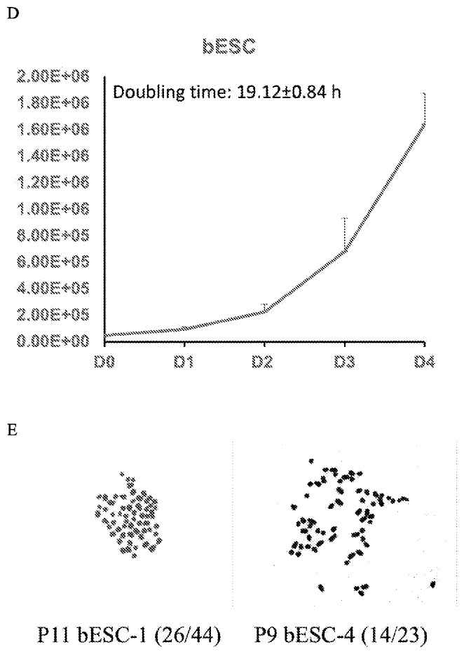

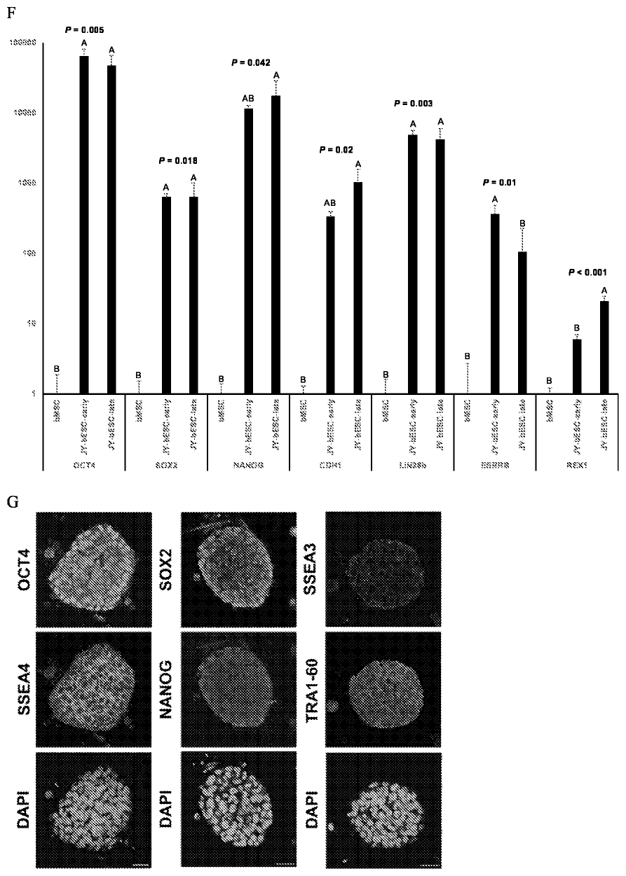

The applicability of TiF2 medium for bESC derivation was tested. By culturing zona pellucida (ZP)-free bovine blastocysts in TiF2 medium on mitomycin C-treated mouse embryonic fibroblasts (MEF), we successfully derived 10 bESC lines (“JY-bESC”) from 15 in vitro fertilized embryos that included 3 female and 7 male lines (FIG. 1A). JY-bESCs exhibited round, compact and dome-shaped colonies (FIG. 1B) and were SSEA4 positive (FIG. 1C). The doubling time of the established JY-bESC lines was 19.1±0.8 h (FIG. 1D), faster than biPSCs (1). JY-bESCs maintained normal karyotype even at high passages (FIG. 1E) and were stably propagated by single cell colonization for over 80 times. Gene-editing by using CRISPR/Cas9 in JY-bESCs was achieved with a 1.0% success rate (FIGS. 6C and 6D).

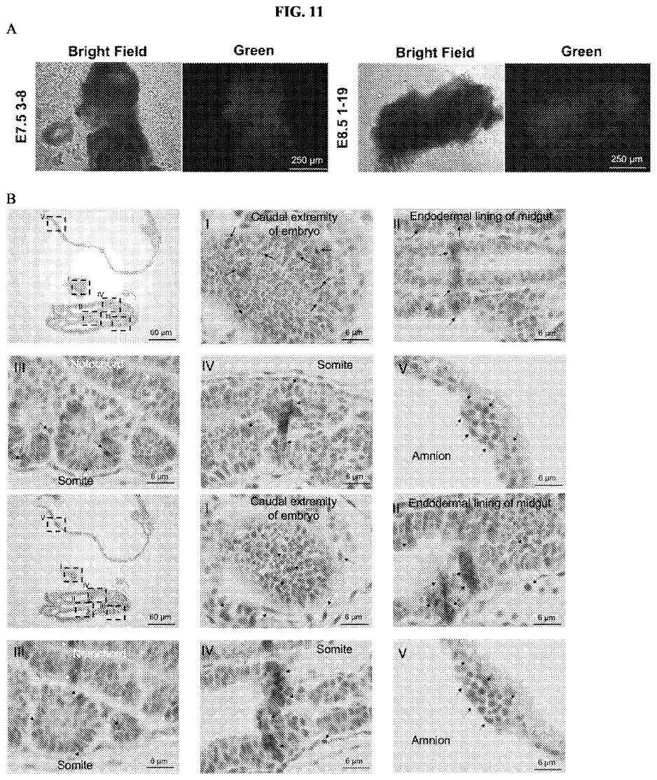

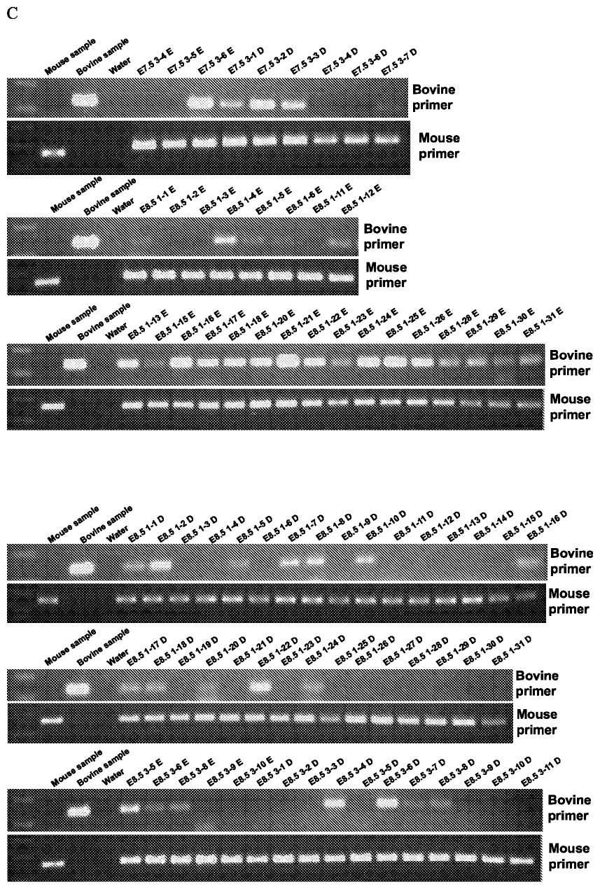

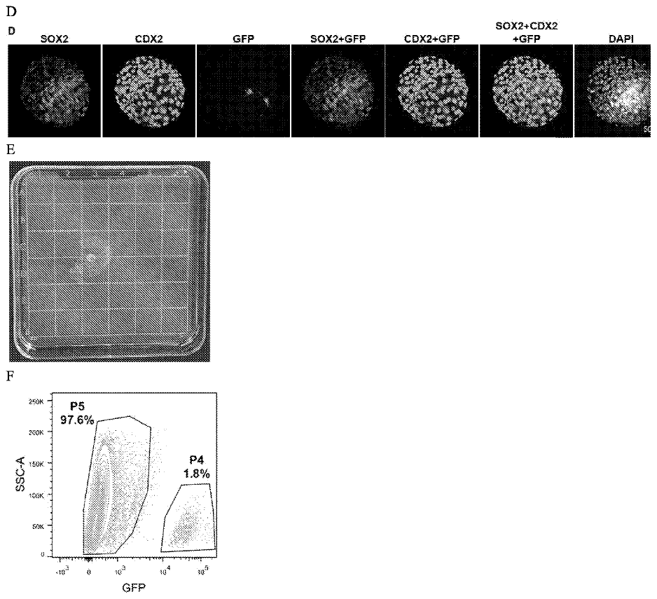

JY-bESCs showed robust expression of pluripotency-related genes (OCT4 [a.k.a. POU5F1], SOX2, NANOG, E-CADHERIN [a.k.a. CDH1], LIN28B, ESRRB and REX1) across all passages examined (FIGS. 1F, 1G and 6F). They were positive for alkaline phosphatase (AP) (FIG. 1H) and ESC surface markers SSEA3, SSEA4 and TRA1-60 (FIGS. 1G and 6F). Bisulfite sequencing revealed demethylation in the OCT4 and NANOG proximal promoters and partial demethylation in the distal enhancer region of OCT4 compared to bovine mesenchymal stem cells (bMSCs) (FIG. 11). After the removal of growth factors, JY-bESCs successfully formed embryoid bodies (EBs) expressing markers for ectoderm, mesoderm, and endoderm (FIGS. 1J, 6E and 6G). When injected into NOD-SCID mice, JY-bESCs formed teratomas with the structures of three germ layers within four to six weeks (FIGS. 1K and 6H). Together, the typical ESC morphology, highly expressed pluripotency-related genes and three-germ layer differentiation both in vitro and in vivo demonstrate successful derivation of bESCs from bovine blastocysts under the TiF2 culture condition.

### Comparative Transcriptomic Analysis of Bovine PSCs

To investigate the molecular signatures among different bovine PSCs, transcriptomic analysis was conducted on JY-bESCs and previously reported bEPSCs (2), bpESC (3) and biPSCs (4) (Table A).

TABLE A
List of Pluripotent Stem Cell and Embryo Samples Retrieved from NCBI

| | | | NAME IN THIS | |
|---|---|---|---|---|
| NCBI_GSM_ID | NCBI_GSE_ID | NCBI_SAMPLE_NAME | STUDY | SPECIES |
| GSM2976768 | GSE110036 | bESC_A_P10 | bpESC_Bogliotti | bovine |
| GSM2976769 | GSE110036 | bESC_A_P13 | bpESC_Bogliotti | bovine |
| GSM2976770 | GSE110036 | bESC_A_P23 | bpESC_Bogliotti | bovine |
| GSM2976771 | GSE110036 | bESC_A_P35 | bpESC_Bogliotti | bovine |
| GSM2976772 | GSE110036 | bESC_A_P45 | bpESC_Bogliotti | bovine |
| GSM2976773 | GSE110036 | bESC_B_P10 | bpESC_Bogliotti | bovine |
| GSM2976774 | GSE110036 | bESC_B_P12 | bpESC_Bogliotti | bovine |
| GSM2976775 | GSE110036 | bESC_B_P24 | bpESC_Bogliotti | bovine |
| GSM2976776 | GSE110036 | bESC_B_P35 | bpESC_Bogliotti | bovine |
| GSM2976777 | GSE110036 | bESC_B_P46 | bpESC_Bogliotti | bovine |
| GSM3721750 | GSE129760 | R_ES_F_rep1 | bEPSC_Zhao | bovine |
| GSM3721751 | GSE129760 | R_ES_F_rep2 | bEPSC_Zhao | bovine |
| GSM3721754 | GSE129760 | R_iPS_rep1 | bEPSC_Zhao | bovine |
| GSM3721755 | GSE129760 | R_iPS_rep2 | bEPSC_Zhao | bovine |
| GSM1290661 | GSE53387 | bEBI5 RNA-Seq | ICM_Bernardo | bovine |
| GSM1290662 | GSE53387 | bEBI7 RNA-Seq | ICM_Bernardo | bovine |
| GSM1290663 | GSE53387 | bEBI8 RNA-Seq | ICM_Bernardo | bovine |
| GSM1290664 | GSE53387 | bERSE12 RNA-Seq | ERSE_Bernardo | bovine |
| GSM1290665 | GSE53387 | bERSE6 RNA-Seq | ERSE_Bernardo | bovine |
| GSM1290666 | GSE53387 | bERSE7 RNA-Seq | ERSE_Bernardo | bovine |
| GSM1290667 | GSE53387 | bAPE17 RNA-Seq | APE_Bernardo | bovine |
| GSM1290668 | GSE53387 | bAPE19 RNA-Seq | APE_Bernardo | bovine |
| GSM1290669 | GSE53387 | bAPE20 RNA-Seq | APE_Bernardo | bovine |
| GSM1265770 | GSE52415 | 16-cell_replicate1 | 16C_Graf | bovine |
| GSM1265771 | GSE52415 | 16-cell_replicate2 | 16C_Graf | bovine |
| GSM1265772 | GSE52415 | 16-cell_replicate3 | 16C_Graf | bovine |
| GSM1265773 | GSE52415 | Blastocyst_replicate1 | BL_Graf | bovine |
| GSM1265774 | GSE52415 | Blastocyst_replicate2 | BL_Graf | bovine |
| GSM1265775 | GSE52415 | Blastocyst_replicate3 | BL_Graf | bovine |
| GSM2976764 | GSE110036 | invivo_BL_A | BL_Bogliotti | bovine |
| GSM2976765 | GSE110036 | invivo_BL_B | BL_Bogliotti | bovine |
| GSM5211161 | GSE169624 | biPSC_1 | biPSC_Pillai | bovine |
| GSM5211162 | GSE169624 | biPSC_2 | biPSC_Pillai | bovine |
| GSM5211163 | GSE169624 | biPSC_3 | biPSC_Pillai | bovine |
| GSM5478616 | GSE180931 | bMSC-1 | MSC-1 | bovine |
| GSM5478617 | GSE180931 | bMSC-2 | MSC-2 | bovine |
| GSM5478618 | GSE180931 | bMSC-3 | MSC-3 | bovine |
| GSM5327365 | GSE174771 | nESC rep1 | hnESC_Bi | human |
| GSM5327366 | GSE174771 | nESC rep2 | hnESC_Bi | human |
| GSM5327367 | GSE174771 | nESC rep3 | hnESC_Bi | human |
| GSM5327368 | GSE174771 | pESC rep1 | hpESC_Bi | human |
| GSM5327369 | GSE174771 | pESC rep2 | hpESC_Bi | human |
| GSM5327370 | GSE174771 | pESC rep3 | hpESC_Bi | human |
| GSM2135526 | GSE89303 | ES1-EPS-1 | hEPSC_Yang | human |
| GSM2135527 | GSE89303 | ES1-EPS-2 | hEPSC_Yang | human |
| GSM4721570 | GSE135989 | FTW-hiPSC#1.r1.RNA- | hFSC_Yu | human |
| | | seq | | |
| GSM4721572 | GSE135989 | FTW-hiPSC#2.r1.RNA- | hFSC_Yu | human |
| | | seq | | |
| GSM3785746 | GSE131551 | HNES1F_p1 | hFSC_Kinoshita | human |
| GSM3785749 | GSE131551 | HNES1FR_p1 | hFSC_Kinoshita | human |
| GSM5911779 | GSE197265 | h_ICM_10_RNA_rep1 | | human |
| GSM5911780 | GSE197265 | h_ICM_15_RNA_rep2 | | human |
| GSM3144752 | GSE114559 | Normal [15274] | | human |
| GSM3144753 | GSE114559 | Normal [15275] | | human |
| GSM3144754 | GSE114559 | Normal [15282] | | human |
| GSM2135522 | GSE80732 | H1-1 | hpESC_Yang | human |
| GSM2135523 | GSE80732 | H1-2 | hpESC_Yang | human |
| GSM2135528 | GSE89303 | mc2i | mESCs_Yang | mouse |
| GSM2135529 | GSE89303 | OG-2i | mESCs_Yang | mouse |
| GSM2433803 | GSE92635 | EpiSC | mpESC1_Liu | mouse |
| GSM2433804 | GSE92635 | ESD-EpiSC | mpESC1_Liu | mouse |
| GSM2433805 | GSE92635 | EpiSCS/F-1 | mpESC2_Liu | mouse |
| GSM2433806 | GSE92635 | EpiSCS/F-2 | mpESC2_Liu | mouse |
| GSM2135530 | GSE89303 | mc6-1 | mEPSC_Yang | mouse |
| GSM2135531 | GSE89303 | OG6-3 | mEPSC_Yang | mouse |
| GSM3787142 | GSE131556 | FS1 [5cdr1_p11] | mFSC_Kinoshita | mouse |
| GSM3787145 | GSE131556 | FS2 [5cdr2_p12] | mFSC_Kinoshita | mouse |
| GSM4039020 | GSE135989 | FTW-ESC#1.r1.RNA-seq | mFSC_Yu | mouse |
| GSM4039022 | GSE135989 | FTW-ESC#2.r1.RNA-seq | mFSC_Yu | mouse |
| GSM3854965 | GSE132254 | WT_ICM_RNA seq_1 | | mouse |
| GSM3854966 | GSE132254 | WT_ICM_RNA seq_2 | | mouse |
| GSM3854967 | GSE132254 | WT_ICM_RNA seq_3 | | mouse |
| GSM3854968 | GSE132254 | WT_ICM_RNA seq_4 | | mouse |
| GSM3854969 | GSE132254 | WT_ICM_RNA seq_5 | | mouse |
| GSM3854980 | GSE132254 | WT_TE_RNA seq_1 | | mouse |
| GSM3854981 | GSE132254 | WT_TE_RNA seq_2 | | mouse |
| GSM3854982 | GSE132254 | WT_TE_RNA seq_3 | | mouse |
| GSM3854983 | GSE132254 | WT_TE_RNA seq_4 | | mouse |
| GSM3854984 | GSE132254 | WT_TE_RNA seq_5 | | mouse |
| GSM5460629 | GSE180307 | WT_flfl [N89_1] | | mouse |
| GSM5460633 | GSE180307 | WT_flfl [N89_7] | | mouse |
| GSM5460635 | GSE180307 | WT_flfl [N89_9] | | mouse |

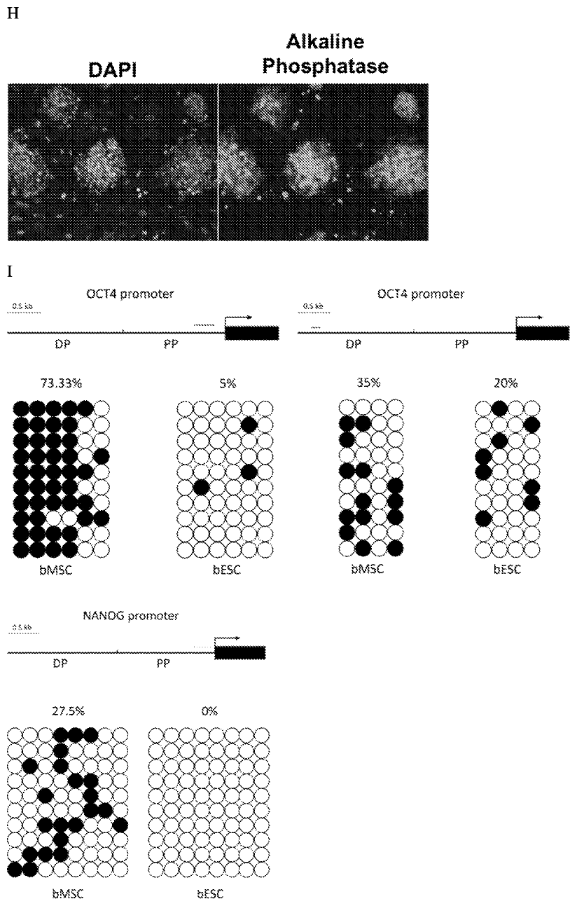

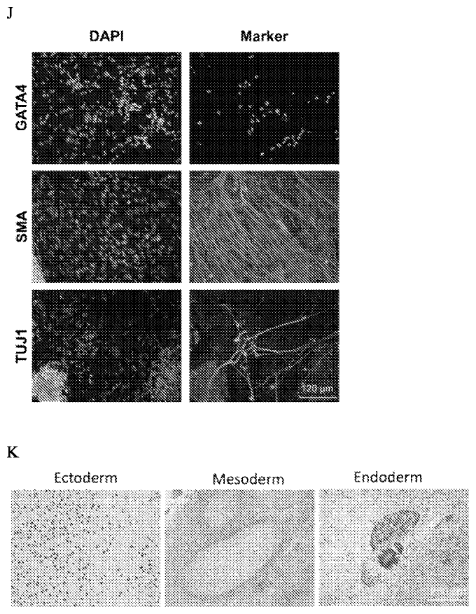

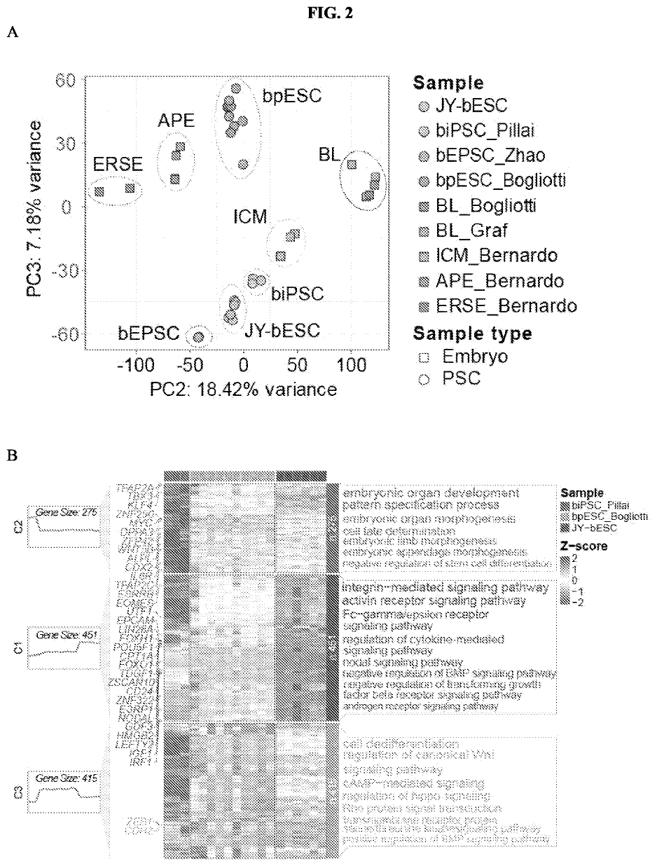

Using principal component analysis (PCA), it was found that six lines of JY-bESCs were clustered between bEPSCs and biPSCs, suggesting their distinct cellular state (FIG. 2A). Next, 643, 1,787, and 2,065 were identified as upregulated and 639, 459, and 528 downregulated genes in JY-bESCs relative to bEPSCs, biPSCs and bpESCs, respectively (Table B).

TABLE B
DEG numbers (Up-regulated and Down-regulated) in comparisons
of JY-bESC vs. biPSC_Pillai, JY-bESC vs. bEPSC_Zhao,
and and JY-bESC vs. bpESC_Bogliotti.

| Comparisons | Up-regulated | Down-regulated |
|---|---|---|
| JY-bESC vs. biPSC_Pillai | 1787 | 459 |
| JY-bESC vs. bEPSC_Zhao | 643 | 639 |
| JY-bESC vs. bpESC_Bogliotti | 2065 | 528 |

Focusing on JY-bESCs, bpESCs and biPSCs, specifically, 451 genes were uniquely expressed in JY-bESCs, 275 in biPSCs, and 415 in bpESCs (FIG. 2B). Enriched GO terms in JY-bESCs included integrin, activin, and Nodal signaling pathways (FIG. 2B). KEGG pathway analysis highlighted ribosome biosynthesis, RAP1, and TGF-beta signaling pathways (Table C).

TABLE C
KEGG pathways enriched by genes specifically expressed in JY-bESCs, biPSCs, and bpESCs

| | | | | | | | | | | Fold | | | |
|---|---|---|---|---|---|---|---|---|---|---|---|---|---|
| Cell | | | | | | | List | Pop | Pop | Enrich- | | | |
| Type | Category | Term | Count | % | PValue | Genes | Total | Hits | Total | ment | Bonferroni | Benjamini | FDR\ |
| JY-bESC | KEGG_PATHWAY | bta03010: | 30 | 6.42398287 | 1.72E−16 | RPL5, | 234 | 168 | 9038 | 6.89713065 | 5.97E−14 | 4.64E−14 | 4.2928349542538646E−14\ |
| | | Ribosome | | | | RPL11, | | | | | | | |
| | | | | | | RPL10L, | | | | | | | |
| | | | | | | RPL36A, | | | | | | | |
| | | | | | | RPS27L, | | | | | | | |
| | | | | | | LOC101906221, | | | | | | | |
| | | | | | | LOC101902561, | | | | | | | |
| | | | | | | RPL10A, | | | | | | | |
| | | | | | | RPL9, | | | | | | | |
| | | | | | | RPL6, | | | | | | | |
| | | | | | | RPL7, | | | | | | | |
| | | | | | | MRPL33, | | | | | | | |
| | | | | | | RPS15A, | | | | | | | |
| | | | | | | LOC112442347, | | | | | | | |
| | | | | | | RPL15, | | | | | | | |
| | | | | | | RPL17, | | | | | | | |
| | | | | | | RPS13, | | | | | | | |
| | | | | | | LOC504858, | | | | | | | |
| | | | | | | RPL21, | | | | | | | |
| | | | | | | RPS6, | | | | | | | |
| | | | | | | RPS3A, | | | | | | | |
| | | | | | | RPL23A, | | | | | | | |
| | | | | | | MRPL23, | | | | | | | |
| | | | | | | LOC100299845, | | | | | | | |
| | | | | | | RPS26, | | | | | | | |
| | | | | | | RPS28, | | | | | | | |
| | | | | | | RPL27A, | | | | | | | |
| | | | | | | RPS20, | | | | | | | |
| | | | | | | RPL22L1, | | | | | | | |
| | | | | | | RPS23 | | | | | | | |
| JY-bESC | KEGG_PATHWAY | bta05171: | 35 | 7.49464668 | 3.06E−14 | RPL5, | 234 | 282 | 9038 | 4.79375038 | 8.21E−12 | 4.11E−12 | 3.805297724719387E−12\ |
| | | Corona | | | | RPL11, | | | | | | | |
| | | virus | | | | RPL10L, | | | | | | | |
| | | disease - | | | | RPL36A, | | | | | | | |
| | | COVID-19 | | | | RPS27L, | | | | | | | |
| | | | | | | TMPRSS2, | | | | | | | |
| | | | | | | LOC101906221, | | | | | | | |
| | | | | | | LOC101902561, | | | | | | | |
| | | | | | | RPL10A, | | | | | | | |
| | | | | | | RPL9, | | | | | | | |
| | | | | | | RPL6, | | | | | | | |
| | | | | | | RPL7, | | | | | | | |
| | | | | | | RPS15A, | | | | | | | |
| | | | | | | LOC112442347, | | | | | | | |
| | | | | | | RPL15, | | | | | | | |
| | | | | | | RPL17, | | | | | | | |
| | | | | | | RPS13, | | | | | | | |
| | | | | | | LOC504858, | | | | | | | |
| | | | | | | ACE, SYK, | | | | | | | |
| | | | | | | RPL21, | | | | | | | |
| | | | | | | RPS6, | | | | | | | |
| | | | | | | CYBB, | | | | | | | |
| | | | | | | RPS3A, | | | | | | | |
| | | | | | | RPL23A, | | | | | | | |
| | | | | | | LOC100299845, | | | | | | | |
| | | | | | | RPS26, | | | | | | | |
| | | | | | | RPS28, | | | | | | | |
| | | | | | | RPL27A, | | | | | | | |
| | | | | | | TRAF6, | | | | | | | |
| | | | | | | RPS20, | | | | | | | |
| | | | | | | RPL22L1, | | | | | | | |
| | | | | | | MASP1, | | | | | | | |
| | | | | | | RPS23, | | | | | | | |
| | | | | | | TLR2 | | | | | | | |
| JY-bESC | KEGG_PATHWAY | bta04216: | 10 | 2.14132762 | 2.50E−06 | MAP1LC3B, | 234 | 47 | 9038 | 8.21785779 | 6.72E−04 | 2.24E−04 | 2.0755706876814703E−4\ |
| | | Ferroptosis | | | | STEAP3, | | | | | | | |
| | | | | | | FTMT, | | | | | | | |
| | | | | | | FTH1, | | | | | | | |
| | | | | | | LOC783993, | | | | | | | |
| | | | | | | SLC40A1, | | | | | | | |
| | | | | | | CYBB, | | | | | | | |
| | | | | | | SLC7A11, | | | | | | | |
| | | | | | | LOC788801, | | | | | | | |
| | | | | | | FTL | | | | | | | |
| JY-bESC | KEGG_PATHWAY | bta04970: | 13 | 2.78372591 | 1.13E−05 | CALM, | 234 | 102 | 9038 | 4.92265795 | 0.00303158 | 7.59E−04 | 7.026074177529523E−4\ |
| | | Salivary | | | | LOC781146, | | | | | | | |
| | | secretion | | | | LYZ1, | | | | | | | |
| | | | | | | GNAS, | | | | | | | |
| | | | | | | LOC112446693, | | | | | | | |
| | | | | | | LYZ, | | | | | | | |
| | | | | | | ADCY7, | | | | | | | |
| | | | | | | LYSB, | | | | | | | |
| | | | | | | LOC617219 | | | | | | | |
| JY-bESC | KEGG_PATHWAY | bta05012: | 21 | 4.49678801 | 3.86E−05 | CALM, | 234 | 282 | 9038 | 2.87625023 | 0.0103345 | 0.00207761 | 0.001923144892304637\ |
| | | Parkinson | | | | COX7B, | | | | | | | |
| | | disease | | | | LOC533307, | | | | | | | |
| | | | | | | NDUFA12, | | | | | | | |
| | | | | | | COX7A2, | | | | | | | |
| | | | | | | SDHC, | | | | | | | |
| | | | | | | COX7C, | | | | | | | |
| | | | | | | ATP5F1E, | | | | | | | |
| | | | | | | SEM1, | | | | | | | |
| | | | | | | TUBA1D, | | | | | | | |
| | | | | | | TUBA1C, | | | | | | | |
| | | | | | | LOC783202, | | | | | | | |
| | | | | | | UBB, | | | | | | | |
| | | | | | | LOC112443216, | | | | | | | |
| | | | | | | PSMA2, | | | | | | | |
| | | | | | | GNAS, | | | | | | | |
| | | | | | | CYCS, | | | | | | | |
| | | | | | | NDUFV2 | | | | | | | |
| JY-bESC | KEGG_PATHWAY | bta04145: | 15 | 3.21199143 | 1.38E−04 | LOC101904667, | 234 | 172 | 9038 | 3.36836613 | 0.03649029 | 0.00619502 | 0.005734427325462683\ |
| | | Phagosome | | | | LOC533307, | | | | | | | |
| | | | | | | ITGB5, | | | | | | | |
| | | | | | | ITGB2, | | | | | | | |
| | | | | | | TAP2, | | | | | | | |
| | | | | | | CYBB, | | | | | | | |
| | | | | | | THBS2, | | | | | | | |
| | | | | | | CTSS, | | | | | | | |
| | | | | | | TUBA1D, | | | | | | | |
| | | | | | | LOC512672, | | | | | | | |
| | | | | | | TUBA1C, | | | | | | | |
| | | | | | | LOC112443216, | | | | | | | |
| | | | | | | MRC1, | | | | | | | |
| | | | | | | TLR2 | | | | | | | |
| JY-bESC | KEGG_PATHWAY | bta05152: | 16 | 3.4261242 | 2.61E−04 | CALM, | 234 | 205 | 9038 | 3.01455076 | 0.06783722 | 0.01003409 | 0.009288063322326241\ |
| | | Tuberculosis | | | | LOC101904667, | | | | | | | |
| | | | | | | FCER1G, | | | | | | | |
| | | | | | | SYK, | | | | | | | |
| | | | | | | NFYB, | | | | | | | |
| | | | | | | ITGB2, | | | | | | | |
| | | | | | | CTSS, | | | | | | | |
| | | | | | | HSPD1, | | | | | | | |
| | | | | | | TRAF6, | | | | | | | |
| | | | | | | MRC1, | | | | | | | |
| | | | | | | CYCS, | | | | | | | |
| | | | | | | CLEC4E, | | | | | | | |
| | | | | | | TLR2 | | | | | | | |
| JY-bESC | KEGG_PATHWAY | bta04512: | 10 | 2.14132762 | 4.56E−04 | COL1A1, | 234 | 89 | 9038 | 4.3397676 | 0.11544151 | 0.01532983 | 0.0141900698201702\ |
| | | ECM-receptor | | | | COL1A2, | | | | | | | |
| | | interaction | | | | ITGB5, | | | | | | | |
| | | | | | | COL6A2, | | | | | | | |
| | | | | | | COL6A1, | | | | | | | |
| | | | | | | TNC, | | | | | | | |
| | | | | | | FN1, | | | | | | | |
| | | | | | | COL6A3, | | | | | | | |
| | | | | | | THBS2, | | | | | | | |
| | | | | | | ITGA9 | | | | | | | |
| JY-bESC | KEGG_PATHWAY | bta04510: | 15 | 3.21199143 | 6.85E−04 | PDGFRA, | 234 | 201 | 9038 | 2.88238296 | 0.16833521 | 0.02047363 | 0.018951427826305562\ |
| | | Focal | | | | ITGB5, | | | | | | | |
| | | adhesion | | | | TNC, | | | | | | | |
| | | | | | | FN1, | | | | | | | |
| | | | | | | IGF1, | | | | | | | |
| | | | | | | THBS2, | | | | | | | |
| | | | | | | VAV1, | | | | | | | |
| | | | | | | COL1A1, | | | | | | | |
| | | | | | | RAP1B, | | | | | | | |
| | | | | | | COL1A2, | | | | | | | |
| | | | | | | COL6A2, | | | | | | | |
| | | | | | | COL6A1, | | | | | | | |
| | | | | | | RAC2, | | | | | | | |
| | | | | | | COL6A3, | | | | | | | |
| | | | | | | ITGA9 | | | | | | | |
| JY-bESC | KEGG_PATHWAY | bta05020: | 18 | 3.85438972 | 9.80E−04 | COX7B, | 234 | 282 | 9038 | 2.46535734 | 0.23182574 | 0.02636094 | 0.02440102131837437\ |
| | | Prion | | | | LOC533307, | | | | | | | |
| | | disease | | | | HSPA1L, | | | | | | | |
| | | | | | | NDUFA12, | | | | | | | |
| | | | | | | CYBB, | | | | | | | |
| | | | | | | COX7A2, | | | | | | | |
| | | | | | | SDHC, | | | | | | | |
| | | | | | | COX7C, | | | | | | | |
| | | | | | | ATP5F1E, | | | | | | | |
| | | | | | | SEM1, | | | | | | | |
| | | | | | | TUBA1D, | | | | | | | |
| | | | | | | TUBA1C, | | | | | | | |
| | | | | | | LOC783202, | | | | | | | |
| | | | | | | LOC112443216, | | | | | | | |
| | | | | | | PSMA2, | | | | | | | |
| | | | | | | RAC2, | | | | | | | |
| | | | | | | CYCS, | | | | | | | |
| | | | | | | NDUFV2 | | | | | | | |
| JY-bESC | KEGG_PATHWAY | bta05415: | 15 | 3.21199143 | 0.001318867 | COX7B, | 234 | 215 | 9038 | 2.6946929 | 0.2988331 | 0.03225229 | 0.02985434872044207\ |
| | | Diabetic | | | | ACE, | | | | | | | |
| | | cardiomyopathy | | | | NDUFA12, | | | | | | | |
| | | | | | | CYBB, | | | | | | | |
| | | | | | | COX7A2, | | | | | | | |
| | | | | | | SDHC, | | | | | | | |
| | | | | | | COX7C, | | | | | | | |
| | | | | | | ATP5F1E, | | | | | | | |
| | | | | | | TGFBR2, | | | | | | | |
| | | | | | | COL1A1, | | | | | | | |
| | | | | | | COL3A1, | | | | | | | |
| | | | | | | COL1A2, | | | | | | | |
| | | | | | | LOC783202, | | | | | | | |
| | | | | | | RAC2, | | | | | | | |
| | | | | | | NDUFV2 | | | | | | | |
| JY-bESC | KEGG_PATHWAY | bta04015: | 15 | 3.21199143 | 0.001440689 | CALM, | 234 | 217 | 9038 | 2.66985702 | 0.32146872 | 0.03229544 | 0.02989429097424148\ |
| | | Rap1 | | | | APBB1IP, | | | | | | | |
| | | signaling | | | | RAP1B, | | | | | | | |
| | | pathway | | | | CSF1R, | | | | | | | |
| | | | | | | PDGFRA, | | | | | | | |
| | | | | | | ITGB2, | | | | | | | |
| | | | | | | GNAS, | | | | | | | |
| | | | | | | RAC2, | | | | | | | |
| | | | | | | IGF1, | | | | | | | |
| | | | | | | PFN1, | | | | | | | |
| | | | | | | ADCY7, | | | | | | | |
| | | | | | | VAV1 | | | | | | | |
| JY-bESC | KEGG_PATHWAY | bta05010: | 22 | 4.71092077 | 0.001601618 | CALM, | 234 | 402 | 9038 | 2.1137475 | 0.35025841 | 0.03314117 | 0.030677139234859648\ |
| | | Alzheimer | | | | COX7B, | | | | | | | |
| | | disease | | | | LOC533307, | | | | | | | |
| | | | | | | NDUFA12, | | | | | | | |
| | | | | | | WNT5A, | | | | | | | |
| | | | | | | CYBB, | | | | | | | |
| | | | | | | COX7A2, | | | | | | | |
| | | | | | | SDHC, | | | | | | | |
| | | | | | | COX7C, | | | | | | | |
| | | | | | | DKK2, | | | | | | | |
| | | | | | | ATP5F1E, | | | | | | | |
| | | | | | | SEM1, | | | | | | | |
| | | | | | | TUBA1D, | | | | | | | |
| | | | | | | TUBA1C, | | | | | | | |
| | | | | | | LOC783202, | | | | | | | |
| | | | | | | LOC112443216, | | | | | | | |
| | | | | | | PSMA2, | | | | | | | |
| | | | | | | CYCS, | | | | | | | |
| | | | | | | NDUFV2 | | | | | | | |
| JY-bESC | KEGG_PATHWAY | bta00190: | 11 | 2.35546039 | 0.003827284 | LOC101904667, | 234 | 143 | 9038 | 2.97107166 | 0.64353335 | 0.06656183 | 0.06161299896263864\ |
| | | Oxidative | | | | COX7B, | | | | | | | |
| | | phosphorylation | | | | LOC783202, | | | | | | | |
| | | | | | | NDUFA12, | | | | | | | |
| | | | | | | CYCS, | | | | | | | |
| | | | | | | SDHC, | | | | | | | |
| | | | | | | COX7A2, | | | | | | | |
| | | | | | | NDUFV2, | | | | | | | |
| | | | | | | COX7C, | | | | | | | |
| | | | | | | ATP5MG, | | | | | | | |
| | | | | | | ATP5F1E | | | | | | | |
| JY-bESC | KEGG_PATHWAY | bta05022: | 24 | 5.1391863 | 0.003847155 | CALM, | 234 | 490 | 9038 | 1.89178441 | 0.64544097 | 0.06656183 | 0.06161299896263864\ |
| | | Pathways of | | | | COX7B, | | | | | | | |
| | | neurodegeneration - | | | | LOC533307, | | | | | | | |
| | | multiple | | | | NDUFA12, | | | | | | | |
| | | diseases | | | | WNT5A, | | | | | | | |
| | | | | | | CYBB, | | | | | | | |
| | | | | | | COX7A2, | | | | | | | |
| | | | | | | SDHC, | | | | | | | |
| | | | | | | COX7C, | | | | | | | |
| | | | | | | DKK2, | | | | | | | |
| | | | | | | ATP5F1E, | | | | | | | |
| | | | | | | SEM1, | | | | | | | |
| | | | | | | TUBA1D, | | | | | | | |
| | | | | | | TUBA1C, | | | | | | | |
| | | | | | | MAP1LC3B, | | | | | | | |
| | | | | | | LOC783202, | | | | | | | |
| | | | | | | UBB, | | | | | | | |
| | | | | | | LOC112443216, | | | | | | | |
| | | | | | | PSMA2, | | | | | | | |
| | | | | | | CYCS, | | | | | | | |
| | | | | | | NDUFV2 | | | | | | | |
| JY-bESC | KEGG_PATHWAY | bta05134: | 7 | 1.49892934 | 0.003962748 | EEF1A1, | 234 | 59 | 9038 | 4.58250036 | 0.65633806 | 0.06656183 | 0.06161299896263864\ |
| | | Legionellosis | | | | ARF1, | | | | | | | |
| | | | | | | HSPA1L, | | | | | | | |
| | | | | | | ITGB2, | | | | | | | |
| | | | | | | CYCS, | | | | | | | |
| | | | | | | TLR2, | | | | | | | |
| | | | | | | HSPD1 | | | | | | | |
| JY-bESC | KEGG_PATHWAY | bta04611: | 10 | 2.14132762 | 0.00420651 | RAP1B, | 234 | 122 | 9038 | 3.16589603 | 0.67823629 | 0.06656183 | 0.06161299896263864\ |
| | | Platelet | | | | APBB1IP, | | | | | | | |
| | | activation | | | | COL1A1, | | | | | | | |
| | | | | | | COL3A1, | | | | | | | |
| | | | | | | FCER1G, | | | | | | | |
| | | | | | | COL1A2, | | | | | | | |
| | | | | | | SYK, | | | | | | | |
| | | | | | | GNAS, | | | | | | | |
| | | | | | | ADCY7, | | | | | | | |
| | | | | | | FERMT3 | | | | | | | |
| JY-bESC | KEGG_PATHWAY | bta04625: | 9 | 1.92719486 | 0.005635434 | CALM, | 234 | 105 | 9038 | 3.31062271 | 0.78133607 | 0.08421843 | 0.07795683435669136\ |
| | | C-type | | | | FCER1G, | | | | | | | |
| | | lectin receptor | | | | SYK, | | | | | | | |
| | | signaling | | | | CLEC6A, | | | | | | | |
| | | pathway | | | | IRF1, | | | | | | | |
| | | | | | | CLEC4E | | | | | | | |
| JY-bESC | KEGG_PATHWAY | bta04650: | 11 | 2.35546039 | 0.0073464260.00758 | TYROBP, | 234 | 157 | 9038 | 2.70613534 | 0.86241085 | 0.10195784 | 0.09437733452892098\ |
| | | Natural | | | | FCER1G, | | | | | | | |
| | | killer cell | | | | SYK, | | | | | | | |
| | | mediated | | | | ITGB2, | | | | | | | |
| | | cytotoxicity | | | | RAC2, | | | | | | | |
| | | | | | | LOC784541, | | | | | | | |
| | | | | | | LOC100850276, | | | | | | | |
| | | | | | | LOC104968444, | | | | | | | |
| | | | | | | LOC783508, | | | | | | | |
| | | | | | | VAV1, | | | | | | | |
| | | | | | | ULBP21 | | | | | | | |
| JY-bESC | KEGG_PATHWAY | bta05132: | 15 | 3.21199143 | 0.007580509 | ARF1, | 234 | 261 | 9038 | 2.21976619 | 0.87086864 | 0.10195784 | 0.09437733452892098\ |
| | | Salmonella | | | | LOC533307, | | | | | | | |
| | | infection | | | | LOC784541, | | | | | | | |
| | | | | | | DYNLL1, | | | | | | | |
| | | | | | | TUBA1D, | | | | | | | |
| | | | | | | TUBA1C, | | | | | | | |
| | | | | | | CYTH4, | | | | | | | |
| | | | | | | LOC112443216, | | | | | | | |
| | | | | | | TRAF6, | | | | | | | |
| | | | | | | CYCS, | | | | | | | |
| | | | | | | NCKAP1L, | | | | | | | |
| | | | | | | LOC104968444, | | | | | | | |
| | | | | | | PFN1, | | | | | | | |
| | | | | | | SKP1, | | | | | | | |
| | | | | | | TLR2 | | | | | | | |
| JY-bESC | KEGG_PATHWAY | bta05417: | 14 | 2.99785867 | 0.008894983 | CALM, | 234 | 239 | 9038 | 2.26248972 | 0.90959499 | 0.11394049 | 0.10546908054471513\ |
| | | Lipid and | | | | RAP1B, | | | | | | | |
| | | atherosclerosis | | | | HSPA1L, | | | | | | | |
| | | | | | | TRAF6, | | | | | | | |
| | | | | | | LOC784541, | | | | | | | |
| | | | | | | CYCS, | | | | | | | |
| | | | | | | CYBB, | | | | | | | |
| | | | | | | LOC104968444, | | | | | | | |
| | | | | | | VAV1, | | | | | | | |
| | | | | | | HSPD1, | | | | | | | |
| | | | | | | TLR2 | | | | | | | |
| JY-bESC | KEGG_PATHWAY | bta05014: | 19 | 4.06852248 | 0.009983691 | HNRNPA3, | 234 | 383 | 9038 | 1.91606972 | 0.93273458 | 0.12207332 | 0.11299723294140694\ |
| | | Amyotrophic | | | | COX7B, | | | | | | | |
| | | lateral | | | | LOC533307, | | | | | | | |
| | | sclerosis | | | | NDUFA12, | | | | | | | |
| | | | | | | COX7A2, | | | | | | | |
| | | | | | | SDHC, | | | | | | | |
| | | | | | | COX7C, | | | | | | | |
| | | | | | | ATP5F1E, | | | | | | | |
| | | | | | | SEM1, | | | | | | | |
| | | | | | | TUBA1D, | | | | | | | |
| | | | | | | TUBA1C, | | | | | | | |
| | | | | | | MAP1LC3B, | | | | | | | |
| | | | | | | LOC783202, | | | | | | | |
| | | | | | | LOC112443216, | | | | | | | |
| | | | | | | PSMA2, | | | | | | | |
| | | | | | | CYCS, | | | | | | | |
| | | | | | | PFN1, | | | | | | | |
| | | | | | | HNRNPA1, | | | | | | | |
| | | | | | | NDUFV2 | | | | | | | |
| JY-bESC | KEGG_PATHWAY | bta04350: | 8 | 1.7130621 | 0.010596469 | GREM1, | 234 | 94 | 9038 | 3.28714312 | 0.94305447 | 0.12393262 | 0.11471829753129652\ |
| | | TGF-beta | | | | LOC100336971, | | | | | | | |
| | | signaling | | | | INHBA, | | | | | | | |
| | | pathway | | | | LEFTY2, | | | | | | | |
| | | | | | | NODAL, | | | | | | | |
| | | | | | | FBN1, | | | | | | | |
| | | | | | | TGFBR2, | | | | | | | |
| | | | | | | SKP1 | | | | | | | |
| JY-bESC | KEGG_PATHWAY | bta04924: | 7 | 1.49892934 | 0.011124175 | CALM, | 234 | 73 | 9038 | 3.70366468 | 0.95066746 | 0.12468346 | 0.11541331165070136\ |
| | | Renin | | | | EDNRA, | | | | | | | |
| | | secretion | | | | ACE, | | | | | | | |
| | | | | | | GNAS | | | | | | | |
| JY-bESC | KEGG_PATHWAY | bta05133: | 7 | 1.49892934 | 0.015103374 | CALM, | 234 | 78 | 9038 | 3.46625027 | 0.98332427 | 0.1625123 | 0.15042960386403353\ |
| | | Pertussis | | | | TRAF6, | | | | | | | |
| | | | | | | IRF1, | | | | | | | |
| | | | | | | ITGB2 | | | | | | | |
| JY-bESC | KEGG_PATHWAY | bta05016: | 16 | 3.4261242 | 0.017887318 | COX7B, | 234 | 319 | 9038 | 1.93725049 | 0.99221256 | 0.18506494 | 0.17130546828911\ |
| | | Huntington | | | | LOC533307, | | | | | | | |
| | | disease | | | | NDUFA12, | | | | | | | |
| | | | | | | COX7A2, | | | | | | | |
| | | | | | | SDHC, | | | | | | | |
| | | | | | | COX7C, | | | | | | | |
| | | | | | | ATP5F1E, | | | | | | | |
| | | | | | | SEM1, | | | | | | | |
| | | | | | | TUBA1D, | | | | | | | |
| | | | | | | TUBA1C, | | | | | | | |
| | | | | | | LOC783202, | | | | | | | |
| | | | | | | LOC112443216, | | | | | | | |
| | | | | | | PSMA2, | | | | | | | |
| | | | | | | CYCS, | | | | | | | |
| | | | | | | NDUFV2, | | | | | | | |
| | | | | | | POLR2K | | | | | | | |
| JY-bESC | KEGG_PATHWAY | bta04714: | 13 | 2.78372591 | 0.021421307 | CPT1A, | 234 | 240 | 9038 | 2.09212963 | 0.99704704 | 0.21341969 | 0.1975520558269339\ |
| | | Thermogenesis | | | | COX7B, | | | | | | | |
| | | | | | | NDUFA12, | | | | | | | |
| | | | | | | RPS6, | | | | | | | |
| | | | | | | COX7A2, | | | | | | | |
| | | | | | | SDHC, | | | | | | | |
| | | | | | | COX7C, | | | | | | | |
| | | | | | | ADCY7, | | | | | | | |
| | | | | | | ATP5F1E, | | | | | | | |
| | | | | | | LOC783202, | | | | | | | |
| | | | | | | GNAS, | | | | | | | |
| | | | | | | NDUFV2, | | | | | | | |
| | | | | | | ATP5MG | | | | | | | |
| JY-bESC | KEGG_PATHWAY | bta04750: | 8 | 1.7130621 | 0.023383294 | CALM, | 234 | 110 | 9038 | 2.80901321 | 0.99827894 | 0.22464665 | 0.20794429387654753\ |
| | | Inflammatory | | | | IL1R1, | | | | | | | |
| | | mediator | | | | GNAS, | | | | | | | |
| | | regulation of | | | | IGF1, | | | | | | | |
| | | TRP channels | | | | ADCY7 | | | | | | | |
| JY-bESC | KEGG_PATHWAY | bta05163: | 13 | 2.78372591 | 0.028997601 | CALM, | 234 | 251 | 9038 | 2.00044267 | 0.99963503 | 0.26897775 | 0.24897940099961466\ |
| | | Human | | | | LOC512672, | | | | | | | |
| | | cytomegalovirus | | | | PDGFRA, | | | | | | | |
| | | infection | | | | IL1R1, | | | | | | | |
| | | | | | | TAP2, | | | | | | | |
| | | | | | | GNAS, | | | | | | | |
| | | | | | | RAC2, | | | | | | | |
| | | | | | | CYCS, | | | | | | | |
| | | | | | | ADCY7 | | | | | | | |
| JY-bESC | KEGG_PATHWAY | bta04540: | 7 | 1.49892934 | 0.031250883 | TUBA1D, | 234 | 92 | 9038 | 2.93877741 | 0.99980464 | 0.27241475 | 0.25216086735873705\ |
| | | Gap | | | | TUBA1C, | | | | | | | |
| | | junction | | | | PDGFRA, | | | | | | | |
| | | | | | | LOC533307, | | | | | | | |
| | | | | | | LOC112443216, | | | | | | | |
| | | | | | | GNAS, | | | | | | | |
| | | | | | | ADCY7 | | | | | | | |
| JY-bESC | KEGG_PATHWAY | bta05146: | 8 | 1.7130621 | 0.031393522 | COL1A1, | 234 | 117 | 9038 | 2.64095259 | 0.99981222 | 0.27241475 | 0.25216086735873705\ |
| | | Amoebiasis | | | | COL3A1, | | | | | | | |
| | | | | | | COL1A2, | | | | | | | |
| | | | | | | IL1R1, | | | | | | | |
| | | | | | | ITGB2, | | | | | | | |
| | | | | | | GNAS, | | | | | | | |
| | | | | | | FN1, | | | | | | | |
| | | | | | | TLR2 | | | | | | | |
| JY-bESC | KEGG_PATHWAY | bta04744: | 4 | 0.85653105 | 0.034483817 | CALM | 234 | 28 | 9038 | 5.51770452 | 0.99992051 | 0.28987959 | 0.2683272025957643\ |
| | | Phototransduction | | | | | | | | | | | |
| JY-bESC | KEGG_PATHWAY | bta05205: | 11 | 2.35546039 | 0.038257096 | COL1A1, | 234 | 204 | 9038 | 2.08266298 | 0.99997228 | 0.30458263 | 0.281937080221436\ |
| | | Proteoglycans | | | | COL1A2, | | | | | | | |
| | | in cancer | | | | ITGB5, | | | | | | | |
| | | | | | | RPS6, | | | | | | | |
| | | | | | | WNT5A, | | | | | | | |
| | | | | | | TWIST2, | | | | | | | |
| | | | | | | FN1, | | | | | | | |
| | | | | | | HCLS1, | | | | | | | |
| | | | | | | IGF1, | | | | | | | |
| | | | | | | VAV1, | | | | | | | |
| | | | | | | TLR2 | | | | | | | |
| JY-bESC | KEGG_PATHWAY | bta05165: | 16 | 3.4261242 | 0.038497433 | LOC101904667, | 234 | 352 | 9038 | 1.75563326 | 0.99997408 | 0.30458263 | 0.281937080221436\ |
| | | Human | | | | ITGB5, | | | | | | | |
| | | papillomavirus | | | | WNT5A, | | | | | | | |
| | | infection | | | | TNC, | | | | | | | |
| | | | | | | FN1, | | | | | | | |
| | | | | | | THBS2, | | | | | | | |
| | | | | | | FOXO1, | | | | | | | |
| | | | | | | COL1A1, | | | | | | | |
| | | | | | | LOC512672, | | | | | | | |
| | | | | | | COL1A2, | | | | | | | |
| | | | | | | IRF1, | | | | | | | |
| | | | | | | COL6A2, | | | | | | | |
| | | | | | | COL6A1, | | | | | | | |
| | | | | | | GNAS, | | | | | | | |
| | | | | | | COL6A3, | | | | | | | |
| | | | | | | ITGA9 | | | | | | | |
| JY-bESC | KEGG_PATHWAY | bta04971: | 6 | 1.28479657 | 0.046016835 | CALM, | 234 | 76 | 9038 | 3.04925776 | 0.99999686 | 0.3251051 | 0.3009337151164872\ |
| | | Gastric | | | | GNAS, | | | | | | | |
| | | acid secretion | | | | ADCY7 | | | | | | | |
| JY-bESC | KEGG_PATHWAY | bta04916: | 7 | 1.49892934 | 0.047909212 | CALM, | 234 | 102 | 9038 | 2.65066197 | 0.99999816 | 0.3251051 | 0.3009337151164872\ |
| | | Melanogenesis | | | | WNT5A, | | | | | | | |
| | | | | | | GNAS, | | | | | | | |
| | | | | | | ADCY7 | | | | | | | |
| JY-bESC | KEGG_PATHWAY | bta05170: | 12 | 2.56959315 | 0.048219113 | CALM, | 234 | 242 | 9038 | 1.91523628 | 0.99999832 | 0.3251051 | 0.3009337151164872\ |
| | | Human | | | | LOC512672, | | | | | | | |
| | | immunodeficiency | | | | TRAF6, | | | | | | | |
| | | virus 1 | | | | RAC2, | | | | | | | |
| | | infection | | | | TAP2, | | | | | | | |
| | | | | | | CYCS, | | | | | | | |
| | | | | | | SKP1, | | | | | | | |
| | | | | | | TLR2 | | | | | | | |
| JY-bESC | KEGG_PATHWAY | bta05214: | 6 | 1.28479657 | 0.048223652 | CALM, | 234 | 77 | 9038 | 3.00965701 | 0.99999832 | 0.3251051 | 0.3009337151164872\ |
| | | Glioma | | | | PDGFRA, | | | | | | | |
| | | | | | | IGF1 | | | | | | | |
| JY-bESC | KEGG_PATHWAY | bta04024: | 12 | 2.56959315 | 0.04971767 | CALM, | 234 | 243 | 9038 | 1.90735465 | 0.9999989 | 0.3251051 | 0.3009337151164872\ |
| | | cAMP | | | | RAP1B, | | | | | | | |
| | | signaling | | | | GLP1R, | | | | | | | |
| | | pathway | | | | EDNRA, | | | | | | | |
| | | | | | | RAC2, | | | | | | | |
| | | | | | | GNAS, | | | | | | | |
| | | | | | | PDE4B, | | | | | | | |
| | | | | | | ADCY7, | | | | | | | |
| | | | | | | VAV1 | | | | | | | |
| JY-bESC | KEGG_PATHWAY | bta04922: | 7 | 1.49892934 | 0.049826713 | CALM, | 234 | 103 | 9038 | 2.62492739 | 0.99999893 | 0.3251051 | 0.3009337151164872\ |
| | | Glucagon | | | | CPT1A, | | | | | | | |
| | | signaling | | | | GNAS, | | | | | | | |
| | | pathway | | | | FOXO1 | | | | | | | |
| JY-bESC | KEGG_PATHWAY | bta04933: | 7 | 1.49892934 | 0.049826713 | COL1A1, | 234 | 103 | 9038 | 2.62492739 | 0.99999893 | 0.3251051 | 0.3009337151164872\ |
| | | AGE-RAGE | | | | COL3A1, | | | | | | | |
| | | signaling | | | | COL1A2, | | | | | | | |
| | | pathway in | | | | FN1, | | | | | | | |
| | | diabetic | | | | CYBB, | | | | | | | |
| | | complications | | | | FOXO1, | | | | | | | |
| | | | | | | TGFBR2 | | | | | | | |
| JY-bESC | KEGG_PATHWAY | bta04974: | 8 | 1.7130621 | 0.050759904 | COL1A1, | 234 | 130 | 9038 | 2.37685733 | 0.99999918 | 0.3251051 | 0.3009337151164872\ |
| | | Protein | | | | COL3A1, | | | | | | | |
| | | digestion and | | | | COL1A2, | | | | | | | |
| | | absorption | | | | COL6A2, | | | | | | | |
| | | | | | | COL12A1, | | | | | | | |
| | | | | | | COL6A1, | | | | | | | |
| | | | | | | COL6A3, | | | | | | | |
| | | | | | | COL8A1 | | | | | | | |
| JY-bESC | KEGG_PATHWAY | bta04151: | 16 | 3.4261242 | 0.065473002 | CSF1R, | 234 | 379 | 9038 | 1.63056176 | 0.99999999 | 0.40005735 | 0.370313311330753\ |
| | | PI3K-Akt | | | | PDGFRA, | | | | | | | |
| | | signaling | | | | ITGB5, | | | | | | | |
| | | pathway | | | | SYK, | | | | | | | |
| | | | | | | RPS6, | | | | | | | |
| | | | | | | TNC, | | | | | | | |
| | | | | | | FN1, | | | | | | | |
| | | | | | | IGF1, | | | | | | | |
| | | | | | | THBS2, | | | | | | | |
| | | | | | | COL1A1, | | | | | | | |
| | | | | | | COL1A2, | | | | | | | |
| | | | | | | COL6A2, | | | | | | | |
| | | | | | | COL6A1, | | | | | | | |
| | | | | | | COL6A3, | | | | | | | |
| | | | | | | ITGA9, | | | | | | | |
| | | | | | | TLR2 | | | | | | | |
| JY-bESC | KEGG_PATHWAY | bta04380: | 8 | 1.7130621 | 0.065768121 | CSF1R, | 234 | 138 | 9038 | 2.2390685 | 0.99999999 | 0.40005735 | 0.370313311330753\ |
| | | Osteoclast | | | | TYROBP, | | | | | | | |
| | | differentiation | | | | SPI1, | | | | | | | |
| | | | | | | SYK, | | | | | | | |
| | | | | | | IL1R1, | | | | | | | |
| | | | | | | CTSK, | | | | | | | |
| | | | | | | TRAF6, | | | | | | | |
| | | | | | | TGFBR2 | | | | | | | |
| JY-bESC | KEGG_PATHWAY | bta05200: | 21 | 4.49678801 | 0.067585629 | CALM, | 234 | 543 | 9038 | 1.49374321 | 0.99999999 | 0.40005735 | 0.370313311330753\ |
| | | Pathways | | | | CSF1R, | | | | | | | |
| | | in cancer | | | | PDGFRA, | | | | | | | |
| | | | | | | SPI1, | | | | | | | |
| | | | | | | WNT5A, | | | | | | | |
| | | | | | | FN1, | | | | | | | |
| | | | | | | IGF1, | | | | | | | |
| | | | | | | ADCY7, | | | | | | | |
| | | | | | | RASGRP4, | | | | | | | |
| | | | | | | FOXO1, | | | | | | | |
| | | | | | | TGFBR2, | | | | | | | |
| | | | | | | EDNRA, | | | | | | | |
| | | | | | | LOC785216, | | | | | | | |
| | | | | | | TRAF6, | | | | | | | |
| | | | | | | GNAS, | | | | | | | |
| | | | | | | RAC2, | | | | | | | |
| | | | | | | CYCS, | | | | | | | |
| | | | | | | SKP1 | | | | | | | |
| JY-bESC | KEGG_PATHWAY | bta04978: | 5 | 1.07066381 | 0.068511389 | FTH1, | 234 | 60 | 9038 | 3.21866097 | 0.99999999 | 0.40005735 | 0.370313311330753\ |
| | | Mineral | | | | LOC783993, | | | | | | | |
| | | absorption | | | | SLC40A1, | | | | | | | |
| | | | | | | LOC788801, | | | | | | | |
| | | | | | | FTL | | | | | | | |
| JY-bESC | KEGG_PATHWAY | bta04270: | 8 | 1.7130621 | 0.069898497 | CALM, | 234 | 140 | 9038 | 2.20708181 | 1 | 0.40005735 | 0.370313311330753\ |
| | | Vascular | | | | EDNRA, | | | | | | | |
| | | smooth muscle | | | | ADM2, | | | | | | | |
| | | contraction | | | | GNAS, | | | | | | | |
| | | | | | | ADCY7 | | | | | | | |
| JY-bESC | KEGG_PATHWAY | bta04371: | 8 | 1.7130621 | 0.072020699 | CALM, | 234 | 141 | 9038 | 2.19142874 | 1 | 0.403616 | 0.37360737854135223\ |
| | | Apelin | | | | MYLA, | | | | | | | |
| | | signaling | | | | MAP1C3B, | | | | | | | |
| | | pathway | | | | RPS6, | | | | | | | |
| | | | | | | ADCY7 | | | | | | | |
| JY-bESC | KEGG_PATHWAY | bta04210: | 8 | 1.7130621 | 0.076379141 | TUBA1D, | 234 | 143 | 9038 | 2.16077939 | 1 | 0.4193059 | 0.38813073783310126\ |
| | | Apoptosis | | | | TUBA1C, | | | | | | | |
| | | | | | | LOC112443216, | | | | | | | |
| | | | | | | CTSK, | | | | | | | |
| | | | | | | LOC784541, | | | | | | | |
| | | | | | | CYCS, | | | | | | | |
| | | | | | | LOC104968444, | | | | | | | |
| | | | | | | CTSS | | | | | | | |
| JY-bESC | KEGG_PATHWAY | bta05418: | 8 | 1.7130621 | 0.080889469 | CALM, | 234 | 145 | 9038 | 2.13097554 | 1 | 0.43518534 | 0.40282955431267914\ |
| | | Fluid | | | | LOC785216, | | | | | | | |
| | | shear stress and | | | | IL1R1, | | | | | | | |
| | | atherosclerosis | | | | RAC2, | | | | | | | |
| | | | | | | ASS1 | | | | | | | |
| JY-bESC | KEGG_PATHWAY | bta04612: | 6 | 1.28479657 | 0.085577718 | LOC512672, | 234 | 91 | 9038 | 2.54663285 | 1 | 0.45138051 | 0.4178206232265254\ |
| | | Antigen | | | | HSPA1L, | | | | | | | |
| | | processing and | | | | NFYB, | | | | | | | |
| | | presentation | | | | TAP2, | | | | | | | |
| | | | | | | CTSS | | | | | | | |
| JY-bESC | KEGG_PATHWAY | bta04260: | 6 | 1.28479657 | 0.09188091 | MYLA, | 234 | 93 | 9038 | 2.49186656 | 1 | 0.45714456 | 0.423156119085459\ |
| | | Cardiac | | | | COX7B, | | | | | | | |
| | | muscle | | | | LOC783202, | | | | | | | |
| | | contraction | | | | SLC9A7, | | | | | | | |
| | | | | | | COX7A2, | | | | | | | |
| | | | | | | COX7C | | | | | | | |
| JY-bESC | KEGG_PATHWAY | bta04912: | 6 | 1.28479657 | 0.09188091 | CALM, | 234 | 93 | 9038 | 2.49186656 | 1 | 0.45714456 | 0.423156119085459\ |
| | | GnRH | | | | GNAS, | | | | | | | |
| | | signaling | | | | ADCY7 | | | | | | | |
| | | pathway | | | | | | | | | | | |
| JY-bESC | KEGG_PATHWAY | bta04114: | 7 | 1.49892934 | 0.092362397 | CALM, | 234 | 121 | 9038 | 2.23444233 | 1 | 0.45714456 | 0.423156119085459\ |
| | | Oocyte | | | | IGF1, | | | | | | | |
| | | meiosis | | | | ADCY7, | | | | | | | |
| | | | | | | SKP1 | | | | | | | |
| JY-bESC | KEGG_PATHWAY | bta04217: | 9 | 1.92719486 | 0.09371923 | FTH1, | 234 | 180 | 9038 | 1.93119658 | 1 | 0.45714456 | 0.423156119085459\ |
| | | Necroptosis | | | | LOC783993, | | | | | | | |
| | | | | | | LOC784541, | | | | | | | |
| | | | | | | CYBB, | | | | | | | |
| | | | | | | CHMP4B, | | | | | | | |
| | | | | | | HMGB1, | | | | | | | |
| | | | | | | LOC788801, | | | | | | | |
| | | | | | | LOC104968444, | | | | | | | |
| | | | | | | FTL | | | | | | | |
| JY-bESC | KEGG_PATHWAY | bta04722: | 7 | 1.49892934 | 0.095167641 | CALM, | 234 | 122 | 9038 | 2.21612722 | 1 | 0.45714456 | 0.423156119085459} |
| | | Neurotrophin | | | | RAP1B, | | | | | | | |
| | | signaling | | | | TRAF6, | | | | | | | |
| | | pathway | | | | ARHGDIB | | | | | | | |
| bpESC | KEGG_PATHWAY | bta05200: | 29 | 6.9047619 | 6.19E−06 | PDGFB, | 188 | 543 | 9038 | 2.56751303 | 0.00166451 | 0.00166589 | 0.001616346188334262\ |
| | | Pathways | | | | MGST1, | | | | | | | |
| | | in cancer | | | | ADCY8, | | | | | | | |
| | | | | | | PTGS2, | | | | | | | |
| | | | | | | RASGRP2, | | | | | | | |
| | | | | | | MECOM, | | | | | | | |
| | | | | | | PIM1, | | | | | | | |
| | | | | | | PMAIP1, | | | | | | | |
| | | | | | | HES1, | | | | | | | |
| | | | | | | CTNNA2, | | | | | | | |
| | | | | | | EGLN3, | | | | | | | |
| | | | | | | JUN, | | | | | | | |
| | | | | | | LAMB3, | | | | | | | |
| | | | | | | GADD45B, | | | | | | | |
| | | | | | | NOS2, | | | | | | | |
| | | | | | | DCC, | | | | | | | |
| | | | | | | GADD45A, | | | | | | | |
| | | | | | | ZBTB16, | | | | | | | |
| | | | | | | IGF2, | | | | | | | |
| | | | | | | FOS, | | | | | | | |
| | | | | | | LOC100295687, | | | | | | | |
| | | | | | | MMP9, | | | | | | | |
| | | | | | | MAPK10, | | | | | | | |
| | | | | | | CDK6, | | | | | | | |
| | | | | | | LPAR6, | | | | | | | |
| | | | | | | COL4A6, | | | | | | | |
| | | | | | | AGTR1, | | | | | | | |
| | | | | | | COL4A5, | | | | | | | |
| | | | | | | FGFR4 | | | | | | | |
| bpESC | KEGG_PATHWAY | bta04010: | 17 | 4.04761905 | 3.54E−04 | DUSP4, | 188 | 292 | 9038 | 2.79885602 | 0.09096868 | 0.04767941 | 0.04626143937466989\ |
| | | MAPK | | | | NGFR, | | | | | | | |
| | | signaling | | | | JUN, | | | | | | | |
| | | pathway | | | | GADD45B, | | | | | | | |
| | | | | | | GADD45A, | | | | | | | |
| | | | | | | PDGFB, | | | | | | | |
| | | | | | | IGF2, | | | | | | | |
| | | | | | | FOS, | | | | | | | |
| | | | | | | NGF, | | | | | | | |
| | | | | | | RASGRP2, | | | | | | | |
| | | | | | | DUSP6, | | | | | | | |
| | | | | | | MAPK10, | | | | | | | |
| | | | | | | NR4A1, | | | | | | | |
| | | | | | | DUSP10, | | | | | | | |
| | | | | | | MECOM, | | | | | | | |
| | | | | | | FGFR4, | | | | | | | |
| | | | | | | PTPN5 | | | | | | | |
| bpESC | KEGG_PATHWAY | bta04514: | 12 | 2.85714286 | 8.25E−04 | CLDN11, | 188 | 171 | 9038 | 3.37364688 | 0.19915883 | 0.07400033 | 0.07179957517914179\ |
| | | Cell | | | | NTNG1, | | | | | | | |
| | | adhesion | | | | NLGN1, | | | | | | | |
| | | molecules | | | | CDH4, | | | | | | | |
| | | | | | | CDH2, | | | | | | | |
| | | | | | | NEGR1, | | | | | | | |
| | | | | | | SDC2, | | | | | | | |
| | | | | | | ITGB8, | | | | | | | |
| | | | | | | BLA-DQB, | | | | | | | |
| | | | | | | BOLA-DRB3, | | | | | | | |
| | | | | | | LRRC4C, | | | | | | | |
| | | | | | | JAM2 | | | | | | | |
| bpESC | KEGG_PATHWAY | bta04360: | 12 | 2.85714286 | 0.001200889 | EPHA5, | 188 | 179 | 9038 | 3.22286937 | 0.27619499 | 0.08075978 | 0.07835800425641322\ |
| | | Axon | | | | ROBO2, | | | | | | | |
| | | guidance | | | | NTNG1, | | | | | | | |
| | | | | | | ROBO3, | | | | | | | |
| | | | | | | RGS3, | | | | | | | |
| | | | | | | DCC, | | | | | | | |
| | | | | | | SEMA3A, | | | | | | | |
| | | | | | | SEMA3B, | | | | | | | |
| | | | | | | SEMA3E, | | | | | | | |
| | | | | | | LRRC4C, | | | | | | | |
| | | | | | | EPHB1, | | | | | | | |
| | | | | | | ROBO1 | | | | | | | |
| bpESC | KEGG_PATHWAY | bta05222: | 8 | 1.9047619 | 0.003262713 | CDK6, | 188 | 94 | 9038 | 4.09144409 | 0.58484556 | 0.1462783 | 0.14192801202543304\ |
| | | Small | | | | NOS2, | | | | | | | |
| | | cell lung | | | | LAMB3, | | | | | | | |
| | | cancer | | | | GADD45B, | | | | | | | |
| | | | | | | GADD45A, | | | | | | | |
| | | | | | | COL4A6, | | | | | | | |
| | | | | | | COL4A5, | | | | | | | |
| | | | | | | PTGS2 | | | | | | | |
| bpESC | KEGG_PATHWAY | bta04657: | 8 | 1.9047619 | 0.003262713 | MAPK10, | 188 | 94 | 9038 | 4.09144409 | 0.58484556 | 0.1462783 | 0.14192801202543304\ |
| | | IL-17 | | | | JUN, | | | | | | | |
| | | signaling | | | | MMP13, | | | | | | | |
| | | pathway | | | | CCL11, | | | | | | | |
| | | | | | | FOSB, | | | | | | | |
| | | | | | | FOS, | | | | | | | |
| | | | | | | PTGS2, | | | | | | | |
| | | | | | | MMP9 | | | | | | | |
| bpESC | KEGG_PATHWAY | bta04068: | 9 | 2.14285714 | 0.005743727 | MAPK10, | 188 | 131 | 9038 | 3.30282605 | 0.78764943 | 0.16485844 | 0.15995558635940815\ |
| | | FoxO | | | | GABARAPL1, | | | | | | | |
| | | signaling | | | | GADD45B, | | | | | | | |
| | | pathway | | | | GADD45A, | | | | | | | |
| | | | | | | PLK2, | | | | | | | |
| | | | | | | FOXG1, | | | | | | | |
| | | | | | | BNIP3, | | | | | | | |
| | | | | | | CCNG2, | | | | | | | |
| | | | | | | IRS4 | | | | | | | |
| bpESC | KEGG_PATHWAY | bta04926: | 9 | 2.14285714 | 0.005743727 | MAPK10, | 188 | 131 | 9038 | 3.30282605 | 0.78764943 | 0.16485844 | 0.15995558635940815\ |
| | | Relaxin | | | | JUN, | | | | | | | |
| | | signaling | | | | MMP13, | | | | | | | |
| | | pathway | | | | NOS2, | | | | | | | |
| | | | | | | COL4A6, | | | | | | | |
| | | | | | | COL4A5, | | | | | | | |
| | | | | | | FOS, | | | | | | | |
| | | | | | | ADCY8, | | | | | | | |
| | | | | | | MMP9 | | | | | | | |
| bpESC | KEGG_PATHWAY | bta04020: | 13 | 3.0952381 | 0.006015425 | NOS2, | 188 | 252 | 9038 | 2.48003208 | 0.80270119 | 0.16485844 | 0.15995558635940815\ |
| | | Calcium | | | | TNNC1, | | | | | | | |
| | | signaling | | | | PDGFB, | | | | | | | |
| | | pathway | | | | ADCY8, | | | | | | | |
| | | | | | | NGF, | | | | | | | |
| | | | | | | GRPR, | | | | | | | |
| | | | | | | SLC8A2, | | | | | | | |
| | | | | | | SLC8A3, | | | | | | | |
| | | | | | | GNAL, | | | | | | | |
| | | | | | | CCKBR, | | | | | | | |
| | | | | | | AGTR1, | | | | | | | |
| | | | | | | HRC, | | | | | | | |
| | | | | | | FGFR4 | | | | | | | |
| bpESC | KEGG_PATHWAY | bta04115: | 7 | 1.66666667 | 0.006128567 | CDK6, | 188 | 80 | 9038 | 4.20651596 | 0.80865111 | 0.16485844 | 0.15995558635940815\ |
| | | p53 | | | | GADD45B, | | | | | | | |
| | | signaling | | | | CD82, | | | | | | | |
| | | pathway | | | | GADD45A, | | | | | | | |
| | | | | | | CCNG2, | | | | | | | |
| | | | | | | PMAIP1, | | | | | | | |
| | | | | | | RPRM | | | | | | | |
| bpESC | KEGG_PATHWAY | bta04371: | 9 | 2.14285714 | 0.008834653 | SLC8A3, | 188 | 141 | 9038 | 3.06858307 | 0.90810253 | 0.202289 | 0.19627296944967199\ |
| | | Apelin | | | | EGR1, | | | | | | | |
| | | signaling | | | | NOS2, | | | | | | | |
| | | pathway | | | | APLNR, | | | | | | | |
| | | | | | | AGTR1, | | | | | | | |
| | | | | | | SPP1, | | | | | | | |
| | | | | | | CCN2, | | | | | | | |
| | | | | | | ADCY8, | | | | | | | |
| | | | | | | SLC8A2 | | | | | | | |
| bpESC | KEGG_PATHWAY | bta04022: | 10 | 2.38095238 | 0.009024045 | SLC8A3, | 188 | 171 | 9038 | 2.8113724 | 0.91270718 | 0.202289 | 0.19627296944967199\ |
| | | cGMP-PKG | | | | GUCY1A1, | | | | | | | |
| | | signaling | | | | KCNMB2, | | | | | | | |
| | | pathway | | | | PDE2A, | | | | | | | |
| | | | | | | AGTR1, | | | | | | | |
| | | | | | | IRS4, | | | | | | | |
| | | | | | | KCNMB4, | | | | | | | |
| | | | | | | ADCY8, | | | | | | | |
| | | | | | | ADRA2B, | | | | | | | |
| | | | | | | SLC8A2 | | | | | | | |
| bpESC | KEGG_PATHWAY | bta05210: | 7 | 1.66666667 | 0.010180639 | MAPK10, | 188 | 89 | 9038 | 3.78113794 | 0.9362399 | 0.21066092 | 0.2043959116045862\ |
| | | Colorectal | | | | JUN, | | | | | | | |
| | | cancer | | | | GADD45B, | | | | | | | |
| | | | | | | DCC, | | | | | | | |
| | | | | | | GADD45A, | | | | | | | |
| | | | | | | PMAIP1, | | | | | | | |
| | | | | | | FOS | | | | | | | |
| bpESC | KEGG_PATHWAY | bta04261: | 9 | 2.14285714 | 0.012554843 | SLC8A3, | 188 | 150 | 9038 | 2.88446809 | 0.96658106 | 0.24123235 | 0.23405815089617138\ |
| | | Adrenergic | | | | TNNC1, | | | | | | | |
| | | signaling in | | | | TPM1, | | | | | | | |
| | | cardiomyocytes | | | | AGTR1, | | | | | | | |
| | | | | | | SCN7A, | | | | | | | |
| | | | | | | SCN5A, | | | | | | | |
| | | | | | | ADCY8, | | | | | | | |
| | | | | | | RAPGEF3, | | | | | | | |
| | | | | | | SLC8A2 | | | | | | | |
| bpESC | KEGG_PATHWAY | bta04933: | 7 | 1.66666667 | 0.019798516 | MAPK10, | 188 | 103 | 9038 | 3.26719686 | 0.99538862 | 0.34617151 | 0.3358764483241537\ |
| | | AGE-RAGE | | | | EGR1, | | | | | | | |
| | | signaling | | | | JUN, | | | | | | | |
| | | pathway in | | | | COL4A6, | | | | | | | |
| | | diabetic | | | | AGTR1, | | | | | | | |
| | | complications | | | | PIM1, | | | | | | | |
| | | | | | | COL4A5 | | | | | | | |
| bpESC | KEGG_PATHWAY | bta04928: | 7 | 1.66666667 | 0.021550025 | NR4A2, | 188 | 105 | 9038 | 3.20496454 | 0.9971497 | 0.34617151 | 0.3358764483241537\ |
| | | Parathyroid | | | | EGR1, | | | | | | | |
| | | hormone | | | | MMP13, | | | | | | | |
| | | synthesis, | | | | MMP16, | | | | | | | |
| | | secretion and | | | | MMP17, | | | | | | | |
| | | action | | | | FOS, | | | | | | | |
| | | | | | | ADCY8 | | | | | | | |
| bpESC | KEGG_PATHWAY | bta05140: | 6 | 1.42857143 | 0.022499818 | JUN, | 188 | 78 | 9038 | 3.69803601 | 0.99780501 | 0.34617151 | 0.3358764483241537\ |
| | | Leishmaniasis | | | | NOS2, | | | | | | | |
| | | | | | | BLA-DQB, | | | | | | | |
| | | | | | | BOLA-DRB3, | | | | | | | |
| | | | | | | FOS, | | | | | | | |
| | | | | | | PTGS2 | | | | | | | |
| bpESC | KEGG_PATHWAY | bta05202: | 10 | 2.38095238 | 0.023163893 | NGFR, | 188 | 200 | 9038 | 2.4037234 | 0.99817174 | 0.34617151 | 0.3358764483241537\ |
| | | Transcriptional | | | | ZEB1, | | | | | | | |
| | | misregulation in | | | | EYA1, | | | | | | | |
| | | cancer | | | | GADD45B, | | | | | | | |
| | | | | | | GADD45A, | | | | | | | |
| | | | | | | ZBTB16, | | | | | | | |
| | | | | | | MLLT3, | | | | | | | |
| | | | | | | MMP9, | | | | | | | |
| | | | | | | CDK14, | | | | | | | |
| | | | | | | DUSP6 | | | | | | | |
| bpESC | KEGG_PATHWAY | bta05418: | 8 | 1.9047619 | 0.030757603 | MAPK10, | 188 | 145 | 9038 | 2.65238445 | 0.99977596 | 0.43546291 | 0.4225123405684193\ |
| | | Fluid | | | | JUN, | | | | | | | |
| | | shear stress and | | | | SDC2, | | | | | | | |
| | | atherosclerosis | | | | MGST1, | | | | | | | |
| | | | | | | PDGFB, | | | | | | | |
| | | | | | | FOS, | | | | | | | |
| | | | | | | LOC100295687, | | | | | | | |
| | | | | | | MMP9 | | | | | | | |
| bpESC | KEGG_PATHWAY | bta04512: | 6 | 1.42857143 | 0.036974912 | LAMB3, | 188 | 89 | 9038 | 3.24097538 | 0.99996033 | 0.49731257 | 0.4825226067337249\ |
| | | ECM-receptor | | | | COL4A6, | | | | | | | |
| | | interaction | | | | SPP1, | | | | | | | |
| | | | | | | COL4A5, | | | | | | | |
| | | | | | | ITGB8, | | | | | | | |
| | | | | | | FREM1 | | | | | | | |
| bpESC | KEGG_PATHWAY | bta00982: | 5 | 1.19047619 | 0.039080598 | LOC508879, | 188 | 62 | 9038 | 3.87697323 | 0.99997798 | 0.50060385 | 0.48571600531782133\ |
| | | Drug | | | | MAOA, | | | | | | | |
| | | metabolism - | | | | MGST1, | | | | | | | |
| | | cytochrome P450 | | | | LOC100295687, | | | | | | | |
| | | | | | | ADH6 | | | | | | | |
| bpESC | KEGG_PATHWAY | bta01522: | 6 | 1.42857143 | 0.045086222 | MAPK10, | 188 | 94 | 9038 | 3.06858307 | 0.99999592 | 0.55128153 | 0.5348865433880958\ |
| | | Endocrine | | | | JUN, | | | | | | | |
| | | resistance | | | | BIK, | | | | | | | |
| | | | | | | FOS, | | | | | | | |
| | | | | | | ADCY8, | | | | | | | |
| | | | | | | MMP9 | | | | | | | |
| bpESC | KEGG_PATHWAY | bta04151: | 14 | 3.33333333 | 0.050696905 | NGFR, | 188 | 379 | 9038 | 1.77583787 | 0.99999916 | 0.58402653 | 0.5666577124016128\ |
| | | PI3K-Akt | | | | LAMB3, | | | | | | | |
| | | signaling | | | | PDGFB, | | | | | | | |
| | | pathway | | | | IGF2, | | | | | | | |
| | | | | | | NGF, | | | | | | | |
| | | | | | | NR4A1, | | | | | | | |
| | | | | | | CDK6, | | | | | | | |
| | | | | | | LPAR6, | | | | | | | |
| | | | | | | DDIT4, | | | | | | | |
| | | | | | | COL4A6, | | | | | | | |
| | | | | | | SPP1, | | | | | | | |
| | | | | | | COL4A5, | | | | | | | |
| | | | | | | ITGB8, | | | | | | | |
| | | | | | | FGFR4 | | | | | | | |
| bpESC | KEGG_PATHWAY | bta05414: | 6 | 1.42857143 | 0.054182679 | SLC8A3, | 188 | 99 | 9038 | 2.91360413 | 0.99999969 | 0.58402653 | 0.5666577124016128\ |
| | | Dilated | | | | TNNC1, | | | | | | | |
| | | cardiomyopathy | | | | TPM1, | | | | | | | |
| | | | | | | ITGB8, | | | | | | | |
| | | | | | | ADCY8, | | | | | | | |
| | | | | | | SLC8A2 | | | | | | | |
| bpESC | KEGG_PATHWAY | bta05031: | 5 | 1.19047619 | 0.054277559 | JUN, | 188 | 69 | 9038 | 3.48365711 | 0.9999997 | 0.58402653 | 0.5666577124016128\ |
| | | Amphetamine | | | | ARC, | | | | | | | |
| | | addiction | | | | MAOA, | | | | | | | |
| | | | | | | FOSB, | | | | | | | |
| | | | | | | FOS | | | | | | | |
| bpESC | KEGG_PATHWAY | bta04137: | 5 | 1.19047619 | 0.064206897 | MAPK10, | 188 | 73 | 9038 | 3.29277179 | 0.99999998 | 0.64692162 | 0.6276823161498646\ |
| | | Mitophagy - | | | | JUN, | | | | | | | |
| | | animal | | | | GABARAPL1, | | | | | | | |
| | | | | | | CITED2, | | | | | | | |
| | | | | | | BNIP3 | | | | | | | |
| bpESC | KEGG_PATHWAY | bta04080: | 14 | 3.33333333 | 0.064932653 | GRIK4, | 188 | 394 | 9038 | 1.70822983 | 0.99999999 | 0.64692162 | 0.6276823161498646\ |
| | | Neuroactive | | | | ADM, | | | | | | | |
| | | ligand- | | | | GRIK2, | | | | | | | |
| | | receptor | | | | GRPR, | | | | | | | |
| | | interaction | | | | ADRA2B, | | | | | | | |
| | | | | | | GABRG1, | | | | | | | |
| | | | | | | CCKBR, | | | | | | | |
| | | | | | | GLRB, | | | | | | | |
| | | | | | | LPAR6, | | | | | | | |
| | | | | | | GRM8, | | | | | | | |
| | | | | | | APLNR, | | | | | | | |
| | | | | | | AGTR1, | | | | | | | |
| | | | | | | TAC3, | | | | | | | |
| | | | | | | S1PR2 | | | | | | | |
| bpESC | KEGG_PATHWAY | bta05412: | 5 | 1.19047619 | 0.072235982 | SLC8A3, | 188 | 76 | 9038 | 3.16279395 | 1 | 0.6939814 | 0.6733425462576451\ |
| | | Arrhythmogenic | | | | CDH2, | | | | | | | |
| | | right | | | | ITGB8, | | | | | | | |
| | | ventricular | | | | CTNNA2, | | | | | | | |
| | | cardiomyopathy | | | | SLC8A2 | | | | | | | |
| bpESC | KEGG_PATHWAY | bta04210: | 7 | 1.66666667 | 0.076123953 | MAPK10, | 188 | 143 | 9038 | 2.35329564 | 1 | 0.70611529 | 0.6851155749853337\ |
| | | Apoptosis | | | | JUN, | | | | | | | |
| | | | | | | GADD45B, | | | | | | | |
| | | | | | | GADD45A, | | | | | | | |
| | | | | | | PMAIP1, | | | | | | | |
| | | | | | | FOS, | | | | | | | |
| | | | | | | NGF | | | | | | | |
| bpESC | KEGG_PATHWAY | bta04724: | 6 | 1.42857143 | 0.084899199 | GRM8, | 188 | 113 | 9038 | 2.55262662 | 1 | 0.75826924 | 0.7357184842843347\ |
| | | Glutamatergic | | | | GRIK4, | | | | | | | |
| | | synapse | | | | SLC17A7, | | | | | | | |
| | | | | | | GRIK2, | | | | | | | |
| | | | | | | ADCY8, | | | | | | | |
| | | | | | | SLC38A3 | | | | | | | |
| bpESC | KEGG_PATHWAY | bta05145: | 6 | 1.42857143 | 0.087384188 | MAPK10, | 188 | 114 | 9038 | 2.53023516 | 1 | 0.75826924 | 0.7357184842843347\ |
| | | Toxoplasmosis | | | | NOS2, | | | | | | | |
| | | | | | | LAMB3, | | | | | | | |
| | | | | | | ALOX5, | | | | | | | |
| | | | | | | BLA-DQB, | | | | | | | |
| | | | | | | BOLA-DRB3 | | | | | | | |
| bpESC | KEGG_PATHWAY | bta04072: | 7 | 1.66666667 | 0.097615975 | LPAR6, | 188 | 153 | 9038 | 2.19948547 | 1 | 0.82058429 | 0.7961802923223262} |
| | | Phospholipase D | | | | GRM8, | | | | | | | |
| | | signaling | | | | PDGFB, | | | | | | | |
| | | pathway | | | | AGTR1, | | | | | | | |
| | | | | | | DGKK, | | | | | | | |
| | | | | | | ADCY8, | | | | | | | |
| | | | | | | RAPGEF3 | | | | | | | |
| biPSC | KEGG_PATHWAY | bta05224: | 11 | 3.98550725 | 1.44E−05 | DLL4, | 112 | 150 | 9038 | 5.9177381 | 0.00312794 | 0.00313282 | 0.003017323992232059\ |
| | | Breast | | | | JAG1, | | | | | | | |
| | | cancer | | | | WNT5B, | | | | | | | |
| | | | | | | MYC, | | | | | | | |
| | | | | | | TCF7, | | | | | | | |
| | | | | | | LEF1, | | | | | | | |
| | | | | | | AXIN2, | | | | | | | |
| | | | | | | FZD10, | | | | | | | |
| | | | | | | DLL1, | | | | | | | |
| | | | | | | ESR2, | | | | | | | |
| | | | | | | GADD45G | | | | | | | |
| biPSC | KEGG_PATHWAY | bta04550: | 10 | 3.62318841 | 6.55E−05 | ZFHX3, | 112 | 144 | 9038 | 5.60391865 | 0.01412086 | 0.00711052 | 0.006848379983832482\ |
| | | Signaling | | | | WNT5B, | | | | | | | |
| | | pathways | | | | DLX5, | | | | | | | |
| | | regulating | | | | MYC, | | | | | | | |
| | | pluripotency of | | | | TCF7, | | | | | | | |
| | | stem cells | | | | ESRRB, | | | | | | | |
| | | | | | | AXIN2, | | | | | | | |
| | | | | | | FZD10, | | | | | | | |
| | | | | | | KLF4, | | | | | | | |
| | | | | | | TBX3 | | | | | | | |
| biPSC | KEGG_PATHWAY | bta05207: | 11 | 3.98550725 | 3.10E−04 | DLL4, | 112 | 216 | 9038 | 4.10954034 | 0.06506402 | 0.02242226 | 0.021595637530123907\ |
| | | Chemical | | | | JAG1, | | | | | | | |
| | | carcinogenesis - | | | | PAQR5, | | | | | | | |
| | | receptor | | | | MYC, | | | | | | | |
| | | activation | | | | CYP1A1, | | | | | | | |
| | | | | | | MGST2, | | | | | | | |
| | | | | | | CYP1B1, | | | | | | | |
| | | | | | | KLF4, | | | | | | | |
| | | | | | | DLL1, | | | | | | | |
| | | | | | | ESR2, | | | | | | | |
| | | | | | | UGT1A6 | | | | | | | |
| biPSC | KEGG_PATHWAY | bta05226: | 9 | 3.26086957 | 5.68E−04 | ABCB1, | 112 | 153 | 9038 | 4.74684874 | 0.11606959 | 0.03083547 | 0.029698675865412673\ |
| | | Gastric | | | | CDX2, | | | | | | | |
| | | cancer | | | | WNT5B, | | | | | | | |
| | | | | | | MYC, | | | | | | | |
| | | | | | | TCF7, | | | | | | | |
| | | | | | | LEF1, | | | | | | | |
| | | | | | | AXIN2, | | | | | | | |
| | | | | | | FZD10, | | | | | | | |
| | | | | | | GADD45G | | | | | | | |
| biPSC | KEGG_PATHWAY | bta05217: | 6 | 2.17391304 | 0.001018213 | WNT5B, | 112 | 63 | 9038 | 7.68537415 | 0.19833532 | 0.04419046 | 0.042561317967461584\ |
| | | Basal | | | | TCF7, | | | | | | | |
| | | cell carcinoma | | | | LEF1, | | | | | | | |
| | | | | | | AXIN2, | | | | | | | |
| | | | | | | FZD10, | | | | | | | |
| | | | | | | GADD45G | | | | | | | |
| biPSC | KEGG_PATHWAY | bta05225: | 9 | 3.26086957 | 0.001315855 | WNT5B, | 112 | 174 | 9038 | 4.1739532 | 0.24853337 | 0.04759007 | 0.045835600462976495\ |
| | | Hepatocellular | | | | CDKN2A, | | | | | | | |
| | | carcinoma | | | | MYC, | | | | | | | |
| | | | | | | TCF7, | | | | | | | |
| | | | | | | LEF1, | | | | | | | |
| | | | | | | MGST2, | | | | | | | |
| | | | | | | AXIN2, | | | | | | | |
| | | | | | | FZD10, | | | | | | | |
| | | | | | | GADD45G | | | | | | | |
| biPSC | KEGG_PATHWAY | bta05200: | 16 | 5.79710145 | 0.002529334 | JAG1, | 112 | 543 | 9038 | 2.37779532 | 0.42279673 | 0.07840935 | 0.07551868720290661\ |
| | | Pathways | | | | WNT5B, | | | | | | | |
| | | in cancer | | | | CDKN2A, | | | | | | | |
| | | | | | | FLT3, | | | | | | | |
| | | | | | | LOC530599, | | | | | | | |
| | | | | | | TCF7, | | | | | | | |
| | | | | | | LEF1, | | | | | | | |
| | | | | | | MGST2, | | | | | | | |
| | | | | | | AXIN2, | | | | | | | |
| | | | | | | FZD10, | | | | | | | |
| | | | | | | DLL1, | | | | | | | |
| | | | | | | ESR2, | | | | | | | |
| | | | | | | GADD45G, | | | | | | | |
| | | | | | | DLL4, | | | | | | | |
| | | | | | | MYC, | | | | | | | |
| | | | | | | IL6R | | | | | | | |
| biPSC | KEGG_PATHWAY | bta04934: | 8 | 2.89855072 | 0.003247531 | WNT5B, | 112 | 158 | 9038 | 4.08589512 | 0.50631671 | 0.08808929 | 0.08484175576615884\ |
| | | Cushing | | | | CDKN2A, | | | | | | | |
| | | syndrome | | | | TCF7, | | | | | | | |
| | | | | | | LEF1, | | | | | | | |
| | | | | | | ITPR3, | | | | | | | |
| | | | | | | AXIN2, | | | | | | | |
| | | | | | | FZD10, | | | | | | | |
| | | | | | | PDE8A | | | | | | | |
| biPSC | KEGG_PATHWAY | bta05213: | 5 | 1.8115942 | 0.005838339 | MYC, | 112 | 59 | 9038 | 6.83868039 | 0.71934588 | 0.14076883 | 0.1355791948475416\ |
| | | Endometrial | | | | TCF7, | | | | | | | |
| | | cancer | | | | LEF1, | | | | | | | |
| | | | | | | AXIN2, | | | | | | | |
| | | | | | | GADD45G | | | | | | | |
| biPSC | KEGG_PATHWAY | bta01100: | 31 | 11.2318841 | 0.009774774 | ND6, | 112 | 1607 | 9038 | 1.55668282 | 0.88134804 | 0.2121126 | 0.20429277769221546\ |
| | | Metabolic | | | | ST6GALNAC1, | | | | | | | |
| | | pathways | | | | GALNT14, | | | | | | | |
| | | | | | | ATP8, | | | | | | | |
| | | | | | | MGST2, | | | | | | | |
| | | | | | | PIK3C2B, | | | | | | | |
| | | | | | | AFMID, | | | | | | | |
| | | | | | | ST8SIA5, | | | | | | | |
| | | | | | | UPP1, | | | | | | | |
| | | | | | | CKB, | | | | | | | |
| | | | | | | PRODH, | | | | | | | |
| | | | | | | PDE8A, | | | | | | | |
| | | | | | | UGT1A6, | | | | | | | |
| | | | | | | TMEM86B, | | | | | | | |
| | | | | | | AOC2, | | | | | | | |
| | | | | | | ENTPD3, | | | | | | | |
| | | | | | | ACSL1, | | | | | | | |
| | | | | | | CKMT1A, | | | | | | | |
| | | | | | | SORD, | | | | | | | |
| | | | | | | PGD, | | | | | | | |
| | | | | | | ASRGL1, | | | | | | | |
| | | | | | | ETNPPL, | | | | | | | |
| | | | | | | RPIA, | | | | | | | |
| | | | | | | PHOSPHO1, | | | | | | | |
| | | | | | | B3GNT6, | | | | | | | |
| | | | | | | LPCAT4, | | | | | | | |
| | | | | | | CYP1A1, | | | | | | | |
| | | | | | | ALPL, | | | | | | | |
| | | | | | | ATP6V0D2, | | | | | | | |
| | | | | | | ATP6V1B1, | | | | | | | |
| | | | | | | ND4 | | | | | | | |
| biPSC | KEGG_PATHWAY | bta04390: | 7 | 2.53623188 | 0.01230511 | WNT5B, | 112 | 156 | 9038 | 3.62099359 | 0.9318997 | 0.23162581 | 0.22308660919895903\ |
| | | Hippo | | | | MYC, | | | | | | | |
| | | signaling | | | | TCF7, | | | | | | | |
| | | pathway | | | | LEF1, | | | | | | | |
| | | | | | | AXIN2, | | | | | | | |
| | | | | | | FZD10, | | | | | | | |
| | | | | | | TP73 | | | | | | | |
| biPSC | KEGG_PATHWAY | bta05216: | 4 | 1.44927536 | 0.012808801 | MYC, | 112 | 40 | 9038 | 8.06964286 | 0.93903555 | 0.23162581 | 0.22308660919895903\ |
| | | Thyroid | | | | TCF7, | | | | | | | |
| | | cancer | | | | LEF1, | | | | | | | |
| | | | | | | GADD45G | | | | | | | |
| biPSC | KEGG_PATHWAY | bta04310: | 7 | 2.53623188 | 0.018596011 | WNT5B, | 112 | 171 | 9038 | 3.30336257 | 0.98297933 | 0.31041034 | 0.29896663962653064\ |
| | | Wnt | | | | MYC, | | | | | | | |
| | | signaling | | | | TCF7, | | | | | | | |
| | | pathway | | | | LEF1, | | | | | | | |
| | | | | | | AXIN2, | | | | | | | |
| | | | | | | NOTUM, | | | | | | | |
| | | | | | | FZD10 | | | | | | | |
| biPSC | KEGG_PATHWAY | bta05210: | 5 | 1.8115942 | 0.023700627 | MYC, | 112 | 89 | 9038 | 4.53350722 | 0.99451075 | 0.36735972 | 0.3538165085687796\ |
| | | Colorectal | | | | TCF7, | | | | | | | |
| | | cancer | | | | LEF1, | | | | | | | |
| | | | | | | AXIN2, | | | | | | | |
| | | | | | | GADD45G | | | | | | | |
| biPSC | KEGG_PATHWAY | bta01522: | 5 | 1.8115942 | 0.028266381 | DLL4, | 112 | 94 | 9038 | 4.29236322 | 0.99801505 | 0.39154128 | 0.37710657624179494\ |
| | | Endocrine | | | | JAG1, | | | | | | | |
| | | resistance | | | | CDKN2A, | | | | | | | |
| | | | | | | DLL1, | | | | | | | |
| | | | | | | ESR2 | | | | | | | |
| biPSC | KEGG_PATHWAY | bta05165: | 10 | 3.62318841 | 0.028869403 | HES6, | 112 | 352 | 9038 | 2.29251218 | 0.99826521 | 0.39154128 | 0.37710657624179494\ |
| | | Human | | | | JAG1, | | | | | | | |
| | | papillomavirus | | | | WNT5B, | | | | | | | |
| | | infection | | | | LOC530599, | | | | | | | |
| | | | | | | TCF7, | | | | | | | |
| | | | | | | AXIN2, | | | | | | | |
| | | | | | | FZD10, | | | | | | | |
| | | | | | | HES2, | | | | | | | |
| | | | | | | ATP6V0D2, | | | | | | | |
| | | | | | | ATP6V1B1 | | | | | | | |
| biPSC | KEGG_PATHWAY | bta04658: | 5 | 1.8115942 | 0.033322437 | DLL4, | 112 | 99 | 9038 | 4.0755772 | 0.99936009 | 0.42535111 | 0.40966996460742855\ |
| | | Th1 | | | | JAG1, | | | | | | | |
| | | and Th2 cell | | | | PRKCQ, | | | | | | | |
| | | differentiation | | | | CD247, | | | | | | | |
| | | | | | | DLL1 | | | | | | | |
| biPSC | KEGG_PATHWAY | bta05132: | 8 | 2.89855072 | 0.04143867 | AHNAK, | 112 | 261 | 9038 | 2.47345375 | 0.99989731 | 0.49956619 | 0.4811489997988883\ |
| | | Salmonella | | | | MYC, | | | | | | | |
| | | infection | | | | TCF7, | | | | | | | |
| | | | | | | MYO6, | | | | | | | |
| | | | | | | LEF1, | | | | | | | |
| | | | | | | RAB7B, | | | | | | | |
| | | | | | | PIK3C2B, | | | | | | | |
| | | | | | | TUBA8 | | | | | | | |
| biPSC | KEGG_PATHWAY | bta05204: | 4 | 1.44927536 | 0.045489231 | CYP1A1, | 112 | 65 | 9038 | 4.96593407 | 0.99995903 | 0.5195349 | 0.5003815393028065\ |
| | | Chemical | | | | MGST2, | | | | | | | |
| | | carcinogenesis - | | | | CYP1B1, | | | | | | | |
| | | DNA adducts | | | | UGT1A6 | | | | | | | |
| biPSC | KEGG_PATHWAY | bta00980: | 4 | 1.44927536 | 0.049029755 | CYP1A1, | 112 | 67 | 9038 | 4.81769723 | 0.99998171 | 0.52544317 | 0.5060719965291135\ |
| | | Metabolism of | | | | MGST2, | | | | | | | |
| | | xenobiotics by | | | | CYP1B1, | | | | | | | |
| | | cytochrome P450 | | | | UGT1A6 | | | | | | | |
| biPSC | KEGG_PATHWAY | bta05221: | 4 | 1.44927536 | 0.050849339 | FLT3, | 112 | 68 | 9038 | 4.74684874 | 0.99998793 | 0.52544317 | 0.5060719965291135\ |
| | | Acute | | | | MYC, | | | | | | | |
| | | myeloid | | | | TCF7, | | | | | | | |
| | | leukemia | | | | LEF1 | | | | | | | |
| biPSC | KEGG_PATHWAY | bta00512: | 3 | 1.08695652 | 0.068420131 | ST6GALNAC1, | 112 | 35 | 9038 | 6.91683673 | 0.99999979 | 0.67487129 | 0.6499912410221917\ |
| | | Mucin | | | | GALNT14, | | | | | | | |
| | | type O-glycan | | | | B3GNT6 | | | | | | | |
| | | biosynthesis | | | | | | | | | | | |
| biPSC | KEGG_PATHWAY | bta04270: | 5 | 1.8115942 | 0.093376621 | PPP1R14A, | 112 | 140 | 9038 | 2.88201531 | 1 | 0.88098812 | 0.8485092949537884\ |
| | | Vascular | | | | RAMP3, | | | | | | | |
| | | smooth muscle | | | | ITPR3, | | | | | | | |
| | | contraction | | | | PRKCQ, | | | | | | | |
| | | | | | | ACTG2 | | | | | | | |
| biPSC | KEGG_PATHWAY | bta00190: | 5 | 1.8115942 | 0.099016838 | ND6, | 112 | 143 | 9038 | 2.82155345 | 1 | 0.89527725 | 0.8622716328282223} |
| | | Oxidative | | | | ATP8, | | | | | | | |
| | | phosphorylation | | | | ATP6V0D2, | | | | | | | |
| | | | | | | ND4, | | | | | | | |
| | | | | | | ATP6V1B1 | | | | | | | |

For bpESCs, enriched GO terms and KEGG pathways included cell dedifferentiation, negative regulation of canonical WNT pathways, MAPK signaling, and FoxO pathways (FIG. 2B and Table C). In addition, biPSCs showed enrichment in embryonic organization and fate determination GO terms, with KEGG analysis revealing Hippo signaling pathways and pluripotency regulation (FIG. 2B and Table C). Notably, biPSCs also exhibited lower expression of NANOG and higher trophectoderm marker CDX2 compared to other cells (FIGS. 2B and 2C).

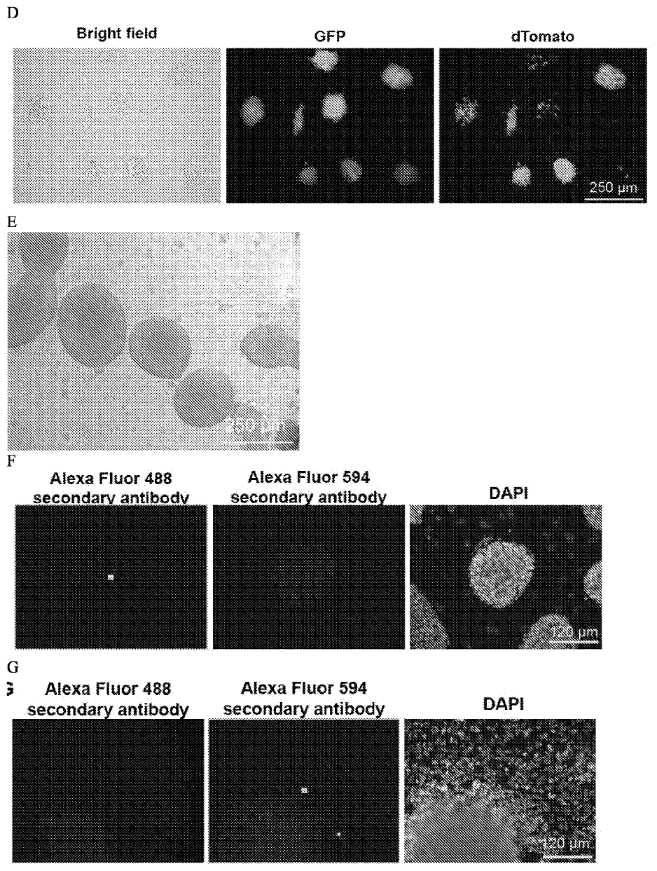

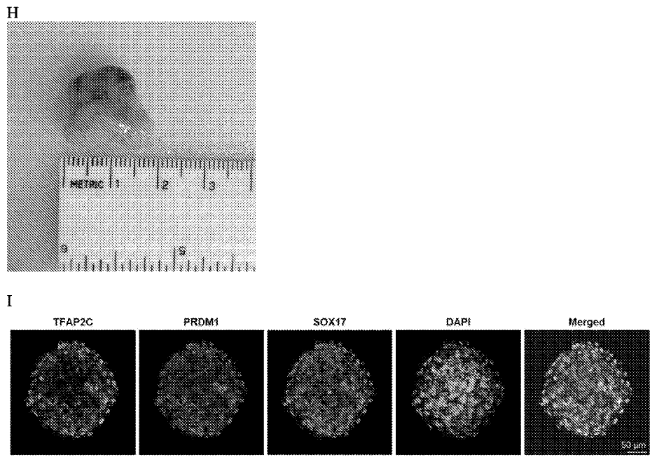

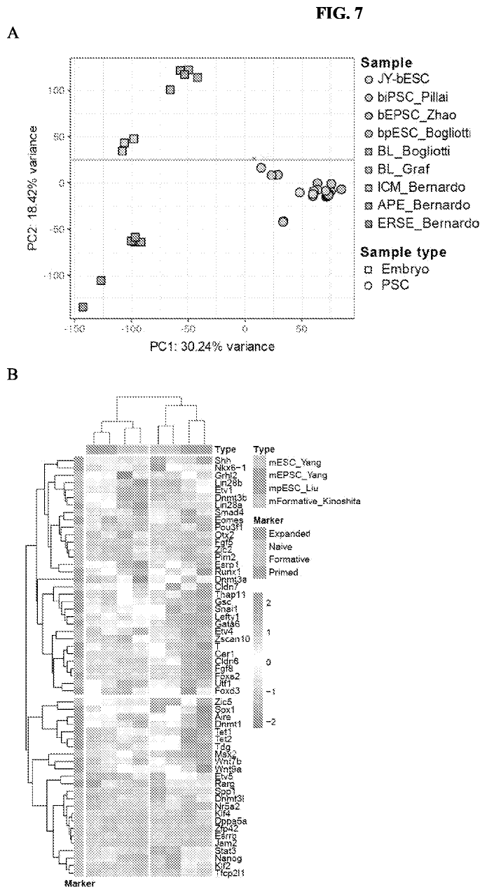

To define the pluripotent state of JY-bESCs, various stages of bovine embryonic development were compared, including pre- and post-implantation embryos. Specifically, different PSCs were compared with bovine dissected inner cell mass (ICM) from Day 7 blastocysts, epithelial radially symmetric epiblast (ERSE) from Day 14 embryos, and anterior to posterior epiblast (APE) from Day 17 embryos (5). The PCA analysis revealed that the bovine PSCs were separated from the bovine embryo samples along PC1/PC2 (FIG. 7A) and the primed state bpESCs exhibit a closer resemblance to APE while the naive state biPSCs align closer with ICM along PC2/PC3 (FIG. 2A). Notably, JY-bESCs were positioned intermediately between ICM and APE stages along PC2/PC3, indicating their transitional state (FIG. 2A). Known markers representing different pluripotency states in these bovine PSCs (FIG. 2C) were examined. These markers were defined in mice and showed species-specific expressions across expanded, naive, formative, and primed PSCs in mice (FIG. 7B) and humans (FIG. 7C). Among bovine PSCs, biPSCs had high expression of naive pluripotency markers (ESRRB, KLF4/2, ZFP42, NR5A2) and upregulated primed markers (GATA6, PAX3) (FIG. 2C). JY-bESCs predominantly expressed formative markers (15 out of 21) such as CLDN6/7, DNMT3B, ESRP1, GRHL2, and LIN28B, while also showing expression of expanded (LIN28A), naive (STAT3, TFCP2L1), and primed markers (EOMES, RUNX1) (FIG. 2C). Bovine primed ESCs were enriched in primed markers and showed scattered gene enrichment across other pluripotency panels (TET1/2, JAM2, FGF5, POU3F1, FOXA2). bEPSCs had one enriched expanded marker (AIRE) and elevated expression of some formative (CLDN6, UTF1) and primed markers (EOMES) (FIG. 2C). The predominant expression of formative markers in JY-bESCs indicated a formative pluripotent status for JY-bESCs compared to other reported bovine PSCs. The cells were compared with bovine somatic cells (bovine mesenchymal stem cells, bMSCs) and found that the bMSCs exhibited enrichment in differentiation genes (GDF6, SST, LAMA3, HAND1, DKK1), whereas the top differentially expressed genes (DEGs) in JY-bESCs were prominently associated with pluripotency (LIN28A, OTX2, OCT4, NANOG) (FIG. 7D). This finding confirms and emphasizes the pluripotency of JY-bESCs.

### Cross-Species Analysis for Bovine-Specific Features

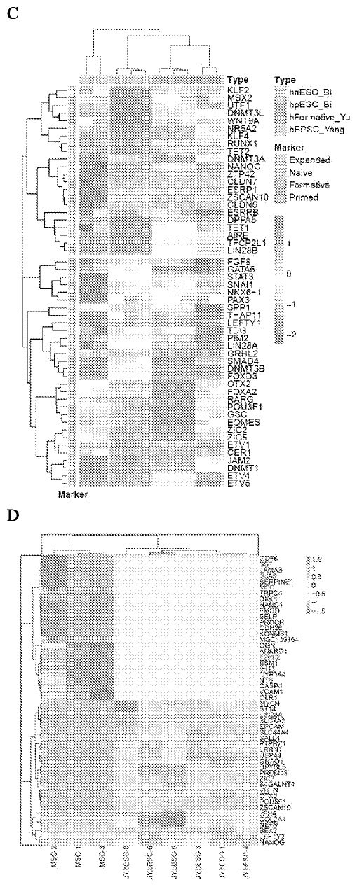

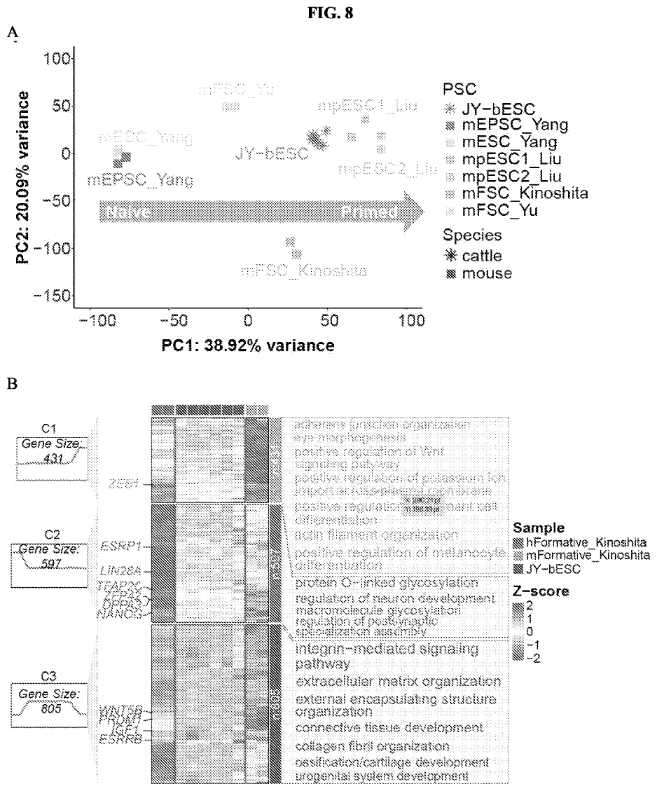

To further identify the pluripotency status of bovine PSCs, the transcriptomic differences among PSCs of the human, mouse and bovine and their blastocysts (Table A) were compared. In general, 2D PCA (FIG. 2D) for all three species revealed a clear cell trajectory based on pluripotency states from expanded/extended, naive, formative, to primed PSCs. Interestingly, mouse expanded/extended PSCs (EPSC) are next to naive PSCs, while human and bovine EPSCs are closer to formative PSCs, indicating similarity between human and bovine PSCs compared to mouse. Further projection of JY-bESC to humans or mice PSCs revealed JY-bESCs to be closest to human formative PSCs (hFSCs, FIG. 2E), and between mouse mFSCs and primed PSCs (mpESC, FIG. 8A).

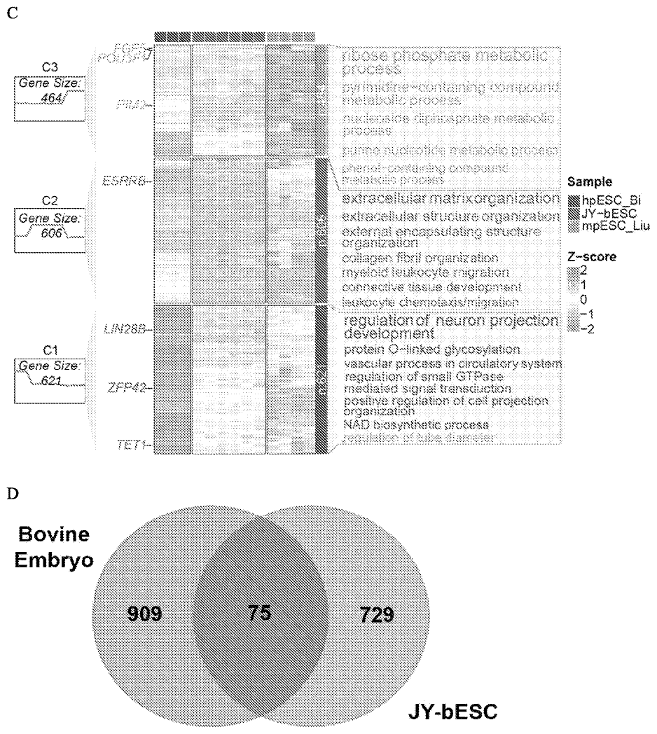

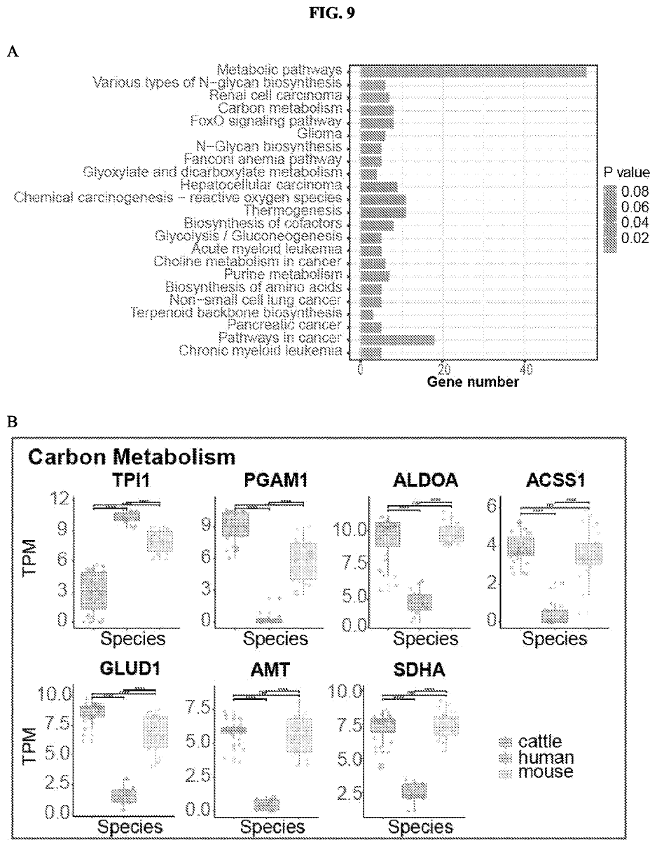

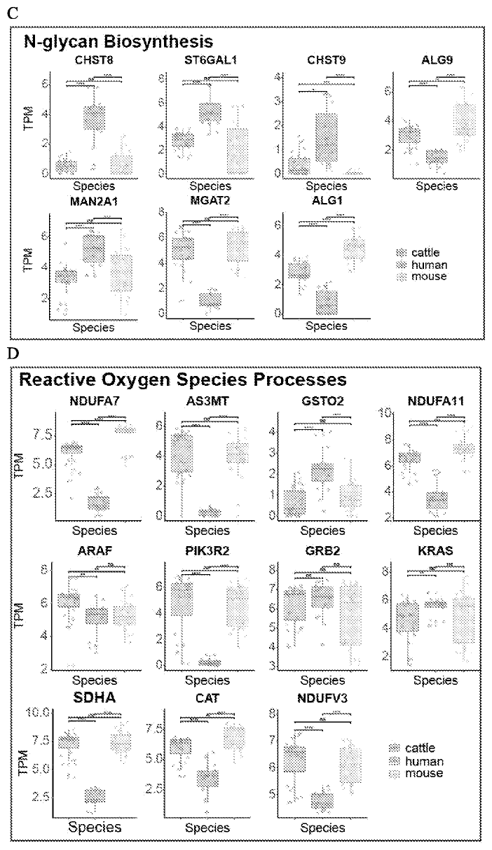

Intriguingly, bovine, mouse, and human PSCs and blastocysts showed clear species-specific separation along PC3 of the 3D PCA (FIG. 2F). KEGG analysis on the top 2,000 PC3 contributors revealed significant enrichment in metabolic pathways, including carbon metabolism, N-glycan biosynthesis, reactive oxygen species, purine synthesis, and biosynthesis of cofactors (FIGS. 9A-9F). Notably, genes that affect carbon metabolism including glycolysis (TPI1, PGAM1, ALDOA) and TCA cycle (ACSS1, GLUD1, AMT, SDHA) displayed species-specific expression patterns, with bovine showing the highest expression for PGAM1 and GLUD1 and comparable expression as mice for the other genes, and with humans having the lowest expression for most of these genes except for TPI1 (FIG. 9B). Additionally, bovine early embryonic development showed a significant shift of glycolysis intermediates towards lipid metabolism, with the lowest expression of TPI1 (FIG. 9B) but greatest expression of GPD1—key gene for cytoplasmic glycerolipid synthesis in bovine compared with humans and mice, and the lowest expression of GPD2 which mediates the reversible interaction of GPD1 in mitochondria (FIG. 9G). Interestingly, TPI1 and GPD1 expression is much higher in bovine PSCs compared to embryos, particularly for TPI1 in JY-bESCs (FIG. 9G-9H). The distinct expression pattern of genes related to carbon metabolism indicate species-specific variations in mitochondria carbon metabolic rates among human, mouse, and bovine early embryos and PSCs.

To further define species-specific pluripotency, JY-bESCs were compared to human and mouse formative (hFSCs and mFSCs, FIG. 8B and Table D) or primed PSCs (hpESC and mpESC, FIG. 8C and Table E).

Table D: DEG Numbers (Up-Regulated and Down-Regulated) in Comparisons of JY-bESC Vs. hFormative_Kinoshita, JY-bESC Vs. MFormative_Kinoshita, and hFormative_Kinoshita Vs. mFormative_Kinoshita

| | Up- | Down- |
|---|---|---|
| Comparisons | regulated | regulated |
| JY-bESC vs. hFormative_Kinoshita | 1843 | 1367 |
| JY-bESC vs. mFormative_Kinoshita | 1847 | 1203 |
| hFormative_Kinoshita vs. | 1654 | 1524 |
| mFormative_Kinoshita | | |

Table E: GO Terms Enriched by Genes Uniquely Expressed in hFSCs, JY-bESCs, and mFSCs

| ID | group | Description | pvalue | ratio |
|---|---|---|---|---|
| GO:0008217 | C1 | regulation of blood pressure | 0.049716 | 0.023224 |
| GO:0051604 | C1 | protein maturation | 0.04946 | 0.034153 |
| GO:1903555 | C1 | regulation of tumor necrosis factor | 0.04946 | 0.025956 |
| | | superfamily cytokine production | | |
| GO:0071706 | C1 | tumor necrosis factor superfamily | 0.04946 | 0.025956 |
| | | cytokine production | | |
| GO:0030099 | C1 | myeloid cell differentiation | 0.04946 | 0.04235 |
| GO:0006023 | C1 | aminoglycan biosynthetic process | 0.047442 | 0.013661 |
| GO:0060351 | C1 | cartilage development involved in | 0.047358 | 0.008197 |
| | | endochondral bone morphogenesis | | |
| GO:0048732 | C1 | gland development | 0.046215 | 0.04235 |
| GO:0032288 | C1 | myelin assembly | 0.045267 | 0.006831 |
| GO:0002062 | C1 | chondrocyte differentiation | 0.043945 | 0.016393 |
| GO:0032911 | C1 | negative regulation of transforming | 0.043945 | 0.004098 |
| | | growth factor betal production | | |
| GO:0010761 | C1 | fibroblast migration | 0.043344 | 0.010929 |
| GO:0032680 | C1 | regulation of tumor necrosis factor | 0.042232 | 0.025956 |
| | | production | | |
| GO:0032640 | C1 | tumor necrosis factor production | 0.042232 | 0.025956 |
| GO:0062012 | C1 | regulation of small molecule | 0.041916 | 0.034153 |
| | | metabolic process | | |
| GO:0072523 | C1 | purine-containing compound catabolic | 0.039834 | 0.010929 |
| | | process | | |
| GO:0002688 | C1 | regulation of leukocyte chemotaxis | 0.039834 | 0.019126 |
| GO:1905477 | C1 | positive regulation of protein | 0.039834 | 0.016393 |
| | | localization to membrane | | |
| GO:0034446 | C1 | substrate adhesion-dependent cell | 0.039834 | 0.016393 |
| | | spreading | | |
| GO:0030201 | C1 | heparan sulfate proteoglycan | 0.039834 | 0.009563 |
| | | metabolic process | | |
| GO:0042692 | C1 | muscle cell differentiation | 0.039834 | 0.039617 |
| GO:0032970 | C1 | regulation of actin filament-based | 0.039834 | 0.039617 |
| | | process | | |
| GO:0090201 | C1 | negative regulation of release of | 0.039834 | 0.006831 |
| | | cytochrome c from mitochondria | | |
| GO:0032930 | C1 | positive regulation of superoxide | 0.039834 | 0.006831 |
| | | anion generation | | |
| GO:0009150 | C1 | purine ribonucleotide metabolic | 0.039834 | 0.043716 |
| | | process | | |
| GO:0044282 | C1 | small molecule catabolic process | 0.039834 | 0.038251 |
| GO:0030178 | C1 | negative regulation of Wnt signaling | 0.039834 | 0.021858 |
| | | pathway | | |
| GO:0006024 | C1 | glycosaminoglycan biosynthetic | 0.039834 | 0.013661 |
| | | process | | |
| GO:0060349 | C1 | bone morphogenesis | 0.039834 | 0.015027 |
| GO:0032908 | C1 | regulation of transforming growth | 0.039434 | 0.005464 |
| | | factor betal production | | |
| GO:0010936 | C1 | negative regulation of macrophage | 0.039434 | 0.005464 |
| | | cytokine production | | |
| GO:0030111 | C1 | regulation of Wnt signaling pathway | 0.038808 | 0.034153 |
| GO:0048568 | C1 | embryonic organ development | 0.038808 | 0.043716 |
| GO:0001558 | C1 | regulation of cell growth | 0.038808 | 0.04235 |
| GO:0071559 | C1 | response to transforming growth | 0.038808 | 0.030055 |
| | | factor beta | | |
| GO:0085029 | C1 | extracellular matrix assembly | 0.038056 | 0.010929 |
| GO:1901342 | C1 | regulation of vasculature development | 0.037804 | 0.036885 |
| GO:0006027 | C1 | glycosaminoglycan catabolic process | 0.037737 | 0.008197 |
| GO:0051222 | C1 | positive regulation of protein transport | 0.037737 | 0.032787 |
| GO:0097485 | C1 | neuron projection guidance | 0.035897 | 0.027322 |
| GO:0030850 | C1 | prostate gland development | 0.035435 | 0.010929 |
| GO:0017157 | C1 | regulation of exocytosis | 0.035435 | 0.025956 |
| GO:0050764 | C1 | regulation of phagocytosis | 0.035435 | 0.01776 |
| GO:0007411 | C1 | axon guidance | 0.035435 | 0.027322 |
| GO:0036075 | C1 | replacement ossification | 0.033851 | 0.008197 |
| GO:0001958 | C1 | endochondral ossification | 0.033851 | 0.008197 |
| GO:0071560 | C1 | cellular response to transforming | 0.033851 | 0.030055 |
| | | growth factor beta stimulus | | |
| GO:0060537 | C1 | muscle tissue development | 0.033851 | 0.040984 |
| GO:0006801 | C1 | superoxide metabolic process | 0.033851 | 0.013661 |
| GO:0045765 | C1 | regulation of angiogenesis | 0.033851 | 0.036885 |
| GO:1904149 | C1 | regulation of microglial cell mediated | 0.033851 | 0.004098 |
| | | cytotoxicity | | |
| GO:0090634 | C1 | microglial cell mediated cytotoxicity | 0.033851 | 0.004098 |
| GO:0048251 | C1 | elastic fiber assembly | 0.033851 | 0.006831 |
| GO:0030903 | C1 | notochord development | 0.033851 | 0.006831 |
| GO:0003007 | C1 | heart morphogenesis | 0.033851 | 0.028689 |
| GO:0006790 | C1 | sulfur compound metabolic process | 0.033851 | 0.035519 |
| GO:0032964 | C1 | collagen biosynthetic process | 0.033519 | 0.010929 |
| GO:0046434 | C1 | organophosphate catabolic process | 0.033431 | 0.021858 |
| GO:0097529 | C1 | myeloid leukocyte migration | 0.033431 | 0.030055 |
| GO:0045055 | C1 | regulated exocytosis | 0.033431 | 0.028689 |
| GO:0006909 | C1 | phagocytosis | 0.033431 | 0.034153 |
| GO:0090132 | C1 | epithelium migration | 0.033431 | 0.038251 |
| GO:1905475 | C1 | regulation of protein localization to | 0.033411 | 0.023224 |
| | | membrane | | |
| GO:0048839 | C1 | inner ear development | 0.032882 | 0.02459 |
| GO:0043583 | C1 | ear development | 0.031747 | 0.027322 |
| GO:0010631 | C1 | epithelial cell migration | 0.031747 | 0.038251 |
| GO:0010935 | C1 | regulation of macrophage cytokine | 0.029661 | 0.010929 |
| | | production | | |
| GO:0010934 | C1 | macrophage cytokine production | 0.029661 | 0.010929 |
| GO:0030307 | C1 | positive regulation of cell growth | 0.029661 | 0.023224 |
| GO:0030167 | C1 | proteoglycan catabolic process | 0.029661 | 0.005464 |
| GO:0060326 | C1 | cell chemotaxis | 0.029424 | 0.036885 |
| GO:1901292 | C1 | nucleoside phosphate catabolic | 0.026085 | 0.015027 |
| | | process | | |
| GO:0043627 | C1 | response to estrogen | 0.023442 | 0.013661 |
| GO:0090130 | C1 | tissue migration | 0.022739 | 0.039617 |
| GO:0032928 | C1 | regulation of superoxide anion | 0.022739 | 0.008197 |
| | | generation | | |
| GO:1903510 | C1 | mucopolysaccharide metabolic | 0.022178 | 0.016393 |
| | | process | | |
| GO:1904151 | C1 | positive regulation of microglial cell | 0.022178 | 0.004098 |
| | | mediated cytotoxicity | | |
| GO:0006772 | C1 | thiamine metabolic process | 0.022178 | 0.004098 |
| GO:0060350 | C1 | endochondral bone morphogenesis | 0.022178 | 0.012295 |
| GO:0006887 | C1 | exocytosis | 0.021782 | 0.039617 |
| GO:2000178 | C1 | negative regulation of neural | 0.020433 | 0.008197 |
| | | precursor cell proliferation | | |
| GO:0016051 | C1 | carbohydrate biosynthetic process | 0.018831 | 0.027322 |
| GO:0032905 | C1 | transforming growth factor beta1 | 0.01772 | 0.006831 |
| | | production | | |
| GO:0060840 | C1 | artery development | 0.01701 | 0.01776 |
| GO:0045444 | C1 | fat cell differentiation | 0.016471 | 0.031421 |
| GO:0016485 | C1 | protein processing | 0.016471 | 0.031421 |
| GO:0048570 | C1 | notochord morphogenesis | 0.015837 | 0.005464 |
| GO:0022617 | C1 | extracellular matrix disassembly | 0.015627 | 0.015027 |
| GO:0009166 | C1 | nucleotide catabolic process | 0.014277 | 0.015027 |
| GO:0043277 | C1 | apoptotic cell clearance | 0.014277 | 0.013661 |
| GO:0002685 | C1 | regulation of leukocyte migration | 0.014277 | 0.031421 |
| GO:0043542 | C1 | endothelial cell migration | 0.014277 | 0.034153 |
| GO:0050900 | C1 | leukocyte migration | 0.014277 | 0.045082 |
| GO:0033627 | C1 | cell adhesion mediated by integrin | 0.013969 | 0.016393 |
| GO:0001649 | C1 | osteoblast differentiation | 0.011976 | 0.032787 |
| GO:0035904 | C1 | aorta development | 0.011695 | 0.013661 |
| GO:0090322 | C1 | regulation of superoxide metabolic | 0.009508 | 0.010929 |
| | | process | | |
| GO:0022612 | C1 | gland morphogenesis | 0.008087 | 0.021858 |
| GO:0006022 | C1 | aminoglycan metabolic process | 0.0077 | 0.021858 |
| GO:0002888 | C1 | positive regulation of myeloid | 0.0077 | 0.009563 |
| | | leukocyte mediated immunity | | |
| GO:0006029 | C1 | proteoglycan metabolic process | 0.006888 | 0.01776 |
| GO:0048771 | C1 | tissue remodeling | 0.006888 | 0.025956 |
| GO:0034329 | C1 | cell junction assembly | 0.006001 | 0.047814 |
| GO:0009187 | C1 | cyclic nucleotide metabolic process | 0.004442 | 0.012295 |
| GO:0007160 | C1 | cell-matrix adhesion | 0.003734 | 0.034153 |
| GO:0030203 | C1 | glycosaminoglycan metabolic process | 0.003734 | 0.021858 |
| GO:0060348 | C1 | bone development | 0.003226 | 0.032787 |
| GO:1901136 | C1 | carbohydrate derivative catabolic | 0.001139 | 0.028689 |
| | | process | | |
| GO:0030574 | C1 | collagen catabolic process | 0.000478 | 0.015027 |
| GO:1903829 | C1 | positive regulation of protein | 0.000344 | 0.054645 |
| | | localization | | |
| GO:0001822 | C1 | kidney development | 0.000268 | 0.04235 |
| GO:0031589 | C1 | cell-substrate adhesion | 0.000216 | 0.04918 |
| GO:0072001 | C1 | renal system development | 0.000187 | 0.043716 |
| GO:0051216 | C1 | cartilage development | 7.31E-05 | 0.034153 |
| GO:0001503 | C1 | ossification | 2.67E-05 | 0.057377 |
| GO:0001655 | C1 | urogenital system development | 1.59E-05 | 0.050546 |
| GO:0030199 | C1 | collagen fibril organization | 1.59E-05 | 0.021858 |
| GO:0032963 | C1 | collagen metabolic process | 7.16E-06 | 0.027322 |
| GO:0045229 | C1 | external encapsulating structure | 3.16E-07 | 0.054645 |
| | | organization | | |
| GO:0043062 | C1 | extracellular structure organization | 3.09E-07 | 0.054645 |
| GO:0030198 | C1 | extracellular matrix organization | 3.09E-07 | 0.054645 |
| GO:0061448 | C1 | connective tissue development | 3.09E-07 | 0.047814 |
| GO:0007229 | C1 | integrin-mediated signaling pathway | 3.09E-07 | 0.031421 |
| GO:0006493 | C2 | protein O-linked glycosylation | 4.57E-05 | 2.059925 |
| GO:0010975. . .2 | C2 | regulation of neuron projection | 0.000182 | 5.05618 |
| | | development | | |
| GO:0016266 | C2 | O-glycan processing | 0.000184 | 1.310861 |
| GO:0006486 | C2 | protein glycosylation | 0.000235 | 3.183521 |
| GO:0043413 | C2 | macromolecule glycosylation | 0.000235 | 3.183521 |
| GO:0099150 | C2 | regulation of postsynaptic | 0.00026 | 0.749064 |
| | | specialization assembly | | |
| GO:00072291 | C3 | integrin-mediated signaling pathway | 5.28E-11 | 3.13779 |
| GO:00301981 | C3 | extracellular matrix organization | 5.51E-11 | 5.457026 |
| GO:00430621 | C3 | extracellular structure organization | 6.07E-11 | 5.457026 |
| GO:00452291 | C3 | external encapsulating structure | 7.36E-11 | 5.457026 |
| | | organization | | |
| GO:00614481 | C3 | connective tissue development | 5.85E-10 | 4.774898 |
| GO:00301991 | C3 | collagen fibril organization | 1.91E-09 | 2.18281 |
| GO:00329631 | C3 | collagen metabolic process | 4.57E-09 | 2.728513 |
| GO:00015031 | C3 | ossification | 1.31E-08 | 5.866303 |
| GO:00016551 | C3 | urogenital system development | 2.35E-08 | 5.184175 |
| GO:00512161 | C3 | cartilage development | 2.82E-07 | 3.410641 |
| GO:00720011 | C3 | renal system development | 2.93E-07 | 4.502046 |
| GO:00315891 | C3 | cell-substrate adhesion | 3.90E-07 | 4.911323 |
| GO:00018221 | C3 | kidney development | 4.63E-07 | 4.365621 |
| GO:00305741 | C3 | collagen catabolic process | 8.41E-07 | 1.500682 |
| GO:19038291 | C3 | positive regulation of protein | 1.06E-06 | 5.457026 |
| | | localization | | |
| GO:19011361 | C3 | carbohydrate derivative catabolic | 4.87E-06 | 2.864939 |
| | | process | | |
| GO:00028881 | C3 | positive regulation of myeloid | 6.38E-06 | 0.95498 |
| | | leukocyte mediated immunity | | |
| GO:00071601 | C3 | cell-matrix adhesion | 6.41E-06 | 3.410641 |
| GO:00016491 | C3 | osteoblast differentiation | 1.14E-05 | 3.410641 |
| GO:00603481 | C3 | bone development | 1.36E-05 | 3.274216 |
| GO:00091871 | C3 | cyclic nucleotide metabolic process | 1.75E-05 | 1.227831 |
| GO:00432771 | C3 | apoptotic cell clearance | 2.19E-05 | 1.364256 |
| GO:00226121 | C3 | gland morphogenesis | 2.20E-05 | 2.18281 |
| GO:00302031 | C3 | glycosaminoglycan metabolic process | 2.20E-05 | 2.18281 |
| GO:00226171 | C3 | extracellular matrix disassembly | 2.31E-05 | 1.500682 |
| GO:00343291 | C3 | cell junction assembly | 3.14E-05 | 4.774898 |
| GO:00482511 | C3 | elastic fiber assembly | 3.29E-05 | 0.682128 |
| GO:00026851 | C3 | regulation of leukocyte migration | 3.51E-05 | 3.13779 |
| GO:00903221 | C3 | regulation of superoxide metabolic | 3.65E-05 | 1.091405 |
| | | process | | |
| GO:00509001 | C3 | leukocyte migration | 3.65E-05 | 4.502046 |
| GO:00060291 | C3 | proteoglycan metabolic process | 4.22E-05 | 1.773533 |
| GO:0001667 | C3 | ameboidal-type cell migration | 4.59E-05 | 5.184175 |
| GO:00060221 | C3 | aminoglycan metabolic process | 6.36E-05 | 2.18281 |
| GO:00109341 | C3 | macrophage cytokine production | 7.00E-05 | 1.091405 |
| GO:00109351 | C3 | regulation of macrophage cytokine | 7.00E-05 | 1.091405 |
| | | production | | |
| GO:00487711 | C3 | tissue remodeling | 7.00E-05 | 2.592087 |
| GO:00436271 | C3 | response to estrogen | 7.42E-05 | 1.500682 |
| GO:00454441 | C3 | fat cell differentiation | 8.20E-05 | 3.13779 |
| GO:00326401 | C3 | tumor necrosis factor production | 0.000101 | 2.592087 |
| GO:00326801 | C3 | regulation of tumor necrosis factor | 0.000101 | 2.592087 |
| | | production | | |
| GO:00435421 | C3 | endothelial cell migration | 0.000104 | 3.410641 |
| GO:00164851 | C3 | protein processing | 0.000118 | 3.13779 |
| GO:00359041 | C3 | aorta development | 0.000125 | 1.364256 |
| GO:00603261 | C3 | cell chemotaxis | 0.00013 | 3.683492 |
| GO:00717061 | C3 | tumor necrosis factor superfamily | 0.000145 | 2.592087 |
| | | cytokine production | | |
| GO:19035551 | C3 | regulation of tumor necrosis factor | 0.000145 | 2.592087 |
| | | superfamily cytokine production | | |
| GO:00336271 | C3 | cell adhesion mediated by integrin | 0.000145 | 1.637108 |
| GO:00329281 | C3 | regulation of superoxide anion | 0.000146 | 0.818554 |
| | | generation | | |
| GO:00091661 | C3 | nucleotide catabolic process | 0.000159 | 1.500682 |
| GO:0002683 | C3 | negative regulation of immune system | 0.000166 | 4.638472 |
| | | process | | |
| GO:00507641 | C3 | regulation of phagocytosis | 0.000173 | 1.773533 |
| GO:00329051 | C3 | transforming growth factor beta1 | 0.000188 | 0.682128 |
| | | production | | |
| GO:20001781 | C3 | negative regulation of neural | 0.000191 | 0.818554 |
| | | precursor cell proliferation | | |
| GO:00975291 | C3 | myeloid leukocyte migration | 0.000196 | 3.001364 |
| GO:00160511 | C3 | carbohydrate biosynthetic process | 0.000231 | 2.728513 |
| GO:0070997 | C3 | neuron death | 0.000254 | 3.956344 |
| GO:00608401 | C3 | artery development | 0.000254 | 1.773533 |
| GO:00068871 | C3 | exocytosis | 0.000266 | 3.956344 |
| GO:00303071 | C3 | positive regulation of cell growth | 0.000274 | 2.319236 |
| GO:0050673 | C3 | epithelial cell proliferation | 0.000285 | 4.774898 |
| GO:00300991 | C3 | myeloid cell differentiation | 0.000287 | 4.229195 |
| GO:19035101 | C3 | mucopolysaccharide metabolic | 0.000302 | 1.637108 |
| | | process | | |
| GO:00901301 | C3 | tissue migration | 0.000332 | 3.956344 |
| GO:00850291 | C3 | extracellular matrix assembly | 0.000348 | 1.091405 |
| GO:00485681 | C3 | embryonic organ development | 0.000353 | 4.502046 |
| GO:0034341 | C3 | response to interferon-gamma | 0.000356 | 2.046385 |
| GO:00603501 | C3 | endochondral bone morphogenesis | 0.000366 | 1.227831 |
| GO:00715601 | C3 | cellular response to transforming | 0.000384 | 3.13779 |
| | | growth factor beta stimulus | | |
| GO:19054751 | C3 | regulation of protein localization to | 0.000388 | 2.319236 |
| | | membrane | | |
| GO:00435831 | C3 | ear development | 0.000399 | 2.728513 |
| GO:1901214 | C3 | regulation of neuron death | 0.000414 | 3.547067 |
| GO:00026881 | C3 | regulation of leukocyte chemotaxis | 0.000414 | 1.909959 |
| GO:19012921 | C3 | nucleoside phosphate catabolic | 0.000433 | 1.500682 |
| | | process | | |
| GO:00301781 | C3 | negative regulation of Wnt signaling | 0.000444 | 2.319236 |
| | | pathway | | |
| GO:0006163 | C3 | purine nucleotide metabolic process | 0.000484 | 4.638472 |
| GO:00309031 | C3 | notochord development | 0.000486 | 0.682128 |
| GO:00450551 | C3 | regulated exocytosis | 0.000506 | 2.864939 |
| GO:1902905 | C3 | positive regulation of supramolecular | 0.000507 | 2.319236 |
| | | fiber organization | | |
| GO:00512221 | C3 | positive regulation of protein transport | 0.00051 | 3.274216 |
| GO:00106311 | C3 | epithelial cell migration | 0.000517 | 3.819918 |
| GO:00715591 | C3 | response to transforming growth | 0.000522 | 3.13779 |
| | | factor beta | | |
| GO:00015581 | C3 | regulation of cell growth | 0.000531 | 4.229195 |
| GO:19054771 | C3 | positive regulation of protein | 0.000534 | 1.637108 |
| | | localization to membrane | | |
| GO:00488391 | C3 | inner ear development | 0.000535 | 2.455662 |
| GO:00464341 | C3 | organophosphate catabolic process | 0.000536 | 2.18281 |
| GO:00457651 | C3 | regulation of angiogenesis | 0.000545 | 3.683492 |
| GO:00068011 | C3 | superoxide metabolic process | 0.000551 | 1.364256 |
| GO:00069091 | C3 | phagocytosis | 0.000569 | 3.410641 |
| GO:00901321 | C3 | epithelium migration | 0.000588 | 3.819918 |
| GO:00329641 | C3 | collagen biosynthetic process | 0.000624 | 1.091405 |
| GO:0061082 | C3 | myeloid leukocyte cytokine | 0.000624 | 1.091405 |
| | | production | | |
| GO:0002283 | C3 | neutrophil activation involved in | 0.000639 | 0.682128 |
| | | immune response | | |
| GO:00329301 | C3 | positive regulation of superoxide | 0.000639 | 0.682128 |
| | | anion generation | | |
| GO:00171571 | C3 | regulation of exocytosis | 0.000658 | 2.592087 |
| GO:0071346 | C3 | cellular response to interferon-gamma | 0.000662 | 1.773533 |
| GO:00091501 | C3 | purine ribonucleotide metabolic | 0.000676 | 4.365621 |
| | | process | | |
| GO:00301111 | C3 | regulation of Wnt signaling pathway | 0.000682 | 3.547067 |
| GO:0045637 | C3 | regulation of myeloid cell | 0.000697 | 2.592087 |
| | | differentiation | | |
| GO:19013421 | C3 | regulation of vasculature development | 0.000706 | 3.683492 |
| GO:00308501 | C3 | prostate gland development | 0.000715 | 1.091405 |
| GO:00019581 | C3 | endochondral ossification | 0.000737 | 0.818554 |
| GO:00360751 | C3 | replacement ossification | 0.000737 | 0.818554 |
| GO:00067901 | C3 | sulfur compound metabolic process | 0.000745 | 3.547067 |
| GO:00060241 | C3 | glycosaminoglycan biosynthetic | 0.000758 | 1.364256 |
| | | process | | |
| GO:0032496 | C3 | response to lipopolysaccharide | 0.000778 | 3.547067 |
| GO:00302011 | C3 | heparan sulfate proteoglycan | 0.000781 | 0.95498 |
| | | metabolic process | | |
| GO:00109361 | C3 | negative regulation of macrophage | 0.000865 | 0.545703 |
| | | cytokine production | | |
| GO:00329081 | C3 | regulation of transforming growth | 0.000865 | 0.545703 |
| | | factor betal production | | |
| GO:0002237 | C3 | response to molecule of bacterial | 0.000871 | 3.683492 |
| | | origin | | |
| GO:0002697 | C3 | regulation of immune effector process | 0.000889 | 3.819918 |
| GO:00060271 | C3 | glycosaminoglycan catabolic process | 0.000891 | 0.818554 |
| GO:00074111 | C3 | axon guidance | 0.000916 | 2.728513 |
| GO:00974851 | C3 | neuron projection guidance | 0.000965 | 2.728513 |
| GO:0032526 | C3 | response to retinoic acid | 0.000977 | 1.637108 |
| GO:0002700 | C3 | regulation of production of molecular | 0.001005 | 2.319236 |
| | | mediator of immune response | | |
| GO:1901888 | C3 | regulation of cell junction assembly | 0.001024 | 2.455662 |
| GO:00902011 | C3 | negative regulation of release of | 0.001049 | 0.682128 |
| | | cytochrome c from mitochondria | | |
| GO:00725231 | C3 | purine-containing compound catabolic | 0.001055 | 1.091405 |
| | | process | | |
| GO:1904951 | C3 | positive regulation of establishment of | 0.001071 | 3.274216 |
| | | protein localization | | |
| GO:00487321 | C3 | gland development | 0.001093 | 4.229195 |
| GO:00605371 | C3 | muscle tissue development | 0.001096 | 4.092769 |
| GO:0002366 | C3 | leukocyte activation involved in | 0.001123 | 3.13779 |
| | | immune response | | |
| GO:0030595 | C3 | leukocyte chemotaxis | 0.001126 | 2.728513 |
| GO:00442821 | C3 | small molecule catabolic process | 0.001127 | 3.819918 |
| GO:00060231 | C3 | aminoglycan biosynthetic process | 0.001131 | 1.364256 |
| GO:00030071 | C3 | heart morphogenesis | 0.001154 | 2.864939 |
| GO:00107611 | C3 | fibroblast migration | 0.001194 | 1.091405 |
| GO:0009214 | C3 | cyclic nucleotide catabolic process | 0.001211 | 0.545703 |
| GO:0042554 | C3 | superoxide anion generation | 0.001221 | 0.95498 |
| GO:00329701 | C3 | regulation of actin filament-based | 0.001224 | 3.956344 |
| | | process | | |
| GO:00344461 | C3 | substrate adhesion-dependent cell | 0.001244 | 1.637108 |
| | | spreading | | |
| GO:00082171 | C3 | regulation of blood pressure | 0.001269 | 2.319236 |
| GO:0045216 | C3 | cell-cell junction organization | 0.001277 | 2.455662 |
| GO:00322881 | C3 | myelin assembly | 0.001314 | 0.682128 |
| GO:0001704 | C3 | formation of primary germ layer | 0.001318 | 1.773533 |
| GO:0045927 | C3 | positive regulation of growth | 0.001334 | 2.864939 |
| GO:0009259 | C3 | ribonucleotide metabolic process | 0.001341 | 4.365621 |
| GO:0002263 | C3 | cell activation involved in immune | 0.001343 | 3.13779 |
| | | response | | |
| GO:00426921 | C3 | muscle cell differentiation | 0.001369 | 3.956344 |
| GO:0032635 | C3 | interleukin-6 production | 0.001397 | 2.18281 |
| GO:0032675 | C3 | regulation of interleukin-6 production | 0.001397 | 2.18281 |
| GO:0060562 | C3 | epithelial tube morphogenesis | 0.001426 | 3.410641 |
| GO:0007272 | C3 | ensheathment of neurons | 0.001442 | 1.909959 |
| GO:0008366 | C3 | axon ensheathment | 0.001442 | 1.909959 |
| GO:0050921 | C3 | positive regulation of chemotaxis | 0.001442 | 1.909959 |
| GO:00603491 | C3 | bone morphogenesis | 0.001443 | 1.500682 |
| GO:00516041 | C3 | protein maturation | 0.001486 | 3.410641 |
| GO:00603511 | C3 | cartilage development involved in | 0.001506 | 0.818554 |
| | | endochondral bone morphogenesis | | |
| GO:1903305 | C3 | regulation of regulated secretory | 0.001541 | 1.909959 |
| | | pathway | | |
| GO:0061564 | C3 | axon development | 0.001564 | 4.502046 |
| GO:00020621 | C3 | chondrocyte differentiation | 0.001568 | 1.637108 |
| GO:0046058 | C3 | CAMP metabolic process | 0.001626 | 0.682128 |
| GO:0010594 | C3 | regulation of endothelial cell | 0.001638 | 2.592087 |
| | | migration | | |
| GO:0050920 | C3 | regulation of chemotaxis | 0.001638 | 2.592087 |
| GO:0050727 | C3 | regulation of inflammatory response | 0.001644 | 3.956344 |
| GO:0018158 | C3 | protein oxidation | 0.001644 | 0.545703 |
| GO:0070307 | C3 | lens fiber cell development | 0.001644 | 0.545703 |
| GO:00620121 | C3 | regulation of small molecule | 0.001678 | 3.410641 |
| | | metabolic process | | |
| GO:0007369 | C3 | gastrulation | 0.001679 | 2.319236 |
| GO:0006575 | C3 | cellular modified amino acid | 0.001765 | 2.18281 |
| | | metabolic process | | |
| GO:0019693 | C3 | ribose phosphate metabolic process | 0.00182 | 4.365621 |
| GO:0048880 | C3 | sensory system development | 0.001842 | 3.819918 |
| GO:0002687 | C3 | positive regulation of leukocyte | 0.001871 | 1.909959 |
| | | migration | | |
| GO:1903078 | C3 | positive regulation of protein | 0.001899 | 1.091405 |
| | | localization to plasma membrane | | |
| GO:0018108 | C3 | peptidyl-tyrosine phosphorylation | 0.001914 | 3.683492 |
| GO:0018212 | C3 | peptidyl-tyrosine modification | 0.002062 | 3.683492 |
| GO:0002718 | C3 | regulation of cytokine production | 0.002106 | 1.637108 |
| | | involved in immune response | | |
| GO:0002886 | C3 | regulation of myeloid leukocyte | 0.002118 | 1.091405 |
| | | mediated immunity | | |
| GO:0002573 | C3 | myeloid leukocyte differentiation | 0.002155 | 2.455662 |
| GO:1903076 | C3 | regulation of protein localization to | 0.002159 | 1.500682 |
| | | plasma membrane | | |
| GO:0035924 | C3 | cellular response to vascular | 0.002192 | 1.227831 |
| | | endothelial growth factor stimulus | | |
| GO:0050766 | C3 | positive regulation of phagocytosis | 0.002192 | 1.227831 |
| GO:0042060 | C3 | wound healing | 0.002228 | 4.092769 |
| GO:0046849 | C3 | bone remodeling | 0.002322 | 1.364256 |
| GO:0051963 | C3 | regulation of synapse assembly | 0.002332 | 1.500682 |
| GO:0030865 | C3 | cortical cytoskeleton organization | 0.002356 | 1.091405 |
| GO:0034113 | C3 | heterotypic cell-cell adhesion | 0.002356 | 1.091405 |
| GO:0006195 | C3 | purine nucleotide catabolic process | 0.002361 | 0.95498 |
| GO:0006026 | C3 | aminoglycan catabolic process | 0.002399 | 0.818554 |
| GO:0018149 | C3 | peptide cross-linking | 0.002399 | 0.818554 |
| GO:0072006 | C3 | nephron development | 0.002401 | 1.909959 |
| GO:0006658 | C3 | phosphatidylserine metabolic process | 0.002408 | 0.682128 |
| GO:0002367 | C3 | cytokine production involved in | 0.002427 | 1.637108 |
| | | immune response | | |
| GO:1903052 | C3 | positive regulation of proteolysis | 0.002443 | 1.773533 |
| | | involved in protein catabolic process | | |
| GO:0001819 | C3 | positive regulation of cytokine | 0.002599 | 4.365621 |
| | | production | | |
| GO:0032355 | C3 | response to estradiol | 0.002602 | 1.637108 |
| GO:0010810 | C3 | regulation of cell-substrate adhesion | 0.002627 | 2.455662 |
| GO:0007178 | C3 | transmembrane receptor protein | 0.002753 | 3.683492 |
| | | serine/threonine kinase signaling | | |
| | | pathway | | |
| GO:0071402 | C3 | cellular response to lipoprotein | 0.002771 | 0.818554 |
| | | particle stimulus | | |
| GO:0030214 | C3 | hyaluronan catabolic process | 0.002811 | 0.545703 |
| GO:0045862 | C3 | positive regulation of proteolysis | 0.002875 | 3.547067 |
| GO:0033598 | C3 | mammary gland epithelial cell | 0.002888 | 0.682128 |
| | | proliferation | | |
| GO:0060343 | C3 | trabecula formation | 0.002888 | 0.682128 |
| GO:0050679 | C3 | positive regulation of epithelial cell | 0.002893 | 2.455662 |
| | | proliferation | | |
| GO:0150063 | C3 | visual system development | 0.002954 | 3.683492 |
| GO:1903533 | C3 | regulation of protein targeting | 0.003155 | 1.227831 |
| GO:0090316 | C3 | positive regulation of intracellular | 0.003232 | 1.909959 |
| | | protein transport | | |
| GO:0048705 | C3 | skeletal system morphogenesis | 0.003334 | 2.455662 |
| GO:0045667 | C3 | regulation of osteoblast differentiation | 0.00335 | 1.773533 |
| GO:0090287 | C3 | regulation of cellular response to | 0.003351 | 3.274216 |
| | | growth factor stimulus | | |
| GO:0030225 | C3 | macrophage differentiation | 0.003358 | 0.95498 |
| GO:1904375 | C3 | regulation of protein localization to | 0.003406 | 1.637108 |
| | | cell periphery | | |
| GO:0036296 | C3 | response to increased oxygen levels | 0.003432 | 0.682128 |
| GO:0045745 | C3 | positive regulation of G protein- | 0.003432 | 0.682128 |
| | | coupled receptor signaling pathway | | |
| GO:0043299 | C3 | leukocyte degranulation | 0.003441 | 1.227831 |
| GO:0032102 | C3 | negative regulation of response to | 0.003493 | 3.956344 |
| | | external stimulus | | |
| GO:0003170 | C3 | heart valve development | 0.003528 | 1.091405 |
| GO:0042552 | C3 | myelination | 0.00356 | 1.773533 |
| GO:0009084 | C3 | glutamine family amino acid | 0.003564 | 0.545703 |
| | | biosynthetic process | | |
| GO:0010763 | C3 | positive regulation of fibroblast | 0.003564 | 0.545703 |
| | | migration | | |
| GO:0016202 | C3 | regulation of striated muscle tissue | 0.003564 | 0.545703 |
| | | development | | |
| GO:0072109 | C3 | glomerular mesangium development | 0.003564 | 0.545703 |
| GO:0010823 | C3 | negative regulation of mitochondrion | 0.003754 | 0.95498 |
| | | organization | | |
| GO:0051099 | C3 | positive regulation of binding | 0.003816 | 2.046385 |
| GO:0051495 | C3 | positive regulation of cytoskeleton | 0.003936 | 2.18281 |
| | | organization | | |
| GO:0002064 | C3 | epithelial cell development | 0.003996 | 2.319236 |
| GO:0071772 | C3 | response to BMP | 0.004022 | 2.046385 |
| GO:0071773 | C3 | cellular response to BMP stimulus | 0.004022 | 2.046385 |
| GO:0002275 | C3 | myeloid cell activation involved in | 0.004047 | 1.364256 |
| | | immune response | | |
| GO:0060512 | C3 | prostate gland morphogenesis | 0.004047 | 0.682128 |
| GO:0010762 | C3 | regulation of fibroblast migration | 0.004148 | 0.818554 |
| GO:0030866 | C3 | cortical actin cytoskeleton | 0.004148 | 0.818554 |
| | | organization | | |
| GO:0071622 | C3 | regulation of granulocyte chemotaxis | 0.004183 | 0.95498 |
| GO:0048608 | C3 | reproductive structure development | 0.004189 | 3.001364 |
| GO:1904377 | C3 | positive regulation of protein | 0.004261 | 1.091405 |
| | | localization to cell periphery | | |
| GO:0002690 | C3 | positive regulation of leukocyte | 0.004359 | 1.364256 |
| | | chemotaxis | | |
| GO:0050714 | C3 | positive regulation of protein | 0.004399 | 1.637108 |
| | | secretion | | |
| GO:0032956 | C3 | regulation of actin cytoskeleton | 0.004441 | 3.410641 |
| | | organization | | |
| GO:0071219 | C3 | cellular response to molecule of | 0.004583 | 2.455662 |
| | | bacterial origin | | |
| GO:0007179 | C3 | transforming growth factor beta | 0.004599 | 2.319236 |
| | | receptor signaling pathway | | |
| GO:0032642 | C3 | regulation of chemokine production | 0.004691 | 1.364256 |
| GO:0042119 | C3 | neutrophil activation | 0.004703 | 0.818554 |
| GO:0032388 | C3 | positive regulation of intracellular | 0.004789 | 2.18281 |
| | | transport | | |
| GO:0031348 | C3 | negative regulation of defense | 0.004813 | 2.728513 |
| | | response | | |
| GO:0046034 | C3 | ATP metabolic process | 0.004816 | 2.319236 |
| GO:0061458 | C3 | reproductive system development | 0.004877 | 3.001364 |
| GO:0046890 | C3 | regulation of lipid biosynthetic | 0.004942 | 2.046385 |
| | | process | | |
| GO:1901215 | C3 | negative regulation of neuron death | 0.005041 | 2.319236 |
| GO:0032602 | C3 | chemokine production | 0.005041 | 1.364256 |
| GO:0010632 | C3 | regulation of epithelial cell migration | 0.005055 | 2.864939 |
| GO:0061180 | C3 | mammary gland epithelium | 0.005107 | 1.091405 |
| | | development | | |
| GO:0003179 | C3 | heart valve morphogenesis | 0.005153 | 0.95498 |
| GO:0038093 | C3 | Fc receptor signaling pathway | 0.005153 | 0.95498 |
| GO:0072132 | C3 | mesenchyme morphogenesis | 0.005153 | 0.95498 |
| GO:0031331 | C3 | positive regulation of cellular | 0.005247 | 3.819918 |
| | | catabolic process | | |
| GO:0030316 | C3 | osteoclast differentiation | 0.005412 | 1.364256 |
| GO:0045618 | C3 | positive regulation of keratinocyte | 0.005457 | 0.545703 |
| | | differentiation | | |
| GO:0061298 | C3 | retina vasculature development in | 0.005457 | 0.545703 |
| | | camera-type eye | | |
| GO:0140354 | C3 | lipid import into cell | 0.005457 | 0.545703 |
| GO:0048562 | C3 | embryonic organ morphogenesis | 0.005458 | 2.864939 |
| GO:0008045 | C3 | motor neuron axon guidance | 0.005506 | 0.682128 |
| GO:0014706 | C3 | striated muscle tissue development | 0.005576 | 2.592087 |
| GO:0002244 | C3 | hematopoietic progenitor cell | 0.005613 | 1.637108 |
| | | differentiation | | |
| GO:0051098 | C3 | regulation of binding | 0.005637 | 3.410641 |
| GO:0019932 | C3 | second-messenger-mediated signaling | 0.005657 | 3.001364 |
| GO:0003183 | C3 | mitral valve morphogenesis | 0.00568 | 0.409277 |
| GO:0007168 | C3 | receptor guanylyl cyclase signaling | 0.00568 | 0.409277 |
| | | pathway | | |
| GO:0060346 | C3 | bone trabecula formation | 0.00568 | 0.409277 |
| GO:0071377 | C3 | cellular response to glucagon stimulus | 0.00568 | 0.409277 |
| GO:2000425 | C3 | regulation of apoptotic cell clearance | 0.00568 | 0.409277 |
| GO:0016049 | C3 | cell growth | 0.00586 | 4.229195 |
| GO:0048639 | C3 | positive regulation of developmental | 0.005897 | 1.909959 |
| | | growth | | |
| GO:0009154 | C3 | purine ribonucleotide catabolic | 0.005974 | 0.818554 |
| | | process | | |
| GO:0045687 | C3 | positive regulation of glial cell | 0.005974 | 0.818554 |
| | | differentiation | | |
| GO:0044242 | C3 | cellular lipid catabolic process | 0.006028 | 2.319236 |
| GO:0071222 | C3 | cellular response to | 0.006028 | 2.319236 |
| | | lipopolysaccharide | | |
| GO:0007015 | C3 | actin filament organization | 0.006108 | 3.956344 |
| GO:0019218 | C3 | regulation of steroid metabolic | 0.006218 | 1.364256 |
| | | process | | |
| GO:0009100 | C3 | glycoprotein metabolic process | 0.006279 | 3.547067 |
| GO:0007043 | C3 | cell-cell junction assembly | 0.006315 | 1.773533 |
| GO:0045766 | C3 | positive regulation of angiogenesis | 0.006323 | 2.046385 |
| GO:1904018 | C3 | positive regulation of vasculature | 0.006323 | 2.046385 |
| | | development | | |
| GO:0061437 | C3 | renal system vasculature development | 0.006359 | 0.682128 |
| GO:0061440 | C3 | kidney vasculature development | 0.006359 | 0.682128 |
| GO:0009205 | C3 | purine ribonucleoside triphosphate | 0.006458 | 2.455662 |
| | | metabolic process | | |
| GO:0001892 | C3 | embryonic placenta development | 0.006531 | 1.227831 |
| GO:0071277 | C3 | cellular response to calcium ion | 0.006531 | 1.227831 |
| GO:0002699 | C3 | positive regulation of immune effector | 0.006553 | 2.592087 |
| | | process | | |
| GO:0071216 | C3 | cellular response to biotic stimulus | 0.006553 | 2.592087 |
| GO:0010927 | C3 | cellular component assembly involved | 0.006555 | 1.500682 |
| | | in morphogenesis | | |
| GO:0097191 | C3 | extrinsic apoptotic signaling pathway | 0.006578 | 2.319236 |
| GO:0009110 | C3 | vitamin biosynthetic process | 0.006615 | 0.545703 |
| GO:0044794 | C3 | positive regulation by host of viral | 0.006615 | 0.545703 |
| | | process | | |
| GO:0006006 | C3 | glucose metabolic process | 0.006633 | 2.046385 |
| GO:1900026 | C3 | positive regulation of substrate | 0.006695 | 0.818554 |
| | | adhesion-dependent cell spreading | | |
| GO:0046395 | C3 | carboxylic acid catabolic process | 0.006731 | 2.455662 |
| GO:0033619 | C3 | membrane protein proteolysis | 0.006912 | 0.95498 |
| GO:0002285 | C3 | lymphocyte activation involved in | 0.006948 | 2.18281 |
| | | immune response | | |
| GO:0002312 | C3 | B cell activation involved in immune | 0.007032 | 1.227831 |
| | | response | | |
| GO:0061035 | C3 | regulation of cartilage development | 0.007177 | 1.091405 |
| GO:0010038 | C3 | response to metal ion | 0.007256 | 3.274216 |
| GO:0006516 | C3 | glycoprotein catabolic process | 0.007301 | 0.682128 |
| GO:0007435 | C3 | salivary gland morphogenesis | 0.007301 | 0.682128 |
| GO:1903955 | C3 | positive regulation of protein targeting | 0.007301 | 0.682128 |
| | | to mitochondrion | | |
| GO:0016052 | C3 | carbohydrate catabolic process | 0.007409 | 1.773533 |
| GO:0030890 | C3 | positive regulation of B cell | 0.007478 | 0.818554 |
| | | proliferation | | |
| GO:0003174 | C3 | mitral valve development | 0.007587 | 0.409277 |
| GO:0006182 | C3 | cGMP biosynthetic process | 0.007587 | 0.409277 |
| GO:0060900 | C3 | embryonic camera-type eye formation | 0.007587 | 0.409277 |
| GO:1904238 | C3 | pericyte cell differentiation | 0.007587 | 0.409277 |
| GO:1905634 | C3 | regulation of protein localization to | 0.007587 | 0.409277 |
| | | chromatin | | |
| GO:0009144 | C3 | purine nucleoside triphosphate | 0.007918 | 2.455662 |
| | | metabolic process | | |
| GO:0016054 | C3 | organic acid catabolic process | 0.007918 | 2.455662 |
| GO:0046364 | C3 | monosaccharide biosynthetic process | 0.008122 | 1.227831 |
| GO:0019318 | C3 | hexose metabolic process | 0.008131 | 2.319236 |
| GO:0071241 | C3 | cellular response to inorganic | 0.008131 | 2.319236 |
| | | substance | | |
| GO:0005996 | C3 | monosaccharide metabolic process | 0.008239 | 2.455662 |
| GO:0038094 | C3 | Fc-gamma receptor signaling pathway | 0.008336 | 0.682128 |
| GO:0045577 | C3 | regulation of B cell differentiation | 0.008336 | 0.682128 |
| GO:0071675 | C3 | regulation of mononuclear cell | 0.008347 | 1.500682 |
| | | migration | | |
| GO:0050810 | C3 | regulation of steroid biosynthetic | 0.008422 | 1.091405 |
| | | process | | |
| GO:0009199 | C3 | ribonucleoside triphosphate metabolic | 0.008571 | 2.455662 |
| | | process | | |
| GO:0002444 | C3 | myeloid leukocyte mediated immunity | 0.008643 | 1.364256 |
| GO:0010975. . .320 | C3 | regulation of neuron projection | 0.008719 | 3.819918 |
| | | development | | |
| GO:0010720 | C3 | positive regulation of cell | 0.008774 | 2.864939 |
| | | development | | |
| GO:0090090 | C3 | negative regulation of canonical Wnt | 0.008828 | 1.637108 |
| | | signaling pathway | | |
| GO:0019216 | C3 | regulation of lipid metabolic process | 0.009035 | 3.13779 |
| GO:0002637 | C3 | regulation of immunoglobulin | 0.009103 | 1.091405 |
| | | production | | |
| GO:0006090 | C3 | pyruvate metabolic process | 0.009205 | 1.364256 |
| GO:0002861 | C3 | regulation of inflammatory response | 0.009237 | 0.818554 |
| | | to antigenic stimulus | | |
| GO:0036230 | C3 | granulocyte activation | 0.009237 | 0.818554 |
| GO:0050919 | C3 | negative chemotaxis | 0.009237 | 0.818554 |
| GO:1902622 | C3 | regulation of neutrophil migration | 0.009237 | 0.818554 |
| GO:0019439 | C3 | aromatic compound catabolic process | 0.009248 | 4.092769 |
| GO:0010977 | C3 | negative regulation of neuron | 0.009314 | 1.637108 |
| | | projection development | | |
| GO:0007517 | C3 | muscle organ development | 0.009333 | 3.13779 |
| GO:0001656 | C3 | metanephros development | 0.009335 | 1.227831 |
| GO:0010811 | C3 | positive regulation of cell-substrate | 0.009374 | 1.500682 |
| | | adhesion | | |
| GO:0001654 | C3 | eye development | 0.009394 | 3.410641 |
| GO:0009190 | C3 | cyclic nucleotide biosynthetic process | 0.009394 | 0.545703 |
| GO:0048634 | C3 | regulation of muscle organ | 0.009394 | 0.545703 |
| | | development | | |
| GO:0050765 | C3 | negative regulation of phagocytosis | 0.009394 | 0.545703 |
| GO:0052652 | C3 | cyclic purine nucleotide metabolic | 0.009394 | 0.545703 |
| | | process | | |
| GO:0071404 | C3 | cellular response to low-density | 0.009394 | 0.545703 |
| | | lipoprotein particle stimulus | | |
| GO:2001044 | C3 | regulation of integrin-mediated | 0.009394 | 0.545703 |
| | | signaling pathway | | |
| GO:0007431 | C3 | salivary gland development | 0.009469 | 0.682128 |
| GO:0034405 | C3 | response to fluid shear stress | 0.009469 | 0.682128 |
| GO:0055094 | C3 | response to lipoprotein particle | 0.009469 | 0.682128 |
| GO:0090022 | C3 | regulation of neutrophil chemotaxis | 0.009469 | 0.682128 |
| GO:0032720 | C3 | negative regulation of tumor necrosis | 0.009823 | 1.091405 |
| | | factor production | | |
| GO:0002551 | C3 | mast cell chemotaxis | 0.009828 | 0.409277 |
| GO:0043312 | C3 | neutrophil degranulation | 0.009828 | 0.409277 |
| GO:0043589 | C3 | skin morphogenesis | 0.009828 | 0.409277 |
| GO:0071635 | C3 | negative regulation of transforming | 0.009828 | 0.409277 |
| | | growth factor beta production | | |
| GO:0072110 | C3 | glomerular mesangial cell | 0.009828 | 0.409277 |
| | | proliferation | | |
| GO:0050803 | C3 | regulation of synapse structure or | 0.009829 | 2.18281 |
| | | activity | | |
| GO:0031102 | C3 | neuron projection regeneration | 0.009903 | 0.95498 |
| GO:0032233 | C3 | positive regulation of actin filament | 0.009903 | 0.95498 |
| | | bundle assembly | | |
| GO:0031641 | C3 | regulation of myelination | 0.01022 | 0.818554 |
| GO:0060976 | C3 | coronary vasculature development | 0.01022 | 0.818554 |
| GO:0006936 | C3 | muscle contraction | 0.010276 | 3.13779 |
| GO:0001818 | C3 | negative regulation of cytokine | 0.010302 | 3.274216 |
| | | production | | |
| GO:0034612 | C3 | response to tumor necrosis factor | 0.010396 | 2.455662 |
| GO:2000060 | C3 | positive regulation of ubiquitin- | 0.010411 | 1.364256 |
| | | dependent protein catabolic process | | |
| GO:0071248 | C3 | cellular response to metal ion | 0.010457 | 2.046385 |
| GO:0051146 | C3 | striated muscle cell differentiation | 0.0106 | 2.728513 |
| GO:0001893 | C3 | maternal placenta development | 0.010702 | 0.682128 |
| GO:0002719 | C3 | negative regulation of cytokine | 0.010702 | 0.682128 |
| | | production involved in immune | | |
| | | response | | |
| GO:0030212 | C3 | hyaluronan metabolic process | 0.010702 | 0.682128 |
| GO:0031667 | C3 | response to nutrient levels | 0.010749 | 3.956344 |
| GO:0060485 | C3 | mesenchyme development | 0.010778 | 2.864939 |
| GO:0051402 | C3 | neuron apoptotic process | 0.010796 | 2.455662 |
| GO:0001935 | C3 | endothelial cell proliferation | 0.010919 | 2.046385 |
| GO:0018958 | C3 | phenol-containing compound | 0.011058 | 1.364256 |
| | | metabolic process | | |
| GO:0006941 | C3 | striated muscle contraction | 0.01106 | 1.909959 |
| GO:0110148 | C3 | biomineralization | 0.01106 | 1.909959 |
| GO:0048706 | C3 | embryonic skeletal system | 0.011095 | 1.500682 |
| | | development | | |
| GO:0043491 | C3 | protein kinase B signaling | 0.011126 | 2.18281 |
| GO:0043523 | C3 | regulation of neuron apoptotic process | 0.011126 | 2.18281 |
| GO:0050678 | C3 | regulation of epithelial cell | 0.011163 | 3.547067 |
| | | proliferation | | |
| GO:0009261 | C3 | ribonucleotide catabolic process | 0.011274 | 0.818554 |
| GO:0043300 | C3 | regulation of leukocyte degranulation | 0.011274 | 0.818554 |
| GO:0002437 | C3 | inflammatory response to antigenic | 0.01139 | 1.091405 |
| | | stimulus | | |
| GO:0048844 | C3 | artery morphogenesis | 0.01139 | 1.091405 |
| GO:0061333 | C3 | renal tubule morphogenesis | 0.01139 | 1.091405 |
| GO:1903556 | C3 | negative regulation of tumor necrosis | 0.01139 | 1.091405 |
| | | factor superfamily cytokine | | |
| | | production | | |
| GO:0008154 | C3 | actin polymerization or | 0.011397 | 2.046385 |
| | | depolymerization | | |
| GO:0016055 | C3 | Wnt signaling pathway | 0.011523 | 3.819918 |
| GO:0016042 | C3 | lipid catabolic process | 0.011993 | 3.001364 |
| GO:0016486 | C3 | peptide hormone processing | 0.012042 | 0.682128 |
| GO:0050869 | C3 | negative regulation of B cell | 0.012042 | 0.682128 |
| | | activation | | |
| GO:0070306 | C3 | lens fiber cell differentiation | 0.012042 | 0.682128 |
| GO:1903749 | C3 | positive regulation of establishment of | 0.012042 | 0.682128 |
| | | protein localization to mitochondrion | | |
| GO:0044000 | C3 | movement in host | 0.012098 | 1.909959 |
| GO:0198738 | C3 | cell-cell signaling by wnt | 0.012163 | 3.819918 |
| GO:0072080 | C3 | nephron tubule development | 0.012168 | 1.227831 |
| GO:0071230 | C3 | cellular response to amino acid | 0.01224 | 1.091405 |
| | | stimulus | | |
| GO:0072028 | C3 | nephron morphogenesis | 0.01224 | 1.091405 |
| GO:0070482 | C3 | response to oxygen levels | 0.012379 | 3.001364 |
| GO:0060100 | C3 | positive regulation of phagocytosis, | 0.012413 | 0.409277 |
| | | engulfment | | |
| GO:0060707 | C3 | trophoblast giant cell differentiation | 0.012413 | 0.409277 |
| GO:0061430 | C3 | bone trabecula morphogenesis | 0.012413 | 0.409277 |
| GO:0072537 | C3 | fibroblast activation | 0.012413 | 0.409277 |
| GO:0097475 | C3 | motor neuron migration | 0.012413 | 0.409277 |
| GO:1901862 | C3 | negative regulation of muscle tissue | 0.012413 | 0.409277 |
| | | development | | |
| GO:1903624 | C3 | regulation of DNA catabolic process | 0.012413 | 0.409277 |
| GO:1905155 | C3 | positive regulation of membrane | 0.012413 | 0.409277 |
| | | invagination | | |
| GO:0007416 | C3 | synapse assembly | 0.012645 | 1.909959 |
| GO:0050728 | C3 | negative regulation of inflammatory | 0.012645 | 1.909959 |
| | | response | | |
| GO:0044272 | C3 | sulfur compound biosynthetic process | 0.012684 | 1.637108 |
| GO:0051962 | C3 | positive regulation of nervous system | 0.012813 | 2.592087 |
| | | development | | |
| GO:0060706 | C3 | cell differentiation involved in | 0.012838 | 0.545703 |
| | | embryonic placenta development | | |
| GO:0060907 | C3 | positive regulation of macrophage | 0.012838 | 0.545703 |
| | | cytokine production | | |
| GO:0090208 | C3 | positive regulation of triglyceride | 0.012838 | 0.545703 |
| | | metabolic process | | |
| GO:0060828 | C3 | regulation of canonical Wnt signaling | 0.012981 | 2.455662 |
| | | pathway | | |
| GO:0046486 | C3 | glycerolipid metabolic process | 0.013005 | 3.410641 |
| GO:0010595 | C3 | positive regulation of endothelial cell | 0.013048 | 1.500682 |
| | | migration | | |
| GO:2000377 | C3 | regulation of reactive oxygen species | 0.013326 | 1.637108 |
| | | metabolic process | | |
| GO:0003205 | C3 | cardiac chamber development | 0.013359 | 1.773533 |
| GO:0030509 | C3 | BMP signaling pathway | 0.013359 | 1.773533 |
| GO:0070665 | C3 | positive regulation of leukocyte | 0.013359 | 1.773533 |
| | | proliferation | | |
| GO:0006734 | C3 | NADH metabolic process | 0.01349 | 0.682128 |
| GO:0031076 | C3 | embryonic camera-type eye | 0.01349 | 0.682128 |
| | | development | | |
| GO:0043010 | C3 | camera-type eye development | 0.013599 | 3.001364 |
| GO:0009132 | C3 | nucleoside diphosphate metabolic | 0.013753 | 1.500682 |
| | | process | | |
| GO:0006109 | C3 | regulation of carbohydrate metabolic | 0.013795 | 1.909959 |
| | | process | | |
| GO:0001938 | C3 | positive regulation of endothelial cell | 0.013953 | 1.364256 |
| | | proliferation | | |
| GO:0045785 | C3 | positive regulation of cell adhesion | 0.013973 | 3.956344 |
| GO:0001890 | C3 | placenta development | 0.013993 | 1.637108 |
| GO:0043588 | C3 | skin development | 0.014374 | 2.728513 |
| GO:0002221 | C3 | pattern recognition receptor signaling | 0.0144 | 1.909959 |
| | | pathway | | |
| GO:0001101 | C3 | response to acid chemical | 0.014487 | 1.500682 |
| GO:0002702 | C3 | positive regulation of production of | 0.014487 | 1.500682 |
| | | molecular mediator of immune | | |
| | | response | | |
| GO:0007568 | C3 | aging | 0.014632 | 1.773533 |
| GO:0061326 | C3 | renal tubule development | 0.014679 | 1.227831 |
| GO:2001237 | C3 | negative regulation of extrinsic | 0.014679 | 1.227831 |
| | | apoptotic signaling pathway | | |
| GO:0046718 | C3 | viral entry into host cell | 0.014684 | 1.637108 |
| GO:0048745 | C3 | smooth muscle tissue development | 0.014825 | 0.545703 |
| GO:0030888 | C3 | regulation of B cell proliferation | 0.01485 | 0.95498 |
| GO:0048013 | C3 | ephrin receptor signaling pathway | 0.014895 | 0.818554 |
| GO:0003203 | C3 | endocardial cushion morphogenesis | 0.015051 | 0.682128 |
| GO:0014037 | C3 | Schwann cell differentiation | 0.015051 | 0.682128 |
| GO:0032885 | C3 | regulation of polysaccharide | 0.015051 | 0.682128 |
| | | biosynthetic process | | |
| GO:0140448 | C3 | signaling receptor ligand precursor | 0.015051 | 0.682128 |
| | | processing | | |
| GO:0006094 | C3 | gluconeogenesis | 0.015066 | 1.091405 |
| GO:0006096 | C3 | glycolytic process | 0.015066 | 1.091405 |
| GO:0050848 | C3 | regulation of calcium-mediated | 0.015066 | 1.091405 |
| | | signaling | | |
| GO:0003188 | C3 | heart valve formation | 0.015349 | 0.409277 |
| GO:0009159 | C3 | deoxyribonucleoside monophosphate | 0.015349 | 0.409277 |
| | | catabolic process | | |
| GO:0070141 | C3 | response to UV-A | 0.015349 | 0.409277 |
| GO:0050867 | C3 | positive regulation of cell activation | 0.01541 | 3.819918 |
| GO:0006165 | C3 | nucleoside diphosphate | 0.015596 | 1.227831 |
| | | phosphorylation | | |
| GO:0072659 | C3 | protein localization to plasma | 0.015724 | 2.592087 |
| | | membrane | | |
| GO:0050730 | C3 | regulation of peptidyl-tyrosine | 0.016048 | 2.455662 |
| | | phosphorylation | | |
| GO:0006757 | C3 | ATP generation from ADP | 0.016106 | 1.091405 |
| GO:0008589 | C3 | regulation of smoothened signaling | 0.016106 | 1.091405 |
| | | pathway | | |
| GO:0002639 | C3 | positive regulation of immunoglobulin | 0.016262 | 0.818554 |
| | | production | | |
| GO:0050777 | C3 | negative regulation of immune | 0.016337 | 1.909959 |
| | | response | | |
| GO:0034655 | C3 | nucleobase-containing compound | 0.016446 | 3.547067 |
| | | catabolic process | | |
| GO:0043200 | C3 | response to amino acid | 0.01647 | 1.364256 |
| GO:1901800 | C3 | positive regulation of proteasomal | 0.01647 | 1.364256 |
| | | protein catabolic process | | |
| GO:0030100 | C3 | regulation of endocytosis | 0.016473 | 2.046385 |
| GO:0045732 | C3 | positive regulation of protein | 0.016473 | 2.046385 |
| | | catabolic process | | |
| GO:0050807 | C3 | regulation of synapse organization | 0.016473 | 2.046385 |
| GO:0009141 | C3 | nucleoside triphosphate metabolic | 0.01661 | 2.455662 |
| | | process | | |
| GO:0032350 | C3 | regulation of hormone metabolic | 0.016729 | 0.682128 |
| | | process | | |
| GO:0044273 | C3 | sulfur compound catabolic process | 0.016729 | 0.682128 |
| GO:0045616 | C3 | regulation of keratinocyte | 0.016729 | 0.682128 |
| | | differentiation | | |
| GO:0044403 | C3 | biological process involved in | 0.016889 | 2.728513 |
| | | symbiotic interaction | | |
| GO:0006929 | C3 | substrate-dependent cell migration | 0.016995 | 0.545703 |
| GO:0060740 | C3 | prostate gland epithelium | 0.016995 | 0.545703 |
| | | morphogenesis | | |
| GO:0055001 | C3 | muscle cell development | 0.017025 | 1.909959 |
| GO:0014015 | C3 | positive regulation of gliogenesis | 0.01725 | 0.95498 |
| GO:0030858 | C3 | positive regulation of epithelial cell | 0.01725 | 0.95498 |
| | | differentiation | | |
| GO:0070830 | C3 | bicellular tight junction assembly | 0.01725 | 0.95498 |
| GO:0032609 | C3 | interferon-gamma production | 0.01738 | 1.364256 |
| GO:0032649 | C3 | regulation of interferon-gamma | 0.01738 | 1.364256 |
| | | production | | |
| GO:0007389 | C3 | pattern specification process | 0.017494 | 3.819918 |
| GO:0045926 | C3 | negative regulation of growth | 0.017499 | 2.319236 |
| GO:0046939 | C3 | nucleotide phosphorylation | 0.017554 | 1.227831 |
| GO:2001236 | C3 | regulation of extrinsic apoptotic | 0.017714 | 1.637108 |
| | | signaling pathway | | |
| GO:0009314 | C3 | response to radiation | 0.017894 | 3.683492 |
| GO:0031214 | C3 | biomineral tissue development | 0.018223 | 1.773533 |
| GO:0050808 | C3 | synapse organization | 0.018274 | 3.547067 |
| GO:0019319 | C3 | hexose biosynthetic process | 0.018338 | 1.091405 |
| GO:0019935 | C3 | cyclic-nucleotide-mediated signaling | 0.018338 | 1.091405 |
| GO:0032092 | C3 | positive regulation of protein binding | 0.018338 | 1.091405 |
| GO:0003018 | C3 | vascular process in circulatory system | 0.018388 | 2.455662 |
| GO:0006836 | C3 | neurotransmitter transport | 0.018503 | 2.046385 |
| GO:0140467 | C3 | integrated stress response signaling | 0.018526 | 0.682128 |
| GO:0032722 | C3 | positive regulation of chemokine | 0.018547 | 0.95498 |
| | | production | | |
| GO:0048144 | C3 | fibroblast proliferation | 0.018598 | 1.227831 |
| GO:0009111 | C3 | vitamin catabolic process | 0.018643 | 0.409277 |
| GO:0032488 | C3 | Cdc42 protein signal transduction | 0.018643 | 0.409277 |
| GO:0032897 | C3 | negative regulation of viral | 0.018643 | 0.409277 |
| | | transcription | | |
| GO:0045579 | C3 | positive regulation of B cell | 0.018643 | 0.409277 |
| | | differentiation | | |
| GO:0060026 | C3 | convergent extension | 0.018643 | 0.409277 |
| GO:0060099 | C3 | regulation of phagocytosis, | 0.018643 | 0.409277 |
| | | engulfment | | |
| GO:0071380 | C3 | cellular response to prostaglandin E | 0.018643 | 0.409277 |
| | | stimulus | | |
| GO:0097531 | C3 | mast cell migration | 0.018643 | 0.409277 |
| GO:1902563 | C3 | regulation of neutrophil activation | 0.018643 | 0.409277 |
| GO:1905153 | C3 | regulation of membrane invagination | 0.018643 | 0.409277 |
| GO:2000052 | C3 | positive regulation of non-canonical | 0.018643 | 0.409277 |
| | | Wnt signaling pathway | | |
| GO:1903706 | C3 | regulation of hemopoiesis | 0.01913 | 3.410641 |
| GO:0031345 | C3 | negative regulation of cell projection | 0.019222 | 1.909959 |
| | | organization | | |
| GO:0002931 | C3 | response to ischemia | 0.019252 | 0.818554 |
| GO:0031103 | C3 | axon regeneration | 0.019252 | 0.818554 |
| GO:0003012 | C3 | muscle system process | 0.019306 | 3.683492 |
| GO:0032682 | C3 | negative regulation of chemokine | 0.019354 | 0.545703 |
| | | production | | |
| GO:0032753 | C3 | positive regulation of interleukin-4 | 0.019354 | 0.545703 |
| | | production | | |
| GO:0042026 | C3 | protein refolding | 0.019354 | 0.545703 |
| GO:0001701 | C3 | in utero embryonic development | 0.019473 | 3.274216 |
| GO:0008277 | C3 | regulation of G protein-coupled | 0.019527 | 1.500682 |
| | | receptor signaling pathway | | |
| GO:0009135 | C3 | purine nucleoside diphosphate | 0.019685 | 1.227831 |
| | | metabolic process | | |
| GO:0009179 | C3 | purine ribonucleoside diphosphate | 0.019685 | 1.227831 |
| | | metabolic process | | |
| GO:0070252 | C3 | actin-mediated cell contraction | 0.019685 | 1.227831 |
| GO:1901607 | C3 | alpha-amino acid biosynthetic process | 0.019911 | 0.95498 |
| GO:0007265 | C3 | Ras protein signal transduction | 0.020031 | 3.001364 |
| GO:0044409 | C3 | entry into host | 0.020277 | 1.637108 |
| GO:0002761 | C3 | regulation of myeloid leukocyte | 0.020331 | 1.364256 |
| | | differentiation | | |
| GO:0030278 | C3 | regulation of ossification | 0.020331 | 1.364256 |
| GO:0017158 | C3 | regulation of calcium ion-dependent | 0.020446 | 0.682128 |
| | | exocytosis | | |
| GO:0019432 | C3 | triglyceride biosynthetic process | 0.020446 | 0.682128 |
| GO:0042417 | C3 | dopamine metabolic process | 0.020446 | 0.682128 |
| GO:0072210 | C3 | metanephric nephron development | 0.020446 | 0.682128 |
| GO:1905314 | C3 | semi-lunar valve development | 0.020446 | 0.682128 |
| GO:0071356 | C3 | cellular response to tumor necrosis | 0.020475 | 2.18281 |
| | | factor | | |
| GO:0002705 | C3 | positive regulation of leukocyte | 0.020479 | 1.500682 |
| | | mediated immunity | | |
| GO:0060048 | C3 | cardiac muscle contraction | 0.020479 | 1.500682 |
| GO:0043534 | C3 | blood vessel endothelial cell | 0.020675 | 1.773533 |
| | | migration | | |
| GO:0006664 | C3 | glycolipid metabolic process | 0.020818 | 1.227831 |
| GO:0032760 | C3 | positive regulation of tumor necrosis | 0.020818 | 1.227831 |
| | | factor production | | |
| GO:0043470 | C3 | regulation of carbohydrate catabolic | 0.020878 | 0.818554 |
| | | process | | |
| GO:0045806 | C3 | negative regulation of endocytosis | 0.020878 | 0.818554 |
| GO:0008064 | C3 | regulation of actin polymerization or | 0.021189 | 1.637108 |
| | | depolymerization | | |
| GO:0007409 | C3 | axonogenesis | 0.021312 | 3.547067 |
| GO:0045600 | C3 | positive regulation of fat cell | 0.021344 | 0.95498 |
| | | differentiation | | |
| GO:1903317 | C3 | regulation of protein maturation | 0.021344 | 0.95498 |
| GO:0030282 | C3 | bone mineralization | 0.021391 | 1.364256 |
| GO:0007626 | C3 | locomotory behavior | 0.021627 | 1.909959 |
| GO:0003416 | C3 | endochondral bone growth | 0.021905 | 0.545703 |
| GO:0010894 | C3 | negative regulation of steroid | 0.021905 | 0.545703 |
| | | biosynthetic process | | |
| GO:0045606 | C3 | positive regulation of epidermal cell | 0.021905 | 0.545703 |
| | | differentiation | | |
| GO:0071624 | C3 | positive regulation of granulocyte | 0.021905 | 0.545703 |
| | | chemotaxis | | |
| GO:0090314 | C3 | positive regulation of protein targeting | 0.021905 | 0.545703 |
| | | to membrane | | |
| GO:1903509 | C3 | liposaccharide metabolic process | 0.021998 | 1.227831 |
| GO:0048678 | C3 | response to axon injury | 0.02209 | 1.091405 |
| GO:0071229 | C3 | cellular response to acid chemical | 0.02209 | 1.091405 |
| GO:1902600 | C3 | proton transmembrane transport | 0.022132 | 1.637108 |
| GO:0018146 | C3 | keratan sulfate biosynthetic process | 0.022297 | 0.409277 |
| GO:0045651 | C3 | positive regulation of macrophage | 0.022297 | 0.409277 |
| | | differentiation | | |
| GO:0046068 | C3 | cGMP metabolic process | 0.022297 | 0.409277 |
| GO:0051127 | C3 | positive regulation of actin nucleation | 0.022297 | 0.409277 |
| GO:0046660 | C3 | female sex differentiation | 0.022492 | 1.364256 |
| GO:0002714 | C3 | positive regulation of B cell mediated | 0.022492 | 0.682128 |
| | | immunity | | |
| GO:0002891 | C3 | positive regulation of immunoglobulin | 0.022492 | 0.682128 |
| | | mediated immune response | | |
| GO:0071604 | C3 | transforming growth factor beta | 0.022492 | 0.682128 |
| | | production | | |
| GO:0010332 | C3 | response to gamma radiation | 0.022595 | 0.818554 |
| GO:0000271 | C3 | polysaccharide biosynthetic process | 0.022847 | 0.95498 |
| GO:0071677 | C3 | positive regulation of mononuclear | 0.022847 | 0.95498 |
| | | cell migration | | |
| GO:0150076 | C3 | neuroinflammatory response | 0.022847 | 0.95498 |
| GO:1903578 | C3 | regulation of ATP metabolic process | 0.022847 | 0.95498 |
| GO:0035265 | C3 | organ growth | 0.023366 | 1.773533 |
| GO:0016032 | C3 | viral process | 0.023533 | 3.410641 |
| GO:0043434 | C3 | response to peptide hormone | 0.023533 | 3.410641 |
| GO:0001952 | C3 | regulation of cell-matrix adhesion | 0.023632 | 1.364256 |
| GO:0009306 | C3 | protein secretion | 0.023701 | 3.001364 |
| GO:0110053 | C3 | regulation of actin filament | 0.02385 | 2.455662 |
| | | organization | | |
| GO:0032103 | C3 | positive regulation of response to | 0.024076 | 3.683492 |
| | | external stimulus | | |
| GO:0030832 | C3 | regulation of actin filament length | 0.024111 | 1.637108 |
| GO:0043393 | C3 | regulation of protein binding | 0.024252 | 1.909959 |
| GO:0035592 | C3 | establishment of protein localization | 0.02436 | 3.001364 |
| | | to extracellular region | | |
| GO:0050670 | C3 | regulation of lymphocyte proliferation | 0.02437 | 2.18281 |
| GO:0001706 | C3 | endoderm formation | 0.024405 | 0.818554 |
| GO:0048010 | C3 | vascular endothelial growth factor | 0.024405 | 0.818554 |
| | | receptor signaling pathway | | |
| GO:0001707 | C3 | mesoderm formation | 0.024421 | 0.95498 |
| GO:0030177 | C3 | positive regulation of Wnt signaling | 0.024631 | 1.500682 |
| | | pathway | | |
| GO:0022011 | C3 | myelination in peripheral nervous | 0.024652 | 0.545703 |
| | | system | | |
| GO:0032292 | C3 | peripheral nervous system axon | 0.024652 | 0.545703 |
| | | ensheathment | | |
| GO:0072012 | C3 | glomerulus vasculature development | 0.024652 | 0.545703 |
| GO:0071548 | C3 | response to dexamethasone | 0.024665 | 0.682128 |
| GO:0090207 | C3 | regulation of triglyceride metabolic | 0.024665 | 0.682128 |
| | | process | | |
| GO:1901216 | C3 | positive regulation of neuron death | 0.024874 | 1.091405 |
| GO:1903557 | C3 | positive regulation of tumor necrosis | 0.025822 | 1.227831 |
| | | factor superfamily cytokine | | |
| | | production | | |
| GO:0043536 | C3 | positive regulation of blood vessel | 0.026068 | 0.95498 |
| | | endothelial cell migration | | |
| GO:0045669 | C3 | positive regulation of osteoblast | 0.026068 | 0.95498 |
| | | differentiation | | |
| GO:0072078 | C3 | nephron tubule morphogenesis | 0.026068 | 0.95498 |
| GO:0120192 | C3 | tight junction assembly | 0.026068 | 0.95498 |
| GO:2000242 | C3 | negative regulation of reproductive | 0.026068 | 0.95498 |
| | | process | | |
| GO:0042445 | C3 | hormone metabolic process | 0.026075 | 2.18281 |
| GO:0007219 | C3 | Notch signaling pathway | 0.026309 | 1.773533 |
| GO:0030042 | C3 | actin filament depolymerization | 0.026309 | 0.818554 |
| GO:0045010 | C3 | actin nucleation | 0.026309 | 0.818554 |
| GO:0036342 | C3 | post-anal tail morphogenesis | 0.026311 | 0.409277 |
| GO:0044539 | C3 | long-chain fatty acid import into cell | 0.026311 | 0.409277 |
| GO:0045019 | C3 | negative regulation of nitric oxide | 0.026311 | 0.409277 |
| | | biosynthetic process | | |
| GO:0055119 | C3 | relaxation of cardiac muscle | 0.026311 | 0.409277 |
| GO:1902916 | C3 | positive regulation of protein | 0.026311 | 0.409277 |
| | | polyubiquitination | | |
| GO:1903729 | C3 | regulation of plasma membrane | 0.026311 | 0.409277 |
| | | organization | | |
| GO:1904406 | C3 | negative regulation of nitric oxide | 0.026311 | 0.409277 |
| | | metabolic process | | |
| GO:0046031 | C3 | ADP metabolic process | 0.026353 | 1.091405 |
| GO:0046700 | C3 | heterocycle catabolic process | 0.02647 | 3.683492 |
| GO:1902903 | C3 | regulation of supramolecular fiber | 0.026585 | 3.13779 |
| | | organization | | |

597, 804 and 431 genes were identified as uniquely expressed in hFSCs, JY-bESCs, and mFSCs (FIG. 8B), while 621, 606, 464 genes were specifically enriched in hpESC, JY-bESC and mpESCs, respectively (FIG. 8C). Comparing JY-bESCs to formative PSCs revealed GO term enrichment in integrin signaling pathway for JY-bESCs and protein glycosylation and WNT signaling pathway for hFSCs and mFSCs, respectively (FIG. 8B and Table F).

Table F: DEG Numbers (Up-Regulated and Down-Regulated) in Comparisons of JY-bESC Vs. hpESC_Bi, JY-bESC Vs. mpESC_Liu, and hpESC_Bi Vs. mpESC_Liu.

| Comparisons | Up-regulated | Down-regulated |
|---|---|---|
| JY-bESC vs. hpESC_Bi | 1512 | 1380 |
| JY-bESC vs. mpESC_Liu | 1478 | 1270 |
| hpESC_Bi vs. mpESC_Liu | 1589 | 1505 |

Comparing JY-bESCs to primed PSCs unveiled enrichment in species-specific GO terms: extracellular matrix organization for JY-bESCs, regulation of neuron projection development for hpESCs, and ribose phosphate metabolic process for mpESCs (FIG. 8C and Table G).

Table G: Table SIG. GO Terms Enriched by Genes Uniquely Expressed in hpESCs, JY-bESCs, and mpESCs

| ID | group | Description | pvalue | ratio |
|---|---|---|---|---|
| GO:0010975 | C1 | regulation of neuron | 6.36E−06 | 5.688073 |
| | | projection development | | |
| GO:0006493 | C1 | protein O-linked | 1.04E−05 | 2.201835 |
| | | glycosylation | | |
| GO:0003018 | C1 | vascular process in | 1.28E−05 | 4.036697 |
| | | circulatory system | | |
| GO:0051056 | C1 | regulation of small | 2.82E−05 | 4.220183 |
| | | GTPase mediated | | |
| | | signal transduction | | |
| GO:0031346 | C1 | positive regulation | 3.49E−05 | 4.587156 |
| | | of cell projection | | |
| | | organization | | |
| GO:0009435 | C1 | NAD biosynthetic | 7.87E−05 | 1.100917 |
| | | process | | |
| GO:0035296 | C1 | regulation of tube | 9.23E−05 | 2.568807 |
| | | diameter | | |
| GO:0097746 | C1 | blood vessel diameter | 9.23E−05 | 2.568807 |
| | | maintenance | | |
| GO:0035150 | C1 | regulation of tube size | 9.93E−05 | 2.568807 |
| GO:0019359 | C1 | nicotinamide nucleotide | 0.000123 | 1.100917 |
| | | biosynthetic process | | |
| GO:0019363 | C1 | pyridine nucleotide | 0.000123 | 1.100917 |
| | | biosynthetic process | | |
| GO:0006303 | C1 | double-strand break | 0.000124 | 1.651376 |
| | | repair via nonhomologous | | |
| | | end joining | | |
| GO:0060041 | C1 | retina development | 0.000212 | 2.568807 |
| | | in camera-type eye | | |
| GO:0009100 | C1 | glycoprotein metabolic | 0.000217 | 4.587156 |
| | | process | | |
| GO:0072525 | C1 | pyridine-containing | 0.000223 | 1.100917 |
| | | compound biosynthetic | | |
| | | process | | |
| GO:0006486 | C1 | protein glycosylation | 0.000298 | 3.119266 |
| GO:0043413 | C1 | macromolecule | 0.000298 | 3.119266 |
| | | glycosylation | | |
| GO:0009101 | C1 | glycoprotein biosynthetic | 0.000441 | 3.853211 |
| | | process | | |
| GO:0033555 | C1 | multicellular organismal | 0.000532 | 1.651376 |
| | | response to stress | | |
| GO:0033692 | C1 | cellular polysaccharide | 0.000599 | 1.46789 |
| | | biosynthetic process | | |
| GO:0045932 | C1 | negative regulation | 0.000644 | 0.917431 |
| | | of muscle contraction | | |
| GO:0120032 | C1 | regulation of plasma | 0.00068 | 2.752294 |
| | | membrane bounded cell | | |
| | | projection assembly | | |
| GO:0070085 | C1 | glycosylation | 0.000753 | 3.119266 |
| GO:0060491 | C1 | regulation of cell | 0.000754 | 2.752294 |
| | | projection assembly | | |
| GO:0019935 | C1 | cyclic-nucleotide- | 0.000825 | 1.651376 |
| | | mediated signaling | | |
| GO:0006813 | C1 | potassium ion transport | 0.000863 | 3.119266 |
| GO:0045986 | C1 | negative regulation of | 0.000944 | 0.733945 |
| | | smooth muscle contraction | | |
| GO:0000271 | C1 | polysaccharide biosynthetic | 0.001176 | 1.46789 |
| | | process | | |
| GO:2001032 | C1 | regulation of double- | 0.001309 | 0.917431 |
| | | strand break repair via | | |
| | | nonhomologous end | | |
| | | joining | | |
| GO:0016266 | C1 | O-glycan processing | 0.001376 | 1.100917 |
| GO:0030198 | C2 | extracellular matrix | 3.80E−14 | 7.039711 |
| | | organization | | |
| GO:0043062 | C2 | extracellular structure | 4.22E−14 | 7.039711 |
| | | organization | | |
| GO:0045229 | C2 | external encapsulating | 5.18E−14 | 7.039711 |
| | | structure organization | | |
| GO:0030199 | C2 | collagen fibril | 3.55E−10 | 2.707581 |
| | | organization | | |
| GO:0097529 | C2 | myeloid leukocyte | 1.25E−08 | 4.693141 |
| | | migration | | |
| GO:0022617 | C2 | extracellular matrix | 2.28E−08 | 2.34657 |
| | | disassembly | | |
| GO:0061448 | C2 | connective tissue | 4.10E−08 | 4.873646 |
| | | development | | |
| GO:0030595 | C2 | leukocyte chemotaxis | 4.40E−08 | 4.512635 |
| GO:0050900 | C2 | leukocyte migration | 8.75E−08 | 5.956679 |
| GO:0032963 | C2 | collagen metabolic | 8.82E−08 | 2.888087 |
| | | process | | |
| GO:0001503 | C2 | ossification | 1.64E−07 | 6.137184 |
| GO:0002688 | C2 | regulation of leukocyte | 1.84E−07 | 3.068592 |
| | | chemotaxis | | |
| GO:0060326 | C2 | cell chemotaxis | 2.64E−07 | 5.054152 |
| GO:0001655 | C2 | urogenital system | 3.05E−07 | 5.415162 |
| | | development | | |
| GO:0002685 | C2 | regulation of leukocyte | 3.45E−07 | 4.151625 |
| | | migration | | |
| GO:0031589 | C2 | cell-substrate adhesion | 5.16E−07 | 5.415162 |
| GO:0002888 | C2 | positive regulation | 9.95E−07 | 1.263538 |
| | | of myeloid leukocyte | | |
| | | mediated immunity | | |
| GO:0007229 | C2 | integrin-mediated | 1.08E−06 | 2.707581 |
| | | signaling pathway | | |
| GO:0051216 | C2 | cartilage development | 2.02E−06 | 3.610108 |
| GO:0090322 | C2 | regulation of superoxide | 4.79E−06 | 1.444043 |
| | | metabolic process | | |
| GO:0030574 | C2 | collagen catabolic process | 5.17E−06 | 1.624549 |
| GO:0072001 | C2 | renal system development | 8.25E−06 | 4.512635 |
| GO:0001822 | C2 | kidney development | 1.53E−05 | 4.33213 |
| GO:0050920 | C2 | regulation of chemotaxis | 1.55E−05 | 3.610108 |
| GO:0042060 | C2 | wound healing | 1.94E−05 | 5.415162 |
| GO:0071622 | C2 | regulation of granulocyte | 2.10E−05 | 1.624549 |
| | | chemotaxis | | |
| GO:0050921 | C2 | positive regulation of | 2.20E−05 | 2.707581 |
| | | chemotaxis | | |
| GO:0002690 | C2 | positive regulation of | 2.64E−05 | 2.166065 |
| | | leukocyte chemotaxis | | |
| GO:0071621 | C2 | granulocyte chemotaxis | 2.83E−05 | 2.527076 |
| GO:0032928 | C2 | regulation of superoxide | 3.08E−05 | 1.083032 |
| | | anion generation | | |
| GO:0070665 | C2 | positive regulation of | 3.36E−05 | 2.888087 |
| | | leukocyte proliferation | | |
| GO:0018158 | C2 | protein oxidation | 3.41E−05 | 0.902527 |
| GO:0030890 | C2 | positive regulation of | 4.29E−05 | 1.444043 |
| | | B cell proliferation | | |
| GO:0001819 | C2 | positive regulation of | 4.63E−05 | 5.595668 |
| | | cytokine production | | |
| GO:0032905 | C2 | transforming growth | 5.00E−05 | 0.902527 |
| | | factor betal production | | |
| GO:0002366 | C2 | leukocyte activation | 5.70E−05 | 3.971119 |
| | | involved in immune | | |
| | | response | | |
| GO:0017157 | C2 | regulation of exocytosis | 5.93E−05 | 3.249097 |
| GO:0002263 | C2 | cell activation involved | 6.97E−05 | 3.971119 |
| | | in immune response | | |
| GO:0018149 | C2 | peptide cross-linking | 7.13E−05 | 1.263538 |
| GO:0007160 | C2 | cell-matrix adhesion | 8.04E−05 | 3.429603 |
| GO:0050671 | C2 | positive regulation of | 0.000102 | 2.527076 |
| | | lymphocyte proliferation | | |
| GO:0043277 | C2 | apoptotic cell clearance | 0.000109 | 1.444043 |
| GO:0002687 | C2 | positive regulation of | 0.000118 | 2.527076 |
| | | leukocyte migration | | |
| GO:0032946 | C2 | positive regulation of | 0.000127 | 2.527076 |
| | | mononuclear cell | | |
| | | proliferation | | |
| GO:0050867 | C2 | positive regulation of | 0.000129 | 5.234657 |
| | | cell activation | | |
| GO:0002062 | C2 | chondrocyte differentiation | 0.00013 | 2.166065 |
| GO:0046486 | C2 | glycerolipid metabolic | 0.000133 | 4.693141 |
| | | process | | |
| GO:0048661 | C2 | positive regulation of | 0.000139 | 1.98556 |
| | | smooth muscle cell | | |
| | | proliferation | | |
| GO:0070663 | C2 | regulation of leukocyte | 0.00014 | 3.610108 |
| | | proliferation | | |
| GO:0030888 | C2 | regulation of B cell | 0.000141 | 1.624549 |
| | | proliferation | | |
| GO:0048146 | C2 | positive regulation of | 0.000144 | 1.444043 |
| | | fibroblast proliferation | | |
| GO:0009150 . . . 82 | C2 | purine ribonucleotide | 0.000145 | 5.054152 |
| | | metabolic process | | |
| GO:0002697 | C2 | regulation of immune | 0.000148 | 4.512635 |
| | | effector process | | |
| GO:0071560 | C2 | cellular response to | 0.000155 | 3.610108 |
| | | transforming growth | | |
| | | factor beta stimulus | | |
| GO:0042119 | C2 | neutrophil activation | 0.000169 | 1.263538 |
| GO:0002283 | C2 | neutrophil activation | 0.000176 | 0.902527 |
| | | involved in immune | | |
| | | response | | |
| GO:0032930 | C2 | positive regulation of | 0.000176 | 0.902527 |
| | | superoxide anion | | |
| | | generation | | |
| GO:0006163 . . . 88 | C2 | purine nucleotide | 0.000179 | 5.234657 |
| | | metabolic process | | |
| GO:0060348 | C2 | bone development | 0.000186 | 3.249097 |
| GO:0071492 | C2 | cellular response | 0.000204 | 0.722022 |
| | | to UV-A | | |
| GO:1905634 | C2 | regulation of protein | 0.000204 | 0.722022 |
| | | localization to | | |
| | | chromatin | | |
| GO:0071559 | C2 | response to transforming | 0.000207 | 3.610108 |
| | | growth factor | | |
| | | beta | | |
| GO:0048144 | C2 | fibroblast proliferation | 0.000217 | 1.98556 |
| GO:0002274 | C2 | myeloid leukocyte | 0.000229 | 3.249097 |
| | | activation | | |
| GO:0042554 | C2 | superoxide anion | 0.00023 | 1.263538 |
| | | generation | | |
| GO:0097530 | C2 | granulocyte migration | 0.000235 | 2.527076 |
| GO:0050670 | C2 | regulation of lymphocyte | 0.000254 | 3.249097 |
| | | proliferation | | |
| GO:0070661 | C2 | leukocyte proliferation | 0.000262 | 4.151625 |
| GO:0002699 | C2 | positive regulation of | 0.000267 | 3.429603 |
| | | immune effector process | | |
| GO:0032103 | C2 | positive regulation | 0.000269 | 5.054152 |
| | | of response to | | |
| | | external stimulus | | |
| GO:0060350 | C2 | endochondral bone | 0.000273 | 1.444043 |
| | | morphogenesis | | |
| GO:0150076 | C2 | neuroinflammatory | 0.000274 | 1.624549 |
| | | response | | |
| GO:1903578 | C2 | regulation of ATP | 0.000274 | 1.624549 |
| | | metabolic process | | |
| GO:0009259 . . . 104 | C2 | ribonucleotide | 0.000288 | 5.054152 |
| | | metabolic process | | |
| GO:0090201 | C2 | negative regulation | 0.000293 | 0.902527 |
| | | of release of | | |
| | | cytochrome c from | | |
| | | mitochondria | | |
| GO:0009205 | C2 | purine ribonucleoside | 0.000296 | 3.249097 |
| | | triphosphate metabolic | | |
| | | process | | |
| GO:0032908 | C2 | regulation of | 0.000299 | 0.722022 |
| | | transforming growth | | |
| | | factor betal | | |
| | | production | | |
| GO:0006801 | C2 | superoxide | 0.000303 | 1.624549 |
| | | metabolic process | | |
| GO:0032944 | C2 | regulation of | 0.000311 | 3.249097 |
| | | mononuclear cell | | |
| | | proliferation | | |
| GO:0002886 | C2 | regulation of | 0.000346 | 1.444043 |
| | | myeloid leukocyte | | |
| | | mediated immunity | | |
| GO:0032640 | C2 | tumor necrosis | 0.000354 | 2.707581 |
| | | factor production | | |
| GO:0032680 | C2 | regulation of tumor | 0.000354 | 2.707581 |
| | | necrosis factor | | |
| | | production | | |
| GO:0001649 | C2 | osteoblast differentiation | 0.000361 | 3.249097 |
| GO:0002683 | C2 | negative regulation of | 0.000363 | 4.873646 |
| | | immune system process | | |
| GO:0009144 | C2 | purine nucleoside | 0.000379 | 3.249097 |
| | | triphosphate | | |
| | | metabolic process | | |
| GO:0048706 | C2 | embryonic skeletal | 0.000386 | 2.166065 |
| | | system development | | |
| GO:0019693 . . . 117 | C2 | ribose phosphate | 0.000391 | 5.054152 |
| | | metabolic process | | |
| GO:0048705 | C2 | skeletal system | 0.000399 | 3.068592 |
| | | morphogenesis | | |
| GO:0036230 | C2 | granulocyte activation | 0.000407 | 1.263538 |
| GO:0009199 | C2 | ribonucleoside | 0.000417 | 3.249097 |
| | | triphosphate | | |
| | | metabolic process | | |
| GO:0045926 | C2 | negative regulation | 0.000417 | 3.249097 |
| | | of growth | | |
| GO:0010038 | C2 | response to | 0.000423 | 4.151625 |
| | | metal ion | | |
| GO:1900542 | C2 | regulation of | 0.000429 | 1.805054 |
| | | purine nucleotide | | |
| | | metabolic process | | |
| GO:2000377 | C2 | regulation of | 0.000451 | 2.34657 |
| | | reactive oxygen | | |
| | | species metabolic | | |
| | | process | | |
| GO:0062012 | C2 | regulation of | 0.000453 | 3.971119 |
| | | small molecule | | |
| | | metabolic process | | |
| GO:0071706 | C2 | tumor necrosis | 0.00047 | 2.707581 |
| | | factor superfamily | | |
| | | cytokine production | | |
| GO:1903555 | C2 | regulation of tumor | 0.00047 | 2.707581 |
| | | necrosis factor | | |
| | | superfamily | | |
| | | cytokine production | | |
| GO:0098657 | C2 | import into cell | 0.000481 | 3.249097 |
| GO:0002275 | C2 | myeloid cell activation | 0.000508 | 1.805054 |
| | | involved in immune | | |
| | | response | | |
| GO:0009132 . . . 130 | C2 | nucleoside diphosphate | 0.000512 | 2.166065 |
| | | metabolic process | | |
| GO:0035987 | C2 | endodermal cell | 0.000528 | 1.263538 |
| | | differentiation | | |
| GO:2001234 | C2 | negative regulation | 0.000538 | 3.068592 |
| | | of apoptotic | | |
| | | signaling pathway | | |
| GO:0070141 | C2 | response to UV-A | 0.000578 | 0.722022 |
| GO:0072593 | C2 | reactive oxygen species | 0.000592 | 3.068592 |
| | | metabolic process | | |
| GO:0006887 | C2 | exocytosis | 0.000595 | 4.151625 |
| GO:0060349 | C2 | bone morphogenesis | 0.000598 | 1.805054 |
| GO:0032964 | C2 | collagen biosynthetic | 0.000599 | 1.263538 |
| | | process | | |
| GO:0006898 | C2 | receptor-mediated | 0.000606 | 3.249097 |
| | | endocytosis | | |
| GO:0006140 | C2 | regulation of | 0.000649 | 1.805054 |
| | | nucleotide | | |
| | | metabolic process | | |
| GO:0016052 | C2 | carbohydrate catabolic | 0.000657 | 2.34657 |
| | | process | | |
| GO:0046034 | C2 | ATP metabolic process | 0.000692 | 2.888087 |
| GO:0006658 | C2 | phosphatidylserine | 0.000694 | 0.902527 |
| | | metabolic process | | |
| GO:0006165 | C2 | nucleoside diphosphate | 0.000702 | 1.805054 |
| | | phosphorylation | | |
| GO:1901136 | C2 | carbohydrate derivative | 0.000702 | 2.527076 |
| | | catabolic process | | |
| GO:0006096 | C2 | glycolytic process | 0.000715 | 1.624549 |
| GO:0010823 | C2 | negative regulation | 0.000762 | 1.263538 |
| | | of mitochondrion | | |
| | | organization | | |
| GO:0006757 | C2 | ATP generation | 0.000781 | 1.624549 |
| | | from ADP | | |
| GO:0046939 | C2 | nucleotide | 0.00082 | 1.805054 |
| | | phosphorylation | | |
| GO:0048659 | C2 | smooth muscle | 0.000829 | 2.527076 |
| | | cell proliferation | | |
| GO:0002696 | C2 | positive regulation | 0.000837 | 4.693141 |
| | | of leukocyte | | |
| | | activation | | |
| GO:0006575 | C2 | cellular modified | 0.000876 | 2.527076 |
| | | amino acid | | |
| | | metabolic process | | |
| GO:0010810 | C2 | regulation of | 0.000884 | 2.888087 |
| | | cell-substrate | | |
| | | adhesion | | |
| GO:0050764 | C2 | regulation of | 0.000885 | 1.805054 |
| | | phagocytosis | | |
| GO:0009141 | C2 | nucleoside | 0.000943 | 3.249097 |
| | | triphosphate | | |
| | | metabolic process | | |
| GO:0009135 | C2 | purine nucleoside | 0.000954 | 1.805054 |
| | | diphosphate | | |
| | | metabolic process | | |
| GO:0009179 | C2 | purine ribonucleoside | 0.000954 | 1.805054 |
| | | diphosphate | | |
| | | metabolic process | | |
| GO:0034329 | C2 | cell junction assembly | 0.000958 | 4.512635 |
| GO:0071675 | C2 | regulation of | 0.000991 | 1.98556 |
| | | mononuclear cell | | |
| | | migration | | |
| GO:0045745 | C2 | positive regulation | 0.001004 | 0.902527 |
| | | of G protein-coupled | | |
| | | receptor signaling | | |
| | | pathway | | |
| GO:0048145 | C2 | regulation of fibroblast | 0.001007 | 1.624549 |
| | | proliferation | | |
| GO:0071277 | C2 | cellular response | 0.001007 | 1.624549 |
| | | to calcium ion | | |
| GO:0006909 | C2 | phagocytosis | 0.001019 | 3.610108 |
| GO:0009410 | C2 | response to xenobiotic | 0.001023 | 4.512635 |
| | | stimulus | | |
| GO:0090287 | C2 | regulation of | 0.001055 | 3.790614 |
| | | cellular response to | | |
| | | growth factor stimulus | | |
| GO:0010761 | C2 | fibroblast migration | 0.001072 | 1.263538 |
| GO:2000379 | C2 | positive regulation | 0.001087 | 1.444043 |
| | | of reactive oxygen | | |
| | | species metabolic | | |
| | | process | | |
| GO:0033627 | C2 | cell adhesion | 0.001093 | 1.624549 |
| | | mediated by | | |
| | | integrin | | |
| GO:0048562 | C2 | embryonic organ | 0.001138 | 3.429603 |
| | | morphogenesis | | |
| GO:0071674 | C2 | mononuclear | 0.001142 | 2.707581 |
| | | cell migration | | |
| GO:1903305 | C2 | regulation of | 0.001176 | 2.166065 |
| | | regulated | | |
| | | secretory pathway | | |
| GO:0003416 | C2 | endochondral | 0.001193 | 0.902527 |
| | | bone growth | | |
| GO:0002285 | C2 | lymphocyte activation | 0.001199 | 2.707581 |
| | | involved in immune | | |
| | | response | | |
| GO:0002221 | C2 | pattern recognition | 0.001266 | 2.527076 |
| | | receptor signaling | | |
| | | pathway | | |
| GO:0002700 | C2 | regulation of | 0.001266 | 2.527076 |
| | | production of | | |
| | | molecular | | |
| | | mediator of | | |
| | | immune response | | |
| GO:0010763 | C2 | positive | 0.001281 | 0.722022 |
| | | regulation of | | |
| | | fibroblast | | |
| | | migration | | |
| GO:0006066 | C2 | alcohol metabolic | 0.001287 | 3.971119 |
| | | process | | |
| GO:0001704 | C2 | formation of | 0.001289 | 1.98556 |
| | | primary germ layer | | |
| GO:0051592 | C2 | response to | 0.001324 | 2.166065 |
| | | calcium ion | | |
| GO:0042116 | C2 | macrophage activation | 0.001369 | 1.805054 |
| GO:0001958 | C2 | endochondral ossification | 0.001408 | 0.902527 |
| GO:0036075 | C2 | replacement ossification | 0.001408 | 0.902527 |
| GO:0050766 | C2 | positive regulation of | 0.001427 | 1.444043 |
| | | phagocytosis | | |
| GO:0001706 | C2 | endoderm formation | 0.001474 | 1.263538 |
| GO:0046031 | C2 | ADP metabolic process | 0.0015 | 1.624549 |
| GO:0034446 | C2 | substrate | 0.001571 | 1.805054 |
| | | adhesion-dependent | | |
| | | cell spreading | | |
| GO:0048880 | C2 | sensory system | 0.00163 | 4.151625 |
| | | development | | |
| GO:0090130 | C2 | tissue migration | 0.001634 | 3.971119 |
| GO:0043542 | C2 | endothelial cell | 0.001675 | 3.249097 |
| | | migration | | |
| GO:1905521 | C2 | regulation of | 0.00169 | 1.083032 |
| | | macrophage | | |
| | | migration | | |
| GO:0002637 | C2 | regulation of | 0.001697 | 1.444043 |
| | | immunoglobulin | | |
| | | production | | |
| GO:0009166 | C2 | nucleotide | 0.001697 | 1.444043 |
| | | catabolic process | | |
| GO:0006631 | C2 | fatty acid | 0.001739 | 4.151625 |
| | | metabolic process | | |
| GO:0046651 | C2 | lymphocyte | 0.00174 | 3.429603 |
| | | proliferation | | |
| GO:0048568 | C2 | embryonic organ | 0.001741 | 4.512635 |
| | | development | | |
| GO:0009953 | C2 | dorsal/ventral | 0.001744 | 1.624549 |
| | | pattern formation | | |
| GO:1905517 | C2 | macrophage migration | 0.0018 | 1.263538 |
| GO:0007179 | C2 | transforming growth | 0.001827 | 2.707581 |
| | | factor beta | | |
| | | receptor signaling | | |
| | | pathway | | |
| GO:1903829 | C2 | positive regulation | 0.001848 | 4.512635 |
| | | of protein | | |
| | | localization | | |
| GO:0060390 | C2 | regulation of | 0.00192 | 0.902527 |
| | | SMAD protein | | |
| | | signal transduction | | |
| GO:0098868 | C2 | bone growth | 0.00192 | 0.902527 |
| GO:0009185 | C2 | ribonucleoside | 0.00192 | 1.805054 |
| | | diphosphate | | |
| | | metabolic process | | |
| GO:0007369 | C2 | gastrulation | 0.001972 | 2.527076 |
| GO:0060340 | C2 | positive regulation | 0.001992 | 0.722022 |
| | | of type I | | |
| | | interferon-mediated | | |
| | | signaling | | |
| | | pathway | | |
| GO:0140354 | C2 | lipid import into cell | 0.001992 | 0.722022 |
| GO:0018108 | C2 | peptidyl-tyrosine | 0.001993 | 3.971119 |
| | | phosphorylation | | |
| GO:0048660 | C2 | regulation of | 0.001996 | 2.34657 |
| | | smooth muscle cell | | |
| | | proliferation | | |
| GO:0018212 | C2 | peptidyl-tyrosine | 0.002126 | 3.971119 |
| | | modification | | |
| GO:0045055 | C2 | regulated exocytosis | 0.00216 | 2.888087 |
| GO:0006650 | C2 | glycerophospholipid | 0.002169 | 3.429603 |
| | | metabolic | | |
| | | process | | |
| GO:0043299 | C2 | leukocyte degranulation | 0.002176 | 1.444043 |
| GO:0006767 | C2 | water-soluble vitamin | 0.00218 | 1.263538 |
| | | metabolic process | | |
| GO:0050808 | C2 | synapse organization | 0.002195 | 4.33213 |
| GO:1903052 | C2 | positive regulation of | 0.002231 | 1.98556 |
| | | proteolysis involved in | | |
| | | protein catabolic process | | |
| GO:0032943 | C2 | mononuclear cell | 0.002248 | 3.429603 |
| | | proliferation | | |
| GO:0030111 | C2 | regulation of Wnt | 0.002276 | 3.610108 |
| | | signaling pathway | | |
| GO:0003002 | C2 | regionalization | 0.002281 | 3.790614 |
| GO:0032642 | C2 | regulation of | 0.002329 | 1.624549 |
| | | chemokine production | | |
| GO:0090092 | C2 | regulation of | 0.002364 | 3.249097 |
| | | transmembrane | | |
| | | receptor protein | | |
| | | serine/threonine | | |
| | | kinase signaling | | |
| | | pathway | | |
| GO:0006110 | C2 | regulation of | 0.002387 | 1.083032 |
| | | glycolytic | | |
| | | process | | |
| GO:0051180 | C2 | vitamin transport | 0.002387 | 1.083032 |
| GO:0090199 | C2 | regulation of release | 0.002387 | 1.083032 |
| | | of cytochrome c from | | |
| | | mitochondria | | |
| GO:0048771 | C2 | tissue remodeling | 0.002435 | 2.34657 |
| GO:0060759 | C2 | regulation of | 0.002435 | 2.34657 |
| | | response to | | |
| | | cytokine stimulus | | |
| GO:0032102 | C2 | negative regulation | 0.002473 | 4.33213 |
| | | of response to | | |
| | | external stimulus | | |
| GO:0032602 | C2 | chemokine production | 0.002497 | 1.624549 |
| GO:0060351 | C2 | cartilage development | 0.002555 | 0.902527 |
| | | involved in | | |
| | | endochondral bone | | |
| | | morphogenesis | | |
| GO:0060346 | C2 | bone trabecula | 0.002576 | 0.541516 |
| | | formation | | |
| GO:0010927 | C2 | cellular component | 0.002637 | 1.805054 |
| | | assembly involved | | |
| | | in morphogenesis | | |
| GO:0051146 | C2 | striated muscle cell | 0.00264 | 3.249097 |
| | | differentiation | | |
| GO:0006644 | C2 | phospholipid metabolic | 0.002655 | 3.971119 |
| | | process | | |
| GO:0032620 | C2 | interleukin-17 production | 0.002662 | 1.083032 |
| GO:0032660 | C2 | regulation of | 0.002662 | 1.083032 |
| | | interleukin-17 | | |
| | | production | | |
| GO:0007178 | C2 | transmembrane | 0.002739 | 3.971119 |
| | | receptor protein | | |
| | | serine/threonine | | |
| | | kinase signaling | | |
| | | pathway | | |
| GO:0010631 | C2 | epithelial cell | 0.002774 | 3.790614 |
| | | migration | | |
| GO:0002705 | C2 | positive regulation | 0.002799 | 1.98556 |
| | | of leukocyte | | |
| | | mediated immunity | | |
| GO:0046434 | C2 | organophosphate | 0.002848 | 2.166065 |
| | | catabolic process | | |
| GO:2001243 | C2 | negative regulation | 0.002862 | 1.624549 |
| | | of intrinsic | | |
| | | apoptotic signaling | | |
| | | pathway | | |
| GO:0060760 | C2 | positive regulation | 0.002863 | 1.263538 |
| | | of response to | | |
| | | cytokine stimulus | | |
| GO:0001667 | C2 | ameboidal-type cell | 0.002908 | 4.693141 |
| | | migration | | |
| GO:0150063 | C2 | visual system development | 0.002914 | 3.971119 |
| GO:0090022 | C2 | regulation of neutrophil | 0.002925 | 0.902527 |
| | | chemotaxis | | |
| GO:1903050 | C2 | regulation of | 0.002946 | 2.707581 |
| | | proteolysis involved | | |
| | | in protein catabolic | | |
| | | process | | |
| GO:0043300 | C2 | regulation of leukocyte | 0.002959 | 1.083032 |
| | | degranulation | | |
| GO:1901654 | C2 | response to ketone | 0.002977 | 2.527076 |
| GO:0007611 | C2 | learning or memory | 0.002983 | 3.068592 |
| GO:0016485 | C2 | protein processing | 0.002987 | 2.888087 |
| GO:0090132 | C2 | epithelium migration | 0.003052 | 3.790614 |
| GO:0019218 | C2 | regulation of steroid | 0.003059 | 1.624549 |
| | | metabolic process | | |
| GO:0001818 | C2 | negative regulation | 0.003251 | 3.790614 |
| | | of cytokine | | |
| | | production | | |
| GO:0030534 | C2 | adult behavior | 0.003297 | 1.98556 |
| GO:0007389 | C2 | pattern specification | 0.003368 | 4.512635 |
| | | process | | |
| GO:0002253 | C2 | activation of immune | 0.003391 | 3.971119 |
| | | response | | |
| GO:0060537 | C2 | muscle tissue | 0.003404 | 4.151625 |
| | | development | | |
| GO:0034103 | C2 | regulation of tissue | 0.003447 | 1.444043 |
| | | remodeling | | |
| GO:1901292 | C2 | nucleoside phosphate | 0.003447 | 1.444043 |
| | | catabolic process | | |
| GO:0038003 | C2 | G protein-coupled | 0.003465 | 0.541516 |
| | | opioid receptor | | |
| | | signaling pathway | | |
| GO:0048251 | C2 | elastic fiber assembly | 0.003465 | 0.541516 |
| GO:0052646 | C2 | alditol phosphate | 0.003465 | 0.541516 |
| | | metabolic process | | |
| GO:0042100 | C2 | B cell proliferation | 0.003487 | 1.624549 |
| GO:0045765 | C2 | regulation of | 0.003506 | 3.610108 |
| | | angiogenesis | | |
| GO:0032288 | C2 | myelin assembly | 0.003507 | 0.722022 |
| GO:0032780 | C2 | negative regulation | 0.003507 | 0.722022 |
| | | of ATP-dependent | | |
| | | activity | | |
| GO:0030225 | C2 | macrophage | 0.003629 | 1.083032 |
| | | differentiation | | |
| GO:0030850 | C2 | prostate gland | 0.003629 | 1.083032 |
| | | development | | |
| GO:1903580 | C2 | positive regulation | 0.003777 | 0.902527 |
| | | of ATP metabolic | | |
| | | process | | |
| GO:0001959 | C2 | regulation of | 0.003835 | 2.166065 |
| | | cytokine-mediated | | |
| | | signaling pathway | | |
| GO:0051047 | C2 | positive regulation | 0.003896 | 3.249097 |
| | | of secretion | | |
| GO:0030593 | C2 | neutrophil chemotaxis | 0.00396 | 1.624549 |
| GO:0042102 | C2 | positive regulation | 0.00396 | 1.624549 |
| | | of T cell | | |
| | | proliferation | | |
| GO:0048013 | C2 | ephrin receptor | 0.004003 | 1.083032 |
| | | signaling pathway | | |
| GO:0003007 | C2 | heart morphogenesis | 0.00406 | 2.888087 |
| GO:0017015 | C2 | regulation of | 0.00407 | 1.98556 |
| | | transforming | | |
| | | growth factor | | |
| | | beta receptor | | |
| | | signaling pathway | | |
| GO:0002444 | C2 | myeloid leukocyte | 0.004214 | 1.624549 |
| | | mediated immunity | | |
| GO:0060840 | C2 | artery development | 0.004214 | 1.624549 |
| GO:1901342 | C2 | regulation of | 0.004238 | 3.610108 |
| | | vasculature | | |
| | | development | | |
| GO:0006734 | C2 | NADH metabolic | 0.004264 | 0.902527 |
| | | process | | |
| GO:0032147 | C2 | activation of | 0.004284 | 1.98556 |
| | | protein kinase | | |
| | | activity | | |
| GO:0032722 | C2 | positive regulation | 0.004351 | 1.263538 |
| | | of chemokine | | |
| | | production | | |
| GO:0002639 | C2 | positive regulation | 0.004404 | 1.083032 |
| | | of immunoglobulin | | |
| | | production | | |
| GO:0006090 | C2 | pyruvate metabolic | 0.004481 | 1.624549 |
| | | process | | |
| GO:0014812 | C2 | muscle cell migration | 0.004481 | 1.624549 |
| GO:0032526 | C2 | response to retinoic | 0.004481 | 1.624549 |
| | | acid | | |
| GO:1902652 | C2 | secondary alcohol | 0.004507 | 1.98556 |
| | | metabolic process | | |
| GO:0002551 | C2 | mast cell chemotaxis | 0.00452 | 0.541516 |
| GO:0043312 | C2 | neutrophil degranulation | 0.00452 | 0.541516 |
| GO:0072110 | C2 | glomerular mesangial | 0.00452 | 0.541516 |
| | | cell proliferation | | |
| GO:0048839 | C2 | inner ear development | 0.004638 | 2.34657 |
| GO:0050731 | C2 | positive regulation of | 0.004638 | 2.34657 |
| | | peptidyl-tyrosine | | |
| | | phosphorylation | | |
| GO:0014902 | C2 | myotube differentiation | 0.004709 | 1.805054 |
| GO:0050714 | C2 | positive regulation of | 0.004709 | 1.805054 |
| | | protein secretion | | |
| GO:1903844 | C2 | regulation of cellular | 0.004739 | 1.98556 |
| | | response to transforming | | |
| | | growth factor beta | | |
| | | stimulus | | |
| GO:0042692 | C2 | muscle cell | 0.004812 | 3.971119 |
| | | differentiation | | |
| GO:0072523 | C2 | purine-containing | 0.004835 | 1.083032 |
| | | compound catabolic | | |
| | | process | | |
| GO:2001242 | C2 | regulation of | 0.004853 | 2.166065 |
| | | intrinsic apoptotic | | |
| | | signaling pathway | | |
| GO:0002755 | C2 | MyD88-dependent toll-like | 0.004864 | 0.722022 |
| | | receptor signaling pathway | | |
| GO:0040037 | C2 | negative regulation of | 0.004864 | 0.722022 |
| | | fibroblast growth factor | | |
| | | receptor signaling pathway | | |
| GO:0046718 | C2 | viral entry into host cell | 0.004981 | 1.98556 |
| GO:0051604 | C2 | protein maturation | 0.005033 | 3.429603 |
| GO:2000060 | C2 | positive regulation of | 0.005055 | 1.624549 |
| | | ubiquitin-dependent | | |
| | | protein catabolic process | | |
| GO:1901216 | C2 | positive regulation of | 0.00523 | 1.444043 |
| | | neuron death | | |
| GO:1904645 | C2 | response to amyloid-beta | 0.005295 | 1.083032 |
| GO:0030178 | C2 | negative regulation of | 0.005316 | 2.166065 |
| | | Wnt signaling pathway | | |
| GO:0001505 | C2 | regulation of | 0.005346 | 2.527076 |
| | | neurotransmitter levels | | |
| GO:0006730 | C2 | one-carbon metabolic | 0.005369 | 0.902527 |
| | | process | | |
| GO:0032350 | C2 | regulation of hormone | 0.005369 | 0.902527 |
| | | metabolic process | | |
| GO:0043403 | C2 | skeletal muscle tissue | 0.005369 | 0.902527 |
| | | regeneration | | |
| GO:0061564 | C2 | axon development | 0.005407 | 4.512635 |
| GO:0050730 | C2 | regulation of peptidyl- | 0.00543 | 2.888087 |
| | | tyrosine phosphorylation | | |
| GO:0050890 | C2 | cognition | 0.005432 | 3.249097 |
| GO:0048545 | C2 | response to steroid | 0.005526 | 3.429603 |
| | | hormone | | |
| GO:0006639 | C2 | acylglycerol metabolic | 0.005532 | 1.805054 |
| | | process | | |
| GO:0060100 | C2 | positive regulation of | 0.00575 | 0.541516 |
| | | phagocytosis, engulfment | | |
| GO:0061430 | C2 | bone trabecula | 0.00575 | 0.541516 |
| | | morphogenesis | | |
| GO:0072537 | C2 | fibroblast activation | 0.00575 | 0.541516 |
| GO:1901857 | C2 | positive regulation of | 0.00575 | 0.541516 |
| | | cellular respiration | | |
| GO:1905155 | C2 | positive regulation of | 0.00575 | 0.541516 |
| | | membrane invagination | | |
| GO:0043583 | C2 | ear development | 0.005783 | 2.527076 |
| GO:0001836 | C2 | release of cytochrome c | 0.005787 | 1.083032 |
| | | from mitochondria | | |
| GO:0043470 | C2 | regulation of carbohydrate | 0.005787 | 1.083032 |
| | | catabolic process | | |
| GO:0002702 | C2 | positive regulation of | 0.005829 | 1.805054 |
| | | production of molecular | | |
| | | mediator of | | |
| | | immune response | | |
| GO:0006638 | C2 | neutral lipid metabolic | 0.005829 | 1.805054 |
| | | process | | |
| GO:0030168 | C2 | platelet activation | 0.005829 | 1.805054 |
| GO:0015850 | C2 | organic hydroxy compound | 0.005839 | 3.068592 |
| | | transport | | |
| GO:2000106 | C2 | regulation of leukocyte | 0.005958 | 1.444043 |
| | | apoptotic process | | |
| GO:0010762 | C2 | regulation of fibroblast | 0.005991 | 0.902527 |
| | | migration | | |
| GO:0030866 | C2 | cortical actin cytoskeleton | 0.005991 | 0.902527 |
| | | organization | | |
| GO:0032965 | C2 | regulation of collagen | 0.005991 | 0.902527 |
| | | biosynthetic process | | |
| GO:0048246 | C2 | macrophage chemotaxis | 0.005991 | 0.902527 |
| GO:0043467 | C2 | regulation of generation | 0.006139 | 1.805054 |
| | | of precursor metabolites | | |
| | | and energy | | |
| GO:0023061 | C2 | signal release | 0.006279 | 4.33213 |
| GO:0045669 | C2 | positive regulation of | 0.00636 | 1.263538 |
| | | osteoblast differentiation | | |
| GO:0033002 | C2 | muscle cell proliferation | 0.006403 | 2.707581 |
| GO:0060343 | C2 | trabecula formation | 0.006535 | 0.722022 |
| GO:1905523 | C2 | positive regulation of | 0.006535 | 0.722022 |
| | | macrophage migration | | |
| GO:0019432 | C2 | triglyceride biosynthetic | 0.006662 | 0.902527 |
| | | process | | |
| GO:0060338 | C2 | regulation of type I | 0.006662 | 0.902527 |
| | | interferon-mediated | | |
| | | signaling pathway | | |
| GO:0090596 | C2 | sensory organ morphogenesis | 0.006687 | 2.888087 |
| GO:0007044 | C2 | cell-substrate junction | 0.006761 | 1.444043 |
| | | assembly | | |
| GO:0032088 | C2 | negative regulation of | 0.006761 | 1.444043 |
| | | NF-kappaB transcription | | |
| | | factor activity | | |
| GO:1903131 | C2 | mononuclear cell | 0.006773 | 4.33213 |
| | | differentiation | | |
| GO:0001961 | C2 | positive regulation of | 0.00687 | 1.083032 |
| | | cytokine-mediated | | |
| | | signaling pathway | | |
| GO:0035265 | C2 | organ growth | 0.00692 | 2.166065 |
| GO:0044409 | C2 | entry into host | 0.006955 | 1.98556 |
| GO:0045862 | C2 | positive regulation of | 0.007025 | 3.610108 |
| | | proteolysis | | |
| GO:1903322 | C2 | positive regulation of | 0.007147 | 1.805054 |
| | | protein modification by | | |
| | | small protein conjugation | | |
| | | or removal | | |
| GO:0008202 | C2 | steroid metabolic process | 0.007208 | 3.249097 |
| GO:0000302 | C2 | response to reactive | 0.007311 | 2.34657 |
| | | oxygen species | | |
| GO:0050864 | C2 | regulation of B cell | 0.007311 | 2.34657 |
| | | activation | | |
| GO:0072522 | C2 | purine-containing compound | 0.007383 | 2.707581 |
| | | biosynthetic process | | |
| GO:0014904 | C2 | myotube cell development | 0.007384 | 0.902527 |
| GO:0071604 | C2 | transforming growth factor | 0.007384 | 0.902527 |
| | | beta production | | |
| GO:2001233 | C2 | regulation of apoptotic | 0.007434 | 3.610108 |
| | | signaling pathway | | |
| GO:0007566 | C2 | embryo implantation | 0.007463 | 1.083032 |
| GO:0032967 | C2 | positive regulation of | 0.007497 | 0.722022 |
| | | collagen biosynthetic | | |
| | | process | | |
| GO:0042278 | C2 | purine nucleoside | 0.007497 | 0.722022 |
| | | metabolic process | | |
| GO:0046890 | C2 | regulation of lipid | 0.007532 | 2.166065 |
| | | biosynthetic process | | |
| GO:0051251 | C2 | positive regulation of | 0.007563 | 3.790614 |
| | | lymphocyte activation | | |
| GO:1901214 | C2 | regulation of neuron death | 0.00766 | 3.249097 |
| GO:0071241 | C2 | cellular response to | 0.007825 | 2.527076 |
| | | inorganic substance | | |
| GO:0044000 | C2 | movement in host | 0.007853 | 2.166065 |
| GO:0050863 | C2 | regulation of T cell | 0.007862 | 3.610108 |
| | | activation | | |
| GO:0008203 | C2 | cholesterol metabolic | 0.007887 | 1.805054 |
| | | process | | |
| GO:0002286 | C2 | T cell activation | 0.007937 | 1.624549 |
| | | involved in immune | | |
| | | response | | |
| GO:0002718 | C2 | regulation of cytokine | 0.007937 | 1.624549 |
| | | production involved | | |
| | | in immune response | | |
| GO:0071346 | C2 | cellular response to | 0.007937 | 1.624549 |
| | | interferon-gamma | | |
| GO:0002753 | C2 | cytoplasmic pattern | 0.008093 | 1.083032 |
| | | recognition receptor | | |
| | | signaling pathway | | |
| GO:0009154 | C2 | purine ribonucleotide | 0.008159 | 0.902527 |
| | | catabolic process | | |
| GO:0071548 | C2 | response to dexamethasone | 0.008159 | 0.902527 |
| GO:1903532 | C2 | positive regulation of | 0.008169 | 2.888087 |
| | | secretion by cell | | |
| GO:0034341 | C2 | response to interferon-gamma | 0.008279 | 1.805054 |
| GO:0031398 | C2 | positive regulation of | 0.008371 | 1.624549 |
| | | protein ubiquitination | | |
| GO:0032609 | C2 | interferon-gamma production | 0.008371 | 1.624549 |
| GO:0032649 | C2 | regulation of interferon- | 0.008371 | 1.624549 |
| | | gamma production | | |
| GO:0045216 | C2 | cell-cell junction | 0.00854 | 2.34657 |
| | | organization | | |
| GO:0010714 | C2 | positive regulation of | 0.008548 | 0.722022 |
| | | collagen metabolic process | | |
| GO:0010758 | C2 | regulation of macrophage | 0.008548 | 0.722022 |
| | | chemotaxis | | |
| GO:0010894 | C2 | negative regulation of | 0.008548 | 0.722022 |
| | | steroid biosynthetic | | |
| | | process | | |
| GO:0021952 | C2 | central nervous system | 0.008548 | 0.722022 |
| | | projection neuron | | |
| | | axonogenesis | | |
| GO:0071624 | C2 | positive regulation of | 0.008548 | 0.722022 |
| | | granulocyte chemotaxis | | |
| GO:0010876 | C2 | lipid localization | 0.008656 | 4.33213 |
| GO:0022411 | C2 | cellular component | 0.008656 | 4.33213 |
| | | disassembly | | |
| GO:0006986 | C2 | response to unfolded protein | 0.008685 | 1.805054 |
| GO:0045667 | C2 | regulation of osteoblast | 0.008685 | 1.805054 |
| | | differentiation | | |
| GO:0045579 | C2 | positive regulation of | 0.008758 | 0.541516 |
| | | B cell differentiation | | |
| GO:0060099 | C2 | regulation of phagocytosis, | 0.008758 | 0.541516 |
| | | engulfment | | |
| GO:0097531 | C2 | mast cell migration | 0.008758 | 0.541516 |
| GO:1902563 | C2 | regulation of neutrophil | 0.008758 | 0.541516 |
| | | activation | | |
| GO:1905153 | C2 | regulation of membrane | 0.008758 | 0.541516 |
| | | invagination | | |
| GO:0030865 | C2 | cortical cytoskeleton | 0.008759 | 1.083032 |
| | | organization | | |
| GO:0002367 | C2 | cytokine production | 0.008822 | 1.624549 |
| | | involved in immune | | |
| | | response | | |
| GO:0007613 | C2 | memory | 0.008822 | 1.624549 |
| GO:0045785 | C2 | positive regulation of | 0.008865 | 4.33213 |
| | | cell adhesion | | |
| GO:0030100 | C2 | regulation of endocytosis | 0.008872 | 2.34657 |
| GO:0032970 | C2 | regulation of actin | 0.008875 | 3.790614 |
| | | filament-based process | | |
| GO:0001504 | C2 | neurotransmitter uptake | 0.008988 | 0.902527 |
| GO:0014911 | C2 | positive regulation of | 0.008988 | 0.902527 |
| | | smooth muscle cell | | |
| | | migration | | |
| GO:0072538 | C2 | T-helper 17 type immune | 0.008988 | 0.902527 |
| | | response | | |
| GO:1900026 | C2 | positive regulation of | 0.008988 | 0.902527 |
| | | substrate adhesion- | | |
| | | dependent cell spreading | | |
| GO:0048608 | C2 | reproductive structure | 0.009124 | 3.068592 |
| | | development | | |
| GO:0034620 | C2 | cellular response to | 0.009127 | 1.444043 |
| | | unfolded protein | | |
| GO:0006869 | C2 | lipid transport | 0.009145 | 3.971119 |
| GO:0031396 | C2 | regulation of protein | 0.009213 | 2.34657 |
| | | ubiquitination | | |
| GO:0031623 | C2 | receptor internalization | 0.009292 | 1.624549 |
| GO:0044403 | C2 | biological process | 0.009406 | 3.068592 |
| | | involved in symbiotic | | |
| | | interaction | | |
| GO:0060393 | C2 | regulation of pathway- | 0.009464 | 1.083032 |
| | | restricted SMAD | | |
| | | protein phosphorylation | | |
| GO:0002833 | C2 | positive regulation of | 0.009625 | 2.166065 |
| | | response to biotic | | |
| | | stimulus | | |
| GO:0030308 | C2 | negative regulation of | 0.009625 | 2.166065 |
| | | cell growth | | |
| GO:0150115 | C2 | cell-substrate junction | 0.009665 | 1.444043 |
| | | organization | | |
| GO:0006656 | C2 | phosphatidylcholine | 0.00969 | 0.722022 |
| | | biosynthetic process | | |
| GO:0010712 | C2 | regulation of collagen | 0.009874 | 0.902527 |
| | | metabolic process | | |
| GO:0062208 | C2 | positive regulation | 0.009874 | 0.902527 |
| | | of pattern recognition | | |
| | | receptor signaling pathway | | |
| GO:0006836 | C2 | neurotransmitter transport | 0.009927 | 2.34657 |
| GO:0030177 | C2 | positive regulation of | 0.009995 | 1.805054 |
| | | Wnt signaling pathway | | |
| GO:0044282 | C2 | small molecule catabolic | 0.010042 | 3.610108 |
| | | process | | |
| GO:0032615 | C2 | interleukin-12 production | 0.010208 | 1.083032 |
| GO:0032655 | C2 | regulation of | 0.010208 | 1.083032 |
| | | interleukin-12 | | |
| | | production | | |
| GO:0060395 | C2 | SMAD protein signal | 0.010237 | 1.263538 |
| | | transduction | | |
| GO:0030282 | C2 | bone mineralization | 0.010288 | 1.624549 |
| GO:0061458 | C2 | reproductive system | 0.010294 | 3.068592 |
| | | development | | |
| GO:1904951 | C2 | positive regulation of | 0.010294 | 3.068592 |
| | | establishment of | | |
| | | protein localization | | |
| GO:0003205 | C2 | cardiac chamber | 0.010338 | 1.98556 |
| | | development | | |
| GO:0007409 | C2 | axonogenesis | 0.010361 | 3.971119 |
| GO:0034375 | C2 | high-density lipoprotein | 0.010547 | 0.541516 |
| | | particle remodeling | | |
| GO:0042472 | C2 | inner ear morphogenesis | 0.010814 | 1.444043 |
| GO:0046365 | C2 | monosaccharide | 0.010818 | 0.902527 |
| | | catabolic process | | |
| GO:0085029 | C2 | extracellular matrix | 0.010818 | 0.902527 |
| | | assembly | | |
| GO:0055001 | C2 | muscle cell development | 0.010829 | 2.166065 |
| GO:0001654 | C2 | eye development | 0.010868 | 3.610108 |
| GO:0050727 | C2 | regulation of | 0.010904 | 3.790614 |
| | | inflammatory | | |
| | | response | | |
| GO:0007492 | C2 | endoderm development | 0.010906 | 1.263538 |
| GO:0050848 | C2 | regulation of | 0.010906 | 1.263538 |
| | | calcium-mediated | | |
| | | signaling | | |
| GO:0001778 | C2 | plasma membrane repair | 0.010927 | 0.722022 |
| GO:0010464 | C2 | regulation of mesenchymal | 0.010927 | 0.722022 |
| | | cell proliferation | | |
| GO:0034368 | C2 | protein-lipid complex | 0.010927 | 0.722022 |
| | | remodeling | | |
| GO:0034369 | C2 | plasma lipoprotein | 0.010927 | 0.722022 |
| | | particle remodeling | | |
| GO:0060292 | C2 | long-term synaptic | 0.010927 | 0.722022 |
| | | depression | | |
| GO:0070168 | C2 | negative regulation of | 0.010927 | 0.722022 |
| | | biomineral tissue | | |
| | | development | | |
| GO:2000108 | C2 | positive regulation of | 0.010927 | 0.722022 |
| | | leukocyte apoptotic | | |
| | | process | | |
| GO:0035306 | C2 | positive regulation of | 0.010993 | 1.083032 |
| | | dephosphorylation | | |
| GO:0001837 | C2 | epithelial to | 0.011238 | 1.98556 |
| | | mesenchymal transition | | |
| GO:0007568 | C2 | aging | 0.011238 | 1.98556 |
| GO:0031099 | C2 | regeneration | 0.011254 | 2.166065 |
| GO:0006641 | C2 | triglyceride metabolic | 0.011424 | 1.444043 |
| | | process | | |
| GO:0032956 | C2 | regulation of actin | 0.01167 | 3.429603 |
| | | cytoskeleton organization | | |
| GO:0003170 | C2 | heart valve development | 0.01182 | 1.083032 |
| GO:0001774 | C2 | microglial cell activation | 0.011822 | 0.902527 |
| GO:1902622 | C2 | regulation of neutrophil | 0.011822 | 0.902527 |
| | | migration | | |
| GO:0043010 | C2 | camera-type eye development | 0.011832 | 3.249097 |
| GO:0002703 | C2 | regulation of leukocyte | 0.011918 | 2.527076 |
| | | mediated immunity | | |
| GO:0006690 | C2 | icosanoid metabolic process | 0.011929 | 1.624549 |
| GO:0032760 | C2 | positive regulation of | 0.01206 | 1.444043 |
| | | tumor necrosis factor | | |
| | | production | | |
| GO:0051588 | C2 | regulation of | 0.01206 | 1.444043 |
| | | neurotransmitter transport | | |
| GO:0045939 | C2 | negative regulation of | 0.012262 | 0.722022 |
| | | steroid metabolic process | | |
| GO:0061437 | C2 | renal system vasculature | 0.012262 | 0.722022 |
| | | development | | |
| GO:0061440 | C2 | kidney vasculature | 0.012262 | 0.722022 |
| | | development | | |
| GO:0110150 | C2 | negative regulation of | 0.012262 | 0.722022 |
| | | biomineralization | | |
| GO:0002573 | C2 | myeloid leukocyte | 0.01233 | 2.34657 |
| | | differentiation | | |
| GO:0007379 | C2 | segment specification | 0.012533 | 0.541516 |
| GO:0038065 | C2 | collagen-activated | 0.012533 | 0.541516 |
| | | signaling pathway | | |
| GO:0044539 | C2 | long-chain fatty acid | 0.012533 | 0.541516 |
| | | import into cell | | |
| GO:0045019 | C2 | negative regulation of | 0.012533 | 0.541516 |
| | | nitric oxide biosynthetic | | |
| | | process | | |
| GO:0072109 | C2 | glomerular mesangium | 0.012533 | 0.541516 |
| | | development | | |
| GO:1904406 | C2 | negative regulation of | 0.012533 | 0.541516 |
| | | nitric oxide metabolic | | |
| | | process | | |
| GO:0070997 | C2 | neuron death | 0.012636 | 3.429603 |
| GO:0060389 | C2 | pathway-restricted SMAD | 0.01269 | 1.083032 |
| | | protein phosphorylation | | |
| GO:0006195 | C2 | purine nucleotide | 0.012887 | 0.902527 |
| | | catabolic process | | |
| GO:0032527 | C2 | protein exit from | 0.012887 | 0.902527 |
| | | endoplasmic reticulum | | |
| GO:0045981 | C2 | positive regulation of | 0.012887 | 0.902527 |
| | | nucleotide metabolic | | |
| | | process | | |
| GO:1900544 | C2 | positive regulation of | 0.012887 | 0.902527 |
| | | purine nucleotide | | |
| | | metabolic process | | |
| GO:0045444 | C2 | fat cell differentiation | 0.013164 | 2.527076 |
| GO:0050804 | C2 | modulation of chemical | 0.013182 | 3.971119 |
| | | synaptic transmission | | |
| GO:0001558 | C2 | regulation of cell growth | 0.013291 | 3.790614 |
| GO:0010717 | C2 | regulation of epithelial | 0.013409 | 1.444043 |
| | | to mesenchymal transition | | |
| GO:0051222 | C2 | positive regulation of | 0.013427 | 2.888087 |
| | | protein transport | | |
| GO:0099177 | C2 | regulation of trans- | 0.013494 | 3.971119 |
| | | synaptic signaling | | |
| GO:0034367 | C2 | protein-containing | 0.013696 | 0.722022 |
| | | complex remodeling | | |
| GO:1901889 | C2 | negative regulation of | 0.013696 | 0.722022 |
| | | cell junction assembly | | |
| GO:1902235 | C2 | regulation of endoplasmic | 0.013696 | 0.722022 |
| | | reticulum stress-induced | | |
| | | intrinsic apoptotic | | |
| | | signaling pathway | | |
| GO:0010232 | C2 | vascular transport | 0.013903 | 1.263538 |
| GO:0150104 | C2 | transport across blood- | 0.013903 | 1.263538 |
| | | brain barrier | | |
| GO:0009261 | C2 | ribonucleotide catabolic | 0.014015 | 0.902527 |
| | | process | | |
| GO:0043457 | C2 | regulation of cellular | 0.014015 | 0.902527 |
| | | respiration | | |
| GO:0045933 | C2 | positive regulation of | 0.014015 | 0.902527 |
| | | muscle contraction | | |
| GO:0046460 | C2 | neutral lipid biosynthetic | 0.014015 | 0.902527 |
| | | process | | |
| GO:0046463 | C2 | acylglycerol biosynthetic | 0.014015 | 0.902527 |
| | | process | | |
| GO:0002819 | C2 | regulation of adaptive | 0.014087 | 2.166065 |
| | | immune response | | |
| GO:0071248 | C2 | cellular response to | 0.014087 | 2.166065 |
| | | metal ion | | |
| GO:0008637 | C2 | apoptotic mitochondrial | 0.014123 | 1.444043 |
| | | changes | | |
| GO:0034308 | C2 | primary alcohol metabolic | 0.014123 | 1.444043 |
| | | process | | |
| GO:0062013 | C2 | positive regulation of | 0.014228 | 1.805054 |
| | | small molecule metabolic | | |
| | | process | | |
| GO:0072006 | C2 | nephron development | 0.014228 | 1.805054 |
| GO:0030217 | C2 | T cell differentiation | 0.014237 | 2.888087 |
| GO:0042542 | C2 | response to hydrogen | 0.014411 | 1.624549 |
| | | peroxide | | |
| GO:1990266 | C2 | neutrophil migration | 0.014411 | 1.624549 |
| GO:0030239 | C2 | myofibril assembly | 0.014562 | 1.083032 |
| GO:0015812 | C2 | gamma-aminobutyric acid | 0.014717 | 0.541516 |
| | | transport | | |
| GO:0030540 | C2 | female genitalia | 0.014717 | 0.541516 |
| | | development | | |
| GO:0030903 | C2 | notochord development | 0.014717 | 0.541516 |
| GO:0060391 | C2 | positive regulation of | 0.014717 | 0.541516 |
| | | SMAD protein signal | | |
| | | transduction | | |
| GO:0032370 | C2 | positive regulation | 0.014735 | 1.263538 |
| | | of lipid transport | | |
| GO:0032635 | C2 | interleukin-6 production | 0.014862 | 1.98556 |
| GO:0032675 | C2 | regulation of interleukin-6 | 0.014862 | 1.98556 |
| | | production | | |
| GO:1902905 | C2 | positive regulation of | 0.014862 | 1.98556 |
| | | supramolecular fiber | | |
| | | organization | | |
| GO:1903557 | C2 | positive regulation of | 0.014864 | 1.444043 |
| | | tumor necrosis factor | | |
| | | superfamily cytokine | | |
| | | production | | |
| GO:0007015 | C2 | actin filament | 0.015148 | 3.971119 |
| | | organization | | |
| GO:0002269 | C2 | leukocyte activation | 0.015208 | 0.902527 |
| | | involved in inflammatory | | |
| | | response | | |
| GO:0061082 | C2 | myeloid leukocyte cytokine | 0.015208 | 0.902527 |
| | | production | | |
| GO:0001782 | C2 | B cell homeostasis | 0.015233 | 0.722022 |
| GO:0033032 | C2 | regulation of myeloid | 0.015233 | 0.722022 |
| | | cell apoptotic process | | |
| GO:0038094 | C2 | Fc-gamma receptor | 0.015233 | 0.722022 |
| | | signaling pathway | | |
| GO:0042219 | C2 | cellular modified amino | 0.015233 | 0.722022 |
| | | acid catabolic process | | |
| GO:0016125 | C2 | sterol metabolic process | 0.015469 | 1.805054 |
| GO:0042113 | C2 | B cell activation | 0.015495 | 3.249097 |
| GO:0003012 | C2 | muscle system process | 0.015498 | 3.971119 |
| GO:0050922 | C2 | negative regulation of | 0.015566 | 1.083032 |
| | | chemotaxis | | |
| GO:0055002 | C2 | striated muscle cell | 0.015566 | 1.083032 |
| | | development | | |
| GO:0001960 | C2 | negative regulation | 0.015603 | 1.263538 |
| | | of cytokine-mediated | | |
| | | signaling pathway | | |
| GO:0046849 | C2 | bone remodeling | 0.015603 | 1.263538 |
| GO:0006766 | C2 | vitamin metabolic process | 0.015634 | 1.444043 |
| GO:0007596 | C2 | blood coagulation | 0.015675 | 2.34657 |
| GO:0031647 | C2 | regulation of protein | 0.015779 | 3.068592 |
| | | stability | | |
| GO:0006936 | C2 | muscle contraction | 0.015905 | 3.249097 |
| GO:0050871 | C2 | positive regulation of | 0.016119 | 1.805054 |
| | | B cell activation | | |
| GO:0098739 | C2 | import across plasma | 0.016257 | 2.166065 |
| | | membrane | | |
| GO:1901222 | C2 | regulation of | 0.016432 | 1.444043 |
| | | NIK/NF-kappaB signaling | | |
| GO:0043627 | C2 | response to estrogen | 0.016618 | 1.083032 |
| GO:0051701 | C2 | biological process | 0.016837 | 2.166065 |
| | | involved in interaction | | |
| | | with host | | |
| GO:0006536 | C2 | glutamate metabolic process | 0.016874 | 0.722022 |
| GO:0032743 | C2 | positive regulation of | 0.016874 | 0.722022 |
| | | interleukin-2 production | | |
| GO:0034405 | C2 | response to fluid shear | 0.016874 | 0.722022 |
| | | stress | | |
| GO:0035025 | C2 | positive regulation of | 0.016874 | 0.722022 |
| | | Rho protein signal | | |
| | | transduction | | |
| GO:1903320 | C2 | regulation of protein | 0.016982 | 2.527076 |
| | | modification by | | |
| | | small protein | | |
| | | conjugation or removal | | |
| GO:0003417 | C2 | growth plate cartilage | 0.017103 | 0.541516 |
| | | development | | |
| GO:0010759 | C2 | positive regulation of | 0.017103 | 0.541516 |
| | | macrophage chemotaxis | | |
| GO:0032695 | C2 | negative regulation of | 0.017103 | 0.541516 |
| | | interleukin-12 production | | |
| GO:0045618 | C2 | positive regulation | 0.017103 | 0.541516 |
| | | of keratinocyte | | |
| | | differentiation | | |
| GO:1901569 | C2 | fatty acid derivative | 0.017103 | 0.541516 |
| | | catabolic process | | |
| GO:1901888 | C2 | regulation of cell | 0.017432 | 2.166065 |
| | | junction assembly | | |
| GO:0030099 | C2 | myeloid cell | 0.017443 | 3.610108 |
| | | differentiation | | |
| GO:0046928 | C2 | regulation of | 0.017446 | 1.263538 |
| | | neurotransmitter | | |
| | | secretion | | |
| GO:0060021 | C2 | roof of mouth development | 0.017446 | 1.263538 |
| GO:0002548 | C2 | monocyte chemotaxis | 0.017717 | 1.083032 |
| GO:0110148 | C2 | biomineralization | 0.017945 | 1.98556 |
| GO:0045017 | C2 | glycerolipid biosynthetic | 0.018056 | 2.527076 |
| | | process | | |
| GO:1905954 | C2 | positive regulation of | 0.018115 | 1.444043 |
| | | lipid localization | | |
| GO:0090101 | C2 | negative regulation of | 0.01819 | 1.805054 |
| | | transmembrane receptor | | |
| | | protein serine/threonine | | |
| | | kinase signaling pathway | | |
| GO:0050817 | C2 | coagulation | 0.018459 | 2.34657 |
| GO:0022407 | C2 | regulation of cell-cell | 0.018528 | 4.151625 |
| | | adhesion | | |
| GO:0002822 | C2 | regulation of adaptive | 0.018615 | 1.98556 |
| | | immune response based | | |
| | | on somatic recombination | | |
| | | of immune receptors | | |
| | | built from immunoglobulin | | |
| | | superfamily domains | | |
| GO:0042129 | C2 | regulation of T cell | 0.018615 | 1.98556 |
| | | proliferation | | |
| GO:0002446 | C2 | neutrophil mediated | 0.018621 | 0.722022 |
| | | immunity | | |
| GO:0070528 | C2 | protein kinase C | 0.018621 | 0.722022 |
| | | signaling | | |
| GO:0007599 | C2 | hemostasis | 0.019057 | 2.34657 |
| GO:0046638 | C2 | positive regulation | 0.019187 | 0.902527 |
| | | of alpha-beta T cell | | |
| | | differentiation | | |
| GO:2000677 | C2 | regulation of | 0.019187 | 0.902527 |
| | | transcription regulatory | | |
| | | region DNA binding | | |
| GO:0031349 | C2 | positive regulation of | 0.019392 | 2.888087 |
| | | defense response | | |
| GO:0009306 | C2 | protein secretion | 0.019498 | 3.249097 |
| GO:0120254 | C2 | olefinic compound | 0.019675 | 1.805054 |
| | | metabolic process | | |
| GO:0009110 | C2 | vitamin biosynthetic | 0.019692 | 0.541516 |
| | | process | | |
| GO:0043691 | C2 | reverse cholesterol | 0.019692 | 0.541516 |
| | | transport | | |
| GO:0055093 | C2 | response to hyperoxia | 0.019692 | 0.541516 |
| GO:0060339 | C2 | negative regulation of | 0.019692 | 0.541516 |
| | | type I interferon- | | |
| | | mediated signaling pathway | | |
| GO:0072079 | C2 | nephron tubule formation | 0.019692 | 0.541516 |
| GO:1902236 | C2 | negative regulation of | 0.019692 | 0.541516 |
| | | endoplasmic reticulum | | |
| | | stress-induced intrinsic | | |
| | | apoptotic signaling | | |
| | | pathway | | |
| GO:2000678 | C2 | negative regulation | 0.019692 | 0.541516 |
| | | of transcription | | |
| | | regulatory region | | |
| | | DNA binding | | |
| GO:0016049 | C2 | cell growth | 0.019739 | 4.151625 |
| GO:0014706 | C2 | striated muscle tissue | 0.019764 | 2.527076 |
| | | development | | |
| GO:0033559 | C2 | unsaturated fatty acid | 0.019918 | 1.444043 |
| | | metabolic process | | |
| GO:0048640 | C2 | negative regulation | 0.019918 | 1.444043 |
| | | of developmental growth | | |
| GO:0007162 | C2 | negative regulation | 0.019924 | 2.888087 |
| | | of cell adhesion | | |
| GO:0035592 | C2 | establishment of protein | 0.019988 | 3.249097 |
| | | localization to | | |
| | | extracellular region | | |
| GO:0043462 | C2 | regulation of ATP- | 0.020064 | 1.083032 |
| | | dependent activity | | |
| GO:0046637 | C2 | regulation of alpha-beta | 0.020064 | 1.083032 |
| | | T cell differentiation | | |
| GO:0050805 | C2 | negative regulation of | 0.020064 | 1.083032 |
| | | synaptic transmission | | |
| GO:0061035 | C2 | regulation of cartilage | 0.020064 | 1.083032 |
| | | development | | |
| GO:1904888 | C2 | cranial skeletal system | 0.020064 | 1.083032 |
| | | development | | |
| GO:0002504 | C2 | antigen processing and | 0.020477 | 0.722022 |
| | | presentation of peptide | | |
| | | or polysaccharide antigen | | |
| | | via MHC class II | | |
| GO:0007616 | C2 | long-term memory | 0.020477 | 0.722022 |
| GO:0010922 | C2 | positive regulation of | 0.020477 | 0.722022 |
| | | phosphatase activity | | |
| GO:0098810 | C2 | neurotransmitter reuptake | 0.020477 | 0.722022 |
| GO:0032436 | C2 | positive regulation of | 0.020491 | 1.263538 |
| | | proteasomal ubiquitin- | | |
| | | dependent protein | | |
| | | catabolic process | | |
| GO:0048704 | C2 | embryonic skeletal | 0.020491 | 1.263538 |
| | | system morphogenesis | | |
| GO:0060761 | C2 | negative regulation of | 0.020491 | 1.263538 |
| | | response to cytokine | | |
| | | stimulus | | |
| GO:0060562 | C2 | epithelial tube morphogenesis | 0.020545 | 3.068592 |
| GO:0048732 | C2 | gland development | 0.020631 | 3.790614 |
| GO:0061900 | C2 | glial cell activation | 0.020651 | 0.902527 |
| GO:0019221 | C2 | cytokine-mediated | 0.021011 | 4.151625 |
| | | signaling pathway | | |
| GO:0008277 | C2 | regulation of G | 0.021363 | 1.624549 |
| | | protein-coupled receptor | | |
| | | signaling pathway | | |
| GO:0014909 | C2 | smooth muscle cell | 0.021583 | 1.263538 |
| | | migration | | |
| GO:1990874 | C2 | vascular associated | 0.021583 | 1.263538 |
| | | smooth muscle cell | | |
| | | proliferation | | |
| GO:0070482 | C2 | response to oxygen levels | 0.021618 | 3.068592 |
| GO:0009206 | C2 | purine ribonucleoside | 0.021845 | 1.444043 |
| | | triphosphate | | |
| | | biosynthetic process | | |
| GO:0071887 | C2 | leukocyte apoptotic process | 0.021845 | 1.444043 |
| GO:0009612 | C2 | response to mechanical | 0.022053 | 2.166065 |
| | | stimulus | | |
| GO:0035966 | C2 | response to topologically | 0.022068 | 1.805054 |
| | | incorrect protein | | |
| GO:0002429 | C2 | immune response-activating | 0.022161 | 2.888087 |
| | | cell surface receptor | | |
| | | signaling pathway | | |
| GO:0002757 | C2 | immune response-activating | 0.022161 | 2.888087 |
| | | signal transduction | | |
| GO:0022604 | C2 | regulation of cell | 0.022161 | 2.888087 |
| | | morphogenesis | | |
| GO:0003179 | C2 | heart valve morphogenesis | 0.022187 | 0.902527 |
| GO:0038093 | C2 | Fc receptor signaling | 0.022187 | 0.902527 |
| | | pathway | | |
| GO:0060632 | C2 | regulation of microtubule- | 0.022187 | 0.902527 |
| | | based movement | | |
| GO:0045766 | C2 | positive regulation of | 0.022246 | 1.98556 |
| | | angiogenesis | | |
| GO:1904018 | C2 | positive regulation of | 0.022246 | 1.98556 |
| | | vasculature development | | |
| GO:0007411 | C2 | axon guidance | 0.022273 | 2.34657 |
| GO:0010934 | C2 | macrophage cytokine | 0.022442 | 0.722022 |
| | | production | | |
| GO:0010935 | C2 | regulation of macrophage | 0.022442 | 0.722022 |
| | | cytokine production | | |
| GO:0021955 | C2 | central nervous system | 0.022442 | 0.722022 |
| | | neuron axonogenesis | | |
| GO:0071402 | C2 | cellular response to | 0.022442 | 0.722022 |
| | | lipoprotein particle | | |
| | | stimulus | | |
| GO:0007413 | C2 | axonal fasciculation | 0.022485 | 0.541516 |
| GO:0038083 | C2 | peptidyl-tyrosine | 0.022485 | 0.541516 |
| | | autophosphorylation | | |
| GO:0045821 | C2 | positive regulation | 0.022485 | 0.541516 |
| | | of glycolytic process | | |
| GO:0071498 | C2 | cellular response to | 0.022485 | 0.541516 |
| | | fluid shear stress | | |
| GO:0106030 | C2 | neuron projection | 0.022485 | 0.541516 |
| | | fasciculation | | |
| GO:0050810 | C2 | regulation of steroid | 0.022613 | 1.083032 |
| | | biosynthetic process | | |
| GO:0045807 | C2 | positive regulation of | 0.022715 | 1.263538 |
| | | endocytosis | | |
| GO:0072080 | C2 | nephron tubule development | 0.022715 | 1.263538 |
| GO:0060485 | C2 | mesenchyme development | 0.022749 | 2.888087 |
| GO:0009145 | C2 | purine nucleoside | 0.022856 | 1.444043 |
| | | triphosphate biosynthetic | | |
| | | process | | |
| GO:1901800 | C2 | positive regulation of | 0.022856 | 1.444043 |
| | | proteasomal protein | | |
| | | catabolic process | | |
| GO:0097485 | C2 | neuron projection guidance | 0.022962 | 2.34657 |
| GO:0030098 | C2 | lymphocyte differentiation | 0.022979 | 3.610108 |
| GO:0006006 | C2 | glucose metabolic process | 0.02303 | 1.98556 |
| GO:0045860 | C2 | positive regulation of | 0.023495 | 3.610108 |
| | | protein kinase activity | | |
| GO:0030258 | C2 | lipid modification | 0.023525 | 2.166065 |
| GO:0031960 | C2 | response to corticosteroid | 0.023776 | 1.805054 |
| GO:0010718 | C2 | positive regulation of | 0.023795 | 0.902527 |
| | | epithelial to mesenchymal | | |
| | | transition | | |
| GO:0051438 | C2 | regulation of ubiquitin- | 0.023795 | 0.902527 |
| | | protein transferase | | |
| | | activity | | |
| GO:0002768 | C2 | immune response-regulating | 0.023892 | 3.068592 |
| | | cell surface receptor | | |
| | | signaling pathway | | |
| GO:0071692 | C2 | protein localization | 0.024256 | 3.249097 |
| | | to extracellular region | | |
| GO:0007020 | C2 | microtubule nucleation | 0.024518 | 0.722022 |
| GO:0010742 | C2 | macrophage derived foam | 0.024518 | 0.722022 |
| | | cell differentiation | | |
| GO:0040036 | C2 | regulation of fibroblast | 0.024518 | 0.722022 |
| | | growth factor receptor | | |
| | | signaling pathway | | |
| GO:0048741 | C2 | skeletal muscle fiber | 0.024518 | 0.722022 |
| | | development | | |
| GO:0140448 | C2 | signaling receptor ligand | 0.024518 | 0.722022 |
| | | precursor processing | | |
| GO:0050777 | C2 | negative regulation of | 0.024659 | 1.98556 |
| | | immune response | | |
| GO:0035967 | C2 | cellular response to | 0.024977 | 1.444043 |
| | | topologically incorrect | | |
| | | protein | | |
| GO:0090288 | C2 | negative regulation of | 0.024977 | 1.444043 |
| | | cellular response to | | |
| | | growth factor stimulus | | |
| GO:0051952 | C2 | regulation of amine transport | 0.0251 | 1.263538 |
| GO:0030968 | C2 | endoplasmic reticulum | 0.025371 | 1.083032 |
| | | unfolded protein | | |
| | | response | | |
| GO:0032720 | C2 | negative regulation of | 0.025371 | 1.083032 |
| | | tumor necrosis | | |
| | | factor production | | |
| GO:0032729 | C2 | positive regulation of | 0.025371 | 1.083032 |
| | | interferon-gamma | | |
| | | production | | |
| GO:0032700 | C2 | negative regulation of | 0.025481 | 0.541516 |
| | | interleukin-17 | | |
| | | production | | |
| GO:0046128 | C2 | purine ribonucleoside | 0.025481 | 0.541516 |
| | | metabolic process | | |
| GO:0050765 | C2 | negative regulation | 0.025481 | 0.541516 |
| | | of phagocytosis | | |
| GO:0051220 | C2 | cytoplasmic sequestering | 0.025481 | 0.541516 |
| | | of protein | | |
| GO:0060065 | C2 | uterus development | 0.025481 | 0.541516 |
| GO:0071404 | C2 | cellular response to | 0.025481 | 0.541516 |
| | | low-density lipoprotein | | |
| | | particle stimulus | | |
| GO:0072111 | C2 | cell proliferation | 0.025481 | 0.541516 |
| | | involved in kidney | | |
| | | development | | |
| GO:0042445 | C2 | hormone metabolic process | 0.025877 | 2.34657 |
| GO:0048738 | C2 | cardiac muscle tissue | 0.025877 | 2.34657 |
| | | development | | |
| GO:0032355 | C2 | response to estradiol | 0.026088 | 1.444043 |
| GO:0090100 | C2 | positive regulation of | 0.026088 | 1.444043 |
| | | transmembrane receptor | | |
| | | protein serine/threonine | | |
| | | kinase signaling pathway | | |
| GO:0030512 | C2 | negative regulation | 0.026354 | 1.263538 |
| | | of transforming growth | | |
| | | factor beta receptor | | |
| | | signaling pathway | | |
| GO:0061326 | C2 | renal tubule development | 0.026354 | 1.263538 |
| GO:2001237 | C2 | negative regulation of | 0.026354 | 1.263538 |
| | | extrinsic apoptotic | | |
| | | signaling pathway | | |
| GO:0031334 | C2 | positive regulation of | 0.026371 | 1.98556 |
| | | protein-containing | | |
| | | complex assembly | | |
| GO:0048639 | C2 | positive regulation of | 0.026513 | 1.805054 |
| | | developmental growth | | |
| GO:0019058 | C2 | viral life cycle | 0.026527 | 2.888087 |
| GO:0032570 | C2 | response to progesterone | 0.026707 | 0.722022 |
| GO:0033028 | C2 | myeloid cell apoptotic | 0.026707 | 0.722022 |
| | | process | | |
| GO:0044273 | C2 | sulfur compound catabolic | 0.026707 | 0.722022 |
| | | process | | |
| GO:0071634 | C2 | regulation of transforming | 0.026707 | 0.722022 |
| | | growth factor beta | | |
| | | production | | |
| GO:0090077 | C2 | foam cell differentiation | 0.026707 | 0.722022 |
| GO:0097242 | C2 | amyloid-beta clearance | 0.026707 | 0.722022 |
| GO:0009064 | C2 | glutamine family amino | 0.02683 | 1.083032 |
| | | acid metabolic process | | |
| GO:1901264 | C2 | carbohydrate derivative | 0.02683 | 1.083032 |
| | | transport | | |
| GO:1901568 | C2 | fatty acid derivative | 0.02683 | 1.083032 |
| | | metabolic process | | |
| GO:1903533 | C2 | regulation of protein | 0.02683 | 1.083032 |
| | | targeting | | |
| GO:0045089 | C2 | positive regulation of | 0.027166 | 1.624549 |
| | | innate immune response | | |
| GO:0030278 | C2 | regulation of ossification | 0.027232 | 1.444043 |
| GO:0044706 | C2 | multi-multicellular | 0.027522 | 2.166065 |
| | | organism process | | |
| GO:0001776 | C2 | leukocyte homeostasis | 0.027651 | 1.263538 |
| GO:0042632 | C2 | cholesterol homeostasis | 0.027651 | 1.263538 |
| GO:0061136 | C2 | regulation of proteasomal | 0.028166 | 1.98556 |
| | | protein catabolic process | | |
| GO:0007517 | C2 | muscle organ development | 0.0283 | 3.068592 |
| GO:0002437 | C2 | inflammatory response to | 0.028344 | 1.083032 |
| | | antigenic stimulus | | |
| GO:0061333 | C2 | renal tubule morphogenesis | 0.028344 | 1.083032 |
| GO:1903556 | C2 | negative regulation of | 0.028344 | 1.083032 |
| | | tumor necrosis factor | | |
| | | superfamily cytokine | | |
| | | production | | |
| GO:0002224 | C2 | toll-like receptor | 0.028411 | 1.444043 |
| | | signaling pathway | | |
| GO:0009201 | C2 | ribonucleoside triphosphate | 0.028411 | 1.444043 |
| | | biosynthetic process | | |
| GO:0022612 | C2 | gland morphogenesis | 0.028411 | 1.444043 |
| GO:0071482 | C2 | cellular response to | 0.028411 | 1.444043 |
| | | light stimulus | | |
| GO:2000058 | C2 | regulation of ubiquitin- | 0.028459 | 1.805054 |
| | | dependent protein | | |
| | | catabolic process | | |
| GO:0002460 | C2 | adaptive immune response | 0.028528 | 3.249097 |
| | | based on somatic | | |
| | | recombination of immune | | |
| | | receptors built from | | |
| | | immunoglobulin | | |
| | | superfamily domains | | |
| GO:0002053 | C2 | positive regulation | 0.028681 | 0.541516 |
| | | of mesenchymal cell | | |
| | | proliferation | | |
| GO:0002689 | C2 | negative regulation of | 0.028681 | 0.541516 |
| | | leukocyte chemotaxis | | |
| GO:0045723 | C2 | positive regulation of | 0.028681 | 0.541516 |
| | | fatty acid biosynthetic | | |
| | | process | | |
| GO:0046058 | C2 | CAMP metabolic process | 0.028681 | 0.541516 |
| GO:0060965 | C2 | negative regulation of | 0.028681 | 0.541516 |
| | | miRNA-mediated gene | | |
| | | silencing | | |
| GO:0061760 | C2 | antifungal innate immune | 0.028681 | 0.541516 |
| | | response | | |
| GO:2000178 | C2 | negative regulation of | 0.028681 | 0.541516 |
| | | neural precursor | | |
| | | cell proliferation | | |
| GO:2001169 | C2 | regulation of ATP | 0.028681 | 0.541516 |
| | | biosynthetic process | | |
| GO:0001666 | C2 | response to hypoxia | 0.0287 | 2.707581 |
| GO:0055092 | C2 | sterol homeostasis | 0.02899 | 1.263538 |
| GO:0003352 | C2 | regulation of cilium | 0.029008 | 0.722022 |
| | | movement | | |
| GO:0009187 | C2 | cyclic nucleotide | 0.029008 | 0.722022 |
| | | metabolic process | | |
| GO:0140467 | C2 | integrated stress | 0.029008 | 0.722022 |
| | | response signaling | | |
| GO:0001912 | C2 | positive regulation | 0.029062 | 0.902527 |
| | | of leukocyte mediated | | |
| | | cytotoxicity | | |
| GO:0030042 | C2 | actin filament | 0.029062 | 0.902527 |
| | | depolymerization | | |
| GO:0007601 | C2 | visual perception | 0.029252 | 2.166065 |
| GO:0042471 | C2 | ear morphogenesis | 0.029625 | 1.444043 |
| GO:0055007 | C2 | cardiac muscle cell | 0.029625 | 1.444043 |
| | | differentiation | | |
| GO:0003151 | C2 | outflow tract morphogenesis | 0.029914 | 1.083032 |
| GO:0008344 | C2 | adult locomotory behavior | 0.029914 | 1.083032 |
| GO:0071230 | C2 | cellular response to | 0.029914 | 1.083032 |
| | | amino acid stimulus | | |
| GO:0072028 | C2 | nephron morphogenesis | 0.029914 | 1.083032 |
| GO:0015849 | C2 | organic acid transport | 0.030019 | 2.888087 |
| GO:0031348 | C2 | negative regulation of | 0.030187 | 2.527076 |
| | | defense response | | |
| GO:0001952 | C2 | regulation of cell- | 0.030873 | 1.444043 |
| | | matrix adhesion | | |
| GO:0010811 | C2 | positive regulation of | 0.030873 | 1.444043 |
| | | cell-substrate adhesion | | |
| GO:0045599 | C2 | negative regulation of | 0.030968 | 0.902527 |
| | | fat cell differentiation | | |
| GO:0051353 | C2 | positive regulation of | 0.030968 | 0.902527 |
| | | oxidoreductase activity | | |
| GO:0072210 | C2 | metanephric nephron | 0.031424 | 0.722022 |
| | | development | | |
| GO:0150077 | C2 | regulation of | 0.031424 | 0.722022 |
| | | neuroinflammatory response | | |
| GO:0001570 | C2 | vasculogenesis | 0.031539 | 1.083032 |
| GO:0005996 | C2 | monosaccharide metabolic | 0.03162 | 2.34657 |
| | | process | | |
| GO:0006979 | C2 | response to oxidative | 0.031687 | 3.610108 |
| | | stress | | |
| GO:0070167 | C2 | regulation of biomineral | 0.031799 | 1.263538 |
| | | tissue development | | |
| GO:0006925 | C2 | inflammatory cell | 0.032084 | 0.541516 |
| | | apoptotic process | | |
| GO:0038095 | C2 | Fc-epsilon receptor | 0.032084 | 0.541516 |
| | | signaling pathway | | |
| GO:0060149 | C2 | negative regulation | 0.032084 | 0.541516 |
| | | of post-transcriptional | | |
| | | gene silencing | | |
| GO:0060967 | C2 | negative regulation of | 0.032084 | 0.541516 |
| | | gene silencing by RNA | | |
| GO:1900369 | C2 | negative regulation | 0.032084 | 0.541516 |
| | | of post-transcriptional | | |
| | | gene silencing by RNA | | |
| GO:1902074 | C2 | response to salt | 0.032084 | 0.541516 |
| GO:0003231 | C2 | cardiac ventricle development | 0.032158 | 1.444043 |
| GO:0006937 | C2 | regulation of muscle | 0.032652 | 1.805054 |
| | | contraction | | |
| GO:0031214 | C2 | biomineral tissue development | 0.032652 | 1.805054 |
| GO:1903039 | C2 | positive regulation of | 0.03268 | 2.527076 |
| | | leukocyte cell-cell | | |
| | | adhesion | | |
| GO:0007612 | C2 | learning | 0.032786 | 1.624549 |
| GO:0010821 | C2 | regulation of mitochondrion | 0.032786 | 1.624549 |
| | | organization | | |
| GO:0034614 | C2 | cellular response to | 0.032786 | 1.624549 |
| | | reactive oxygen species | | |
| GO:0050953 | C2 | sensory perception of | 0.032944 | 2.166065 |
| | | light stimulus | | |
| GO:0034113 | C2 | heterotypic cell-cell | 0.032951 | 0.902527 |
| | | adhesion | | |
| GO:1900024 | C2 | regulation of substrate | 0.032951 | 0.902527 |
| | | adhesion-dependent cell | | |
| | | spreading | | |
| GO:0001542 | C2 | ovulation from ovarian | 0.033013 | 0.361011 |
| | | follicle | | |
| GO:0003418 | C2 | growth plate cartilage | 0.033013 | 0.361011 |
| | | chondrocyte differentiation | | |
| GO:0014012 | C2 | peripheral nervous system | 0.033013 | 0.361011 |
| | | axon regeneration | | |
| GO:0015851 | C2 | nucleobase transport | 0.033013 | 0.361011 |
| GO:0021561 | C2 | facial nerve development | 0.033013 | 0.361011 |
| GO:0021604 | C2 | cranial nerve structural | 0.033013 | 0.361011 |
| | | organization | | |
| GO:0021610 | C2 | facial nerve morphogenesis | 0.033013 | 0.361011 |
| GO:0022400 | C2 | regulation of rhodopsin | 0.033013 | 0.361011 |
| | | mediated signaling pathway | | |
| GO:0032353 | C2 | negative regulation of | 0.033013 | 0.361011 |
| | | hormone biosynthetic | | |
| | | process | | |
| GO:0033034 | C2 | positive regulation of | 0.033013 | 0.361011 |
| | | myeloid cell apoptotic | | |
| | | process | | |
| GO:0048263 | C2 | determination of dorsal | 0.033013 | 0.361011 |
| | | identity | | |
| GO:0051342 | C2 | regulation of cyclic- | 0.033013 | 0.361011 |
| | | nucleotide phosphodiesterase | | |
| | | activity | | |
| GO:0051933 | C2 | amino acid neurotransmitter | 0.033013 | 0.361011 |
| | | reuptake | | |
| GO:0060068 | C2 | vagina development | 0.033013 | 0.361011 |
| GO:0070862 | C2 | negative regulation of | 0.033013 | 0.361011 |
| | | protein exit from | | |
| | | endoplasmic reticulum | | |
| GO:0071377 | C2 | cellular response to | 0.033013 | 0.361011 |
| | | glucagon stimulus | | |
| GO:0099566 | C2 | regulation of postsynaptic | 0.033013 | 0.361011 |
| | | cytosolic calcium ion | | |
| | | concentration | | |
| GO:1901620 | C2 | regulation of smoothened | 0.033013 | 0.361011 |
| | | signaling pathway | | |
| | | involved in dorsal/ventral | | |
| | | neural tube patterning | | |
| GO:1905809 | C2 | negative regulation of | 0.033013 | 0.361011 |
| | | synapse organization | | |
| GO:2000425 | C2 | regulation of apoptotic | 0.033013 | 0.361011 |
| | | cell clearance | | |
| GO:0051963 | C2 | regulation of synapse | 0.03327 | 1.263538 |
| | | assembly | | |
| GO:0010469 | C2 | regulation of signaling | 0.033765 | 1.805054 |
| | | receptor activity | | |
| GO:0002714 | C2 | positive regulation of | 0.033955 | 0.722022 |
| | | B cell mediated immunity | | |
| GO:0002891 | C2 | positive regulation of | 0.033955 | 0.722022 |
| | | immunoglobulin mediated | | |
| | | immune response | | |
| GO:0010463 | C2 | mesenchymal cell | 0.033955 | 0.722022 |
| | | proliferation | | |
| GO:0019320 | C2 | hexose catabolic process | 0.033955 | 0.722022 |
| GO:0050870 | C2 | positive regulation of | 0.034344 | 2.34657 |
| | | T cell activation | | |
| GO:0032479 | C2 | regulation of type I | 0.034784 | 1.263538 |
| | | interferon production | | |
| GO:0032606 | C2 | type I interferon | 0.034784 | 1.263538 |
| | | production | | |
| GO:0110149 | C2 | regulation of | 0.034784 | 1.263538 |
| | | biomineralization | | |
| GO:0060538 | C2 | skeletal muscle organ | 0.034903 | 1.805054 |
| | | development | | |
| GO:0030500 | C2 | regulation of bone | 0.034959 | 1.083032 |
| | | mineralization | | |
| GO:0034340 | C2 | response to type I | 0.034959 | 1.083032 |
| | | interferon | | |
| GO:0000381 | C2 | regulation of | 0.035011 | 0.902527 |
| | | alternative mRNA | | |
| | | splicing, via | | |
| | | spliceosome | | |
| GO:0031102 | C2 | neuron projection | 0.035011 | 0.902527 |
| | | regeneration | | |
| GO:0032233 | C2 | positive regulation of | 0.035011 | 0.902527 |
| | | actin filament bundle | | |
| | | assembly | | |
| GO:0035904 | C2 | aorta development | 0.035011 | 0.902527 |
| GO:0043030 | C2 | regulation of macrophage | 0.035011 | 0.902527 |
| | | activation | | |
| GO:0070228 | C2 | regulation of lymphocyte | 0.035011 | 0.902527 |
| | | apoptotic process | | |
| GO:0007043 | C2 | cell-cell junction | 0.035244 | 1.624549 |
| | | assembly | | |
| GO:0048762 | C2 | mesenchymal cell | 0.035289 | 2.34657 |
| | | differentiation | | |
| GO:0006007 | C2 | glucose catabolic process | 0.035687 | 0.541516 |
| GO:0043302 | C2 | positive regulation of | 0.035687 | 0.541516 |
| | | leukocyte degranulation | | |
| GO:0046885 | C2 | regulation of hormone | 0.035687 | 0.541516 |
| | | biosynthetic process | | |
| GO:0048745 | C2 | smooth muscle tissue | 0.035687 | 0.541516 |
| | | development | | |
| GO:0072273 | C2 | metanephric nephron | 0.035687 | 0.541516 |
| | | morphogenesis | | |
| GO:0072530 | C2 | purine-containing | 0.035687 | 0.541516 |
| | | compound transmembrane | | |
| | | transport | | |
| GO:0090023 | C2 | positive regulation of | 0.035687 | 0.541516 |
| | | neutrophil chemotaxis | | |
| GO:2000311 | C2 | regulation of AMPA | 0.035687 | 0.541516 |
| | | receptor activity | | |
| GO:0043534 | C2 | blood vessel endothelial | 0.036069 | 1.805054 |
| | | cell migration | | |
| GO:1903706 | C2 | regulation of hemopoiesis | 0.036131 | 3.429603 |
| GO:0033674 | C2 | positive regulation of | 0.036158 | 3.971119 |
| | | kinase activity | | |
| GO:0015837 | C2 | amine transport | 0.036345 | 1.263538 |
| GO:0045687 | C2 | positive regulation of | 0.0366 | 0.722022 |
| | | glial cell differentiation | | |
| GO:1900371 | C2 | regulation of purine | 0.0366 | 0.722022 |
| | | nucleotide biosynthetic | | |
| | | process | | |
| GO:0031663 | C2 | lipopolysaccharide-mediated | 0.037149 | 0.902527 |
| | | signaling pathway | | |
| GO:0032623 | C2 | interleukin-2 production | 0.037149 | 0.902527 |
| GO:0032663 | C2 | regulation of interleukin-2 | 0.037149 | 0.902527 |
| | | production | | |
| GO:0032757 | C2 | positive regulation of | 0.037149 | 0.902527 |
| | | interleukin-8 production | | |
| GO:0045453 | C2 | bone resorption | 0.037149 | 0.902527 |
| GO:0046579 | C2 | positive regulation of | 0.037149 | 0.902527 |
| | | Ras protein signal | | |
| | | transduction | | |
| GO:0006694 | C2 | steroid biosynthetic | 0.037261 | 1.805054 |
| | | process | | |
| GO:1903828 | C2 | negative regulation of | 0.037342 | 1.98556 |
| | | protein localization | | |
| GO:0044089 | C2 | positive regulation of | 0.03753 | 3.971119 |
| | | cellular component | | |
| | | biogenesis | | |
| GO:0032612 | C2 | interleukin-1 production | 0.037659 | 1.444043 |
| GO:0032652 | C2 | regulation of interleukin-1 | 0.037659 | 1.444043 |
| | | production | | |
| GO:0045621 | C2 | positive regulation of | 0.037659 | 1.444043 |
| | | lymphocyte differentiation | | |
| GO:0003279 | C2 | cardiac septum development | 0.03795 | 1.263538 |
| GO:0062207 | C2 | regulation of pattern | 0.03795 | 1.263538 |
| | | recognition receptor | | |
| | | signaling pathway | | |
| GO:0019318 | C2 | hexose metabolic process | 0.038014 | 2.166065 |
| GO:0042176 | C2 | regulation of protein | 0.038058 | 3.068592 |
| | | catabolic process | | |
| GO:0021954 | C2 | central nervous system | 0.03861 | 1.083032 |
| | | neuron development | | |
| GO:0048041 | C2 | focal adhesion assembly | 0.03861 | 1.083032 |
| GO:0007127 | C2 | meiosis I | 0.039127 | 1.444043 |
| GO:0032368 | C2 | regulation of lipid | 0.039164 | 1.624549 |
| | | transport | | |
| GO:0090257 | C2 | regulation of muscle | 0.039252 | 2.34657 |
| | | system process | | |
| GO:0030808 | C2 | regulation of nucleotide | 0.039362 | 0.722022 |
| | | biosynthetic process | | |
| GO:0000768 | C2 | syncytium formation by | 0.039366 | 0.902527 |
| | | plasma membrane fusion | | |
| GO:0001947 | C2 | heart looping | 0.039366 | 0.902527 |
| GO:0002712 | C2 | regulation of B cell | 0.039366 | 0.902527 |
| | | mediated immunity | | |
| GO:0002889 | C2 | regulation of immunoglobulin | 0.039366 | 0.902527 |
| | | mediated immune response | | |
| GO:0050918 | C2 | positive chemotaxis | 0.039366 | 0.902527 |
| GO:0051893 | C2 | regulation of focal | 0.039366 | 0.902527 |
| | | adhesion assembly | | |
| GO:0090109 | C2 | regulation of | 0.039366 | 0.902527 |
| | | cell-substrate | | |
| | | junction assembly | | |
| GO:0140253 | C2 | cell-cell fusion | 0.039366 | 0.902527 |
| GO:0036119 | C2 | response to platelet- | 0.03949 | 0.541516 |
| | | derived growth factor | | |
| GO:0060740 | C2 | prostate gland | 0.03949 | 0.541516 |
| | | epithelium morphogenesis | | |
| GO:1902455 | C2 | negative regulation | 0.03949 | 0.541516 |
| | | of stem cell population | | |
| | | maintenance | | |
| GO:0036293 | C2 | response to decreased | 0.039563 | 2.707581 |
| | | oxygen levels | | |
| GO:0002158 | C2 | osteoclast proliferation | 0.03958 | 0.361011 |
| GO:0003139 | C2 | secondary heart field | 0.03958 | 0.361011 |
| | | specification | | |
| GO:0006183 | C2 | GTP biosynthetic process | 0.03958 | 0.361011 |
| GO:0007494 | C2 | midgut development | 0.03958 | 0.361011 |
| GO:0010968 | C2 | regulation of microtubule | 0.03958 | 0.361011 |
| | | nucleation | | |
| GO:0014831 | C2 | gastro-intestinal system | 0.03958 | 0.361011 |
| | | smooth muscle contraction | | |
| GO:0015810 | C2 | aspartate transmembrane | 0.03958 | 0.361011 |
| | | transport | | |
| GO:0019372 | C2 | lipoxygenase pathway | 0.03958 | 0.361011 |
| GO:0031293 | C2 | membrane protein | 0.03958 | 0.361011 |
| | | intracellular domain | | |
| | | proteolysis | | |
| GO:0032096 | C2 | negative regulation of | 0.03958 | 0.361011 |
| | | response to food | | |
| GO:0032099 | C2 | negative regulation of | 0.03958 | 0.361011 |
| | | appetite | | |
| GO:0032105 | C2 | negative regulation of | 0.03958 | 0.361011 |
| | | response to extracellular | | |
| | | stimulus | | |
| GO:0032108 | C2 | negative regulation of | 0.03958 | 0.361011 |
| | | response to nutrient | | |
| | | levels | | |
| GO:0032329 | C2 | serine transport | 0.03958 | 0.361011 |
| GO:0033869 | C2 | nucleoside bisphosphate | 0.03958 | 0.361011 |
| | | catabolic process | | |
| GO:0034031 | C2 | ribonucleoside bisphosphate | 0.03958 | 0.361011 |
| | | catabolic process | | |
| GO:0034034 | C2 | purine nucleoside | 0.03958 | 0.361011 |
| | | bisphosphate catabolic | | |
| | | process | | |
| GO:0039530 | C2 | MDA-5 signaling pathway | 0.03958 | 0.361011 |
| GO:0042451 | C2 | purine nucleoside | 0.03958 | 0.361011 |
| | | biosynthetic process | | |
| GO:0042455 | C2 | ribonucleoside biosynthetic | 0.03958 | 0.361011 |
| | | process | | |
| GO:0046129 | C2 | purine ribonucleoside | 0.03958 | 0.361011 |
| | | biosynthetic process | | |
| GO:0048262 | C2 | determination of | 0.03958 | 0.361011 |
| | | dorsal/ventral asymmetry | | |
| GO:0060900 | C2 | embryonic camera-type | 0.03958 | 0.361011 |
| | | eye formation | | |
| GO:0070778 | C2 | L-aspartate transmembrane | 0.03958 | 0.361011 |
| | | transport | | |
| GO:0072124 | C2 | regulation of glomerular | 0.03958 | 0.361011 |
| | | mesangial cell | | |
| | | proliferation | | |
| GO:0072488 | C2 | ammonium transmembrane | 0.03958 | 0.361011 |
| | | transport | | |
| GO:0098883 | C2 | synapse pruning | 0.03958 | 0.361011 |
| GO:1990440 | C2 | positive regulation | 0.03958 | 0.361011 |
| | | of transcription from | | |
| | | RNA polymerase II | | |
| | | promoter in response | | |
| | | to endoplasmic reticulum | | |
| | | stress | | |
| GO:2000109 | C2 | regulation of | 0.03958 | 0.361011 |
| | | macrophage apoptotic | | |
| | | process | | |
| GO:2000727 | C2 | positive regulation | 0.03958 | 0.361011 |
| | | of cardiac muscle | | |
| | | cell differentiation | | |
| GO:0090263 | C2 | positive regulation | 0.039601 | 1.263538 |
| | | of canonical Wnt | | |
| | | signaling pathway | | |
| GO:0019693 . . . 855 | C3 | ribose phosphate | 2.45E−05 | 6.329114 |
| | | metabolic process | | |
| GO:0009259 . . . 856 | C3 | ribonucleotide metabolic | 5.01E−05 | 6.075949 |
| | | process | | |
| GO:0072527 | C3 | pyrimidine-containing | 5.29E−05 | 2.278481 |
| | | compound metabolic | | |
| | | process | | |
| GO:0072528 | C3 | pyrimidine-containing | 0.000108 | 1.518987 |
| | | compound biosynthetic | | |
| | | process | | |
| GO:0009150 . . . 859 | C3 | purine ribonucleotide | 0.000196 | 5.56962 |
| | | metabolic process | | |
| GO:0009132 . . . 860 | C3 | nucleoside diphosphate | 0.00046 | 2.531646 |
| | | metabolic process | | |
| GO:0006163 . . . 861 | C3 | purine nucleotide | 0.000465 | 5.56962 |
| | | metabolic process | | |
| GO:0006221 | C3 | pyrimidine nucleotide | 0.000491 | 1.265823 |
| | | biosynthetic process | | |
| GO:0035094 | C3 | response to nicotine | 0.000525 | 1.518987 |
| GO:0018958 | C3 | phenol-containing | 0.000537 | 2.278481 |
| | | compound metabolic process | | |
| GO:0009148 | C3 | pyrimidine nucleoside | 0.000567 | 1.012658 |
| | | triphosphate biosynthetic | | |
| | | process | | |

Analyzing blastocysts in three species revealed 984 messages that were detected in only in the bovine embryos, with 75 genes overlapping with JY-bESC uniquely expressed genes among FSCs (FIG. 8D). These bovine specifically expressed genes were enriched in KEGG pathways including RAP1, VEGF, and MAPK signaling pathways (Table H).

### Table H: KEGG Pathways Enriched by 75 Genes Shared Between the JY-bESC Uniquely Expressed Genes and Bovine-Specific Embryonic Genes.

TABLE H
KEGG pathways enriched by 75 genes shared between the JY-bESC uniquely expressed genes and bovine-specific embryonic genes.

| ID | Description | GeneRatio | BgRatio | pvalue | p. adjust | qvalue | geneID | count |
|---|---|---|---|---|---|---|---|---|
| hsa04015 | Rap1 signaling pathway | 4/33 | 210/8466 | 0.00856855 | 0.45628173 | 0.43381624 | 3690/1500/1436/51466 | 4 |
| hsa04724 | Glutamatergic synapse | 3/33 | 115/8466 | 0.00990442 | 0.45628173 | 0.43381624 | 9762/57030/5533 | 3 |
| hsa04380 | Osteoclast differentiation | 3/33 | 128/8466 | 0.01323329 | 0.45628173 | 0.43381624 | 3690/1436/5533 | 3 |
| hsa04370 | VEGF signaling pathway | 2/33 | 59/8466 | 0.02195168 | 0.45628173 | 0.43381624 | 9261/5533 | 2 |
| hsa04218 | Cellular senescence | 3/33 | 156/8466 | 0.022352 | 0.45628173 | 0.43381624 | 85417/9261/5533 | 3 |
| hsa04010 | MAPK signaling pathway | 4/33 | 301/8466 | 0.02847191 | 0.45628173 | 0.43381624 | 1436/9261/3303/5533 | 4 |
| hsa03320 | PPAR signaling pathway | 2/33 | 75/8466 | 0.03425634 | 0.45628173 | 0.43381624 | 2710/3611 | 2 |
| hsa03266 | Virion - Herpesvirus | 1/33 | 9/8466 | 0.03455554 | 0.45628173 | 0.43381624 | 3690 | 1 |
| hsa03018 | RNA degradation | 2/33 | 79/8466 | 0.03766992 | 0.45628173 | 0.43381624 | 10140/8761 | 2 |
| hsa04510 | Focal adhesion | 3/33 | 203/8466 | 0.04375118 | 0.45628173 | 0.43381624 | 1278/3690/3611 | 3 |
| hsa05205 | Proteoglycans in cancer | 3/33 | 205/8466 | 0.0448292 | 0.45628173 | 0.43381624 | 4060/1278/3690 | 3 |
| hsa04512 | ECM-receptor interaction | 2/33 | 89/8466 | 0.04674402 | 0.45628173 | 0.43381624 | 1278/3690 | 2 |
| hsa04520 | Adherens junction | 2/33 | 93/8466 | 0.05057821 | 0.45628173 | 0.43381624 | 1500/6615 | 2 |
| hsa04640 | Hematopoietic cell lineage | 2/33 | 99/8466 | 0.05653541 | 0.45628173 | 0.43381624 | 3690/1436 | 2 |
| hsa04933 | AGE-RAGE signaling pathway in | 2/33 | 100/8466 | 0.05755147 | 0.45628173 | 0.43381624 | 1278/1958 | 2 |
| | diabetic complications | | | | | | | |
| hsa04625 | C-type lectin receptor signaling | 2/33 | 104/8466 | 0.06167972 | 0.45628173 | 0.43381624 | 9261/5533 | 2 |
| | pathway | | | | | | | |
| hsa00910 | Nitrogen metabolism | 1/33 | 17/8466 | 0.06429731 | 0.45628173 | 0.43381624 | 2746 | 1 |
| hsa05131 | Shigellosis | 3/33 | 247/8466 | 0.07047513 | 0.45628173 | 0.43381624 | 7335/3611/2931 | 3 |
| hsa01200 | Carbon metabolism | 2/33 | 115/8466 | 0.07353347 | 0.45628173 | 0.43381624 | 2746/5091 | 2 |
| hsa00220 | Arginine biosynthesis | 1/33 | 22/8466 | 0.08243266 | 0.45628173 | 0.43381624 | 2746 | 1 |
| hsa04611 | Platelet activation | 2/33 | 124/8466 | 0.08373636 | 0.45628173 | 0.43381624 | 1278/3690 | 2 |
| hsa03060 | Protein export | 1/33 | 23/8466 | 0.08601861 | 0.45628173 | 0.43381624 | 55176 | 1 |
| hsa04964 | Proximal tubule bicarbonate | 1/33 | 23/8466 | 0.08601861 | 0.45628173 | 0.43381624 | 2746 | 1 |
| | reclamation | | | | | | | |
| hsa05020 | Prion disease | 3/33 | 272/8466 | 0.08831259 | 0.45628173 | 0.43381624 | 3303/1958/5533 | 3 |
| hsa04728 | Dopaminergic synapse | 2/33 | 132/8466 | 0.09315115 | 0.46202972 | 0.43928123 | 5533/2931 | 2 |
| hsa05418 | Fluid shear stress and atherosclerosis | 2/33 | 139/8466 | 0.10163356 | 0.48471391 | 0.46084854 | 3690/2947 | 2 |
| hsa00020 | Citrate cycle (TCA cycle) | 1/33 | 30/8466 | 0.11074245 | 0.50859497 | 0.4835538 | 5091 | 1 |
| hsa04145 | Phagosome | 2/33 | 152/8466 | 0.11793096 | 0.5167655 | 0.49132204 | 3690/55176 | 2 |
| hsa00250 | Alanine, aspartate and glutamate | 1/33 | 37/8466 | 0.13481724 | 0.5167655 | 0.49132204 | 2746 | 1 |
| | metabolism | | | | | | | |
| hsa05340 | Primary immunodeficiency | 1/33 | 38/8466 | 0.13820447 | 0.5167655 | 0.49132204 | 7374 | 1 |
| hsa05033 | Nicotine addiction | 1/33 | 40/8466 | 0.14494042 | 0.5167655 | 0.49132204 | 57030 | 1 |
| hsa04141 | Protein processing in endoplasmic | 2/33 | 173/8466 | 0.14552468 | 0.5167655 | 0.49132204 | 55176/3303 | 2 |
| | reticulum | | | | | | | |
| hsa04360 | Axon guidance | 2/33 | 182/8466 | 0.15774609 | 0.5167655 | 0.49132204 | 3611/5533 | 2 |
| hsa04151 | PI3K-Akt signaling pathway | 3/33 | 354/8466 | 0.15809236 | 0.5167655 | 0.49132204 | 1278/3690/1436 | 3 |
| hsa03410 | Base excision repair | 1/33 | 44/8466 | 0.15825948 | 0.5167655 | 0.49132204 | 7374 | 1 |
| hsa00620 | Pyruvate metabolism | 1/33 | 47/8466 | 0.16811652 | 0.5167655 | 0.49132204 | 5091 | 1 |
| hsa05167 | Kaposi sarcoma-associated | 2/33 | 194/8466 | 0.17434057 | 0.5167655 | 0.49132204 | 9261/5533 | 2 |
| | herpesvirus infection | | | | | | | |
| hsa00565 | Ether lipid metabolism | 1/33 | 50/8466 | 0.1778616 | 0.5167655 | 0.49132204 | 254531 | 1 |
| hsa05110 | Vibrio cholerae infection | 1/33 | 50/8466 | 0.1778616 | 0.5167655 | 0.49132204 | 55176 | 1 |
| hsa05134 | Legionellosis | 1/33 | 56/8466 | 0.19702075 | 0.5167655 | 0.49132204 | 3303 | 1 |
| hsa05170 | Human immunodeficiency virus 1 | 2/33 | 212/8466 | 0.19974715 | 0.5167655 | 0.49132204 | 85417/5533 | 2 |
| | infection | | | | | | | |
| hsa00480 | Glutathione metabolism | 1/33 | 57/8466 | 0.20017156 | 0.5167655 | 0.49132204 | 2947 | 1 |
| hsa05417 | Lipid and atherosclerosis | 2/33 | 214/8466 | 0.20260106 | 0.5167655 | 0.49132204 | 3303/5533 | 2 |
| hsa05213 | Endometrial cancer | 1/33 | 58/8466 | 0.20331038 | 0.5167655 | 0.49132204 | 3611 | 2 |
| hsa04213 | Longevity regulating pathway - | 1/33 | 61/8466 | 0.21265534 | 0.5167655 | 0.49132204 | 3303 | 1 |
| | multiple species | | | | | | | |
| hsa05166 | Human T-cell leukemia virus 1 | 2/33 | 222/8466 | 0.21406682 | 0.5167655 | 0.49132204 | 1958/5533 | 2 |
| | infection | | | | | | | |
| hsa05163 | Human cytomegalovirus infection | 2/33 | 225/8466 | 0.21838503 | 0.5167655 | 0.49132204 | 3690/5533 | 2 |
| hsa00561 | Glycerolipid metabolism | 1/33 | 63/8466 | 0.21882617 | 0.5167655 | 0.49132204 | 2710 | 1 |
| hsa04720 | Long-term potentiation | 1/33 | 67/8466 | 0.23102744 | 0.5167655 | 0.49132204 | 5533 | 1 |
| hsa05221 | Acute myeloid leukemia | 1/33 | 67/8466 | 0.23102744 | 0.5167655 | 0.49132204 | 1436 | 1 |
| hsa04924 | Renin secretion | 1/33 | 69/8466 | 0.23705858 | 0.5167655 | 0.49132204 | 5533 | 1 |
| hsa05031 | Amphetamine addiction | 1/33 | 69/8466 | 0.23705858 | 0.5167655 | 0.49132204 | 5533 | 1 |
| hsa05204 | Chemical carcinogenesis - DNA | 1/33 | 69/8466 | 0.23705858 | 0.5167655 | 0.49132204 | 2947 | 1 |
| | adducts | | | | | | | |
| hsa00982 | Drug metabolism - cytochrome P450 | 1/33 | 72/8466 | 0.24601936 | 0.5167655 | 0.49132204 | 2947 | 1 |
| hsa01524 | Platinum drug resistance | 1/33 | 73/8466 | 0.24898354 | 0.5167655 | 0.49132204 | 2947 | 1 |
| hsa04144 | Endocytosis | 2/33 | 250/8466 | 0.25464558 | 0.5167655 | 0.49132204 | 9744/3303 | 2 |
| hsa01230 | Biosynthesis of amino acids | 1/33 | 75/8466 | 0.25487805 | 0.5167655 | 0.49132204 | 5091 | 1 |
| hsa04612 | Antigen processing and presentation | 1/33 | 77/8466 | 0.26072768 | 0.5167655 | 0.49132204 | 3303 | 1 |
| hsa05100 | Bacterial invasion of epithelial cells | 1/33 | 77/8466 | 0.26072768 | 0.5167655 | 0.49132204 | 3611 | 1 |
| hsa05412 | Arrhythmogenic right ventricular | 1/33 | 77/8466 | 0.26072768 | 0.5167655 | 0.49132204 | 3690 | 1 |
| | cardiomyopathy | | | | | | | |
| hsa00980 | Metabolism of xenobiotics by | 1/33 | 78/8466 | 0.26363577 | 0.5167655 | 0.49132204 | 2947 | 1 |
| | cytochrome P450 | | | | | | | |
| hsa04721 | Synaptic vesicle cycle | 1/33 | 78/8466 | 0.26363577 | 0.5167655 | 0.49132204 | 57030 | 1 |
| hsa00983 | Drug metabolism - other enzymes | 1/33 | 80/8466 | 0.26941871 | 0.5167655 | 0.49132204 | 2947 | 1 |
| hsa04662 | B cell receptor signaling pathway | 1/33 | 84/8466 | 0.28085277 | 0.5167655 | 0.49132204 | 5533 | 1 |
| hsa04610 | Complement and coagulation cascades | 1/33 | 86/8466 | 0.28650454 | 0.5167655 | 0.49132204 | 10747 | 1 |
| hsa05235 | PD-L1 expression and PD-1 | 1/33 | 89/8466 | 0.29490151 | 0.5167655 | 0.49132204 | 5533 | 1 |
| | checkpoint pathway in cancer | | | | | | | |
| hsa05410 | Hypertrophic cardiomyopathy | 1/33 | 90/8466 | 0.29767915 | 0.5167655 | 0.49132204 | 3690 | 1 |
| hsa04658 | Th1 and Th2 cell differentiation | 1/33 | 92/8466 | 0.30320262 | 0.5167655 | 0.49132204 | 5533 | 1 |
| hsa04912 | GnRH signaling pathway | 1/33 | 93/8466 | 0.30594854 | 0.5167655 | 0.49132204 | 1958 | 1 |
| hsa05150 | Staphylococcus aureus infection | 1/33 | 96/8466 | 0.31412348 | 0.5167655 | 0.49132204 | 10747 | 1 |
| hsa05414 | Dilated cardiomyopathy | 1/33 | 96/8466 | 0.31412348 | 0.5167655 | 0.49132204 | 3690 | 1 |
| hsa03015 | mRNA surveillance pathway | 1/33 | 97/8466 | 0.31682765 | 0.5167655 | 0.49132204 | 8761 | 1 |
| hsa00564 | Glycerophospholipid metabolism | 1/33 | 99/8466 | 0.32220502 | 0.5167655 | 0.49132204 | 254531 | 1 |
| hsa04060 | Cytokine-cytokine receptor interaction | 2/33 | 297/8466 | 0.32311759 | 0.5167655 | 0.49132204 | 1436/3597 | 2 |
| hsa04061 | Viral protein interaction with cytokine | 1/33 | 100/8466 | 0.32487829 | 0.5167655 | 0.49132204 | 1436 | 1 |
| | and cytokine receptor | | | | | | | |
| hsa04914 | Progesterone-mediated oocyte | 1/33 | 102/8466 | 0.33019418 | 0.5167655 | 0.49132204 | 85417 | 1 |
| | maturation | | | | | | | |
| hsa05146 | Amoebiasis | 1/33 | 102/8466 | 0.33019418 | 0.5167655 | 0.49132204 | 1278 | 1 |
| hsa04974 | Protein digestion and absorption | 1/33 | 103/8466 | 0.33283689 | 0.5167655 | 0.49132204 | 1278 | 1 |
| hsa04660 | T cell receptor signaling pathway | 1/33 | 104/8466 | 0.33546948 | 0.5167655 | 0.49132204 | 5533 | 1 |
| hsa04928 | Parathyroid hormone synthesis, | 1/33 | 106/8466 | 0.34070449 | 0.5167655 | 0.49132204 | 1958 | 1 |
| | secretion and action | | | | | | | |
| hsa04922 | Glucagon signaling pathway | 1/33 | 107/8466 | 0.34330697 | 0.5167655 | 0.49132204 | 5533 | 1 |
| hsa03013 | Nucleocytoplasmic transport | 1/33 | 108/8466 | 0.34589949 | 0.5167655 | 0.49132204 | 91181 | 1 |
| hsa04659 | Th17 cell differentiation | 1/33 | 108/8466 | 0.34589949 | 0.5167655 | 0.49132204 | 5533 | 1 |
| hsa05145 | Toxoplasmosis | 1/33 | 111/8466 | 0.35361764 | 0.52200699 | 0.49630546 | 3303 | 1 |
| hsa04670 | Leukocyte transendothelial migration | 1/33 | 115/8466 | 0.36377125 | 0.53067806 | 0.5045496 | 1500 | 1 |
| hsa05165 | Human papillomavirus infection | 2/33 | 331/8466 | 0.37192231 | 0.53272984 | 0.50650035 | 1278/3690 | 2 |
| hsa04722 | Neurotrophin signaling pathway | 1/33 | 119/8466 | 0.37377013 | 0.53272984 | 0.50650035 | 9261 | 1 |
| hsa04919 | Thyroid hormone signaling pathway | 1/33 | 121/8466 | 0.37871226 | 0.53364 | 0.5073657 | 3690 | 1 |
| hsa04926 | Relaxin signaling pathway | 1/33 | 129/8466 | 0.39810526 | 0.54446632 | 0.51765898 | 1278 | 1 |
| hsa04068 | FoxO signaling pathway | 1/33 | 131/8466 | 0.40286103 | 0.54446632 | 0.51765898 | 85417 | 1 |
| hsa04114 | Oocyte meiosis | 1/33 | 131/8466 | 0.40286103 | 0.54446632 | 0.51765898 | 5533 | 1 |
| hsa04650 | Natural killer cell mediated | 1/33 | 132/8466 | 0.40522522 | 0.54446632 | 0.51765898 | 5533 | 1 |
| | cytotoxicity | | | | | | | |
| hsa04915 | Estrogen signaling pathway | 1/33 | 137/8466 | 0.41691072 | 0.54446632 | 0.51765898 | 3303 | 1 |
| hsa05014 | Amyotrophic lateral sclerosis | 2/33 | 364/8466 | 0.41806495 | 0.54446632 | 0.51765898 | 91181/5533 | 2 |
| hsa05162 | Measles | 1/33 | 138/8466 | 0.41922095 | 0.54446632 | 0.51765898 | 3303 | 1 |
| hsa04371 | Apelin signaling pathway | 1/33 | 139/8466 | 0.42152231 | 0.54446632 | 0.51765898 | 1958 | 1 |
| hsa04550 | Signaling pathways regulating | 1/33 | 143/8466 | 0.43063963 | 0.55050839 | 0.52340357 | 2103 | 1 |
| | pluripotency of stem cells | | | | | | | |
| hsa04723 | Retrograde endocannabinoid signaling | 1/33 | 148/8466 | 0.44184048 | 0.55906347 | 0.53153742 | 57030 | 1 |
| hsa04921 | Oxytocin signaling pathway | 1/33 | 154/8466 | 0.45499963 | 0.56618805 | 0.53831122 | 5533 | 1 |
| hsa04932 | Non-alcoholic fatty liver disease | 1/33 | 155/8466 | 0.45716337 | 0.56618805 | 0.53831122 | 2931 | 1 |
| hsa04110 | Cell cycle | 1/33 | 157/8466 | 0.46146589 | 0.56618805 | 0.53831122 | 85417 | 1 |
| hsa04217 | Necroptosis | 1/33 | 159/8466 | 0.46573533 | 0.56618805 | 0.53831122 | 2746 | 1 |
| hsa04630 | JAK-STAT signaling pathway | 1/33 | 166/8466 | 0.48042145 | 0.57222466 | 0.54405061 | 3597 | 1 |
| hsa04022 | cGMP-PKG signaling pathway | 1/33 | 167/8466 | 0.48248724 | 0.57222466 | 0.54405061 | 5533 | 1 |
| hsa05225 | Hepatocellular carcinoma | 1/33 | 168/8466 | 0.48454507 | 0.57222466 | 0.54405061 | 2947 | 1 |
| hsa04310 | Wnt signaling pathway | 1/33 | 171/8466 | 0.49067106 | 0.57399256 | 0.54573147 | 5533 | 1 |
| hsa05152 | Tuberculosis | 1/33 | 180/8466 | 0.50862844 | 0.58943856 | 0.56041697 | 5533 | 1 |
| hsa05034 | Alcoholism | 1/33 | 188/8466 | 0.52407395 | 0.59596768 | 0.56662462 | 5569 | 1 |
| hsa04613 | Neutrophil extracellular trap formation | 1/33 | 191/8466 | 0.52974377 | 0.59596768 | 0.56662462 | 3690 | 1 |
| hsa04062 | Chemokine signaling pathway | 1/33 | 192/8466 | 0.53161911 | 0.59596768 | 0.56662462 | 2931 | 1 |
| hsa05202 | Transcriptional misregulation in | 1/33 | 193/8466 | 0.5334872 | 0.59596768 | 0.56662462 | 1436 | 1 |
| | cancer | | | | | | | |
| hsa05415 | Diabetic cardiomyopathy | 1/33 | 203/8466 | 0.55177511 | 0.60745206 | 0.57754356 | 1278 | 1 |
| hsa05203 | Viral carcinogenesis | 1/33 | 204/8466 | 0.55356519 | 0.60745206 | 0.57754356 | 9261 | 1 |
| hsa05207 | Chemical carcinogenesis - receptor | 1/33 | 212/8466 | 0.56763839 | 0.61743124 | 0.5870314 | 2947 | 1 |
| | activation | | | | | | | |
| hsa03040 | Spliceosome | 1/33 | 216/8466 | 0.57451271 | 0.61947458 | 0.58897413 | 3303 | 1 |
| hsa05208 | Chemical carcinogenesis - reactive | 1/33 | 223/8466 | 0.58628857 | 0.62672227 | 0.59586498 | 2947 | 1 |
| | oxygen species | | | | | | | |
| hsa04810 | Regulation of actin cytoskeleton | 1/33 | 229/8466 | 0.59613011 | 0.63152292 | 0.60042926 | 3690 | 1 |
| hsa05171 | Coronavirus disease - COVID-19 | 1/33 | 232/8466 | 0.60096536 | 0.63152292 | 0.60042926 | 10747 | 1 |
| hsa04014 | Ras signaling pathway | 1/33 | 236/8466 | 0.60732511 | 0.63284298 | 0.60168432 | 1436 | 1 |
| hsa04020 | Calcium signaling pathway | 1/33 | 253/8466 | 0.63327468 | 0.65438384 | 0.6221646 | 5533 | 1 |
| hsa05016 | Huntington disease | 1/33 | 306/8466 | 0.70394381 | 0.72032296 | 0.68485714 | 7052 | 1 |
| hsa05206 | MicroRNAs in cancer | 1/33 | 310/8466 | 0.70870485 | 0.72032296 | 0.68485714 | 3690 | 1 |
| hsa05010 | Alzheimer disease | 1/33 | 384/8466 | 0.78449919 | 0.79087724 | 0.75193761 | 5533 | 1 |
| hsa05022 | Pathways of neurodegeneration - | 1/33 | 476/8466 | 0.85241469 | 0.85241469 | 0.81044521 | 5533 | 1 |
| | multiple diseases | | | | | | | |

The species-specific transcriptomic features we identified in bovine, human, and mouse PSCs and blastocysts provide insights into unique biological processes underlying early development of bovine PSC and embryos.

### Dosage Compensation of the X Chromosome in Bovine Female ESCs

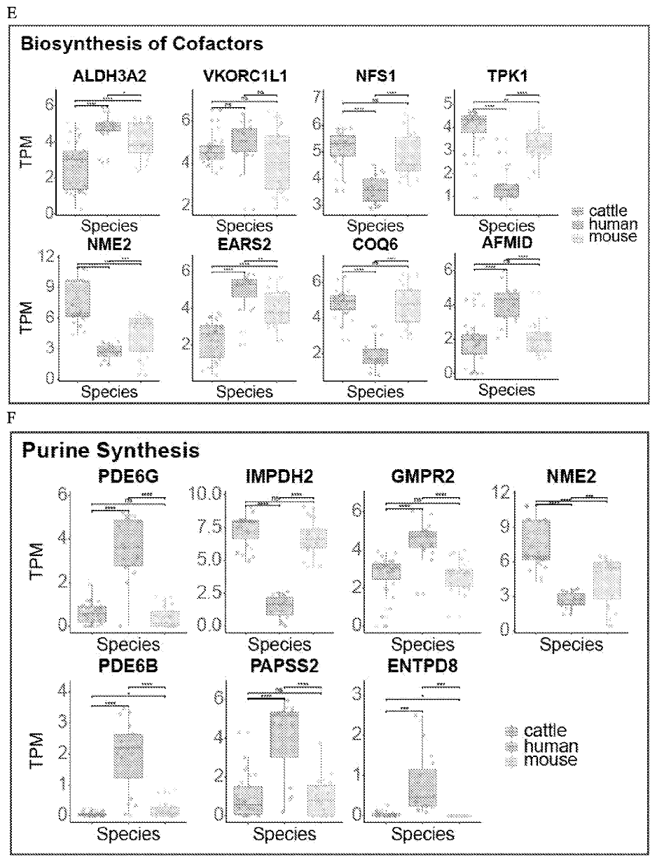

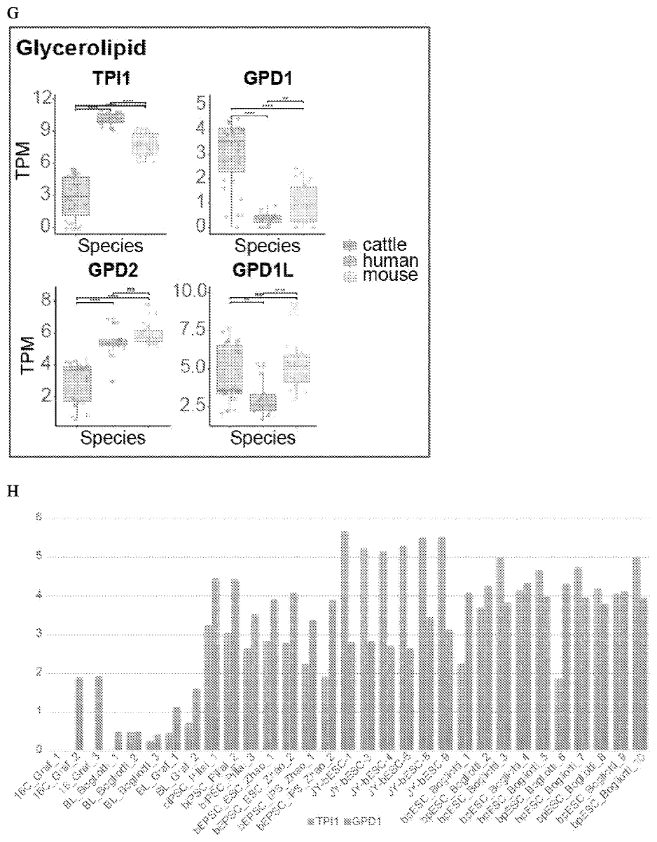

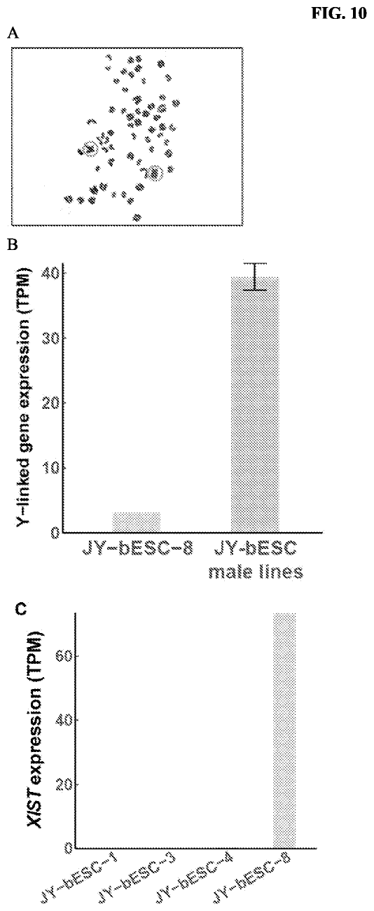

Using RNA-FISH and immunostaining, we found that the female JY-bESC Line (JY-bESC-8, FIGS. 10A and 10B) was enriched in H3K27me3, and expressed XIST and a single copy of the X-linked gene HUWE1, suggesting X-chromosome inactivation (XaXi) (FIGS. 2G and 2H). After another six passages, the X chromosome status remained unchanged (FIGS. 2G, 2H and 2I). RNA-seq analysis of six JY-bESC lines (one female and five male lines) confirmed the XIST expression pattern (FIG. 10C) and complete X chromosome dosage compensation (FIG. 2J). To assess X chromosome inactivation, we further analyzed the X:A allelic ratio for bi-allelic and monoallelic single-nucleotide polymorphisms (SNPs) in the JY-bESC-8 line (FIG. 2K). Consistent with RNA-FISH results, JY-bESC-8 showed strong monoallelic tendency (X:A allelic ratio higher than 2) and a low X:A ratio for bi-allelic genes, further supporting the establishment of XaXi status in female JY-bESCs.

Direct Differentiation of bESCs to Neural Progenitor Cells and Primordial Germ Cell-Like Cells

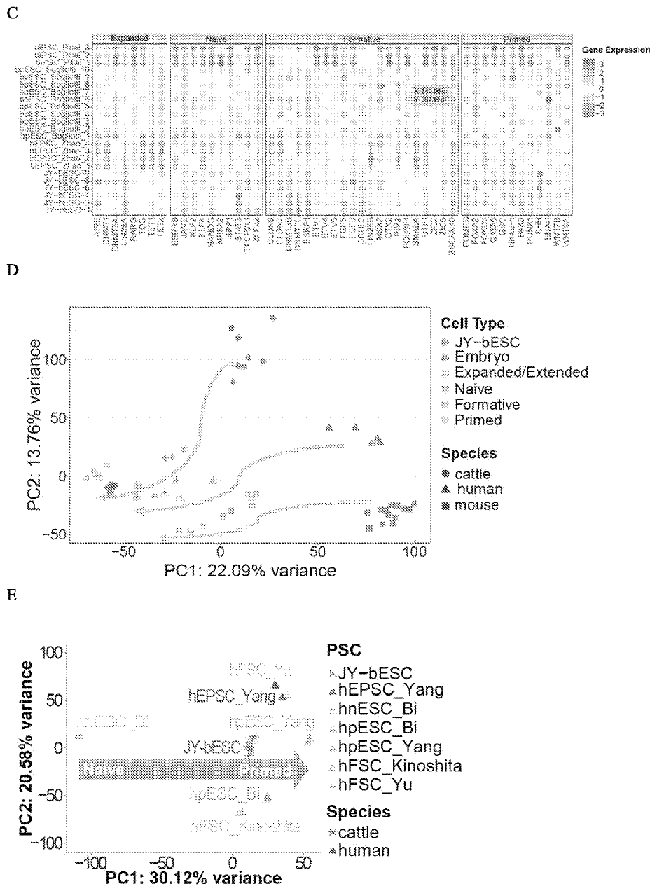

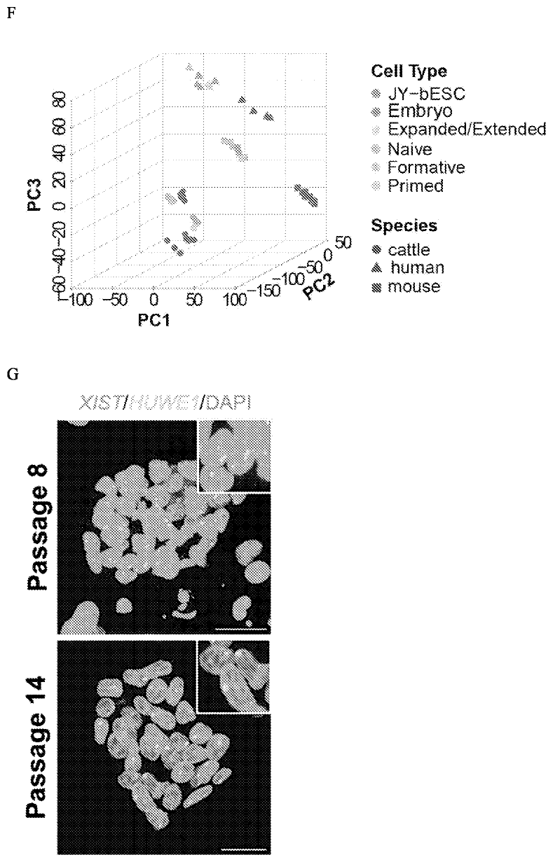

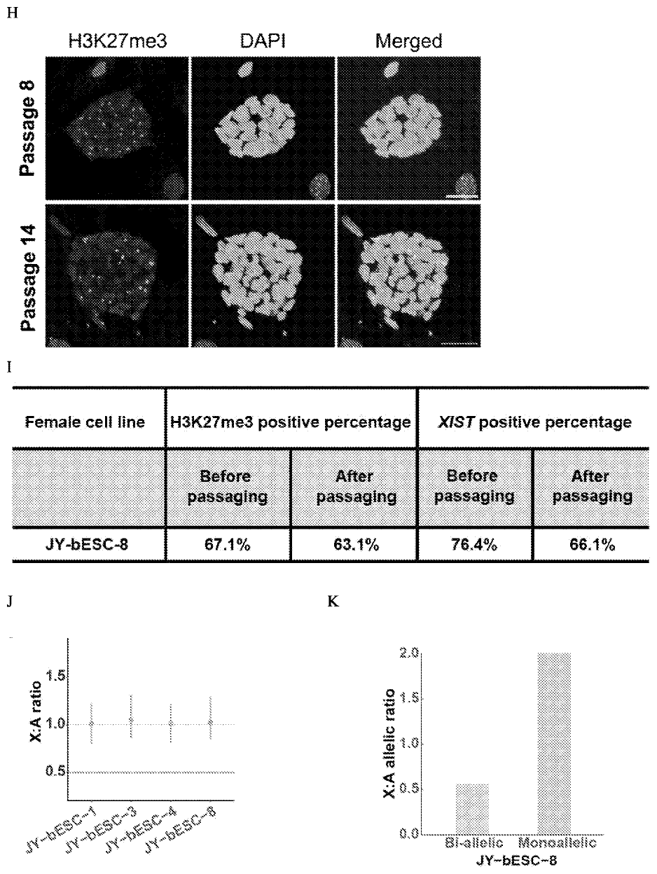

The direct differentiation of bESCs such as the induction of artificial gametes and neural progenitor cells (NPCs) is critical for cell-based models to study bovine spongiform encephalopathy and perform drug screening. Also, a key feature of formative human and mouse PSCs is direct in vitro differentiation to PGCLCs. By blocking TGF-β and BMP-dependent SMAD signaling, bovine NPCs were induced within two passages (19 days), confirmed by immunostaining for NPC markers (SOX2, NESTIN, and PAX6) and elevated NPC marker gene expression (FIGS. 3A-C). Additionally, treatment with germ cell stimulator BMP4 for four days increased expression of PGC-related genes and BMP8A further strengthened PGCLC induction (FIGS. 3D and 3E) as demonstrated by positive staining of PGC markers (TFAP2C, PRDM1, and SOX17) (FIGS. 3F and 6I). Moreover, the PRDM1+ cells exhibited co-expression with H3K27me3, while showing minimal H3K9me2 staining (FIG. 3G). This staining pattern aligns well with previous reports on PGCs. The triple positive ratio of TFAP2C, PRDM1 and SOX17 in bovine induced PGCLC is 21.5±2.8%, which is comparable to the rates of PGCLC induction of human and mouse formative PSCs. Therefore, the elevated and co-expression of NPC and PGC markers indicated efficient direct differentiation of JY-bESCs into NPC and PGCLC in vitro.

### Bovine ESCs Contributed to the Embryonic and Extra-Embryonic Tissues of Chimeras

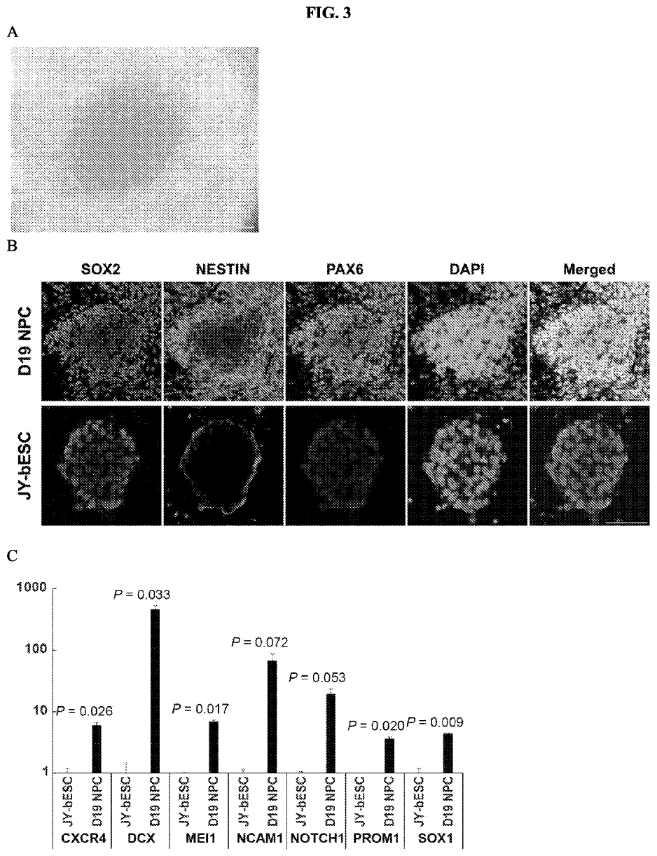

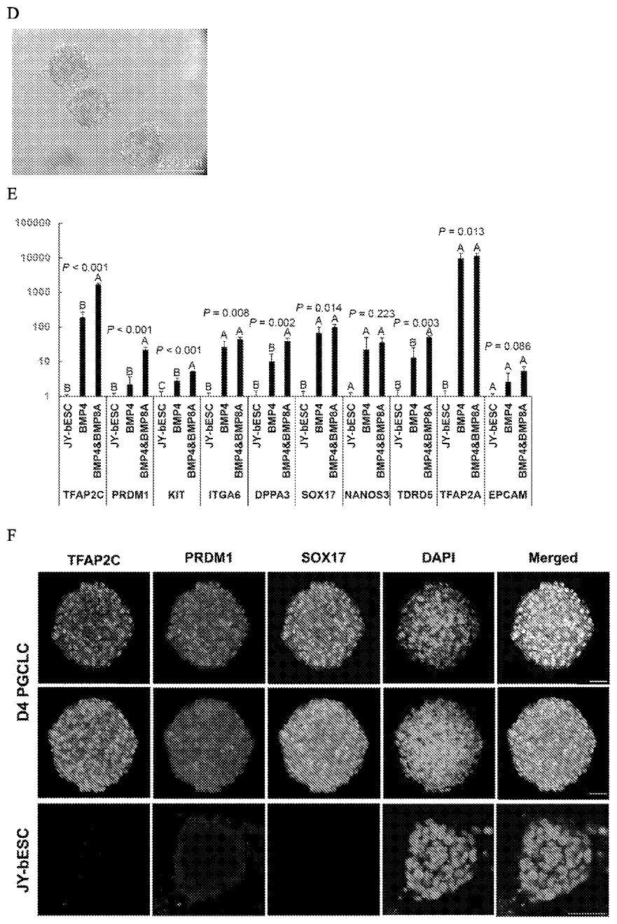

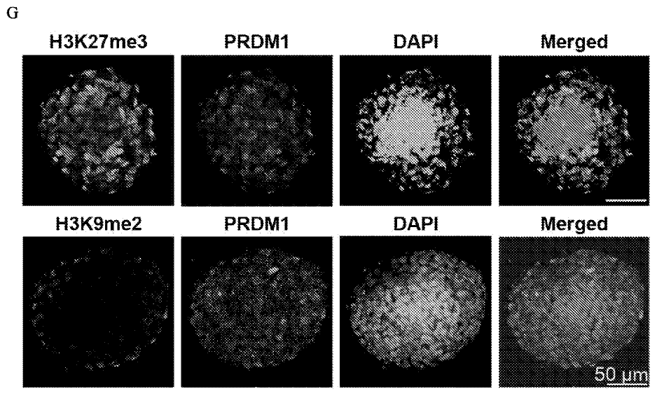

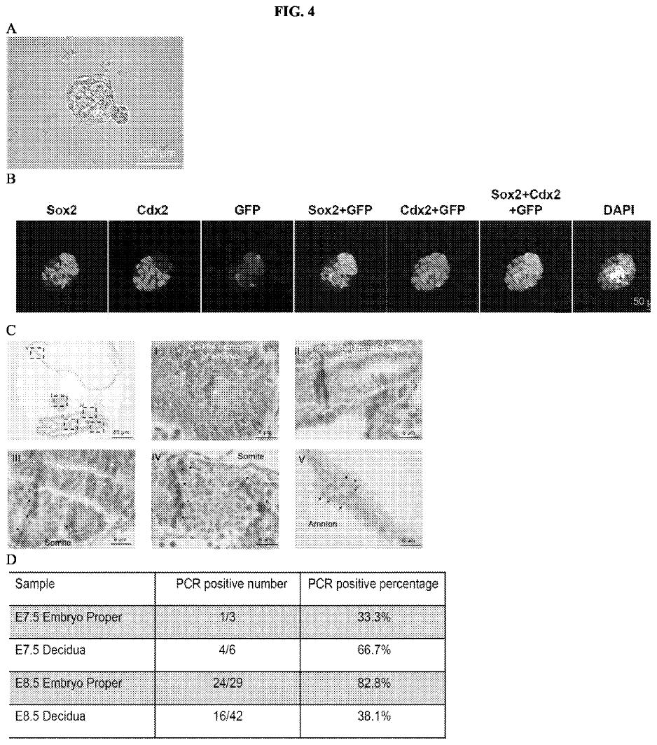

To determine the in vivo developmental potential of JY-bESCs, GFP-expressing (GFP+) JY-bESCs were established and aggregated with mouse early morulae for the creation of interspecies chimeras (1, 6). GFP signals were observed in the mouse blastocysts 24 hours after aggregation (FIG. 4A) and were verified by immunofluorescence staining (FIG. 4B), which showed that GFP+ cells were co-stained with markers of both ICM, Sox2, and trophectoderm (TE), Cdx2. Chimeric blastocysts were transferred into pseudopregnant females and retrieved on E7.5 and E8.5. Two out of eight E7.5 and three out of 44 E8.5 mouse embryos contained obvious GFP signals (FIG. 11A). GFP signals were further detected by immunohistochemistry (IHC) with serial sections in caudal extremity, midgut, notochord, somite, and amnion of E7.5 chimeric embryos (FIGS. 4C and 11B). PCR analysis also confirmed the presence of bovine DNA in both embryonic and extraembryonic tissues of the remaining chimeric embryos at high frequency (33.3% embryo proper and 66.7% decidua of E7.5 and 82.8% embryo proper and 38.1% decidua of E8.5 embryos, respectively) (FIGS. 4D and 11C), in accordance with the blastocyst staining result. The detection of bovine cells in various tissues of mouse-bovine chimeras demonstrated the successful contribution of JY-bESCs to both embryonic and extraembryonic tissues in interspecies chimeric embryos.

To date, extraembryonic differentiation has only been reported in bovine expanded PSCs (2). To this end, GFP+JY-bESCs were aggregated with bovine embryos at the 8-cell and morula stages and further cultured them in vitro for 48 or 96 hours, respectively. GFP+ cells were identified in both ICM and TE of the bovine blastocysts (FIGS. 4E, 4F and 11D). To investigate the in vivo development potential of the aggregated bovine chimeric embryos, 20 Day 8 GFP+ embryos were transferred to five recipient cows and five embryos from four recipients on Day 25 (Day 0=estrus) were retrieved (FIG. 11E). All embryos (100%) showed strong GFP signals in their extraembryonic tissues, and two out of five embryos had visible clusters of GFP+ cells in embryonic discs by using a fluorescent microscope with 4× magnification (FIG. 4G). FACS analysis confirmed 1.8% of the total dissociated cells from the two embryos (42,358 out of 2.2×10⁶) were GFP+(FIG. 11F).

### Single Cell Transcriptome Profiling of JY-bESC Derived Bovine Chimeras

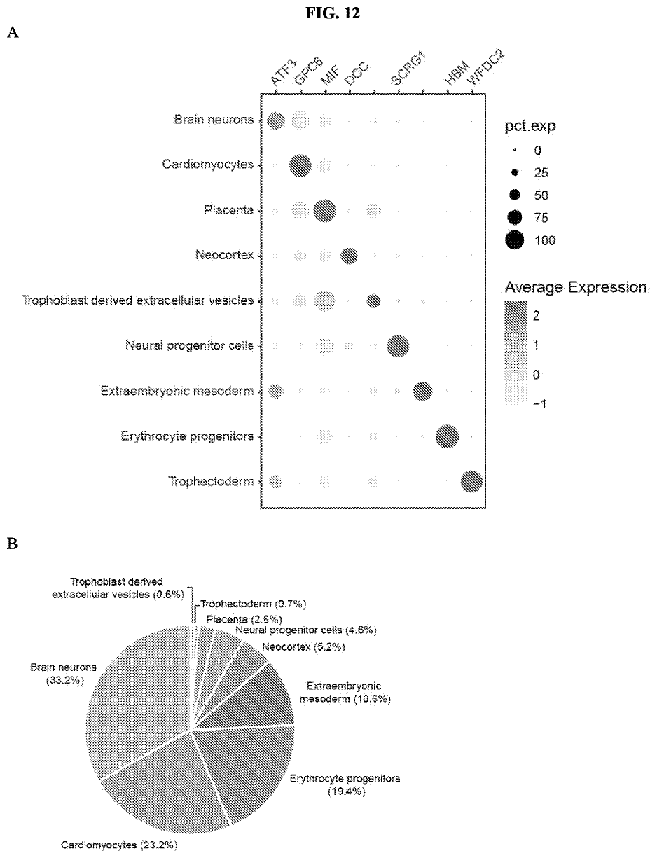

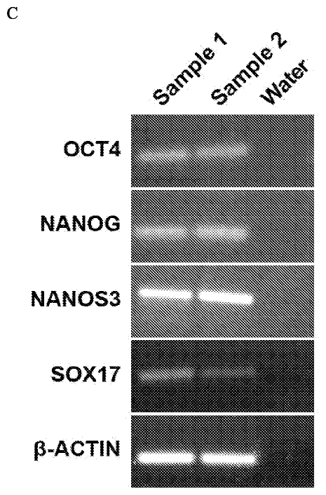

Using 10× Genomics Chromium platform for scRNA-seq, 3,354 GFP+JY-bESCs-derived cells and 1,083 GFP-host cells were examined from the two Day 25 embryonic disks after spiking-in cells from one extraembryonic sample. 9 clusters were identified, representing progenitors of the nerves system (Clusters 0, 4 and 5), cardiomyocytes (Cluster 1) and erythroid progenitor cells (Cluster 2) (FIGS. 4H, 4I, 12A, 12B and Table I).

### Table I: Gene Markers and References Used for Bovine Cell Type Annotating.

TABLE I
Gene markers and references used for bovine cell type annotating.

| | Major cell | |
|---|---|---|
| Cluster | cluster | Marker genes |
| 0 | Brain neurons | RMND5A, ATF3, ID2 |
| 1 | Cardiomyocytes | GPC6, SHROOM3, HMGA2 |
| 2 | Placenta | SSR4, MIF, TPI1 |
| 3 | Neocortex | DCC, CTNNA2, NRXN1 |
| 4 | Trophoblast | S100A11, CD63, AKR1B1 |
| | derived | |
| | extracellular | |
| | vesicles | |
| 5 | Neural | SCRG1, NCAM2, PCDH9 |
| | progenitor cells | |
| 6 | Extraembryonic | COL6A1, COL6A3, PITX1, PITX2 |
| | mesoderm | |
| 7 | Erythrocyte | HBM, ALAS2, GYPA, GATA1 |
| | progenitors | |
| 8 | Trophectoderm | WFDC2, KRT7, EPCAM, GATA2 |

Extraembryonic mesoderm (Cluster 3) of the hypoblast origin and trophectoderm cells (Clusters 6-8) were also found, presumably from the spiked-in extraembryonic cells, which contained strong GFP signals (FIGS. 4H, 4I, 12A, 12B and Table I). GFP+ and GFP-cells overlapped in all the identified cell types (FIG. 4I), indicating similar developmental trajectory by JY-bESCs and cells of the host embryos. 33 GFP+ and 8 GFP-cells were found that expressed at least three PGC-specific markers (OCT4, DPPA3, TFAP2C, PRDM1, SOX15, KIT, ITGA6, PDPN, EPCAM or ALPL) termed “putative PGCs” (Tables J and K).

### Table J: The Expression Level of PGC Markers in Putative Migrating PGCs

| Sample | | POU5F1 | ITGA6 | ALPL | KIT | DPPA3 | PRDM1 | EPCAM | TFAP2C | PDPN | SOX15 |
|---|---|---|---|---|---|---|---|---|---|---|---|
| GFP | sam1_CAAGCTATCGTTCTC | 0 | 1 | 0 | 1 | 1 | 0 | 0 | 0 | 0 | 0 |
| + | G-1 | | | | | | | | | | |
| | sam1_CGTGTCTAGACACAC | 0 | 1 | 1 | 0 | 0 | 1 | 0 | 0 | 0 | 0 |
| | G-1 | | | | | | | | | | |
| | sam1_GAGACTTGTACTGGG | 0 | 0 | 1 | 1 | 0 | 0 | 0 | 0 | 1 | 0 |
| | A-1 | | | | | | | | | | |
| | sam1_TGAATCGAGCTGAGC | 1 | 0 | 0 | 1 | 0 | 1 | 0 | 0 | 0 | 0 |
| | A-1 | | | | | | | | | | |
| | sam2_ACATTTCAGATTGAG | 0 | 1 | 1 | 0 | 0 | 0 | 0 | 0 | 1 | 0 |
| | T-1 | | | | | | | | | | |
| | sam2_CATGCGGGTGAGATT | 0 | 0 | 0 | 1 | 0 | 1 | 0 | 0 | 1 | 0 |
| | A-1 | | | | | | | | | | |
| | sam2 CCACGAGCAATTCGT | 0 | 1 | 0 | 1 | 0 | 1 | 0 | 0 | 0 | 0 |
| | G-1 | | | | | | | | | | |
| | sam2_GGTGTCGCAAGCTGT | 0 | 1 | 0 | 1 | 0 | 0 | 0 | 0 | 1 | 0 |
| | T-1 | | | | | | | | | | |
| | sam2_TGAGGAGCAATTGAG | 0 | 1 | 0 | 0 | 0 | 0 | 0 | 0 | 1 | 1 |
| | A-1 | | | | | | | | | | |
| | sam1_AACAAAGAGCTTCAT | 0 | 1 | 1 | 2 | 0 | 0 | 0 | 0 | 0 | 0 |
| | G-1 | | | | | | | | | | |
| | sam1_ACCAAACGTTTAAGG | 0 | 1 | 1 | 2 | 0 | 0 | 0 | 0 | 0 | 0 |
| | A-1 | | | | | | | | | | |
| | sam1_CCCATTGTCTGCGGA | 0 | 1 | 0 | 2 | 0 | 0 | 0 | 0 | 1 | 0 |
| | C-1 | | | | | | | | | | |
| | sam1_CGGGTGTTCAAGAAA | 0 | 1 | 0 | 2 | 0 | 0 | 1 | 0 | 0 | 0 |
| | C-1 | | | | | | | | | | |
| | sam1_CTGATCCCACATGAA | 0 | 1 | 1 | 2 | 0 | 0 | 0 | 0 | 0 | 0 |
| | A-1 | | | | | | | | | | |
| | sam1_GAAGGACCAATCTAG | 0 | 0 | 1 | 2 | 0 | 0 | 0 | 0 | 0 | 1 |
| | C-1 | | | | | | | | | | |
| | sam1_TAGAGTCCAGGCATT | 0 | 2 | 1 | 1 | 0 | 0 | 0 | 0 | 0 | 0 |
| | T-1 | | | | | | | | | | |
| | sam2_ACACCAAGTCCTCCA | 0 | 0 | 0 | 2 | 0 | 1 | 1 | 0 | 0 | 0 |
| | T-1 | | | | | | | | | | |
| | sam2_GCACGGTGTGTGTCG | 0 | 1 | 2 | 0 | 0 | 1 | 0 | 0 | 0 | 0 |
| | C-1 | | | | | | | | | | |
| | sam2_GCCAACGTCACATTG | 0 | 2 | 0 | 0 | 0 | 1 | 0 | 0 | 1 | 0 |
| | G-1 | | | | | | | | | | |
| | sam2_GTATTGGAGACGGTT | 0 | 1 | 1 | 2 | 0 | 0 | 0 | 0 | 0 | 0 |
| | G-1 | | | | | | | | | | |
| | sam2_TCACACCAGTGCCCG | 0 | 1 | 0 | 2 | 0 | 0 | 0 | 0 | 1 | 0 |
| | T-1 | | | | | | | | | | |
| | sam2_TTGTTCAGTTCAGGT | 0 | 1 | 0 | 1 | 0 | 2 | 0 | 0 | 0 | 0 |
| | T-1 | | | | | | | | | | |
| | sam1_GATGTTGCAATAGGG | 0 | 3 | 0 | 1 | 0 | 0 | 0 | 0 | 1 | 0 |
| | C-1 | | | | | | | | | | |
| | sam2_AGTTCCCAGGAGGTT | 0 | 1 | 1 | 3 | 0 | 0 | 0 | 0 | 0 | 0 |
| | C-1 | | | | | | | | | | |
| | sam2_CTGCATCTCGGTGTA | 0 | 3 | 1 | 1 | 0 | 0 | 0 | 0 | 0 | 0 |
| | T-1 | | | | | | | | | | |
| | sam2_TCAGCAAGTTGTCAG | 0 | 1 | 0 | 3 | 0 | 1 | 0 | 0 | 0 | 0 |
| | T-1 | | | | | | | | | | |
| | sam2_GTTACCCAGGAAAGG | 0 | 0 | 2 | 0 | 0 | 0 | 3 | 1 | 0 | 0 |
| | T-1 | | | | | | | | | | |
| | sam2_CGTAATGCATCAGTG | 0 | 0 | 0 | 4 | 0 | 2 | 12 | 0 | 0 | 0 |
| | T-1 | | | | | | | | | | |
| | sam1_GCTACCTAGCCTCTG | 0 | 1 | 0 | 0 | 0 | 1 | 10 | 0 | 0 | 0 |
| | G-1 | | | | | | | | | | |
| | sam2_ATTTACCGTATGGGA | 0 | 1 | 0 | 0 | 0 | 0 | 8 | 1 | 0 | 0 |
| | C-1 | | | | | | | | | | |
| | sam1_GTTACGACACAACAT | 0 | 1 | 0 | 1 | 0 | 0 | 6 | 0 | 0 | 0 |
| | C-1 | | | | | | | | | | |
| | sam1_TTTAGTCTCTTCGGA | 0 | 3 | 0 | 2 | 0 | 0 | 0 | 0 | 2 | 0 |
| | A-1 | | | | | | | | | | |
| | sam1_CGAGAAGCAGAGAA | 0 | 1 | 0 | 0 | 0 | 0 | 3 | 2 | 1 | 0 |
| | TT-1 | | | | | | | | | | |
| GFP | sam1_ACAGAAATCAGAAC | 0 | 3 | 0 | 1 | 0 | 0 | 0 | 0 | 1 | 0 |
| − | CT-1 | | | | | | | | | | |
| | sam1_CATCGTCTCGTCCTT | 0 | 2 | 0 | 1 | 0 | 0 | 4 | 0 | 0 | 0 |
| | G-1 | | | | | | | | | | |
| | sam1_TCGACCTGTCGTATG | 0 | 1 | 0 | 2 | 0 | 1 | 0 | 0 | 0 | 0 |
| | T-1 | | | | | | | | | | |
| | sam1_TTCCGGTCAGCTGCC | 0 | 1 | 0 | 2 | 0 | 0 | 1 | 0 | 0 | 0 |
| | A-1 | | | | | | | | | | |
| | sam2_AGAGAGCCATGTCTA | 0 | 1 | 0 | 0 | 0 | 0 | 0 | 1 | 0 | 1 |
| | G-1 | | | | | | | | | | |
| | sam2_CTCACTGGTACCCAC | 0 | 1 | 1 | 1 | 0 | 0 | 0 | 0 | 0 | 0 |
| | G-1 | | | | | | | | | | |
| | sam2_GACCAATGTCACCCT | 0 | 1 | 1 | 1 | 0 | 0 | 0 | 0 | 0 | 0 |
| | T-1 | | | | | | | | | | |
| | sam2_GTAACACGTCAACCT | 0 | 1 | 0 | 7 | 0 | 0 | 0 | 0 | 1 | 0 |
| | A-1 | | | | | | | | | | |

Table K: Distribution of Putative Migrating PGC Numbers with at Least Three PGC-Markers Expression

TABLE K
Distribution of putative migrating PDC numbers
with at least three PGC-markers expression

| Cluster | PGC GFP+ | PGC GFP− |
|---|---|---|
| 0 | 21 | 3 |
| 1 | 3 | 2 |
| 2 | 1 | 0 |
| 3 | 1 | 0 |
| 4 | 2 | 2 |
| 5 | 1 | 0 |
| 6 | 0 | 0 |
| 7 | 0 | 0 |
| 8 | 4 | 1 |
| Total | 33 | 8 |

Certain PGC markers that were barely shown in the scRNA-seq results, such as OCT4, NANOG, NANOS3, and SOX17 were also detected in the scRNA-seq library through PCR with strong signals (FIG. 12C), suggesting the existence of PGCs among the cell population. Together, the different tissues or cell types identified in the GFP+ Day 25 bovine chimeric embryos demonstrate, for the first time, the efficient incorporation of formative bESC into bovine embryonic and extraembryonic tissues in vivo.

### Chemical-Defined Medium Sustains the Pluripotency of Bovine ESCs

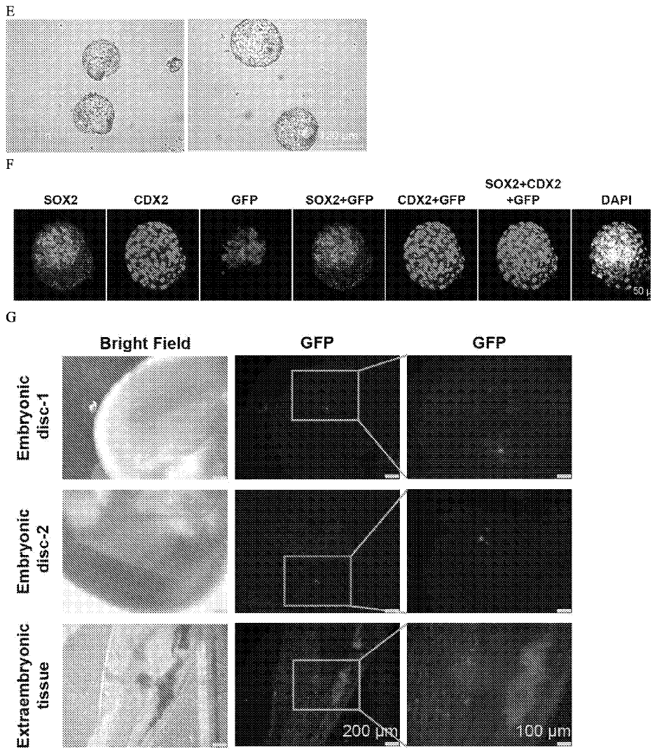

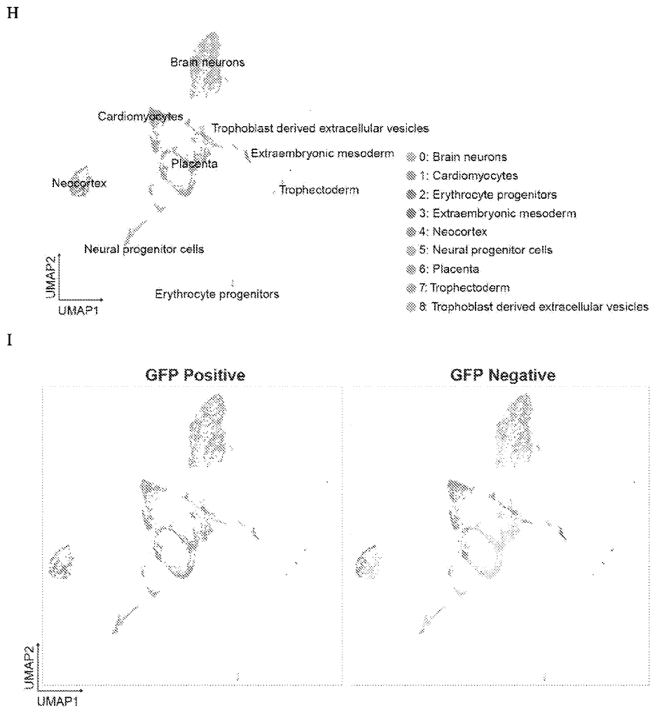

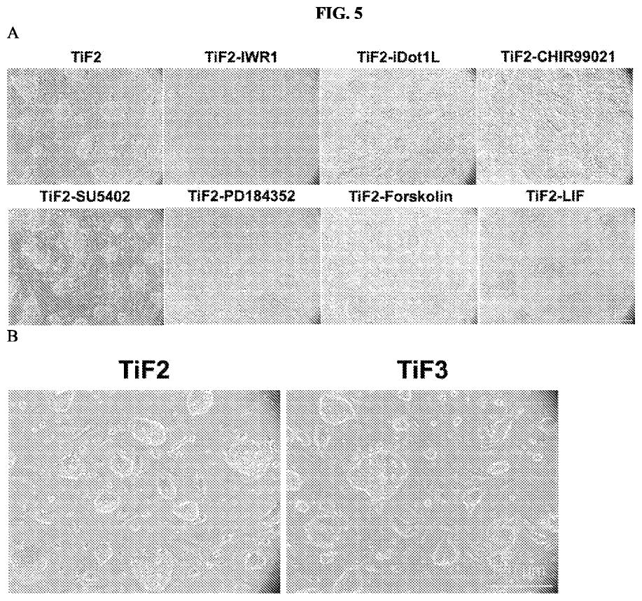

To determine the essential components in the TiF2 medium, supplemented small molecules and growth factors were systematically eliminated from TiF2 and screened the morphology of bESCs. Remarkably, within just three days of culture, cells began either differentiating or succumbing to cell death (FIG. 5A), suggesting all the components are indispensable for JY-bESC culture. The basal medium of TiF2, mTeSR plus, is a commercially available product with a complex formulation. Thus, it was endeavored to discover a chemically defined medium capable of sustaining the growth and pluripotency of bESCs. The investigation led to replacing mTeSR plus with N2B27 basal medium (7) supplemented with 1% BSA, alongside equal amounts of FGF2 and TGFβ1 in mTeSR medium (8) (designated as TiF3 medium). Notably, JY-bESCs maintained robust colony morphology and exhibited comparable expression levels of pluripotency genes for a minimum of eight passages (FIGS. 5B and 5C) in TiF3 medium. Furthermore, these cells demonstrated the capacity to spontaneously differentiate into all three germ layers in vitro (FIG. 5D). These results highlight the efficacy of TiF3 medium as a simplified yet effective alternative to mTeSR plus based TiF2 for maintaining bESC pluripotency.

Derivation of bESCs from bovine early embryos has been challenging for decades. The previously reported bESC lines showed limited propagation ability, refractory to single-cell colonization, confined pluripotent gene expression, and restricted differentiation capacity. While some progress has been made in recent years with primed bESCs cultured on feeders and feeder-free substrates, they lacked the demonstration of chimera formation. Similarly, newly reported bovine EPSCs (2) showed limited interspecies chimera capability. Furthermore, unlike mouse naive PSCs, which are restricted from trophectoderm differentiation, the extraembryonic plasticity of human epiblast prior to implantation has not been reported in other species. Here, successfully derived JY-bESCs in a TiF2 medium is described, resulting in defined colony morphology, high level expression of pluripotent genes, and in vitro and in vivo differentiation ability into three germ-layers. Notably, JY-bESCs demonstrated high rates of chimera formation to both embryonic and extraembryonic tissues in both interspecies (bovine-mouse) and intraspecies (bovine-bovine) chimeric embryos. Moreover, JY-bESCs exhibited species-specific formative transcriptomic profiles and could be directly differentiated into both NPCs and PGCLCs in vitro. Inter- and intraspecies chimerism further demonstrated the development potential of JY-bESCs to both embryonic and extraembryonic lineages. Taken together, JY-ESCs represent a significant advancement in extending the extraembryonic differentiation plasticity of human naive ESCs to formative bovine PSCs, offering great promises for bioengineering and development studies.

The following terms are used to describe the invention of the present disclosure. In instances where a term is not specifically defined herein, that term is given an art-recognized meaning by those of ordinary skill applying that term in context to its use in describing the present disclosure.

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

Compounds and materials are described using standard nomenclature. Unless defined otherwise, all technical and scientific terms used herein have the same meaning as is commonly understood by one of skill in the art to which this invention belongs. The following terms are used to describe the invention of the present disclosure. In instances where a term is not specifically defined herein, that term is given an art-recognized meaning by those of ordinary skill applying that term in context to its use in describing the present disclosure.

The use of the terms “a” and “an” and “the” and similar referents (especially in the context of the following claims) are to be construed to cover both the singular and the plural, unless otherwise indicated herein or clearly contradicted by context. By way of example, “an element” means one element or more than one element.

As used herein, the term “substantially” means to a great or significant extent, but not completely.

It should also be understood that, in certain methods described herein that include more than one step or act, the order of the steps or acts of the method is not necessarily limited to the order in which the steps or acts of the method are recited unless the context indicates otherwise. Furthermore, the terms first, second, etc., as used herein are not meant to denote any particular ordering, but simply for convenience to denote a plurality of, for example, layers.

The terms “comprising”, “having”, “including”, and “containing” are to be construed as open-ended terms (i.e., meaning “including, but not limited to”) unless otherwise noted.

The terms “about” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. All ranges disclosed herein include both end points as discrete values as well as all integers and fractions specified within the range. For example, a range of 0.1-2.0 includes 0.1, 0.2, 0.3, 0.4 . . . 2.0. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “one or more,” as used herein, means at least one, and thus includes individual components as well as mixtures/combinations of the listed components in any combination.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

As used herein, the term “administering” means the actual physical introduction of a composition into or onto (as appropriate) a subject, a host, or cell. Any and all methods of introducing the composition into the subject, host or cell are contemplated according to the invention; the method is not dependent on any particular means of introduction and is not to be so construed. Means of introduction are well-known to those skilled in the art, and also are exemplified herein. “Providing” means giving, administering, selling, distributing, transferring (for profit or not), manufacturing, compounding, or dispensing.

As used herein, “optional” or “optionally” means that the subsequently described event or circumstance may or may not occur, and that the description includes instances where said event or circumstance occurs and instances where it does not.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

A significant change is any detectable change that is statistically significant in a standard parametric test of statistical significance such as Student's T-test, where p<0.05.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein. No functional language used in claims appended herein is to be construed as invoking 35 U.S.C. § 112(f) interpretations as “means-plus-function” language unless specifically expressed as such by use of the words “means for” or “steps for” within the respective claim.

When introducing elements of the present invention or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. The term “exemplary” is not intended to be construed as a superlative example but merely one of many possible examples.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

### INCORPORATION BY REFERENCE

All U.S. and PCT patent publications and U.S. patents mentioned herein are hereby incorporated by reference in their entirety as if each individual patent publication or patent was specifically and individually indicated to be incorporated by reference. In case of conflict, the present application, including any definitions herein, will control.

### Other Embodiments

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

### REFERENCES

1. Y. Su et al., Establishment of Bovine-Induced Pluripotent Stem Cells. International journal of molecular sciences 22, 10489 (2021).

What is claimed is:

1. A composition to grow a cell line, the composition comprising:
a cell culture medium; and
a supplement comprising:
a Wingless/Integrated (WNT) inhibitor;
a histone methyltransferase DOT1L inhibitor (iDOT1L);
an adenyl cyclase activator;
a Mitogen-activated protein kinase kinases 1 and 2 (MEK1/2) inhibitor;
a fibroblast growth factor receptor (FGFR) inhibitor;
a glycogen synthase kinase (GSK-3) inhibitor; and
a leukemia inhibitory factor (LIF),
wherein the composition maintains pluripotency and self-renew capacity of the cell line.

2. The composition of claim 1, wherein the supplement further comprises:
bovine serum albumin (BSA);
a basic fibroblast growth factor (bFGF); and
a transforming growth factor β1 (TGF β1).

3. The composition of claim 1, wherein the culture medium is mTeSR-plus or N2B27.

4. The composition of claim 3, wherein the culture medium is N2B27 and the supplement further comprises:
bovine serum albumin (BSA);
a basic fibroblast growth factor (bFGF); and
a transforming growth factor β1 (TGF β1).

5. The composition of claim 1, wherein the composition comprises at least 99% of the cell culture medium and about 1% of the supplement.

6. The composition of claim 1, wherein the supplement comprises:
from about 0.1 μM to about 25 μM of the WNT inhibitor;
from about 0.1 μM to about 25 μM of the histone methyltransferase DOT1L inhibitor;
from about 0.1 μM to about 10 μM of the MEK1/2 inhibitor;
about 0.1 μM to about 10 μM of the FGFR inhibitor;
from about 0.5 μM to about 10 μM of the GSK-3 inhibitor;
from about 1 μM to about 50 μM of the adenyl cyclase activator; and
about 100 U/ml to about 5000 U/ml of LIF.

7. The composition of claim 2, wherein the supplement comprises:
from about 0.01 to about 5% (v/v) of BSA;
from about 10 to about 500 ng/ml of bFGF; and
from about 0.1 to about 5 ng/ml of TGF β1.

8. The composition of claim 6, wherein:
the WNT inhibitor is IWR1;
the histone methyltransferase DOT1L inhibitor is EPZ004777;
the adenyl cyclase activator is forskolin;
the MEK1/2 inhibitor is PD184352;
the FGFR inhibitor is SU5402;
the GSK-3 inhibitor is CHIR99021;
the LIF is a human LIF or a bovine LIF;

9. The composition of claim 7, wherein
the bFGF is a human bFGF or a bovine bFGF; and
the TGF β1 is a human TGF β1 or a bovine TGF β1.

10. The composition of claim 8, wherein:
the IWR1 is present in an amount of about 2.5 μM;
the EPZ004777 is present in an amount of about 3.3 μM;
the forskolin is present in an amount of about 10 μM
the PD184352 is present in an amount of about 0.8 μM;
the SU5402 is present in an amount of about 2 μM;
the CHIR99021 is present in an amount of about 3 μM; and
the human LIF or bovine LIF is present in an amount of 1000 U/mL.

11. The composition of claim 9, wherein:
BSA is present in an amount of about 1% (v/v);
the human bFGF or bovine bFGF is present in an amount of about 100 ng/mL; and
the human TGF β1 or bovine TGF β1 is present in an amount of about 1.74 ng/mL.

12. A method of producing stem cells, the method comprising the steps of:
a) providing bovine pre-implantation embryos;
b) treating the bovine pre-implantation embryos with an enzyme to remove the zone pellucida;
c) culturing treated bovine pre-implantation embryos with a composition to form a culture, the composition comprising:
i) a cell culture medium; and
ii) a supplement comprising:
a Wingless/Integrated (WNT) inhibitor;
a histone methyltransferase DOT1L inhibitor (iDOT1L);
an adenyl cyclase activator;
a Mitogen-activated protein kinase kinases 1 and 2 (MEK1/2) inhibitor;
a fibroblast growth factor receptor (FGFR) inhibitor;
a glycogen synthase kinase (GSK-3) inhibitor;
a leukemia inhibitory factor (LIF);
bovine serum albumin (BSA);
a basic fibroblast growth factor (bFGF); and
a transforming growth factor β1 (TGF β1); and
d) harvesting stem cells resulting from the culture.

13. The method of claim 12, wherein the supplement further comprises:
bovine serum albumin (BSA);
a basic fibroblast growth factor (bFGF); and
a transforming growth factor β1 (TGF β1).

14. The method of claim 12, wherein the method is used to grow a bovine embryonic stem cell line (bESC).

15. The method of claim 12, wherein the enzyme is a pronase, a hyaluronidase, an acrosin or any combinations thereof.

16. The method of claim 12, wherein the culture medium is a serum free culture medium for culturing stem cell lines.

17. The method of claim 16, wherein the culture medium is mTeSR-plus or N2B27.

18. The method of claim 12, wherein the composition comprises at least 99% of the cell culture medium and about 1% of the supplement.

19. A stem cell line comprising a bovine embryonic stem cell (bESC), wherein the bESC maintains its pluripotency and self-renewal capacity.
